SEQUENCE LISTING (147 sequences)

SEQ ID NO: 1 - DNA, 30 bp, synthetic construct
  misc_feature 1..30: note = Adenovirus pIX fragment nucleotides
  source 1..30: mol_type = other DNA
agctctttggtggcgagcggcgcggcctct

SEQ ID NO: 2 - DNA, 439 bp, synthetic construct
  misc_feature 1..439: note = Adenovirus DNA polymerase fragment nucleotides
  source 1..439: mol_type = other DNA
aacatcaatacctcaaagtcatggtcagggacactttcgccctcacccacacctccctccgcaaggcggcgcaggcctacgcgctgcccgtggagaagggctgttgcccctaccaggccgtcaaccagttctacatgctaggctcttaccgttcggacacggacgggtttcccctccaagagtactggaaagaccgcgaagagttcgtcctcaaccgcgagctgtggaaaaagaagggggaggataagtatgacatcatccgcgagaccctcgactactgcgcgctcgacgtccaggtcaccgccgagctggtgcacaagctgcgcgagtcctacgcctccttcgtcagggactcggtgggcttgcaagaagcaagcttcaacgtcttccagcggcccaccatctcctccaactcccatgccatcttcaggcagatcgc

SEQ ID NO: 3 - DNA, 34 bp, synthetic construct
  misc_feature 1..34: note = Adenovirus penton base protein fragment nucleotides
  source 1..34: mol_type = other DNA
actgaggctgcggctaaggctgaggtcgaagcca

SEQ ID NO: 4 - DNA, 45 bp, synthetic construct
  misc_feature 1..45: note = Adenovirus hexon protein fragment nucleotides
  source 1..45: mol_type = other DNA
ataggtgtggatgccacacaggcgggagataaccctatatatgct

SEQ ID NO: 5 - DNA, 30 bp, synthetic construct
  misc_feature 1..30: note = Adenovirus fiber protein fragment nucleotides
  source 1..30: mol_type = other DNA
gtagcaggccccctagctgtggccaatggc

SEQ ID NO: 6 - DNA, 399 bp, synthetic construct
  misc_feature 1..399: note = Adenovirus pIX nucleotides
  source 1..399: mol_type = other DNA
atgagcgacaccggcaacagctttgatggaagcatctttagcccctatctgacagtgcgcatgcctcactgggctggagtgcgtcagaatgtgatgggttccaacgtggatggacgccccgttctgccttcaaattcgtctacaatggcctacgcgaccgtgggaggaactccgctggacgccgcgacctccgccgccgcctccgccgccgccgcgaccgcgcgcagcatggctacggacctttacagctctttggtggcgagcggcgcggcctctcgcgcgtctgctcgggatgagaaactgaccgctctgctgcttaaactggaagacttgacccgggagctgggtcaactgacccagcaggtctccagcttgcgtgagagcagccttgcctccccc

SEQ ID NO: 7 - DNA, 3168 bp, synthetic construct
  misc_feature 1..3168: note = Adenovirus DNA polymerase nucleotides
  source 1..3168: mol_type = other DNA
atggacagctccaatgtgcgcgatgtcgtcatcaaactccgcccgccgagcgccgagatctggacctgcggctctcgcggcgtggtggtctgctccaccatcgccctccaggagacagatgctggcggccagacaaccaaagtagaagaccaccagccacacgggaccccaggcgggggacttagattcccgctgcgcttcctcgtcagaggtcgccaggttcacctcgtgcaagatatacaacccgtgcagcgctgccagtactgcggtcgcttttacaaaagccagcacgagtgctcggcccgcagacgggacttctactttcaccacatcaacagccaatcctccaactggtggcgggagatccagttcttcccgatcggctctcatcctcgcacggagcgcctctttgtcacctacgatgtagagacctacacttggatgggagcctttggcaagcagctcgtgcccttcatgctggtcatgaaactggggggcgacgaggctctggtcgccgccgcgcgcgacctcgcccgagagctcagatgggacccctgggagaaagaccccctcaccttctactgcatcacccccgaaaagatggccgtggggcgacagttcagaaccttccgcgaccgcctgcagaccctcatggcccgcgacctctggcgatccttcctggcggccaaccctcacttgcaagactgggccctggaggagcacggcctggaatcgcccgaggagctcacctacgaggaactcaaaaagctcccctccatcaagggccagccccgctttttggagctctacatcgtgggccacaacataaacggctttgacgagatcgtcctggccgcccaggtcatcaacaaccgctcctcggtcccagggccctttcgcatcaccagaaacttcatgcctcgagcggggaagatcctcttcaatgacctcaccttctccctgcccaacccgcgctccaaaaagcgcacggactacaccctgtgggaacagggcggctgcgatgacacagacttcaaacatcaatacctcaaagtcatggtcagggacactttcgccctcacccacacctccctccgcaaggcggcgcaggcctacgcgctgcccgtggagaagggctgttgcccctaccaggccgtcaaccagttctacatgctaggctcttaccgttcggacacggacgggtttcccctccaagagtactggaaagaccgcgaagagttcgtcctcaaccgcgagctgtggaaaaagaagggggaggataagtatgacatcatccgcgagaccctcgactactgcgcgctcgacgtccaggtcaccgccgagctggtgcacaagctgcgcgagtcctacgcctccttcgtcagggactcggtgggcttgcaagaagcaagcttcaacgtcttccagcggcccaccatctcctccaactcccatgccatcttcaggcagatcgccttccgcgccgagcgcccccagcgcaccaacctcgggcccaacatgctggccccctcccacgagctctatgactacgtgcgcgccagcatccgcggggggcgctgctaccccacctacctcggcatcctcagggaacccctgtacgtgtatgacatctgcggcatgtacgcctccgcgctcacccaccccatgccctggggcccgcccctcaacccctacgagcgcgcgctcgccgcccgcgaatggcagcgggctctggacatgcaagcttgcaagatcgactactttgacccgcgcttgctccccggggtcttcaccatcgacgcggaccccccaaacgaggaccagctggaccccctaccccccttctgctcgcgcaagggcggccgcctctgctggaccaacgagcgcctgcgcggcgaggtcgccaccagcgtcgacatggtcaccctgcacaaccgaggctggagggtgcgcctaatcccagacgagcgcaccaccgtcttccccgagtggaagtgcgtggcccgcgagtacgtgcaactcaacatcgcggccaaggagcgagccgaccgcgacaaaaaccagaccctgcgctccatcgccaagctgctctccaacgccctctacgggtcgttcgccaccaagcttgacaacaaaaaaatagtgttttctgaccagatggacccaggtaccctcaaaggtatcacctccggacaggtgaacatcaaatcctcctcatttttagaaactgacaacctgagcgctgaggtcatgcccgccttcgagagggaatacttaccccagcagctggccctcgcagacagcgatgcggaagagagtgaagatgaaagggcgcccacccccttttatacccccccgtcgggaacccccggtcacgtgtcctacacctacaagccaatcacttttctggacgcggaggagggggacatgtgcctgcacaccctggagaaggtggacccgctagtggacaacgaccgctacccctcccacgtggcctccttcgtcctggcctggacgcgggccttcgtctcagagtggtcagagtttctctacgaggaggacagaggcactccgctggaagacaggcccctgaagtcggtctacggggacacggacagcctcttcgtcaccgagaagggacaccgcctcatggagagccgaggtaagaaacgcatcaaaaagcatgggggcaacctggtttttgaccctgaccgcccggagctcacttggctggtggaatgcgagacggtctgcgcttcctgcggcgcggacgcctactccccagagtccgtgtttctcgctcccaagctctacgccctgaagagcctgcagtgcccctcgtgcggcgccacctccaagggaaagctccgcgccaaggggcacgccgccgagggtctcgactacgagaccatggtcaaatgctacctggccgacgcgcagggcgaagagcggcagcgattcagcaccagcagaaccagcctcaagcgcaccctggccagcgcccaacccggagcgcaccccttcaccgtgacccagaccaccctgacgaggaccctgcgcccatggaaggacatgactctggccccgctggacgcccatcggctggtgccctacagcgaaagccgccccaacccgcgaaacgaggagatctgctggatcgagatgccg

SEQ ID NO: 8 - DNA, 1974 bp, synthetic construct
  misc_feature 1..1974: note = Adenovirus penton base protein nucleotides
  source 1..1974: mol_type = other DNA
atgcggcgcgcggcgatgttcgaggaggggcctcccccctcttacgagagcgcgatggggatttctcctgcggcgcccctgcagcctccctacgtgcctcctcggtacctgcaacctacaggggggagaaatagcatctgttactctgagctgcagcccctgtacgataccaccagactgtacctggtggacaacaagtccgcggacgtggcctccctgaactaccagaacgaccacagcgattttttgaccacggtgatccaaaacaacgacttcaccccaaccgaggccagcacccagaccataaacctggataacaggtcgaactggggcggcgacctgaagaccatcttgcacaccaacatgcccaacgtgaacgagttcatgttcaccaactcttttaaggcgcgggtgatggtggcgcgcgagcagggggaggcgaagtacgagtgggtggacttcacgctgcccgagggcaactactcagagaccatgactctcgacctgatgaacaatgcgatcgtggaacactatctgaaagtgggcaggcagaacggggtgaaggaaagcgatatcggggtcaagtttgacaccagaaacttccgtctgggctgggaccccgtgaccgggctggtcatgccgggggtctacaccaacgaggcctttcatcccgacatagtgcttctgcccggctgtggggtggacttcacccagagccggctgagcaacctgctgggcattcgcaagcggcagcctttccaggagggtttcaagatcacctatgaggatctgaaggggggcaacattcccgcgctccttgatctggacgcctacgaggagagcttgaaacccgaggagagcgctggcgacagcggcgagagtggcgaggagcaagccggcggcggtggcggcgcgtcggtagaaaacgaaagtacgcccgcagtggcggcggacgctgcggaggtcgagccggaggccatgcagcaggacgcagaggagggcgcacaggagggcgcgcagaaggacatgaacgatggggagatcaggggagacacattcgccacccggggcgaagaaaaagaggcagaggcggcggcggcggcgacggcggaggccgaaaccgaggttgaggcagaggcagagcccgagaccgaagttatggaagacatgaatgatggagaacgtaggggcgacacgttcgccacccggggcgaagagaaggcggcggaggcagaagccgcggctgaggaggcggctgcggctgcggccaagactgaggctgcggctaaggctgaggtcgaagccaatgttgcggttgaggctcaggctgaggaggaggcggcggctgaagcagttaaggaaaaggcccaggcagagcaggaagagaaaaaacctgtcattcaacctctaaaagaagatagcaaaaagcgcagttacaacgtcatcgagggcagcacctttacccagtaccgcagctggtacctggcgtacaactacggcgacccggtcaagggggtgcgctcgtggaccctgctctgcacgccggacgtcacctgcggctccgagcagatgtactggtcgctgccgaacatgatgcaagacccggtgaccttccgctccacgcggcaggttagcaacttcccggtggtgggcgccgaactgctgcccgtgcactccaagagtttttacaacgagcaggccgtctactcccagctgatccgccaggccacctctctgacccacgtgttcaatcgctttcccgagaaccagattttggcgcgcccgccggcccccaccatcaccaccgtgagtgaaaacgttcctgccctcacagatcacgggacgctaccgctgcgcaacagcatctcaggagtccagcgagtgaccattactgacgccagacgccggacctgcccctacgtttacaaggccttgggcatagtctcgccgcgcgtcctctccagtcgcactttt

SEQ ID NO: 9 - DNA, 2877 bp, synthetic construct
  misc_feature 1..2877: note = Adenovirus hexon protein nucleotides
  source 1..2877: mol_type = other DNA
atggcgaccccatcgatgatgcctcagtggtcgtacatgcacatctcgggccaggacgcttcggagtacctgagccccgggctggtgcagttcgcccgcgccacagacacctacttcaacatgagtaacaagttcaggaaccccactgtggcgcccacccacgatgtgaccacggaccggtcgcagcgcctgacgctgcggttcatccccgtggatcgggaggacaccgcctactcttacaaggcgcggttcacgctggccgtgggcgacaaccgcgtgctggacatggcctccacttactttgacatcaggggggtgctggacaggggccccaccttcaagccctactcgggtactgcctacaactccctggcccccaagggcgctcccaattcttgcgagtgggaacaagatgaaccagctcaggcagcaatagctgaagatgaagaagaacttgaagaagaacaagctcaggacgaacaggcgcccactaagaaaacccatgtatacgcccaggcacctctttctggtgaaaaaattactaaggatggtttgcaaataggtgtggatgccacacaggcgggagataaccctatatatgctgataaaacattccaacccgaacctcagataggtgagtctcagtggaacgaggctgatgccacagtagcaggaggcagagtcttaaaaaagaccacccctatgagaccttgctatggatcctatgccaaacctactaatgccaatggcggtcaagggatcatggtggccaatgatcagggagcgcttgaatctaaagttgagatgcaatttttctccaccacaacgtctcttaatgtaagggaaggtgaaaacaatcttcagccaaaagtagtgctatacagcgaagatgttaacttggaatcccctgacactcatttgtcttacaaacctaaaaaggatgacaccaactctaaaatcatgttgggtcagcaagccatgcccaacagacccaacctcattgcttttagggacaactttattggacttatgtactacaacagcacaggcaacatgggagtgctggcaggacaggcctcccagctaaacgctgtggtagacttgcaagacagaaacacagagctgtcataccaactgatgcttgattccattggagacagatcaagatacttttccatgtggaaccaggcagtggacagctatgacccagatgtcagaatcattgaaaaccatggggttgaagatgagctgcccaactattgctttcccctgggcggtattggaattacagacacataccagtgcataaaaccaaccgcagctgctaataacactacatggtctaaggatgaagaatttagtgatcgcaatgaaataggggtgggaaacaacttcgccatggagatcaacatccaggccaacctctggaggaacttcctctatgcgaacgtggggctctacctgccagacaagctcaagtacaaccccaccaacgtggacatctctgacaaccccaacacctatgactacatgaacaagcgtgtggtggctcccggcctggtggactgctttgtcaatgtgggagccaggtggtccctggactacatggacaacgtcaaccccttcaaccaccaccgcaatgcgggtctgcgctaccgctccatgatcctgggcaacgggcgctacgtgcccttccacattcaggtgccccagaagttctttgccatcaagaacctcctcctcctgccgggctcctacacttacgagtggaacttcaggaaggatgtcaacatggtcctgcagagctctctgggcaatgaccttagggtggacggggccagcatcaagtttgacagcgtcaccctctatgctaccttcttccccatggctcacaacaccgcctccacgctcgaggccatgctgaggaacgacaccaacgaccagtccttcaatgactacctctctggggccaacatgctctaccccatccccgccaaggccaccaacgtgcccatctccattccctctcgcaactgggccgccttcagaggctgggcctttacccgccttaagaccaaggaaaccccctccctgggctcgggttttgacccctactttgtctactcgggatccatcccctacctggatggcaccttctacctcaaccacacttttaagaagatatccatcatgtatgactcctccgtcagctggccgggcaatgaccgcctgctcacccccaatgagttcgaggtcaagcgcgccgtggacggcgagggctacaacgtggcccagtgcaacatgaccaaggactggttcctggtgcagatgctggccaactacaacataggctaccagggcttctacatcccagagagctacaaggacaggatgtactccttcttcagaaatttccaacccatgagcaggcaggtggtggacgagaccaaatacaaggactatcaggccattggcatcactcaccagcacaacaactcgggattcgtgggctacctggctcccaccatgcgcgaggggcaggcctaccccgccaacttcccctacccgttgataggcaaaaccgcggtcgacagcgtcacccagaaaaagttcctctgcgaccgcaccctctggcgcatccccttctctagcaacttcatgtccatgggtgcgctcacggacctgggccagaacctgctctatgccaactccgcccatgcgctggacatgacttttgaggtggaccccatggacgagcccacccttctctatattgtgtttgaagtgttcgacgtggtcagagtgcaccagccgcaccgcggtgtcatcgagaccgtgtacctgcgcacgcccttctcggccggcaacgccaccacc

SEQ ID NO: 10 - DNA, 1749 bp, synthetic construct
  misc_feature 1..1749: note = Adenovirus fiber protein nucleotides
  source 1..1749: mol_type = other DNA
atgaaacgcgcgagatcgtctgacgagaccttcaaccccgtgtacccctacgataccgagatcgctccgacttctgtccctttccttacccctccctttgtgtcatccgcaggaatgcaagaaaatccagctggggtgctgtccctgcacttgtcagagccccttaccacccacaatggggccctgactctaaaaatggggggcggcctgaccctggacaaggaagggaatctcacttcccaaaacatcaccagtgtcgatccccctctcaaaaaaagcaagaacaacatcagccttcagaccgccgcacccctcgccgtcagctccggggccctaacactttttgccactccccccctagcggtcagtggtgacaaccttactgtgcagtctcaggcccctctcactttggaagactcaaaactaactctggccaccaaaggacccctaactgtgtccgaaggcaaacttgtcctagaaacagaggctcccctgcatgcaagtgacagcagcagcctgggccttagcgttacggccccacttagcattaacaatgacagcctaggactagatctgcaggcacccattgtctctcaaaatggaaaactggctctaaatgtagcaggccccctagctgtggccaatggcattaatgctttgacagtaggcacaggcaaaggtattggtctaaatgaaaccagcactcacttgcaagcaaagttggtcgcccccctaggctttgataccaatggcaacattaagctaagcgttgcaggaggcatgagactaaataatgacacacttatactagatgtaaactacccatttgaagctcaaggccaactaagtctaagagtgggccagggtccgctgtatgtagattctagcagccataacctgaccattagatgccttagaggattatacataacatcgtctaataaccaaaccggtctagaggccaacataaaactaacaaaaggccttgtctatgatggaaatgccatagcagtcaatgttggtcaaggattgcaatacagcactactgccacatcggaaggtgtgtatcctatacagtctaagataggtttgggaatggaatatgataccaacggagccatgatgacaaaactaggctctggactaagctttgacaattcaggagccattgtagtgggaaacaaaaatgatgacaggcttactctgtggactacaccagacccatctcctaactgtagaatttattctgaaaaagatactaaactaaccttggtgctgactaagtgtggcagccaaatcctaggcacagtatctgcccttgctgtcagaggcagccttgcgcccatcactaatgcatccagcatagtccaaatatttctaagatttgatgaaaatggactattgatgagcaactcatcgctagacggtgattactggaattacagaaatggggactccactaatagcacaccatatacaaatgcagtaggctttatgcctaatctagcagcctatcctaaaggtcaggctacagctgcaaaaagcagtattgtaagccaggtatacatggatggtgacactactaaacctataacactaaaaataaacttcaatggcattgatgaaacaacagaaaatacccctgttagtaaatattccatgacattctcatggagctggcccaccgcaagctacataggccacacttttgcaacaaactcttttactttctcctacatcgcccaagaa

SEQ ID NO: 11 - protein, 10 aa, synthetic construct
  REGION 1..10: note = Adenovirus pIX protein fragment
SSLVASGAAS

SEQ ID NO: 12 - protein, 147 aa, synthetic construct
  REGION 1..147: note = Adenovirus DNA polymerase fragment
KHQYLKVMVRDTFALTHTSLRKAAQAYALPVEKGCCPYQAVNQFYMLGSYRSDTDGFPLQEYWKDREEFVLNRELWKKKGEDKYDIIRETLDYCALDVQVTAELVHKLRESYASFVRDSVGLQEASFNVFQRPTISSNSHAIFRQIA

SEQ ID NO: 13 - protein, 15 aa, synthetic construct
  REGION 1..15: note = Adenovirus penton base protein fragment
KTEAAAKAEVEANVA

SEQ ID NO: 14 - protein, 15 aa, synthetic construct
  REGION 1..15: note = Adenovirus hexon protein fragment
IGVDATQAGDNPIYA

SEQ ID NO: 15 - protein, 15 aa, synthetic construct
  REGION 1..15: note = Adenovirus fiber protein fragment
LNVAGPLAVANGINA

SEQ ID NO: 16 - protein, 133 aa, synthetic construct
  REGION 1..133: note = Adenovirus pIX amino acids
MSDTGNSFDGSIFSPYLTVRMPHWAGVRQNVMGSNVDGRPVLPSNSSTMAYATVGGTPLDAATSAAASAAAATARSMATDLYSSLVASGAASRASARDEKLTALLLKLEDLTRELGQLTQQVSSLRESSLASP

SEQ ID NO: 17 - protein, 1056 aa, synthetic construct
  REGION 1..1056: note = Adenovirus DNA polymerase amino acids
MDSSNVRDVVIKLRPPSAEIWTCGSRGVVVCSTIALQETDAGGQTTKVEDHQPHGTPGGGLRFPLRFLVRGRQVHLVQDIQPVQRCQYCGRFYKSQHECSARRRDFYFHHINSQSSNWWREIQFFPIGSHPRTERLFVTYDVETYTWMGAFGKQLVPFMLVMKLGGDEALVAAARDLARELRWDPWEKDPLTFYCITPEKMAVGRQFRTFRDRLQTLMARDLWRSFLAANPHLQDWALEEHGLESPEELTYEELKKLPSIKGQPRFLELYIVGHNINGFDEIVLAAQVINNRSSVPGPFRITRNFMPRAGKILFNDLTFSLPNPRSKKRTDYTLWEQGGCDDTDFKHQYLKVMVRDTFALTHTSLRKAAQAYALPVEKGCCPYQAVNQFYMLGSYRSDTDGFPLQEYWKDREEFVLNRELWKKKGEDKYDIIRETLDYCALDVQVTAELVHKLRESYASFVRDSVGLQEASFNVFQRPTISSNSHAIFRQIAFRAERPQRTNLGPNMLAPSHELYDYVRASIRGGRCYPTYLGILREPLYVYDICGMYASALTHPMPWGPPLNPYERALAAREWQRALDMQACKIDYFDPRLLPGVFTIDADPPNEDQLDPLPPFCSRKGGRLCWTNERLRGEVATSVDMVTLHNRGWRVRLIPDERTTVFPEWKCVAREYVQLNIAAKERADRDKNQTLRSIAKLLSNALYGSFATKLDNKKIVFSDQMDPGTLKGITSGQVNIKSSSFLETDNLSAEVMPAFEREYLPQQLALADSDAEESEDERAPTPFYTPPSGTPGHVSYTYKPITFLDAEEGDMCLHTLEKVDPLVDNDRYPSHVASFVLAWTRAFVSEWSEFLYEEDRGTPLEDRPLKSVYGDTDSLFVTEKGHRLMESRGKKRIKKHGGNLVFDPDRPELTWLVECETVCASCGADAYSPESVFLAPKLYALKSLQCPSCGATSKGKLRAKGHAAEGLDYETMVKCYLADAQGEERQRFSTSRTSLKRTLASAQPGAHPFTVTQTTLTRTLRPWKDMTLAPLDAHRLVPYSESRPNPRNEEICWIEMP

SEQ ID NO: 18 - protein, 658 aa, synthetic construct
  REGION 1..658: note = Adenovirus penton base protein
MRRAAMFEEGPPPSYESAMGISPAAPLQPPYVPPRYLQPTGGRNSICYSELQPLYDTTRLYLVDNKSADVASLNYQNDHSDFLTTVIQNNDFTPTEASTQTINLDNRSNWGGDLKTILHTNMPNVNEFMFTNSFKARVMVAREQGEAKYEWVDFTLPEGNYSETMTLDLMNNAIVEHYLKVGRQNGVKESDIGVKFDTRNFRLGWDPVTGLVMPGVYTNEAFHPDIVLLPGCGVDFTQSRLSNLLGIRKRQPFQEGFKITYEDLKGGNIPALLDLDAYEESLKPEESAGDSGESGEEQAGGGGGASVENESTPAVAADAAEVEPEAMQQDAEEGAQEGAQKDMNDGEIRGDTFATRGEEKEAEAAAAATAEAETEVEAEAEPETEVMEDMNDGERRGDTFATRGEEKAAEAEAAAEEAAAAAAKTEAAAKAEVEANVAVEAQAEEEAAAEAVKEKAQAEQEEKKPVIQPLKEDSKKRSYNVIEGSTFTQYRSWYLAYNYGDPVKGVRSWTLLCTPDVTCGSEQMYWSLPNMMQDPVTFRSTRQVSNFPVVGAELLPVHSKSFYNEQAVYSQLIRQATSLTHVFNRFPENQILARPPAPTITTVSENVPALTDHGTLPLRNSISGVQRVTITDARRRTCPYVYKALGIVSPRVLSSRTF

SEQ ID NO: 19 - protein, 959 aa, synthetic construct
  REGION 1..959: note = Adenovirus hexon protein
MATPSMMPQWSYMHISGQDASEYLSPGLVQFARATDTYFNMSNKFRNPTVAPTHDVTTDRSQRLTLRFIPVDREDTAYSYKARFTLAVGDNRVLDMASTYFDIRGVLDRGPTFKPYSGTAYNSLAPKGAPNSCEWEQDEPAQAAIAEDEEELEEEQAQDEQAPTKKTHVYAQAPLSGEKITKDGLQIGVDATQAGDNPIYADKTFQPEPQIGESQWNEADATVAGGRVLKKTTPMRPCYGSYAKPTNANGGQGIMVANDQGALESKVEMQFFSTTTSLNVREGENNLQPKVVLYSEDVNLESPDTHLSYKPKKDDTNSKIMLGQQAMPNRPNLIAFRDNFIGLMYYNSTGNMGVLAGQASQLNAVVDLQDRNTELSYQLMLDSIGDRSRYFSMWNQAVDSYDPDVRIIENHGVEDELPNYCFPLGGIGITDTYQCIKPTAAANNTTWSKDEEFSDRNEIGVGNNFAMEINIQANLWRNFLYANVGLYLPDKLKYNPTNVDISDNPNTYDYMNKRVVAPGLVDCFVNVGARWSLDYMDNVNPFNHHRNAGLRYRSMILGNGRYVPFHIQVPQKFFAIKNLLLLPGSYTYEWNFRKDVNMVLQSSLGNDLRVDGASIKFDSVTLYATFFPMAHNTASTLEAMLRNDTNDQSFNDYLSGANMLYPIPAKATNVPISIPSRNWAAFRGWAFTRLKTKETPSLGSGFDPYFVYSGSIPYLDGTFYLNHTFKKISIMYDSSVSWPGNDRLLTPNEFEVKRAVDGEGYNVAQCNMTKDWFLVQMLANYNIGYQGFYIPESYKDRMYSFFRNFQPMSRQVVDETKYKDYQAIGITHQHNNSGFVGYLAPTMREGQAYPANFPYPLIGKTAVDSVTQKKFLCDRTLWRIPFSSNFMSMGALTDLGQNLLYANSAHALDMTFEVDPMDEPTLLYIVFEVFDVVRVHQPHRGVIETVYLRTPFSAGNATT

SEQ ID NO: 20 - protein, 583 aa, synthetic construct
  REGION 1..583: note = Adenovirus fiber protein
MKRARSSDETFNPVYPYDTEIAPTSVPFLTPPFVSSAGMQENPAGVLSLHLSEPLTTHNGALTLKMGGGLTLDKEGNLTSQNITSVDPPLKKSKNNISLQTAAPLAVSSGALTLFATPPLAVSGDNLTVQSQAPLTLEDSKLTLATKGPLTVSEGKLVLETEAPLHASDSSSLGLSVTAPLSINNDSLGLDLQAPIVSQNGKLALNVAGPLAVANGINALTVGTGKGIGLNETSTHLQAKLVAPLGFDTNGNIKLSVAGGMRLNNDTLILDVNYPFEAQGQLSLRVGQGPLYVDSSSHNLTIRCLRGLYITSSNNQTGLEANIKLTKGLVYDGNAIAVNVGQGLQYSTTATSEGVYPIQSKIGLGMEYDTNGAMMTKLGSGLSFDNSGAIVVGNKNDDRLTLWTTPDPSPNCRIYSEKDTKLTLVLTKCGSQILGTVSALAVRGSLAPITNASSIVQIFLRFDENGLLMSNSSLDGDYWNYRNGDSTNSTPYTNAVGFMPNLAAYPKGQATAAKSSIVSQVYMDGDTTKPITLKINFNGIDETTENTPVSKYSMTFSWSWPTASYIGHTFATNSFTFSYIAQE

SEQ ID NO: 21 - DNA, 32728 bp, synthetic construct
  misc_feature 1..32728: note = Adenovirus vector nucleotide sequences
  source 1..32728: mol_type = other DNA
catcatcaataatataccttattttggattgtggccaatatgataatgaggtgggcggggagaggcggggcgggtgacgtaggacgcgcgagtagggttgggaggtgtggcggaagtgtggcatttgcaagtgggaggagctcacatgcaagcttccgtcgcggaaaatgtgacgtttttgatgagcgccgcctacctccggaagtgccaattttcgcgcgcttttcaccggatatcgtagtaattttgggcgggaccatgtaagatttggccattttcgcgcgaaaagtgaaacggggaagtgaaaactgaataatagggcgttagtcatagcgcgtaatatttaccgagggccgagggactttgaccgattacgtggaggactcgcccaggtgttttttacgtgaatttccgcgttccgggtcaaagtctccgtttttattgtcaccgtcatttgacgcggagggtatttaaacccgctgcgctcctcaagaggccactcttgagtgccagcgagaagagttttctcctctgctccgcttcggtgatcgaaaaatgagacacatagcctgcactccgggtcttttgtccggtcgggcggcggccgagcttttggacgctttgatcaatgatgtcctaagcgatgattttccgtctactacccactttagcccacctactcttcacgaactgtacgatctggatgtactggtggatgtgaacgatcccaacgaggaggcggtttctgcgttttttcccgagtctgcgctgttggccgctcaggagggatttgacctacacactccgccgcctattttagagtctccgctgccggagcccagtggtataccttatatgcctgaactgcttcccgaagtggtagacctgacctgccacgagcctggctttccgcccagcgacgatgagggtgagccttttgttttagactttgctgagatacctgggcacggttgcaggtcttgtgcatatcatcagagggttaccggagaccccgaggttaagtgttcgctgtgctatatgaggatgacctcttcctttatctacagtaagtttttgtctaggtgggcttttgggtaggtgggttttgtgtcagaacaggtgtaaacgttgcttgtgttttttgtacctgtaggtccggtgtccgagccagacccggagcccgaccgcgatcccgagccggatcccgagcctcctcgcaggacaaggaaactaccttccattctgtgcaagtctcagacacctgtaaggaccagcgaggcagacagcaccgactctggcacttctacctctccccctgaaattcacccagtggttcctctgggtatacataaacctgttgctgttaaagtttgcgggcgacgccctgcagtacagtgcattgaggacttgcttcacgatcccgaggaacctttggacttgagccttaaacgccctaggcaataaaccccacctaagtaataaaccccacctaagtaataaaccctgccgcccttggttattgagatgacgcccaatgtttgcttttgaatgacttcatgtgtgtaataaaagtgagtgtgatcataggtctcttgtttgtctgggcggggcttaagggtatataagtctcttggggctaaacttggttacacttgaccccaatggaggcgtgggggtgcttggaggagtttgcggacgtgcgccgtttgctggacgagagctctagcaatacctatactatttggaggtatctgtggggctctactcaggccaagttggtttccagaattaagcaggattacaagtgcgattttgaagagctttttagttcctgcggtgagcttttgcaatccttgaatctgggccatcaggctattttccaggaaaaggttctctcgactttggatttttccactcccgggcgcaccgccgcttgtgtggcttttgtgtcttttgtgcaagataaatggagcgaggagacccacctgagtcacggctacgtactggatttcatggcgatggctctttggagggctcacaacaaatggaagattcagaaggaactgtacggttccgccctacgtcgtccacttctgtcgcgacaggggctgaggtttcccgaccatcggcagcatcagaatctggaagacgagtcggaggagcgagcggaggagaagatcagcttgagagccggcctggaccctcctcaggaggaatgaatctcccgcaggtggttgacctgtttccagaactgagacgggtcctgactatcagggaggatggtcagtttgtgaagaagtttaagagggatcggggtgagggagatgatgaggcggctagcaatttagcttttagtctgatgactcgccaccgaccggaatgtattacctatcagcagattaaggagagttgtgccaacgagctggatcttttgggtcagaagtatagcatagaacagcttaccacttactggcttcagcctggggatgattgggaagaggcgatcagggtgtatgcaaaggtggccctgcggcccgattgcaagtataagattactaagttggttaatattagaaactgctgctatatttctgggaacggggccgaagtggagatagatactcaggacagggtggcttttaggtgttgcatgataaacatgtggcccgggatactggggatggatggggtggtattcatgaatgtgaggtttacgggccccaactttaatggcacggtgttcatgggcaacaccaacttgctcctgcatggtgcgagtttctatgggtttaataacacctgtatagaggcctggaccgatgtaaaggttcgaggttgttccttttatagctgttggaaggcggtggtgtgtcgccctaaaagcaggggttctgtgaaaaaatgcttgtttgaaaggtgcaccttaggcatcctctctgagggcaactccagggtgcgccataatgtggcttcgaactgcggttgcttcatgcaagtgaagggggtgagcgttatcaagcataactcggtgtgtggaaactgcgaggatcgcgcctcccagatgctgacctgctttgatggcaactgtcacctgttgaagaccattcatataagcagccaccccagaaaggcctggcccgtgtttgagcataacatcttgacccgctgctccttgcatctgggggtcaggaggggtatgttcctgccttaccagtgtaactttagccacactaaaatcctgctggaacccgagtgcatgaccaaggtcagcctgaatggtgtgtttgatgtgactctgaaaatctggaaggtgctgaggtatgatgagaccaggaccaggtgccgaccctgcgagtgcggcggcaagcacatgagaaatcagcctgtgatgttggatgtgaccgaggagcttaggcctgaccatctggtgctggcctgcaccagggccgagtttgggtctagcgatgaggataccgattgaggtgggtaaggtgggcgtggctagaagggtggggcgtgtataaattgggggtctaagggtctctctgttttgtcttgcaacagccgccgccatgagcgacaccggcaacagctttgatggaagcatctttagcccctatctgacagtgcgcatgcctcactgggctggagtgcgtcagaatgtgatgggttccaacgtggatggacgccccgttctgccttcaaattcgtctacaatggcctacgcgaccgtgggaggaactccgctggacgccgcgacctccgccgccgcctccgccgccgccgcgaccgcgcgcagcatggctacggacctttacagctctttggtggcgagcggcgcggcctctcgcgcgtctgctcgggatgagaaactgaccgctctgctgcttaaactggaagacttgacccgggagctgggtcaactgacccagcaggtctccagcttgcgtgagagcagccttgcctccccctaatggcccataatataaataaaagccagtctgtttggattaagcaagtgtatgttctttatttaactctccgcgcgcggtaagcccgggaccagcggtctcggtcgtttagggtgcggtggattctttccaacacgtggtacaggtggctctggatgtttagatacatgggcatgagtccatccctggggtggaggtagcaccactgcagagcttcgtgctcgggggtggtgttgtatatgatccagtcgtagcaggagcgctgggcgtggtgctgaaaaatgtccttaagcaagaggcttatagctagggggaggcccttggtgtaagtgtttacaaatctgctcagttgggaggggtgcatccggggggatataatgtgcatcttggactggatttttaggttggctatgttcccacccagatcccttctgggattcatgttgtgcaggaccaccagcacggtatatccagtgcacttgggaaatttatcgtggagcttagacgggaatgcatggaagaacttggagacgcccttgtggcctcccagattttccatacattcgtccatgatgatggcaatgggcccgtgggaagctgcctgagcaaaaatgtttctgggatcgctcacatcgtagttatgttccagggtgaggtcatcataggacatctttacgaatcgggggcggagggtcccggactgggggatgatggtaccctcgggccccggggcgtagttcccctcacagatctgcatctcccaggctttcatttcagagggagggatcatatccacctgcggagcgatgaaaaacacagtttctggcgcaggggagattaactgggatgagagcaggtttctgagcagctgtgactttccacagccggtgggcccatatatcacgcctatcaccggctgcagctggtagttaagagagctgcagctgccgtcctcccggagcaggggggccacctcgttcagcatatccctgacgtggatgttctccctgaccaattccgccagaaggcgctcgccgcccagcgaaagcagctcttgcaaggaagcaaaatttttcagcggttttaggccgtcggccgtgggcatgtttttcagcgtctgggtcagcagttccagcctgtcccacagctcggtgatgtgctctacggcatctcgatccagcagatctcctcgtttcgcgggttggggcggctttcgctgtagggcaccagccgatgggcgtccagcggggccagagtcatgtccttccatgggcgcagggtcctcgtcagggtggtctgggtcacggtgaaggggtgcgctccgggttgggcgctggccagggtgcgcttgaggctggttctgctggtgctgaatcgctgccgctcttcgccctgcgcgtcggccaggtagcatttgaccatggtctcgtagtcgagaccctcggcggcgtgccccttggcgcggagctttcccttggaggtggcgccgcacgaggggcactgcaggctcttcagggcgtagagcttgggagcgagaaacacggactctggggagtaggcgtccgcgccgcaggaagcgcagaccgtctcgcattccaccagccaagtgagctccgggcggtcagggtcaaaaaccaggttgcccccatgctttttgatgcgtttcttacctcggctctccatgaggcggtgtcccttctcggtgacgaagaggctgtccgtgtccccgtagaccgacttcaggggcctgtcttccagcggagtgcctctgtcctcctcgtagagaaactctgaccactctgagacgaaggcccgcgtccaggccaggacgaaggaggccacgtgggaggggtagcggtcgttgtccactagcgggtccaccttctccagggtgtgcaggcacatgtccccctcctccgcgtccagaaaagtgattggcttgtaggtgtaggacacgtgaccgggggttcccgacgggggggtataaaagggggtgggcgccctttcatcttcactctcttccgcatcgctgtctgcgagggccagctgctggggtaagtattccctctcgaaggcgggcatgacctcagcgctcaggttgtcagtttctaaaaatgaggaggatttgatgttcacctgtccggaggtgatacctttgagggtacctgggtccatctggtcagaaaacactatttttttgttgtcaagcttggtggcgaacgacccgtagagggcgttggagagcagcttggcgatggagcgcagggtctggtttttgtcgcggtcggctcgctccttggccgcgatgttgagttgcacgtactcgcgggccacgcacttccactcggggaagacggtggtgcgctcgtctgggattaggcgcaccctccagcctcggttgtgcagggtgaccatgtcgacgctggtggcgacctcgccgcgcaggcgctcgttggtccagcagaggcggccgcccttgcgcgagcagaaggggggtagggggtccagctggtcctcgtttggggggtccgcgtcgatggtgaagaccccggggagcaagcgcgggtcaaagtagtcgatcttgcaagcttgcatgtccagagcccgctgccattcgcgggcggcgagcgcgcgctcgtaggggttgaggggcgggccccagggcatggggtgggtgagcgcggaggcgtacatgccgcagatgtcatacacgtacaggggttccctgaggatgccgaggtaggtggggtagcagcgccccccgcggatgctggcgcgcacgtagtcatagagctcgtgggagggggccagcatgttgggcccgaggttggtgcgctgggggcgctcggcgcggaaggcgatctgcctgaagatggcatgggagttggaggagatggtgggccgctggaagacgttgaagcttgcttcttgcaagcccaccgagtccctgacgaaggaggcgtaggactcgcgcagcttgtgcaccagctcggcggtgacctggacgtcgagcgcgcagtagtcgagggtctcgcggatgatgtcatacttatcctcccccttctttttccacagctcgcggttgaggacgaactcttcgcggtctttccagtactcttggaggggaaacccgtccgtgtccgaacggtaagagcctagcatgtagaactggttgacggcctggtaggggcaacagcccttctccacgggcagcgcgtaggcctgcgccgccttgcggagggaggtgtgggtgagggcgaaagtgtccctgaccatgactttgaggtattgatgtttgaagtctgtgtcatcgcagccgccctgttcccacagggtgtagtccgtgcgctttttggagcgcgggttgggcagggagaaggtgaggtcattgaagaggatcttccccgctcgaggcatgaagtttctggtgatgcgaaagggccctgggaccgaggagcggttgttgatgacctgggcggccaggacgatctcgtcaaagccgtttatgttgtggcccacgatgtagagctccaaaaagcggggctggcccttgatggaggggagctttttgagttcctcgtaggtgagctcctcgggcgattccaggccgtgctcctccagggcccagtcttgcaagtgagggttggccgccaggaaggatcgccagaggtcgcgggccatgagggtctgcaggcggtcgcggaaggttctgaactgtcgccccacggccatcttttcgggggtgatgcagtagaaggtgagggggtctttctcccaggggtcccatctgagctctcgggcgaggtcgcgcgcggcggcgaccagagcctcgtcgccccccagtttcatgaccagcatgaagggcacgagctgcttgccaaaggctcccatccaagtgtaggtctctacatcgtaggtgacaaagaggcgctccgtgcgaggatgagagccgatcgggaagaactggatctcccgccaccagttggaggattggctgttgatgtggtgaaagtagaagtcccgtctgcgggccgagcactcgtgctggcttttgtaaaagcgaccgcagtactggcagcgctgcacgggttgtatatcttgcacgaggtgaacctggcgacctctgacgaggaagcgcagcgggaatctaagtcccccgcctggggtcccgtgtggctggtggtcttctactttggttgtctggccgccagcatctgtctcctggagggcgatggtggagcagaccaccacgccgcgagagccgcaggtccagatctcggcgctcggcgggcggagtttgatgacgacatcgcgcacattggagctgtccatggtctccagctcccgcggcggcaggtcagctgggagttcctggaggttcacctcgcagagacgggtcaaggcgcgggcagtgttgagatggtatctgatttcaaggggcgtgttggcggcggagtcgatggcttgcaggaggccgcagccccggggggccacgatggttccccgcggggcgcgaggggaggcggaagctgggggtgtgttcagaagcggtgacgcgggcgggcccccggaggtagggggggttccggccccacaggcatgggcggcaggggcacgtcttcgccgcgcgcgggcaggggctggtgctggctccgaagagcgcttgcgtgcgcgacgacgcgacggttggtgtcctgtatctgacgcctctgagtgaagaccacgggtcccgtgaccttgaacctgaaagagagttcgacagaatcaatctcggcatcgttgacagcggcctggcgcaggatctcctgcacgtcgcccgagttgtcctggtaggcgatctctgccatgaactgctcgatctcttcttcctggagatctcctcgtccggcgcgctccacggtggccgccaggtcgttggagatgcgacccatgagctgtgagaaggcgttgagcccgccctcgttccagacccggctgtagaccacgcccccctcggcgtcgcgagcgcgcatgaccacctgggccaggttgagctccacgtgtcgcgtgaagacggcgtagttgcgcaggcgctggaaaaggtagttcagggtggtggcggtgtgctcggcgacgaagaagtacatgacccagcgccgcaacgtggattcattgatgtcccccaaggcctccaggcgctccatggcctcgtagaagtccacggcgaagttgaaaaactgggagttgcgagcggacacggtcaactcctcctccagaagacggatgagctcggcgacagtgttgcgcacctcgcgctcgaaggccacggggggcgcttcttcctcttccacctcttcttccatgatcgcttcttcttcttcctcagccgggacgggagggggcggcggcggcgggggaggggcgcggcggcggcggcggcgcaccgggaggcggtcgatgaagcgctcgatcatctccccccgcatgcggcgcatggtctcggtgacggcgcggccgttctcccgggggcgcagctcgaagacgccgcctctcatctcgccgcggggcgagcggccgtgaggtagcgagacggcgctgactatgcatcttaacaattgctgtgtaggtacaccgccgagggacctgattgagtccagatccaccggatccgaaaacctttggaggaaagcgtctatccagtcgcagtcgcaaggtaggctgagcaccgtggcgggcgggggcgggtctggagagttcctggcggagatgctgctgatgatgtaattaaagtaggcggtcttgagaaggcggatggtggacaggagcaccatgtctttgggtccggcctgttggatgcggaggcggtcggccatgccccaggcctcgttctgacaccggcgcaggtctttgtagtagtcttgcatgagtctttccaccggcacctcttctccttcctcttctccatctcgccggtggtttctcgcgccgcccatgcgcgtgaccccaaagcccctgagcggctgcagcagggccaggtcggcgaccacgcgctcggccaagatggcctgctgcacctgagtgagggtcctctcgaagtcatccatgtccacgaagcggtggtaggcgcccgtgttgatggtgtaggtgcagttggccatgacggaccagttgacggtctggtgtcccggctgcgagagctccgtgtaccgcaggcgcgagaaggcgcgggaatcgaacacgtagtcgttgcaagtccgcaccagatactggtagcccaccaggaagtgcggcggaggttggcgatagaggggccagcgctgggtggcgggggcgccgggcgccaggtcttccagcatgaggcggtggtatccgtagatgtacctggacatccaggtgatgccggcggcggtggtggtggcgcgcgcgtagtcgcggacccggttccagatgtttcgcaggggcgagaagtgttccatggtcggcacgctctggccggtgaggcgcgcgcagtcgttgacgctctatacacacacaaaaacgaaagcgtttacagggctttcgttctgtagcctggaggaaagtaaatgggttgggttgcggtgtgccccggttcgagaccaagctgagctcggccggctgaagccgcagctaacgtggtattggcagtcccgtctcgacccaggccctgtatcctccaggatacggtcgagagcccttttgctttcttggccaagcgcccgtggcgcgatctgggatagatggtcgcgatgagaggacaaaagcggctcgcttccgtagtctggagaaacaatcgccagggttgcgttgcggcgtaccccggttcgagcccctatggcggcttgaatcggccggaaccgcggctaacgagggccgtggcagccccgtcctcaggaccccgccagccgacttctccagttacgggagcgagccccttttgttttttattttttagatgcatcccgtgctgcggcagatgcgcccctcgccccggcccgatcagcagcagcaacagcaggcatgcagacccccctctcccctttccgccccggtcaccacggccgcggcggccgtgtcgggcgcggggggcgcgctggagtcagatgagccaccgcggcggcgacctaggcagtatctggacttggaagagggcgagggactggcgcggctgggggcgaactctccagagcgccacccgcgggtgcagttgaaaagggacgcgcgcgaggcgtacctgccgcggcagaacctgtttcgcgaccgcgggggcgaggagcccgaggagatgcgagactgcaggttccaagcggggcgcgagctgcggcgcgggctggacagacagcgcctgctgcgcgaggaggactttgagcccgacacgcagacgggcatcagccccgcgcgcgcgcacgtagccgcggccgacctggtgaccgcctacgagcagacggtaaaccaggagcgcaacttccaaaagagcttcaacaaccacgtgcgcacgctggtggcgcgcgaggaggtgaccctgggtctcatgcatctgtgggacctggtggaggcgatcgtgcagaaccccagcagcaagcccctgaccgcgcagctgttcctggtggtgcagcacagcagggacaacgaggccttcagggaggcgctgctgaacatcaccgagccggaggggcgctggctcctggacctgataaacatcctgcagagcatagtggtgcaggagcgcagcctgagcctggccgagaaggtggcggccatcaactactctatgctgagcctgggcaagttctacgcccgcaagatctacaagaccccctacgtgcccatagacaaggaggtgaagatagacagcttctacatgcgcatggcgctgaaggtgctgaccctgagcgacgacctgggagtgtaccgcaacgagcgcatccacaaggccgtgagcgccagccggcggcgcgagctgagcgaccgcgagctgatgcacagtctgcagcgcgcgctgaccggcgcgggcgagggcgacagggaggtcgagtcctacttcgacatgggggccgacctgcactggcagccgagccgccgcgccctggaggcggcgggggcgtacggcggccccctggcggccgatgaccaggaagaggaggactatgagctagaggagggcgagtacctggaggactgacctggctggtggtgttttggtatagatgcaagatccgaacgtggcggacccggcggtccgggcggcgctgcaaagccagccgtccggcattaactcctctgacgactgggccgcggccatgggtcgcatcatggccctgaccgcgcgcaaccccgaggctttcaggcagcagcctcaggccaaccggctggcggccatcttggaagcggtagtgcccgcgcgctccaaccccacccacgagaaggtgctggccatagtcaacgcgctggcggagagcagggccatccgcgcggacgaggccggactggtgtacgatgcgctgctgcagcgggtggcgcggtacaacagcggcaacgtgcagaccaacctggaccgcctggtgacggacgtgcgcgaggccgtggcgcagcgcgagcgcttgcatcaggacggtaacctgggctcgctggtggcgctaaacgccttcctcagcacccagccggccaacgtaccgcgggggcaggaggactacaccaactttttgagcgcgctgcggctgatggtgaccgaggtccctcagagcgaggtgtaccagtcggggcccgactacttcttccagaccagcagacagggcttgcaaaccgtgaacctgagccaggctttcaagaacctgcgggggctgtggggagtgaaggcgcccaccggcgaccgggctacggtgtccagcctgctaacccccaactcgcgcctgctgctgctgctgatcgcgcccttcacggacagcgggagcgtctcgcgggagacctatctgggccacctgctgacgctgtaccgcgaggccatcgggcaggcgcaggtggacgagcacaccttccaagagatcaccagcgtgagccacgcgctggggcaggaggacacgggcagcctgcaggcgaccctgaactacctgctgaccaacaggcggcagaagattcccacgctgcacagcctgacccaggaggaggagcgcatcttgcgctacgtgcagcagagcgtgagcctgaacctgatgcgcgacggcgtgacgcccagcgtggcgctggacatgaccgcgcgcaacatggaaccgggcatgtacgcctcccaccggccgtttatcaaccgcctgatggactacttgcatcgggcggcggccgtgaaccccgagtacttcactaatgccattctgaatccccactggatgccccctccgggtttctacaacggggactttgaggtgcccgaggtcaacgacgggttcctctgggatgacatggatgacagtgtgttctcacccaacccgctgcgcgccgcgtctctgcgattgaaggagggctctgacagggaaggaccgaggagtctggcctcctccctggctctgggagcggtgggcgccacgggcgcggcggcgcggggcagtagccccttccccagcctggcagactctctgaacagcgggcgggtgagcaggccccgcttgctaggcgaggaggagtatctgaacaactccctgctgcagcccgcgagggacaagaacgctcagcggcagcagtttcccaacaatgggatagagagcctggtggacaagatgtccagatggaagacgtatgcgcaggagtacaaggagtgggaggaccgccagccgcggcccttgccgccccctaggcagcgctggcagcggcgcgcgtccaaccgccgctggaggcaggggcccgaggacgatgatgactctgcagatgacagcagcgtgttggacctgggcgggagcgggaaccccttttcgcacctgcgcccacgcctgggcaagatgttttaaaagaaaaaaaaaataaaactcaccaaggccatggcgacgagcgttggttttttgttcccttccttagtatgcggcgcgcggcgatgttcgaggaggggcctcccccctcttacgagagcgcgatggggatttctcctgcggcgcccctgcagcctccctacgtgcctcctcggtacctgcaacctacaggggggagaaatagcatctgttactctgagctgcagcccctgtacgataccaccagactgtacctggtggacaacaagtccgcggacgtggcctccctgaactaccagaacgaccacagcgattttttgaccacggtgatccaaaacaacgacttcaccccaaccgaggccagcacccagaccataaacctggataacaggtcgaactggggcggcgacctgaagaccatcttgcacaccaacatgcccaacgtgaacgagttcatgttcaccaactcttttaaggcgcgggtgatggtggcgcgcgagcagggggaggcgaagtacgagtgggtggacttcacgctgcccgagggcaactactcagagaccatgactctcgacctgatgaacaatgcgatcgtggaacactatctgaaagtgggcaggcagaacggggtgaaggaaagcgatatcggggtcaagtttgacaccagaaacttccgtctgggctgggaccccgtgaccgggctggtcatgccgggggtctacaccaacgaggcctttcatcccgacatagtgcttctgcccggctgtggggtggacttcacccagagccggctgagcaacctgctgggcattcgcaagcggcagcctttccaggagggtttcaagatcacctatgaggatctgaaggggggcaacattcccgcgctccttgatctggacgcctacgaggagagcttgaaacccgaggagagcgctggcgacagcggcgagagtggcgaggagcaagccggcggcggtggcggcgcgtcggtagaaaacgaaagtacgcccgcagtggcggcggacgctgcggaggtcgagccggaggccatgcagcaggacgcagaggagggcgcacaggagggcgcgcagaaggacatgaacgatggggagatcaggggagacacattcgccacccggggcgaagaaaaagaggcagaggcggcggcggcggcgacggcggaggccgaaaccgaggttgaggcagaggcagagcccgagaccgaagttatggaagacatgaatgatggagaacgtaggggcgacacgttcgccacccggggcgaagagaaggcggcggaggcagaagccgcggctgaggaggcggctgcggctgcggccaagactgaggctgcggctaaggctgaggtcgaagccaatgttgcggttgaggctcaggctgaggaggaggcggcggctgaagcagttaaggaaaaggcccaggcagagcaggaagagaaaaaacctgtcattcaacctctaaaagaagatagcaaaaagcgcagttacaacgtcatcgagggcagcacctttacccagtaccgcagctggtacctggcgtacaactacggcgacccggtcaagggggtgcgctcgtggaccctgctctgcacgccggacgtcacctgcggctccgagcagatgtactggtcgctgccgaacatgatgcaagacccggtgaccttccgctccacgcggcaggttagcaacttcccggtggtgggcgccgaactgctgcccgtgcactccaagagtttttacaacgagcaggccgtctactcccagctgatccgccaggccacctctctgacccacgtgttcaatcgctttcccgagaaccagattttggcgcgcccgccggcccccaccatcaccaccgtgagtgaaaacgttcctgccctcacagatcacgggacgctaccgctgcgcaacagcatctcaggagtccagcgagtgaccattactgacgccagacgccggacctgcccctacgtttacaaggccttgggcatagtctcgccgcgcgtcctctccagtcgcactttttaaaacacatctacccacacgttccaaaatcatgtccgtactcatctcacccagcaacaacaccggctgggggctgcgcgcgcccagcaagatgtttggaggggcgaggaagcgctccgaccagcaccctgtgcgcgtgcgcggccactaccgcgcgccctggggagcgcacaagcgcgggcgcacagggcgcaccactgtggacgacgtcattgactccgtagtggagcaagcgcgccactacacacccggcgcgccgaccgcccccgccgtgtccaccgtggaccaggcgatcgaaagcgtggtacagggcgcgcggcactatgccaaccttaaaagtcgccgccgccgcgtggcccgccgccatcgccggagaccccgggccaccgccgccgcgcgccttactaaggctctgctcaggcgcgccaggcgaactggccaccgggccgccatgagggccgcacggcgggctgccgctgccgcaagcgtcgtggccccgcgggcacgaaggcgcgcggccgctgccgccgccgccgccatttccagcttggcctcgacgcggcgcggtaacatatactgggtgcgcgactcggtaaccggcacgcgggtacccgtgcgctttcgccccccgcggaattagcacaagacaacatacacactgagtctcctgctgttgtgtatcccagcggcgaccgtcagcagcggcgacatgtccaagcgcaaaattaaagaagagatgctccaggtcatcgcgccggagatctatgggcccccgaagaaggaggaggatgattacaagccccgcaagctaaagcgggtcaaaaagaaaaagaaagatgatgatgacgaggcggtggagtttgtccgccgcatggcacccaggcgccccgtgcagtggaagggccggcgcgtgcagcgcgttttgcgccccggcaccgcggtggtcttcacgcccggcgagcgctccacgcgcactttcaagcgggtgtacgatgaggtgtacggcgacgaggacctgttggagcaggccaaccagcgctttggggagtttgcatatgggaaacggccccgcgagagtctaaaagaggacctgctggcgctaccgctggacgagggcaatcccaccccgagtctgaagccggtaaccctgcaacaggtgctgcctttgagcgcgcccagcgagcataagcgagggttgaagcgcgaaggcggggacctggcgcccaccgtgcagttgatggtgcccaagcggcagaagctggaggacgtgctggagaaaatgaaagtagagcccgggatccagcccgagatcaaggtccgccccatcaagcaggtggcgcccggcgtgggagtccagaccgtggacgttaggattcccacggaggagatggaaacccaaaccgccactccctcttcggcggccagcgccaccaccggcaccgcttcggtagaggtgcagacggacccctggctacccgccaccgctgttgccgccgccgccccccgttcgcgcgggcgcaagagaaattatccagcggccagcgcgctcatgccccagtacgcactgcatccatccatcgtgcccacccccggctaccgcgggtactcgtaccgcccgcgcagatcagccggcactcgcggccgccgccgccgtgcgaccacaaccagccgccgccgtcgccgccgccgccagccagtgctgacccccgtgtctgtaaggaaggtggctcgctcggggagcacgctggtggtgcccagagcgcgctaccaccccagcatcgtttaaagccggtctctgtatggttcttgcagatatggccctcacttgtcgcctccgcttcccggtgccgggataccgaggaagaactcaccgccgcagaggcatggcgggcagcggtctccgcggcggccgtcgccatcgccggcgcgcaaaaagcaggcgcatgcgcggcggtgtgctgcctctgctaatcccgctaatcgccgcggcgatcggtgccgtacccgggatcgcctccgtggccctgcaggcgtcccagaaacgttgactcttgcaaccttgcaagcttgcattttttggaggaaaaataaaaaaaagtctagactctcacgctcgcttggtcctgtgactattttgtagaaaaaaagatggaagacatcaactttgcgtcgctggccccgcgtcacggctcgcgcccgttcatgggagactggacagatatcggcaccagcaatatgagcggtggcgccttcagctggggcagtctgtggagcggccttaaaaattttggttccaccattaagaactatggcaacaaagcgtggaacagcagcacgggccagatgctgagagacaagttgaaagagcagaacttccaggagaaggtggcgcagggcctggcctctggcatcagcggggtggtggacatagctaaccaggccgtgcagaaaaagataaacagtcatctggacccccgtcctcaggtggaggaaatgcctccagcgatggagacggtgtctcccgagggcaaaggcgaaaagcgcccgcggcccgacagagaagagaccctggtgtcacacaccgaggagccgccctcttacgaggaggcagtcaaggccggcctgcccaccactcgccccatagcccccatggccaccggtgtggtgggccacaggcaacacactcccgcaacactagatctgcccccgccgtccgagccgccgcgccagccaaaggcggcgacggtgcccgctccctccacttccgccgccaacagagtgcccctgcgccgcgccgcgagcggcccccgggcctcgcgagttagcggcaactggcagagcacactgaacagcatcgtgggcctgggagtgaggagtgtgaagcgccgccgttgctactgaatgagcaagctagctaacgtgttgtatgtgtgtatgcgtcctatgtcgccgccagaggagctgttgagccgccggcgccgtctgcactccagcgaatttcaagatggcgaccccatcgatgatgcctcagtggtcgtacatgcacatctcgggccaggacgcttcggagtacctgagccccgggctggtgcagttcgcccgcgccacagacacctacttcaacatgagtaacaagttcaggaaccccactgtggcgcccacccacgatgtgaccacggaccggtcgcagcgcctgacgctgcggttcatccccgtggatcgggaggacaccgcctactcttacaaggcgcggttcacgctggccgtgggcgacaaccgcgtgctggacatggcctccacttactttgacatcaggggggtgctggacaggggccccaccttcaagccctactcgggtactgcctacaactccctggcccccaagggcgctcccaattcttgcgagtgggaacaagatgaaccagctcaggcagcaatagctgaagatgaagaagaacttgaagaagaacaagctcaggacgaacaggcgcccactaagaaaacccatgtatacgcccaggcacctctttctggtgaaaaaattactaaggatggtttgcaaataggtgtggatgccacacaggcgggagataaccctatatatgctgataaaacattccaacccgaacctcagataggtgagtctcagtggaacgaggctgatgccacagtagcaggaggcagagtcttaaaaaagaccacccctatgagaccttgctatggatcctatgccaaacctactaatgccaatggcggtcaagggatcatggtggccaatgatcagggagcgcttgaatctaaagttgagatgcaatttttctccaccacaacgtctcttaatgtaagggaaggtgaaaacaatcttcagccaaaagtagtgctatacagcgaagatgttaacttggaatcccctgacactcatttgtcttacaaacctaaaaaggatgacaccaactctaaaatcatgttgggtcagcaagccatgcccaacagacccaacctcattgcttttagggacaactttattggacttatgtactacaacagcacaggcaacatgggagtgctggcaggacaggcctcccagctaaacgctgtggtagacttgcaagacagaaacacagagctgtcataccaactgatgcttgattccattggagacagatcaagatacttttccatgtggaaccaggcagtggacagctatgacccagatgtcagaatcattgaaaaccatggggttgaagatgagctgcccaactattgctttcccctgggcggtattggaattacagacacataccagtgcataaaaccaaccgcagctgctaataacactacatggtctaaggatgaagaatttagtgatcgcaatgaaataggggtgggaaacaacttcgccatggagatcaacatccaggccaacctctggaggaacttcctctatgcgaacgtggggctctacctgccagacaagctcaagtacaaccccaccaacgtggacatctctgacaaccccaacacctatgactacatgaacaagcgtgtggtggctcccggcctggtggactgctttgtcaatgtgggagccaggtggtccctggactacatggacaacgtcaaccccttcaaccaccaccgcaatgcgggtctgcgctaccgctccatgatcctgggcaacgggcgctacgtgcccttccacattcaggtgccccagaagttctttgccatcaagaacctcctcctcctgccgggctcctacacttacgagtggaacttcaggaaggatgtcaacatggtcctgcagagctctctgggcaatgaccttagggtggacggggccagcatcaagtttgacagcgtcaccctctatgctaccttcttccccatggctcacaacaccgcctccacgctcgaggccatgctgaggaacgacaccaacgaccagtccttcaatgactacctctctggggccaacatgctctaccccatccccgccaaggccaccaacgtgcccatctccattccctctcgcaactgggccgccttcagaggctgggcctttacccgccttaagaccaaggaaaccccctccctgggctcgggttttgacccctactttgtctactcgggatccatcccctacctggatggcaccttctacctcaaccacacttttaagaagatatccatcatgtatgactcctccgtcagctggccgggcaatgaccgcctgctcacccccaatgagttcgaggtcaagcgcgccgtggacggcgagggctacaacgtggcccagtgcaacatgaccaaggactggttcctggtgcagatgctggccaactacaacataggctaccagggcttctacatcccagagagctacaaggacaggatgtactccttcttcagaaatttccaacccatgagcaggcaggtggtggacgagaccaaatacaaggactatcaggccattggcatcactcaccagcacaacaactcgggattcgtgggctacctggctcccaccatgcgcgaggggcaggcctaccccgccaacttcccctacccgttgataggcaaaaccgcggtcgacagcgtcacccagaaaaagttcctctgcgaccgcaccctctggcgcatccccttctctagcaacttcatgtccatgggtgcgctcacggacctgggccagaacctgctctatgccaactccgcccatgcgctggacatgacttttgaggtggaccccatggacgagcccacccttctctatattgtgtttgaagtgttcgacgtggtcagagtgcaccagccgcaccgcggtgtcatcgagaccgtgtacctgcgcacgcccttctcggccggcaacgccaccacctaaggagacagcgccgccgcctgcatgacgggttccaccgagcaagagctcagggccatcgccagagacctgggatgcggaccctattttttgggcacctatgacaaacgcttcccgggcttcatctcccgagacaagctcgcctgcgccatcgtcaacacggccgcgcgcgagaccgggggcgtgcactggctggcctttggctgggacccgcgctccaaaacctgctacctcttcgacccctttggcttctccgatcagcgcctcagacagatctatgagtttgagtacgaggggctgctgcgccgcagcgcgcttgcctcctcgcccgaccgctgcatcacccttgagaagtccaccgagaccgtgcaggggccccactcggccgcctgcggtctcttctgctgcatgtttttgcacgcctttgtgcgctggccccagagtcccatggatcgcaaccccaccatgaacttgctcaagggagtgcccaacgccatgctccagagcccccaggtccagcccaccctgcgccacaaccaggaacagctctaccgcttcctggagcgccactccccctacttccgcagtcacagcgcgcacatccggggggccacctctttctgccacttgcaagaaaacatgcaagacggaaaatgatgtacagctcgctttttaataaatgtaaagactgtgcactttatttatacacgggctctttctggttatttattcaacaccgccgtcgccatctagaaatcgaaagggttctgccgcgcgtcgccgtgcgccacgggcagagacacgttgcgatactggaagcggctcgcccacttaaactcgggcaccaccatgcggggcagtggttcctcggggaagttctcgccccacagggtgcgggtcagctgcagcgcgctcaggaggtcgggagccgagatcttgaagtcgcagttggggccggaaccctgcgcgcgcgagttgcggtacacggggttgcagcactggaacaccagcagggccggattatgcacgctggccagcaggctctcgtcgctgatcatgtcgctgtccagatcctccgcgttgctcagggcgaacggggtcatcttgcagacctgcctgcccaggaaaggcggcagcccgggcttgccgttgcagtcgcagcgcaggggcatcagcaggtgcccgcggcccgactgcgcctgcgggtacagcgcgcgcatgaaggcttcgatctgcctgaaagccacctgcgtcttggctccctccgaaaagaacatcccacaggacttgctggagaactggttcgcgggacagctggcatcgtgcaggcagcagcgcgcgtcggtgttggcgatctgcaccacgttgcgaccccaccggttcttcactatcttggccttggaagcctgctccttcagcgcgcgctggccgttctcgctggtcacatccatctctatcacctgctccttgttgatcatgtttgtaccgtgcagacacttcaggtcgccctccgtctgggtgcagcggtgctcccacagcgcgcaaccggtgggctcccaatttttgtgggtcacccccgcgtaggcctgcaggtaggcctgcaagaagcgccccatcatggccacaaaggtcttctggctcgtaaaggtcagctgcaggccgcgatgctcttcgttcagccaggtcttgcagatggcggccagcgcctcggtctgctcgggcagcatcctaaaatttgtcttcaggtcgttatccacgtggtacttgtccatcatggcgcgcgccgcctccatgcccttctcccaggcggacaccatgggcaggcttagggggtttatcacttccaccggcgaggacaccgtactttcgatttcttcttcctccccctcttcccggcgcgcgcccacgctgctgcgcgctctcaccgcctgcaccaaggggtcgtcttcaggcaagcgccgcaccgagcgcttgccgcccttgacctgcttaatcagcaccggcgggttgctgaagcccaccatggtcagcgccgcctgctcttcttcgtcttcgctgtctaccactatctctggggaagggcttctccgctctgcggcggcgcgcttcttttttttcttgggagcggccgtgatggagtccgccacggcgacggaggtcgagggcgtggggctgggggtgcgcggtaccagggcctcgtcgccctcggactcttcctctgactccaggcggcggcggagtcgcttctttgggggcgcgcgcgtcagcggcggcggagacggggacggggacggggacgggacgccctccacagggggtggtcttcgcgcagacccgcggccgcgctcgggggtcttctcgagctggtcttggtcccgactggccattgtatcctcctcctcctaggcagagagacataaggagtctatcatgcaagtcgagaaggaggagagcttaaccaccccctctgagaccgccgatgcgcccgccgtcgccgtcgcccccgctgccgccgacgcgcccgccacaccgagcgacacccccgcggacccccccgccgacgcacccctgttcgaggaagcggccgtggagcaggacccgggctttgtctcggcagaggaggatttgcgagaggaggaggataaggagaagaagccctcagtgccaaaagatgataaagagcaagacgagcacgacgcagatgcacaccagggtgaagtcgggcggggggacggagggcatgacggcgccgactacctagacgaagggaacgacgtgctcttgaagcacctgcatcgtcagtgcgccattgtttgcgacgctctgcaggagcgcagcgaagtgcccctcagcgtggcggaggtcagccacgcctacgagctcagcctcttctccccccgggtgcccccccgccgccgcgaaaacggcacatgcgagcccaacccgcgcctcaacttctaccccgcctttgtggtacccgaggtcctggccacctatcacatcttctttcaaaattgcaagatccccctctcgtgccgcgccaaccgtagccgcgccgataagatgctggccctgcgccagggcgaccacatacctgatatcgccgctttggaagatgtaccaaagatcttcgagggtctgggtcgcaacgagaagcgggcagcaaactctctgcaacaggaaaacagcgaaaatgagagtcacaccggggtactggtggagctcgagggcgacaacgcccgcctggcggtggtcaagcgcagcatcgaggtcacccactttgcctaccccgcgctaaacctgccccccaaagtcatgaacgcggccatggacgggctgatcatgcgccgcggccggcccctcgctccagatgcaaacttgcatgaggagaccgaggacggccagcccgtggtcagcgacgagcagctggcgcgctggctggagaccgcggaccccgccgaactggaggagcggcgcaagatgatgatggccgtggtgctggtcaccgtagagctggagtgtctgcagcgcttcttcggcgaccccgagatgcagagaaaggtcgaggagaccctgcactacaccttccgccagggctacgtgcgccaggcttgcaagatctccaacgtggagctcagcaacctggtgtcctacctgggcatcttgcatgagaaccgcctcgggcagagcgtgctgcactccaccctgcgcggggaggcgcgccgcgactacgtgcgcgactgcgtttacctcttcctctgctacacctggcagacggccatgggggtctggcagcagtgcctggaggagcgcaacctcaaggagctggagaagctcctgcagcgcgcgctcaaagatctctggacgggctacaacgagcgctcggtggccgccgcgctggccgacctcatcttccccgagcgcctgctcaaaaccctccagcaggggctgcccgacttcaccagccaaagcatgttgcaaaacttcaggaactttatcctggagcgttctggcatcctacccgccacctgctgcgccctgcccagcgactttgtccccctcgtgtaccgcgagtgccccccgccgctgtggggtcactgctacctgttccaactggccaactacctgtcctaccacgcggacctcatggaggactccagcggcgaggggctcatggagtgccactgccgctgcaacctctgcacgccccaccgctccctggtctgcaacacccaactgctcagcgagagtcagattatcggtaccttcgagctacagggtccgtcctcctcagacgagaagtccgcggctccggggctaaaactcactccggggctgtggacttccgcctacctgcgcaaatttgtacctgaagactaccacgcccacgagatcaggttttacgaagaccaatcccgcccgcccaaggcggagctgaccgcctgcgtcatcacccagggcgagatcctaggccaattgcaagccatccaaaaagcccgccaagactttttgctgaagaagggtcggggggtgtatctggacccccagtcgggtgaggagctcaacccggttcccccgctgccgccgccgcgggaccttgcttcccaggataagcatcgccatggctcccagaaagaagcagcagcggccgccactgccgccaccccacatgctggaggaagaggaggaatactgggacagtcaggcagaggaggtttcggacgaggaggagccggagacggagatggaagagtgggaggaggacagcttagacgaggaggcttccgaagccgaagaggcagacgcaacaccgtcaccctcggccgcagccccctcgcaggcgcccccgaagtccgctcccagcatcagcagcaacagcagcgctataacctccgctcctccaccgccgcgacccacggccgaccgcagacccaaccgtagatgggacaccaccggaaccggggccggtaagtcctccgggagaggcaagcaagcgcagcgccaaggctaccgctcgtggcgcgctcacaagaacgccatagtcgcttgcttgcaagactgcggggggaacatctccttcgcccgccgcttcctgctcttccaccacggtgtggccttcccccgtaacgtcctgcattactaccgtcatctctacagcccctactgcggcggcagtgagccagagacggtcggcggcggcggcggcgcccgtttcggcgcctaggaagacccagggcaagacttcagccaagaaactcgcggcggccgcggcgaacgcggtcgcgggggccctgcgcctgacggtgaacgaacccctgtcgacccgcgaactgaggaaccgaatcttccccactctctatgccatcttccagcagagcagagggcaggatcaggaactgaaagtaaaaaacaggtctctgcgctccctcacccgcagctgtctgtatcacaagagcgaagaccagcttcggcgcacgctggaggacgctgaggcactcttcagcaaatactgcgcgctcactcttaaggactagctccgcgcccttctcgaatttaggcgggaacgcctacgtcatcgcagcgccgccgtcatgagcaaggacattcccacgccatacatgtggagctatcagccgcagatgggactcgcggcgggcgcctcccaagactactccacccgcatgaactggctcagtgccggcccacacatgatctcacaggttaatgatatccgcacccatcgaaaccaaatattggtggagcaggcggcaattaccaccacgccccgcaataatcccaaccccagggagtggcccgcgtccctggtgtatcaggaaattcccggccccaccaccgtactacttccgcgtgattcccaggccgaagtccaaatgactaactcaggggcacagctcgcgggcggctgtcgtcacagggtgcggcctcctcgccagggtataactcacctggagatccgaggcagaggtattcagctcaacgacgagtcggtgagctcctcgctcggtctcagacctgacgggaccttccagatagccggagccggccgatcttccttcacgccccgccaggcgtacctgactctgcaaagctcgtcctcggcgccgcgctcgggcggcatcgggactctccagttcgtgcaggagtttgtgccctcggtctacttcaaccccttctcgggctctcccggtcgctacccggaccagttcatctcgaactttgacgccgcgagggactcggtggacggctacgactgaatgtcgggtggacccggtgcagagcaacttcgcctgaagcacctcgaccactgccgccgccctcagtgctttgcccgctgtcagaccggtgagttccagtacttttccctgcccgactcgcacccggacggcccggcgcacggggtgcgctttttcatcccgagtcaggtgcgctctaccctaatcagggagtttaccgcccgtcccctactggcggagttggaaaaggggccttctatcctaaccattgcctgcatctgctctaaccctggattgcaccaagatctttgctgtcatttgtgtgctgagtataataaaggctgagatcagaatctactcgggctcctgtcgccatcctgtcaacgccaccgtccaagcccggcccgatcagcccgaggtgaacctcacctgcggtctgcaccggcgcctgaggaaatacctagcttggtactacaacagcactccctttgtggtttacaacagctttgaccaggacggggtctcactgagggataacctctcgaacctgagctactccatcaggaagaacagcaccctcgagctacttcctccttacctgcccgggacttaccagtgtgtcaccggtccctgcacccacacccacctgttgatcgtaaacgactctcttccgagaacagacctcaataactcctcttcgcagttccccagaacaggaggtgagctcaggaaaccccgggtaaagaagggtggacgagagttaacacttgtggggtttctggtgtatgtgacgctggtggtggctcttttgattaaggcttttccttccatgtctgaactctccctcttcttttatgaacaactcgactagtgctaacgggaccctacccaacgaatcgggattgaatatcggtaaccaggttgcagtttcacttttgattaccttcatagtcctcttcctgctagtgctgtcgcttctgtgcctgcggatcgggggctgctgcatccacgtttatatctggtgctggctgtttagaaggttcggagaccatcgcaggtagaataaacatgctgctgcttaccctctttgtcctggcgctggccgccagctgccaagccttttccgaggctgactttatagagccccagtgtaatgtgacttttaaagcccatgcacagcgttgtcatactataatcaaatgtgccaccgaacacgatgaataccttatccagtataaagataaatcacacaaagtggcacttgttgacatctggaaacccgaagaccctttggaatacaatgtgaccgttttccagggtgacctcttcaaaatttacaattacactttcccatttgaccagatgtgtgactttgtcatgtacatggaaaagcagcacaagctgtggcctccgactccccagggctgtgtggaaaatccaggctctttctgcatgatctctctctgtgtaactgtgctggcactaatactcacgcttttgtatatcagatttaaatcaaggcaaagcttcattgatgaaaagaaaatgccttaatcgctttcacgcttgattgctaacaccgggtttttatccgcagaatgattggaatcaccctactaatcacctccctccttgcgattgcccatgggttggaacgaatcgaagtccctgtgggggccaatgttaccctggtggggcctgtcggcaatgctacattaatgtgggaaaaatatactaaaaatcaatgggtctcttactgcactaacaaaaatagccacaagcccagagccatctgcgatgggcaaaatctaaccttgattgatgttcaattgctggatgcgggctactattatgggcagctgggtacaatgattaattactggagaccccacagagattacatgctccacgtagtaaagggtccccttagcagcccacccactaccacctctactacccccactaccaccactactcccaccaccagcactgccgcccagcctcctcatagcagaacaaccacttttatcaattccaagtcccactccccccacattgccggcgggccctccgcctcagactccgaaaccaccgagatctgcttctgcaaatgctctgacgccattgcccaggatttggaagatcacgaggaagatgagcatgacttcgcagatgcatgccaggcatcagagccagaagcgctgccggtggccctcaaacagtatgcagacccccacaccacccccgaccttcctccaccttcccagaagccaagtttcctgggggaaaatgaaactctgcctctctccatactcgctctgacatctgttgctatgttgaccgctctgctggtgcttctatgctctatatgctacctgatctgctgcagaaagaaaaaatctcacggccatgctcaccagcccctcatgcacttcccttaccctccagagctgggcgaccacaaactttaagtctgcagtaactatctgcccatcccttgtcagtcgacagcgatgagccccactaatctaacggcctctggacttacaacatcgtctcttaatgagaccaccgctcctcaagacctgtacgatggtgtctccgcgctggttaaccagtgggatcacctgggcatatggtggctcctcataggagcagtgaccctgtgcctaatcctggtctggatcatctgctgcatcaaaagcagaagacccaggcggcggcccatctacaggccctttgtcatcacacctgaagatgatgatgacaccacttccaggctgcagaggctaaagcagctactcttctcttttacagcatggtaaattgaatcatgcctcgcattttcatctacttgtctctccttccactttttctgggctcttctacattggccgctgtgtcccacatcgaggtagactgcctcacgcccttcacagtctacctgcttttcggctttgtcatctgcacctttgtctgcagcgttatcactgtagtgatctgcttcatacagtgcatcgactacgtctgcgtgcgggtggcttactttagacaccacccccagtatcgcaacagggacatagcggctctcctaagacttgtttaaaatcatggccaaattaactgtgattggtcttctgatcatctgctgcgtcctagccgcgattgggactcaagctcctaccaccaccagcgctcccagaaagagacatgtatcctgcagcttcaagcgtccctggaatataccccaatgctttactgatgaacctgaaatctctttggcttggtacttcagcgtcaccgcccttcttatcttctgcagtacggttattgcccttgccatctacccttcccttgacctgggctggaatgctgtcaactctatggaatatcccaccttcccagaaccagacctgccagacctggttgttctaaacgcgtttcctcctcctgctcccgttcaaaatcagtttcgccctccgtcccccacgcccactgaggtcagctactttaatctaacaggcggagatgactgaaaacctagacctagaaatggacggtctctgcagcgagcaacgcacactagagaggcgccggcaaaaagagctcgagcgtcttaaacaagagctccaagacgcggtggccatacaccagtgcaaaaaaggtgtcttctgtctggtaaaacaggccacgctcacctatgaaaaaacaggtgacacccaccgcctaggatacaagctgcccacacagcgccaaaagttcgccctcatgataggcgaacaacccatcaccgtgacccagcactccgtggagacagaaggctgcatacatgctccctgtaggggcgctgactgcctctacaccttgatcaaaaccctctgcggtctcagagaccttatccctttcaattaatcataactgtaatcaataaaaaatcacttacttgaaatctgatagcaagcctctgtccaattttttcagcaacacttccttcccctcctcccaactctggtactctaggcgcctcctagctgcaaacttcctccacagtctgaagggaatgtcagattcctcctcctgtccctccgcacccacgatcttcatgttgttgcagatgaaacgcgcgagatcgtctgacgagaccttcaaccccgtgtacccctacgataccgagatcgctccgacttctgtccctttccttacccctccctttgtgtcatccgcaggaatgcaagaaaatccagctggggtgctgtccctgcacttgtcagagccccttaccacccacaatggggccctgactctaaaaatggggggcggcctgaccctggacaaggaagggaatctcacttcccaaaacatcaccagtgtcgatccccctctcaaaaaaagcaagaacaacatcagccttcagaccgccgcacccctcgccgtcagctccggggccctaacactttttgccactccccccctagcggtcagtggtgacaaccttactgtgcagtctcaggcccctctcactttggaagactcaaaactaactctggccaccaaaggacccctaactgtgtccgaaggcaaacttgtc

SEQ ID NO: 22 - DNA, 32728 bp, synthetic construct
  misc_feature 1..32728: note = Adenovirus vector nucleotide sequences
  source 1..32728: mol_type = other DNA
catcatcaataatataccttattttggattgtggccaatatgataatgaggtgggcggggagaggcggggcgggtgacgtaggacgcgcgagtagggttgggaggtgtggcggaagtgtggcatttgcaagtgggaggagctcacatgcaagcttccgtcgcggaaaatgtgacgtttttgatgagcgccgcctacctccggaagtgccaattttcgcgcgcttttcaccggatatcgtagtaattttgggcgggaccatgtaagatttggccattttcgcgcgaaaagtgaaacggggaagtgaaaactgaataatagggcgttagtcatagcgcgtaatatttaccgagggccgagggactttgaccgattacgtggaggactcgcccaggtgttttttacgtgaatttccgcgttccgggtcaaagtctccgtttttattgtcaccgtcatttgacgcggagggtatttaaacccgctgcgctcctcaagaggccactcttgagtgccagcgagaagagttttctcctctgctccgcttcggtgatcgaaaaatgagacacatagcctgcactccgggtcttttgtccggtcgggcggcggccgagcttttggacgctttgatcaatgatgtcctaagcgatgattttccgtctactacccactttagcccacctactcttcacgaactgtacgatctggatgtactggtggatgtgaacgatcccaacgaggaggcggtttctgcgttttttcccgagtctgcgctgttggccgctcaggagggatttgacctacacactccgccgcctattttagagtctccgctgccggagcccagtggtataccttatatgcctgaactgcttcccgaagtggtagacctgacctgccacgagcctggctttccgcccagcgacgatgagggtgagccttttgttttagactttgctgagatacctgggcacggttgcaggtcttgtgcatatcatcagagggttaccggagaccccgaggttaagtgttcgctgtgctatatgaggatgacctcttcctttatctacagtaagtttttgtctaggtgggcttttgggtaggtgggttttgtgtcagaacaggtgtaaacgttgcttgtgttttttgtacctgtaggtccggtgtccgagccagacccggagcccgaccgcgatcccgagccggatcccgagcctcctcgcaggacaaggaaactaccttccattctgtgcaagtctcagacacctgtaaggaccagcgaggcagacagcaccgactctggcacttctacctctccccctgaaattcacccagtggttcctctgggtatacataaacctgttgctgttaaagtttgcgggcgacgccctgcagtacagtgcattgaggacttgcttcacgatcccgaggaacctttggacttgagccttaaacgccctaggcaataaaccccacctaagtaataaaccccacctaagtaataaaccctgccgcccttggttattgagatgacgcccaatgtttgcttttgaatgacttcatgtgtgtaataaaagtgagtgtgatcataggtctcttgtttgtctgggcggggcttaagggtatataagtctcttggggctaaacttggttacacttgaccccaatggaggcgtgggggtgcttggaggagtttgcggacgtgcgccgtttgctggacgagagctctagcaatacctatactatttggaggtatctgtggggctctactcaggccaagttggtttccagaattaagcaggattacaagtgcgattttgaagagctttttagttcctgcggtgagcttttgcaatccttgaatctgggccatcaggctattttccaggaaaaggttctctcgactttggatttttccactcccgggcgcaccgccgcttgtgtggcttttgtgtcttttgtgcaagataaatggagcgaggagacccacctgagtcacggctacgtactggatttcatggcgatggctctttggagggctcacaacaaatggaagattcagaaggaactgtacggttccgccctacgtcgtccacttctgtcgcgacaggggctgaggtttcccgaccatcggcagcatcagaatctggaagacgagtcggaggagcgagcggaggagaagatcagcttgagagccggcctggaccctcctcaggaggaatgaatctcccgcaggtggttgacctgtttccagaactgagacgggtcctgactatcagggaggatggtcagtttgtgaagaagtttaagagggatcggggtgagggagatgatgaggcggctagcaatttagcttttagtctgatgactcgccaccgaccggaatgtattacctatcagcagattaaggagagttgtgccaacgagctggatcttttgggtcagaagtatagcatagaacagcttaccacttactggcttcagcctggggatgattgggaagaggcgatcagggtgtatgcaaaggtggccctgcggcccgattgcaagtataagattactaagttggttaatattagaaactgctgctatatttctgggaacggggccgaagtggagatagatactcaggacagggtggcttttaggtgttgcatgataaacatgtggcccgggatactggggatggatggggtggtattcatgaatgtgaggtttacgggccccaactttaatggcacggtgttcatgggcaacaccaacttgctcctgcatggtgcgagtttctatgggtttaataacacctgtatagaggcctggaccgatgtaaaggttcgaggttgttccttttatagctgttggaaggcggtggtgtgtcgccctaaaagcaggggttctgtgaaaaaatgcttgtttgaaaggtgcaccttaggcatcctctctgagggcaactccagggtgcgccataatgtggcttcgaactgcggttgcttcatgcaagtgaagggggtgagcgttatcaagcataactcggtgtgtggaaactgcgaggatcgcgcctcccagatgctgacctgctttgatggcaactgtcacctgttgaagaccattcatataagcagccaccccagaaaggcctggcccgtgtttgagcataacatcttgacccgctgctccttgcatctgggggtcaggaggggtatgttcctgccttaccagtgtaactttagccacactaaaatcctgctggaacccgagtgcatgaccaaggtcagcctgaatggtgtgtttgatgtgactctgaaaatctggaaggtgctgaggtatgatgagaccaggaccaggtgccgaccctgcgagtgcggcggcaagcacatgagaaatcagcctgtgatgttggatgtgaccgaggagcttaggcctgaccatctggtgctggcctgcaccagggccgagtttgggtctagcgatgaggataccgattgaggtgggtaaggtgggcgtggctagaagggtggggcgtgtataaattgggggtctaagggtctctctgttttgtcttgcaacagccgccgccatgagcgacaccggcaacagctttgatggaagcatctttagcccctatctgacagtgcgcatgcctcactgggctggagtgcgtcagaatgtgatgggttccaacgtggatggacgccccgttctgccttcaaattcgtctacaatggcctacgcgaccgtgggaggaactccgctggacgccgcgacctccgccgccgcctccgccgccgccgcgaccgcgcgcagcatggctacggacctttacagctctttggtggcgagcggcgcggcctctcgcgcgtctgctcgggatgagaaactgaccgctctgctgcttaaactggaagacttgacccgggagctgggtcaactgacccagcaggtctccagcttgcgtgagagcagccttgcctccccctaatggcccataatataaataaaagccagtctgtttggattaagcaagtgtatgttctttatttaactctccgcgcgcggtaagcccgggaccagcggtctcggtcgtttagggtgcggtggattctttccaacacgtggtacaggtggctctggatgtttagatacatgggcatgagtccatccctggggtggaggtagcaccactgcagagcttcgtgctcgggggtggtgttgtatatgatccagtcgtagcaggagcgctgggcgtggtgctgaaaaatgtccttaagcaagaggcttatagctagggggaggcccttggtgtaagtgtttacaaatctgctcagttgggaggggtgcatccggggggatataatgtgcatcttggactggatttttaggttggctatgttcccacccagatcccttctgggattcatgttgtgcaggaccaccagcacggtatatccagtgcacttgggaaatttatcgtggagcttagacgggaatgcatggaagaacttggagacgcccttgtggcctcccagattttccatacattcgtccatgatgatggcaatgggcccgtgggaagctgcctgagcaaaaatgtttctgggatcgctcacatcgtagttatgttccagggtgaggtcatcataggacatctttacgaatcgggggcggagggtcccggactgggggatgatggtaccctcgggccccggggcgtagttcccctcacagatctgcatctcccaggctttcatttcagagggagggatcatatccacctgcggagcgatgaaaaacacagtttctggcgcaggggagattaactgggatgagagcaggtttctgagcagctgtgactttccacagccggtgggcccatatatcacgcctatcaccggctgcagctggtagttaagagagctgcagctgccgtcctcccggagcaggggggccacctcgttcagcatatccctgacgtggatgttctccctgaccaattccgccagaaggcgctcgccgcccagcgaaagcagctcttgcaaggaagcaaaatttttcagcggttttaggccgtcggccgtgggcatgtttttcagcgtctgggtcagcagttccagcctgtcccacagctcggtgatgtgctctacggcatctcgatccagcagatctcctcgtttcgcgggttggggcggctttcgctgtagggcaccagccgatgggcgtccagcggggccagagtcatgtccttccatgggcgcagggtcctcgtcagggtggtctgggtcacggtgaaggggtgcgctccgggttgggcgctggccagggtgcgcttgaggctggttctgctggtgctgaatcgctgccgctcttcgccctgcgcgtcggccaggtagcatttgaccatggtctcgtagtcgagaccctcggcggcgtgccccttggcgcggagctttcccttggaggtggcgccgcacgaggggcactgcaggctcttcagggcgtagagcttgggagcgagaaacacggactctggggagtaggcgtccgcgccgcaggaagcgcagaccgtctcgcattccaccagccaagtgagctccgggcggtcagggtcaaaaaccaggttgcccccatgctttttgatgcgtttcttacctcggctctccatgaggcggtgtcccttctcggtgacgaagaggctgtccgtgtccccgtagaccgacttcaggggcctgtcttccagcggagtgcctctgtcctcctcgtagagaaactctgaccactctgagacgaaggcccgcgtccaggccaggacgaaggaggccacgtgggaggggtagcggtcgttgtccactagcgggtccaccttctccagggtgtgcaggcacatgtccccctcctccgcgtccagaaaagtgattggcttgtaggtgtaggacacgtgaccgggggttcccgacgggggggtataaaagggggtgggcgccctttcatcttcactctcttccgcatcgctgtctgcgagggccagctgctggggtaagtattccctctcgaaggcgggcatgacctcagcgctcaggttgtcagtttctaaaaatgaggaggatttgatgttcacctgtccggaggtgatacctttgagggtacctgggtccatctggtcagaaaacactatttttttgttgtcaagcttggtggcgaacgacccgtagagggcgttggagagcagcttggcgatggagcgcagggtctggtttttgtcgcggtcggctcgctccttggccgcgatgttgagttgcacgtactcgcgggccacgcacttccactcggggaagacggtggtgcgctcgtctgggattaggcgcaccctccagcctcggttgtgcagggtgaccatgtcgacgctggtggcgacctcgccgcgcaggcgctcgttggtccagcagaggcggccgcccttgcgcgagcagaaggggggtagggggtccagctggtcctcgtttggggggtccgcgtcgatggtgaagaccccggggagcaagcgcgggtcaaagtagtcgatcttgcaagcttgcatgtccagagcccgctgccattcgcgggcggcgagcgcgcgctcgtaggggttgaggggcgggccccagggcatggggtgggtgagcgcggaggcgtacatgccgcagatgtcatacacgtacaggggttccctgaggatgccgaggtaggtggggtagcagcgccccccgcggatgctggcgcgcacgtagtcatagagctcgtgggagggggccagcatgttgggcccgaggttggtgcgctgggggcgctcggcgcggaaggcgatctgcctgaagatggcatgggagttggaggagatggtgggccgctggaagacgttgaagcttgcttcttgcaagcccaccgagtccctgacgaaggaggcgtaggactcgcgcagcttgtgcaccagctcggcggtgacctggacgtcgagcgcgcagtagtcgagggtctcgcggatgatgtcatacttatcctcccccttctttttccacagctcgcggttgaggacgaactcttcgcggtctttccagtactcttggaggggaaacccgtccgtgtccgaacggtaagagcctagcatgtagaactggttgacggcctggtaggggcaacagcccttctccacgggcagcgcgtaggcctgcgccgccttgcggagggaggtgtgggtgagggcgaaagtgtccctgaccatgactttgaggtattgatgtttgaagtctgtgtcatcgcagccgccctgttcccacagggtgtagtccgtgcgctttttggagcgcgggttgggcagggagaaggtgaggtcattgaagaggatcttccccgctcgaggcatgaagtttctggtgatgcgaaagggccctgggaccgaggagcggttgttgatgacctgggcggccaggacgatctcgtcaaagccgtttatgttgtggcccacgatgtagagctccaaaaagcggggctggcccttgatggaggggagctttttgagttcctcgtaggtgagctcctcgggcgattccaggccgtgctcctccagggcccagtcttgcaagtgagggttggccgccaggaaggatcgccagaggtcgcgggccatgagggtctgcaggcggtcgcggaaggttctgaactgtcgccccacggccatcttttcgggggtgatgcagtagaaggtgagggggtctttctcccaggggtcccatctgagctctcgggcgaggtcgcgcgcggcggcgaccagagcctcgtcgccccccagtttcatgaccagcatgaagggcacgagctgcttgccaaaggctcccatccaagtgtaggtctctacatcgtaggtgacaaagaggcgctccgtgcgaggatgagagccgatcgggaagaactggatctcccgccaccagttggaggattggctgttgatgtggtgaaagtagaagtcccgtctgcgggccgagcactcgtgctggcttttgtaaaagcgaccgcagtactggcagcgctgcacgggttgtatatcttgcacgaggtgaacctggcgacctctgacgaggaagcgcagcgggaatctaagtcccccgcctggggtcccgtgtggctggtggtcttctactttggttgtctggccgccagcatctgtctcctggagggcgatggtggagcagaccaccacgccgcgagagccgcaggtccagatctcggcgctcggcgggcggagtttgatgacgacatcgcgcacattggagctgtccatggtctccagctcccgcggcggcaggtcagctgggagttcctggaggttcacctcgcagagacgggtcaaggcgcgggcagtgttgagatggtatctgatttcaaggggcgtgttggcggcggagtcgatggcttgcaggaggccgcagccccggggggccacgatggttccccgcggggcgcgaggggaggcggaagctgggggtgtgttcagaagcggtgacgcgggcgggcccccggaggtagggggggttccggccccacaggcatgggcggcaggggcacgtcttcgccgcgcgcgggcaggggctggtgctggctccgaagagcgcttgcgtgcgcgacgacgcgacggttggtgtcctgtatctgacgcctctgagtgaagaccacgggtcccgtgaccttgaacctgaaagagagttcgacagaatcaatctcggcatcgttgacagcggcctggcgcaggatctcctgcacgtcgcccgagttgtcctggtaggcgatctctgccatgaactgctcgatctcttcttcctggagatctcctcgtccggcgcgctccacggtggccgccaggtcgttggagatgcgacccatgagctgtgagaaggcgttgagcccgccctcgttccagacccggctgtagaccacgcccccctcggcgtcgcgagcgcgcatgaccacctgggccaggttgagctccacgtgtcgcgtgaagacggcgtagttgcgcaggcgctggaaaaggtagttcagggtggtggcggtgtgctcggcgacgaagaagtacatgacccagcgccgcaacgtggattcattgatgtcccccaaggcctccaggcgctccatggcctcgtagaagtccacggcgaagttgaaaaactgggagttgcgagcggacacggtcaactcctcctccagaagacggatgagctcggcgacagtgttgcgcacctcgcgctcgaaggccacggggggcgcttcttcctcttccacctcttcttccatgatcgcttcttcttcttcctcagccgggacgggagggggcggcggcggcgggggaggggcgcggcggcggcggcggcgcaccgggaggcggtcgatgaagcgctcgatcatctccccccgcatgcggcgcatggtctcggtgacggcgcggccgttctcccgggggcgcagctcgaagacgccgcctctcatctcgccgcggggcgagcggccgtgaggtagcgagacggcgctgactatgcatcttaacaattgctgtgtaggtacaccgccgagggacctgattgagtccagatccaccggatccgaaaacctttggaggaaagcgtctatccagtcgcagtcgcaaggtaggctgagcaccgtggcgggcgggggcgggtctggagagttcctggcggagatgctgctgatgatgtaattaaagtaggcggtcttgagaaggcggatggtggacaggagcaccatgtctttgggtccggcctgttggatgcggaggcggtcggccatgccccaggcctcgttctgacaccggcgcaggtctttgtagtagtcttgcatgagtctttccaccggcacctcttctccttcctcttctccatctcgccggtggtttctcgcgccgcccatgcgcgtgaccccaaagcccctgagcggctgcagcagggccaggtcggcgaccacgcgctcggccaagatggcctgctgcacctgagtgagggtcctctcgaagtcatccatgtccacgaagcggtggtaggcgcccgtgttgatggtgtaggtgcagttggccatgacggaccagttgacggtctggtgtcccggctgcgagagctccgtgtaccgcaggcgcgagaaggcgcgggaatcgaacacgtagtcgttgcaagtccgcaccagatactggtagcccaccaggaagtgcggcggaggttggcgatagaggggccagcgctgggtggcgggggcgccgggcgccaggtcttccagcatgaggcggtggtatccgtagatgtacctggacatccaggtgatgccggcggcggtggtggtggcgcgcgcgtagtcgcggacccggttccagatgtttcgcaggggcgagaagtgttccatggtcggcacgctctggccggtgaggcgcgcgcagtcgttgacgctctatacacacacaaaaacgaaagcgtttacagggctttcgttctgtagcctggaggaaagtaaatgggttgggttgcggtgtgccccggttcgagaccaagctgagctcggccggctgaagccgcagctaacgtggtattggcagtcccgtctcgacccaggccctgtatcctccaggatacggtcgagagcccttttgctttcttggccaagcgcccgtggcgcgatctgggatagatggtcgcgatgagaggacaaaagcggctcgcttccgtagtctggagaaacaatcgccagggttgcgttgcggcgtaccccggttcgagcccctatggcggcttgaatcggccggaaccgcggctaacgagggccgtggcagccccgtcctcaggaccccgccagccgacttctccagttacgggagcgagccccttttgttttttattttttagatgcatcccgtgctgcggcagatgcgcccctcgccccggcccgatcagcagcagcaacagcaggcatgcagacccccctctcccctttccgccccggtcaccacggccgcggcggccgtgtcgggcgcggggggcgcgctggagtcagatgagccaccgcggcggcgacctaggcagtatctggacttggaagagggcgagggactggcgcggctgggggcgaactctccagagcgccacccgcgggtgcagttgaaaagggacgcgcgcgaggcgtacctgccgcggcagaacctgtttcgcgaccgcgggggcgaggagcccgaggagatgcgagactgcaggttccaagcggggcgcgagctgcggcgcgggctggacagacagcgcctgctgcgcgaggaggactttgagcccgacacgcagacgggcatcagccccgcgcgcgcgcacgtagccgcggccgacctggtgaccgcctacgagcagacggtaaaccaggagcgcaacttccaaaagagcttcaacaaccacgtgcgcacgctggtggcgcgcgaggaggtgaccctgggtctcatgcatctgtgggacctggtggaggcgatcgtgcagaaccccagcagcaagcccctgaccgcgcagctgttcctggtggtgcagcacagcagggacaacgaggccttcagggaggcgctgctgaacatcaccgagccggaggggcgctggctcctggacctgataaacatcctgcagagcatagtggtgcaggagcgcagcctgagcctggccgagaaggtggcggccatcaactactctatgctgagcctgggcaagttctacgcccgcaagatctacaagaccccctacgtgcccatagacaaggaggtgaagatagacagcttctacatgcgcatggcgctgaaggtgctgaccctgagcgacgacctgggagtgtaccgcaacgagcgcatccacaaggccgtgagcgccagccggcggcgcgagctgagcgaccgcgagctgatgcacagtctgcagcgcgcgctgaccggcgcgggcgagggcgacagggaggtcgagtcctacttcgacatgggggccgacctgcactggcagccgagccgccgcgccctggaggcggcgggggcgtacggcggccccctggcggccgatgaccaggaagaggaggactatgagctagaggagggcgagtacctggaggactgacctggctggtggtgttttggtatagatgcaagatccgaacgtggcggacccggcggtccgggcggcgctgcaaagccagccgtccggcattaactcctctgacgactgggccgcggccatgggtcgcatcatggccctgaccgcgcgcaaccccgaggctttcaggcagcagcctcaggccaaccggctggcggccatcttggaagcggtagtgcccgcgcgctccaaccccacccacgagaaggtgctggccatagtcaacgcgctggcggagagcagggccatccgcgcggacgaggccggactggtgtacgatgcgctgctgcagcgggtggcgcggtacaacagcggcaacgtgcagaccaacctggaccgcctggtgacggacgtgcgcgaggccgtggcgcagcgcgagcgcttgcatcaggacggtaacctgggctcgctggtggcgctaaacgccttcctcagcacccagccggccaacgtaccgcgggggcaggaggactacaccaactttttgagcgcgctgcggctgatggtgaccgaggtccctcagagcgaggtgtaccagtcggggcccgactacttcttccagaccagcagacagggcttgcaaaccgtgaacctgagccaggctttcaagaacctgcgggggctgtggggagtgaaggcgcccaccggcgaccgggctacggtgtccagcctgctaacccccaactcgcgcctgctgctgctgctgatcgcgcccttcacggacagcgggagcgtctcgcgggagacctatctgggccacctgctgacgctgtaccgcgaggccatcgggcaggcgcaggtggacgagcacaccttccaagagatcaccagcgtgagccacgcgctggggcaggaggacacgggcagcctgcaggcgaccctgaactacctgctgaccaacaggcggcagaagattcccacgctgcacagcctgacccaggaggaggagcgcatcttgcgctacgtgcagcagagcgtgagcctgaacctgatgcgcgacggcgtgacgcccagcgtggcgctggacatgaccgcgcgcaacatggaaccgggcatgtacgcctcccaccggccgtttatcaaccgcctgatggactacttgcatcgggcggcggccgtgaaccccgagtacttcactaatgccattctgaatccccactggatgccccctccgggtttctacaacggggactttgaggtgcccgaggtcaacgacgggttcctctgggatgacatggatgacagtgtgttctcacccaacccgctgcgcgccgcgtctctgcgattgaaggagggctctgacagggaaggaccgaggagtctggcctcctccctggctctgggagcggtgggcgccacgggcgcggcggcgcggggcagtagccccttccccagcctggcagactctctgaacagcgggcgggtgagcaggccccgcttgctaggcgaggaggagtatctgaacaactccctgctgcagcccgcgagggacaagaacgctcagcggcagcagtttcccaacaatgggatagagagcctggtggacaagatgtccagatggaagacgtatgcgcaggagtacaaggagtgggaggaccgccagccgcggcccttgccgccccctaggcagcgctggcagcggcgcgcgtccaaccgccgctggaggcaggggcccgaggacgatgatgactctgcagatgacagcagcgtgttggacctgggcgggagcgggaaccccttttcgcacctgcgcccacgcctgggcaagatgttttaaaagaaaaaaaaaaataaaactcaccaaggccatggcgacgagcgttggttttttgttcccttccttagtatgcggcgcgcggcgatgttcgaggaggggcctcccccctcttacgagagcgcgatggggatttctcctgcggcgcccctgcagcctccctacgtgcctcctcggtacctgcaacctacaggggggagaaatagcatctgttactctgagctgcagcccctgtacgataccaccagactgtacctggtggacaacaagtccgcggacgtggcctccctgaactaccagaacgaccacagcgattttttgaccacggtgatccaaaacaacgacttcaccccaaccgaggccagcacccagaccataaacctggataacaggtcgaactggggcggcgacctgaagaccatcttgcacaccaacatgcccaacgtgaacgagttcatgttcaccaactcttttaaggcgcgggtgatggtggcgcgcgagcagggggaggcgaagtacgagtgggtggacttcacgctgcccgagggcaactactcagagaccatgactctcgacctgatgaacaatgcgatcgtggaacactatctgaaagtgggcaggcagaacggggtgaaggaaagcgatatcggggtcaagtttgacaccagaaacttccgtctgggctgggaccccgtgaccgggctggtcatgccgggggtctacaccaacgaggcctttcatcccgacatagtgcttctgcccggctgtggggtggacttcacccagagccggctgagcaacctgctgggcattcgcaagcggcagcctttccaggagggtttcaagatcacctatgaggatctgaaggggggcaacattcccgcgctccttgatctggacgcctacgaggagagcttgaaacccgaggagagcgctggcgacagcggcgagagtggcgaggagcaagccggcggcggtggcggcgcgtcggtagaaaacgaaagtacgcccgcagtggcggcggacgctgcggaggtcgagccggaggccatgcagcaggacgcagaggagggcgcacaggagggcgcgcagaaggacatgaacgatggggagatcaggggagacacattcgccacccggggcgaagaaaaagaggcagaggcggcggcggcggcgacggcggaggccgaaaccgaggttgaggcagaggcagagcccgagaccgaagttatggaagacatgaatgatggagaacgtaggggcgacacgttcgccacccggggcgaagagaaggcggcggaggcagaagccgcggctgaggaggcggctgcggctgcggccaagactgaggctgcggctaaggctgaggtcgaagccaatgttgcggttgaggctcaggctgaggaggaggcggcggctgaagcagttaaggaaaaggcccaggcagagcaggaagagaaaaaacctgtcattcaacctctaaaagaagatagcaaaaagcgcagttacaacgtcatcgagggcagcacctttacccagtaccgcagctggtacctggcgtacaactacggcgacccggtcaagggggtgcgctcgtggaccctgctctgcacgccggacgtcacctgcggctccgagcagatgtactggtcgctgccgaacatgatgcaagacccggtgaccttccgctccacgcggcaggttagcaacttcccggtggtgggcgccgaactgctgcccgtgcactccaagagtttttacaacgagcaggccgtctactcccagctgatccgccaggccacctctctgacccacgtgttcaatcgctttcccgagaaccagattttggcgcgcccgccggcccccaccatcaccaccgtgagtgaaaacgttcctgccctcacagatcacgggacgctaccgctgcgcaacagcatctcaggagtccagcgagtgaccattactgacgccagacgccggacctgcccctacgtttacaaggccttgggcatagtctcgccgcgcgtcctctccagtcgcactttttaaaacacatctacccacacgttccaaaatcatgtccgtactcatctcacccagcaacaacaccggctgggggctgcgcgcgcccagcaagatgtttggaggggcgaggaagcgctccgaccagcaccctgtgcgcgtgcgcggccactaccgcgcgccctggggagcgcacaagcgcgggcgcacagggcgcaccactgtggacgacgtcattgactccgtagtggagcaagcgcgccactacacacccggcgcgccgaccgcccccgccgtgtccaccgtggaccaggcgatcgaaagcgtggtacagggcgcgcggcactatgccaaccttaaaagtcgccgccgccgcgtggcccgccgccatcgccggagaccccgggccaccgccgccgcgcgccttactaaggctctgctcaggcgcgccaggcgaactggccaccgggccgccatgagggccgcacggcgggctgccgctgccgcaagcgtcgtggccccgcgggcacgaaggcgcgcggccgctgccgccgccgccgccatttccagcttggcctcgacgcggcgcggtaacatatactgggtgcgcgactcggtaaccggcacgcgggtacccgtgcgctttcgccccccgcggaattagcacaagacaacatacacactgagtctcctgctgttgtgtatcccagcggcgaccgtcagcagcggcgacatgtccaagcgcaaaattaaagaagagatgctccaggtcatcgcgccggagatctatgggcccccgaagaaggaggaggatgattacaagccccgcaagctaaagcgggtcaaaaagaaaaagaaagatgatgatgacgaggcggtggagtttgtccgccgcatggcacccaggcgccccgtgcagtggaagggccggcgcgtgcagcgcgttttgcgccccggcaccgcggtggtcttcacgcccggcgagcgctccacgcgcactttcaagcgggtgtacgatgaggtgtacggcgacgaggacctgttggagcaggccaaccagcgctttggggagtttgcatatgggaaacggccccgcgagagtctaaaagaggacctgctggcgctaccgctggacgagggcaatcccaccccgagtctgaagccggtaaccctgcaacaggtgctgcctttgagcgcgcccagcgagcataagcgagggttgaagcgcgaaggcggggacctggcgcccaccgtgcagttgatggtgcccaagcggcagaagctggaggacgtgctggagaaaatgaaagtagagcccgggatccagcccgagatcaaggtccgccccatcaagcaggtggcgcccggcgtgggagtccagaccgtggacgttaggattcccacggaggagatggaaacccaaaccgccactccctcttcggcggccagcgccaccaccggcaccgcttcggtagaggtgcagacggacccctggctacccgccaccgctgttgccgccgccgccccccgttcgcgcgggcgcaagagaaattatccagcggccagcgcgctcatgccccagtacgcactgcatccatccatcgtgcccacccccggctaccgcgggtactcgtaccgcccgcgcagatcagccggcactcgcggccgccgccgccgtgcgaccacaaccagccgccgccgtcgccgccgccgccagccagtgctgacccccgtgtctgtaaggaaggtggctcgctcggggagcacgctggtggtgcccagagcgcgctaccaccccagcatcgtttaaagccggtctctgtatggttcttgcagatatggccctcacttgtcgcctccgcttcccggtgccgggataccgaggaagaactcaccgccgcagaggcatggcgggcagcggtctccgcggcggccgtcgccatcgccggcgcgcaaaaagcaggcgcatgcgcggcggtgtgctgcctctgctaatcccgctaatcgccgcggcgatcggtgccgtacccgggatcgcctccgtggccctgcaggcgtcccagaaacgttgactcttgcaaccttgcaagcttgcattttttggaggaaaaaataaaaaaaaagtctagactctcacgctcgcttggtcctgtgactattttgtagaaaaaaagatggaagacatcaactttgcgtcgctggccccgcgtcacggctcgcgcccgttcatgggagactggacagatatcggcaccagcaatatgagcggtggcgccttcagctggggcagtctgtggagcggccttaaaaattttggttccaccattaagaactatggcaacaaagcgtggaacagcagcacgggccagatgctgagagacaagttgaaagagcagaacttccaggagaaggtggcgcagggcctggcctctggcatcagcggggtggtggacatagctaaccaggccgtgcagaaaaagataaacagtcatctggacccccgtcctcaggtggaggaaatgcctccagcgatggagacggtgtctcccgagggcaaaggcgaaaagcgcccgcggcccgacagagaagagaccctggtgtcacacaccgaggagccgccctcttacgaggaggcagtcaaggccggcctgcccaccactcgccccatagcccccatggccaccggtgtggtgggccacaggcaacacactcccgcaacactagatctgcccccgccgtccgagccgccgcgccagccaaaggcggcgacggtgcccgctccctccacttccgccgccaacagagtgcccctgcgccgcgccgcgagcggcccccgggcctcgcgagttagcggcaactggcagagcacactgaacagcatcgtgggcctgggagtgaggagtgtgaagcgccgccgttgctactgaatgagcaagctagctaacgtgttgtatgtgtgtatgcgtcctatgtcgccgccagaggagctgttgagccgccggcgccgtctgcactccagcgaatttcaagatggcgaccccatcgatgatgcctcagtggtcgtacatgcacatctcgggccaggacgcttcggagtacctgagccccgggctggtgcagttcgcccgcgccacagacacctacttcaacatgagtaacaagttcaggaaccccactgtggcgcccacccacgatgtgaccacggaccggtcgcagcgcctgacgctgcggttcatccccgtggatcgggaggacaccgcctactcttacaaggcgcggttcacgctggccgtgggcgacaaccgcgtgctggacatggcctccacttactttgacatcaggggggtgctggacaggggccccaccttcaagccctactcgggtactgcctacaactccctggcccccaagggcgctcccaattcttgcgagtgggaacaagatgaaccagctcaggcagcaatagctgaagatgaagaagaacttgaagaagaacaagctcaggacgaacaggcgcccactaagaaaacccatgtatacgcccaggcacctctttctggtgaaaaaattactaaggatggtttgcaaataggtgtggatgccacacaggcgggagataaccctatatatgctgataaaacattccaacccgaacctcagataggtgagtctcagtggaacgaggctgatgccacagtagcaggaggcagagtcttaaaaaagaccacccctatgagaccttgctatggatcctatgccaaacctactaatgccaatggcggtcaagggatcatggtggccaatgatcagggagcgcttgaatctaaagttgagatgcaatttttctccaccacaacgtctcttaatgtaagggaaggtgaaaacaatcttcagccaaaagtagtgctatacagcgaagatgttaacttggaatcccctgacactcatttgtcttacaaacctaaaaaggatgacaccaactctaaaatcatgttgggtcagcaagccatgcccaacagacccaacctcattgcttttagggacaactttattggacttatgtactacaacagcacaggcaacatgggagtgctggcaggacaggcctcccagctaaacgctgtggtagacttgcaagacagaaacacagagctgtcataccaactgatgcttgattccattggagacagatcaagatacttttccatgtggaaccaggcagtggacagctatgacccagatgtcagaatcattgaaaaccatggggttgaagatgagctgcccaactattgctttcccctgggcggtattggaattacagacacataccagtgcataaaaccaaccgcagctgctaataacactacatggtctaaggatgaagaatttagtgatcgcaatgaaataggggtgggaaacaacttcgccatggagatcaacatccaggccaacctctggaggaacttcctctatgcgaacgtggggctctacctgccagacaagctcaagtacaaccccaccaacgtggacatctctgacaaccccaacacctatgactacatgaacaagcgtgtggtggctcccggcctggtggactgctttgtcaatgtgggagccaggtggtccctggactacatggacaacgtcaaccccttcaaccaccaccgcaatgcgggtctgcgctaccgctccatgatcctgggcaacgggcgctacgtgcccttccacattcaggtgccccagaagttctttgccatcaagaacctcctcctcctgccgggctcctacacttacgagtggaacttcaggaaggatgtcaacatggtcctgcagagctctctgggcaatgaccttagggtggacggggccagcatcaagtttgacagcgtcaccctctatgctaccttcttccccatggctcacaacaccgcctccacgctcgaggccatgctgaggaacgacaccaacgaccagtccttcaatgactacctctctggggccaacatgctctaccccatccccgccaaggccaccaacgtgcccatctccattccctctcgcaactgggccgccttcagaggctgggcctttacccgccttaagaccaaggaaaccccctccctgggctcgggttttgacccctactttgtctactcgggatccatcccctacctggatggcaccttctacctcaaccacacttttaagaagatatccatcatgtatgactcctccgtcagctggccgggcaatgaccgcctgctcacccccaatgagttcgaggtcaagcgcgccgtggacggcgagggctacaacgtggcccagtgcaacatgaccaaggactggttcctggtgcagatgctggccaactacaacataggctaccagggcttctacatcccagagagctacaaggacaggatgtactccttcttcagaaatttccaacccatgagcaggcaggtggtggacgagaccaaatacaaggactatcaggccattggcatcactcaccagcacaacaactcgggattcgtgggctacctggctcccaccatgcgcgaggggcaggcctaccccgccaacttcccctacccgttgataggcaaaaccgcggtcgacagcgtcacccagaaaaagttcctctgcgaccgcaccctctggcgcatccccttctctagcaacttcatgtccatgggtgcgctcacggacctgggccagaacctgctctatgccaactccgcccatgcgctggacatgacttttgaggtggaccccatggacgagcccacccttctctatattgtgtttgaagtgttcgacgtggtcagagtgcaccagccgcaccgcggtgtcatcgagaccgtgtacctgcgcacgcccttctcggccggcaacgccaccacctaaggagacagcgccgccgcctgcatgacgggttccaccgagcaagagctcagggccatcgccagagacctgggatgcggaccctattttttgggcacctatgacaaacgcttcccgggcttcatctcccgagacaagctcgcctgcgccatcgtcaacacggccgcgcgcgagaccgggggcgtgcactggctggcctttggctgggacccgcgctccaaaacctgctacctcttcgacccctttggcttctccgatcagcgcctcagacagatctatgagtttgagtacgaggggctgctgcgccgcagcgcgcttgcctcctcgcccgaccgctgcatcacccttgagaagtccaccgagaccgtgcaggggccccactcggccgcctgcggtctcttctgctgcatgtttttgcacgcctttgtgcgctggccccagagtcccatggatcgcaaccccaccatgaacttgctcaagggagtgcccaacgccatgctccagagcccccaggtccagcccaccctgcgccacaaccaggaacagctctaccgcttcctggagcgccactccccctacttccgcagtcacagcgcgcacatccggggggccacctctttctgccacttgcaagaaaacatgcaagacggaaaatgatgtacagctcgctttttaataaatgtaaagactgtgcactttatttatacacgggctctttctggttatttattcaacaccgccgtcgccatctagaaatcgaaagggttctgccgcgcgtcgccgtgcgccacgggcagagacacgttgcgatactggaagcggctcgcccacttaaactcgggcaccaccatgcggggcagtggttcctcggggaagttctcgccccacagggtgcgggtcagctgcagcgcgctcaggaggtcgggagccgagatcttgaagtcgcagttggggccggaaccctgcgcgcgcgagttgcggtacacggggttgcagcactggaacaccagcagggccggattatgcacgctggccagcaggctctcgtcgctgatcatgtcgctgtccagatcctccgcgttgctcagggcgaacggggtcatcttgcagacctgcctgcccaggaaaggcggcagcccgggcttgccgttgcagtcgcagcgcaggggcatcagcaggtgcccgcggcccgactgcgcctgcgggtacagcgcgcgcatgaaggcttcgatctgcctgaaagccacctgcgtcttggctccctccgaaaagaacatcccacaggacttgctggagaactggttcgcgggacagctggcatcgtgcaggcagcagcgcgcgtcggtgttggcgatctgcaccacgttgcgaccccaccggttcttcactatcttggccttggaagcctgctccttcagcgcgcgctggccgttctcgctggtcacatccatctctatcacctgctccttgttgatcatgtttgtaccgtgcagacacttcaggtcgccctccgtctgggtgcagcggtgctcccacagcgcgcaaccggtgggctcccaatttttgtgggtcacccccgcgtaggcctgcaggtaggcctgcaagaagcgccccatcatggccacaaaggtcttctggctcgtaaaggtcagctgcaggccgcgatgctcttcgttcagccaggtcttgcagatggcggccagcgcctcggtctgctcgggcagcatcctaaaatttgtcttcaggtcgttatccacgtggtacttgtccatcatggcgcgcgccgcctccatgcccttctcccaggcggacaccatgggcaggcttagggggtttatcacttccaccggcgaggacaccgtactttcgatttcttcttcctccccctcttcccggcgcgcgcccacgctgctgcgcgctctcaccgcctgcaccaaggggtcgtcttcaggcaagcgccgcaccgagcgcttgccgcccttgacctgcttaatcagcaccggcgggttgctgaagcccaccatggtcagcgccgcctgctcttcttcgtcttcgctgtctaccactatctctggggaagggcttctccgctctgcggcggcgcgcttcttttttttcttgggagcggccgtgatggagtccgccacggcgacggaggtcgagggcgtggggctgggggtgcgcggtaccagggcctcgtcgccctcggactcttcctctgactccaggcggcggcggagtcgcttctttgggggcgcgcgcgtcagcggcggcggagacggggacggggacggggacgggacgccctccacagggggtggtcttcgcgcagacccgcggccgcgctcgggggtcttctcgagctggtcttggtcccgactggccattgtatcctcctcctcctaggcagagagacataaggagtctatcatgcaagtcgagaaggaggagagcttaaccaccccctctgagaccgccgatgcgcccgccgtcgccgtcgcccccgctgccgccgacgcgcccgccacaccgagcgacacccccgcggacccccccgccgacgcacccctgttcgaggaagcggccgtggagcaggacccgggctttgtctcggcagaggaggatttgcgagaggaggaggataaggagaagaagccctcagtgccaaaagatgataaagagcaagacgagcacgacgcagatgcacaccagggtgaagtcgggcggggggacggagggcatgacggcgccgactacctagacgaagggaacgacgtgctcttgaagcacctgcatcgtcagtgcgccattgtttgcgacgctctgcaggagcgcagcgaagtgcccctcagcgtggcggaggtcagccacgcctacgagctcagcctcttctccccccgggtgcccccccgccgccgcgaaaacggcacatgcgagcccaacccgcgcctcaacttctaccccgcctttgtggtacccgaggtcctggccacctatcacatcttctttcaaaattgcaagatccccctctcgtgccgcgccaaccgtagccgcgccgataagatgctggccctgcgccagggcgaccacatacctgatatcgccgctttggaagatgtaccaaagatcttcgagggtctgggtcgcaacgagaagcgggcagcaaactctctgcaacaggaaaacagcgaaaatgagagtcacaccggggtactggtggagctcgagggcgacaacgcccgcctggcggtggtcaagcgcagcatcgaggtcacccactttgcctaccccgcgctaaacctgccccccaaagtcatgaacgcggccatggacgggctgatcatgcgccgcggccggcccctcgctccagatgcaaacttgcatgaggagaccgaggacggccagcccgtggtcagcgacgagcagctggcgcgctggctggagaccgcggaccccgccgaactggaggagcggcgcaagatgatgatggccgtggtgctggtcaccgtagagctggagtgtctgcagcgcttcttcggcgaccccgagatgcagagaaaggtcgaggagaccctgcactacaccttccgccagggctacgtgcgccaggcttgcaagatctccaacgtggagctcagcaacctggtgtcctacctgggcatcttgcatgagaaccgcctcgggcagagcgtgctgcactccaccctgcgcggggaggcgcgccgcgactacgtgcgcgactgcgtttacctcttcctctgctacacctggcagacggccatgggggtctggcagcagtgcctggaggagcgcaacctcaaggagctggagaagctcctgcagcgcgcgctcaaagatctctggacgggctacaacgagcgctcggtggccgccgcgctggccgacctcatcttccccgagcgcctgctcaaaaccctccagcaggggctgcccgacttcaccagccaaagcatgttgcaaaacttcaggaactttatcctggagcgttctggcatcctacccgccacctgctgcgccctgcccagcgactttgtccccctcgtgtaccgcgagtgccccccgccgctgtggggtcactgctacctgttccaactggccaactacctgtcctaccacgcggacctcatggaggactccagcggcgaggggctcatggagtgccactgccgctgcaacctctgcacgccccaccgctccctggtctgcaacacccaactgctcagcgagagtcagattatcggtaccttcgagctacagggtccgtcctcctcagacgagaagtccgcggctccggggctaaaactcactccggggctgtggacttccgcctacctgcgcaaatttgtacctgaagactaccacgcccacgagatcaggttttacgaagaccaatcccgcccgcccaaggcggagctgaccgcctgcgtcatcacccagggcgagatcctaggccaattgcaagccatccaaaaagcccgccaagactttttgctgaagaagggtcggggggtgtatctggacccccagtcgggtgaggagctcaacccggttcccccgctgccgccgccgcgggaccttgcttcccaggataagcatcgccatggctcccagaaagaagcagcagcggccgccactgccgccaccccacatgctggaggaagaggaggaatactgggacagtcaggcagaggaggtttcggacgaggaggagccggagacggagatggaagagtgggaggaggacagcttagacgaggaggcttccgaagccgaagaggcagacgcaacaccgtcaccctcggccgcagccccctcgcaggcgcccccgaagtccgctcccagcatcagcagcaacagcagcgctataacctccgctcctccaccgccgcgacccacggccgaccgcagacccaaccgtagatgggacaccaccggaaccggggccggtaagtcctccgggagaggcaagcaagcgcagcgccaaggctaccgctcgtggcgcgctcacaagaacgccatagtcgcttgcttgcaagactgcggggggaacatctccttcgcccgccgcttcctgctcttccaccacggtgtggccttcccccgtaacgtcctgcattactaccgtcatctctacagcccctactgcggcggcagtgagccagagacggtcggcggcggcggcggcgcccgtttcggcgcctaggaagacccagggcaagacttcagccaagaaactcgcggcggccgcggcgaacgcggtcgcgggggccctgcgcctgacggtgaacgaacccctgtcgacccgcgaactgaggaaccgaatcttccccactctctatgccatcttccagcagagcagagggcaggatcaggaactgaaagtaaaaaacaggtctctgcgctccctcacccgcagctgtctgtatcacaagagcgaagaccagcttcggcgcacgctggaggacgctgaggcactcttcagcaaatactgcgcgctcactcttaaggactagctccgcgcccttctcgaatttaggcgggaacgcctacgtcatcgcagcgccgccgtcatgagcaaggacattcccacgccatacatgtggagctatcagccgcagatgggactcgcggcgggcgcctcccaagactactccacccgcatgaactggctcagtgccggcccacacatgatctcacaggttaatgatatccgcacccatcgaaaccaaatattggtggagcaggcggcaattaccaccacgccccgcaataatcccaaccccagggagtggcccgcgtccctggtgtatcaggaaattcccggccccaccaccgtactacttccgcgtgattcccaggccgaagtccaaatgactaactcaggggcacagctcgcgggcggctgtcgtcacagggtgcggcctcctcgccagggtataactcacctggagatccgaggcagaggtattcagctcaacgacgagtcggtgagctcctcgctcggtctcagacctgacgggaccttccagatagccggagccggccgatcttccttcacgccccgccaggcgtacctgactctgcaaagctcgtcctcggcgccgcgctcgggcggcatcgggactctccagttcgtgcaggagtttgtgccctcggtctacttcaaccccttctcgggctctcccggtcgctacccggaccagttcatctcgaactttgacgccgcgagggactcggtggacggctacgactgaatgtcgggtggacccggtgcagagcaacttcgcctgaagcacctcgaccactgccgccgccctcagtgctttgcccgctgtcagaccggtgagttccagtacttttccctgcccgactcgcacccggacggcccggcgcacggggtgcgctttttcatcccgagtcaggtgcgctctaccctaatcagggagtttaccgcccgtcccctactggcggagttggaaaaggggccttctatcctaaccattgcctgcatctgctctaaccctggattgcaccaagatctttgctgtcatttgtgtgctgagtataataaaggctgagatcagaatctactcgggctcctgtcgccatcctgtcaacgccaccgtccaagcccggcccgatcagcccgaggtgaacctcacctgcggtctgcaccggcgcctgaggaaatacctagcttggtactacaacagcactccctttgtggtttacaacagctttgaccaggacggggtctcactgagggataacctctcgaacctgagctactccatcaggaagaacagcaccctcgagctacttcctccttacctgcccgggacttaccagtgtgtcaccggtccctgcacccacacccacctgttgatcgtaaacgactctcttccgagaacagacctcaataactcctcttcgcagttccccagaacaggaggtgagctcaggaaaccccgggtaaagaagggtggacgagagttaacacttgtggggtttctggtgtatgtgacgctggtggtggctcttttgattaaggcttttccttccatgtctgaactctccctcttcttttatgaacaactcgactagtgctaacgggaccctacccaacgaatcgggattgaatatcggtaaccaggttgcagtttcacttttgattaccttcatagtcctcttcctgctagtgctgtcgcttctgtgcctgcggatcgggggctgctgcatccacgtttatatctggtgctggctgtttagaaggttcggagaccatcgcaggtagaataaacatgctgctgcttaccctctttgtcctggcgctggccgccagctgccaagccttttccgaggctgactttatagagccccagtgtaatgtgacttttaaagcccatgcacagcgttgtcatactataatcaaatgtgccaccgaacacgatgaataccttatccagtataaagataaatcacacaaagtggcacttgttgacatctggaaacccgaagaccctttggaatacaatgtgaccgttttccagggtgacctcttcaaaatttacaattacactttcccatttgaccagatgtgtgactttgtcatgtacatggaaaagcagcacaagctgtggcctccgactccccagggctgtgtggaaaatccaggctctttctgcatgatctctctctgtgtaactgtgctggcactaatactcacgcttttgtatatcagatttaaatcaaggcaaagcttcattgatgaaaagaaaatgccttaatcgctttcacgcttgattgctaacaccgggtttttatccgcagaatgattggaatcaccctactaatcacctccctccttgcgattgcccatgggttggaacgaatcgaagtccctgtgggggccaatgttaccctggtggggcctgtcggcaatgctacattaatgtgggaaaaatatactaaaaatcaatgggtctcttactgcactaacaaaaatagccacaagcccagagccatctgcgatgggcaaaatctaaccttgattgatgttcaattgctggatgcgggctactattatgggcagctgggtacaatgattaattactggagaccccacagagattacatgctccacgtagtaaagggtccccttagcagcccacccactaccacctctactacccccactaccaccactactcccaccaccagcactgccgcccagcctcctcatagcagaacaaccacttttatcaattccaagtcccactccccccacattgccggcgggccctccgcctcagactccgaaaccaccgagatctgcttctgcaaatgctctgacgccattgcccaggatttggaagatcacgaggaagatgagcatgacttcgcagatgcatgccaggcatcagagccagaagcgctgccggtggccctcaaacagtatgcagacccccacaccacccccgaccttcctccaccttcccagaagccaagtttcctgggggaaaatgaaactctgcctctctccatactcgctctgacatctgttgctatgttgaccgctctgctggtgcttctatgctctatatgctacctgatctgctgcagaaagaaaaaatctcacggccatgctcaccagcccctcatgcacttcccttaccctccagagctgggcgaccacaaactttaagtctgcagtaactatctgcccatcccttgtcagtcgacagcgatgagccccactaatctaacggcctctggacttacaacatcgtctcttaatgagaccaccgctcctcaagacctgtacgatggtgtctccgcgctggttaaccagtgggatcacctgggcatatggtggctcctcataggagcagtgaccctgtgcctaatcctggtctggatcatctgctgcatcaaaagcagaagacccaggcggcggcccatctacaggccctttgtcatcacacctgaagatgatgatgacaccacttccaggctgcagaggctaaagcagctactcttctcttttacagcatggtaaattgaatcatgcctcgcattttcatctacttgtctctccttccactttttctgggctcttctacattggccgctgtgtcccacatcgaggtagactgcctcacgcccttcacagtctacctgcttttcggctttgtcatctgcacctttgtctgcagcgttatcactgtagtgatctgcttcatacagtgcatcgactacgtctgcgtgcgggtggcttactttagacaccacccccagtatcgcaacagggacatagcggctctcctaagacttgtttaaaatcatggccaaattaactgtgattggtcttctgatcatctgctgcgtcctagccgcgattgggactcaagctcctaccaccaccagcgctcccagaaagagacatgtatcctgcagcttcaagcgtccctggaatataccccaatgctttactgatgaacctgaaatctctttggcttggtacttcagcgtcaccgcccttcttatcttctgcagtacggttattgcccttgccatctacccttcccttgacctgggctggaatgctgtcaactctatggaatatcccaccttcccagaaccagacctgccagacctggttgttctaaacgcgtttcctcctcctgctcccgttcaaaatcagtttcgccctccgtcccccacgcccactgaggtcagctactttaatctaacaggcggagatgactgaaaacctagacctagaaatggacggtctctgcagcgagcaacgcacactagagaggcgccggcaaaaagagctcgagcgtcttaaacaagagctccaagacgcggtggccatacaccagtgcaaaaaaggtgtcttctgtctggtaaaacaggccacgctcacctatgaaaaaacaggtgacacccaccgcctaggatacaagctgcccacacagcgccaaaagttcgccctcatgataggcgaacaacccatcaccgtgacccagcactccgtggagacagaaggctgcatacatgctccctgtaggggcgctgactgcctctacaccttgatcaaaaccctctgcggtctcagagaccttatccctttcaattaatcataactgtaatcaataaaaaatcacttacttgaaatctgatagcaagcctctgtccaattttttcagcaacacttccttcccctcctcccaactctggtactctaggcgcctcctagctgcaaacttcctccacagtctgaagggaatgtcagattcctcctcctgtccctccgcacccacgatcttcatgttgttgcagatgaaacgcgcgagatcgtctgacgagaccttcaaccccgtgtacccctacgataccgagatcgctccgacttctgtccctttccttacccctccctttgtgtcatccgcaggaatgcaagaaaatccagctggggtgctgtccctgcacttgtcagagccccttaccacccacaatggggccctgactctaaaaatggggggcggcctgaccctggacaaggaagggaatctcacttcccaaaacatcaccagtgtcgatccccctctcaaaaaaagcaagaacaacatcagccttcagaccgccgcacccctcgccgtcagctccggggccctaacactttttgccactccccccctagcggtcagtggtgacaaccttactgtgcagtctcaggcccctctcactttggaagactcaaaactaactctggccaccaaaggacccctaactgtgtccgaaggcaaactt

SEQ ID NO: 23 - DNA, 32728 bp, synthetic construct
  misc_feature 1..32728: note = Adenovirus vector nucleotide sequences
  source 1..32728: mol_type = other DNA
ataatataccttattttggattgtggccaatatgataatgaggtgggcggggagaggcggggcgggtgacgtaggacgcgcgagtagggttgggaggtgtggcggaagtgtggcatttgcaagtgggaggagctcacatgcaagcttccgtcgcggaaaatgtgacgtttttgatgagcgccgcctacctccggaagtgccaattttcgcgcgcttttcaccggatatcgtagtaattttgggcgggaccatgtaagatttggccattttcgcgcgaaaagtgaaacggggaagtgaaaactgaataatagggcgttagtcatagcgcgtaatatttaccgagggccgagggactttgaccgattacgtggaggactcgcccaggtgttttttacgtgaatttccgcgttccgggtcaaagtctccgtttttattgtcaccgtcatttgacgcggagggtatttaaacccgctgcgctcctcaagaggccactcttgagtgccagcgagaagagttttctcctctgctccgcttcggtgatcgaaaaatgagacacatagcctgcactccgggtcttttgtccggtcgggcggcggccgagcttttggacgctttgatcaatgatgtcctaagcgatgattttccgtctactacccactttagcccacctactcttcacgaactgtacgatctggatgtactggtggatgtgaacgatcccaacgaggaggcggtttctgcgttttttcccgagtctgcgctgttggccgctcaggagggatttgacctacacactccgccgcctattttagagtctccgctgccggagcccagtggtataccttatatgcctgaactgcttcccgaagtggtagacctgacctgccacgagcctggctttccgcccagcgacgatgagggtgagccttttgttttagactttgctgagatacctgggcacggttgcaggtcttgtgcatatcatcagagggttaccggagaccccgaggttaagtgttcgctgtgctatatgaggatgacctcttcctttatctacagtaagtttttgtctaggtgggcttttgggtaggtgggttttgtgtcagaacaggtgtaaacgttgcttgtgttttttgtacctgtaggtccggtgtccgagccagacccggagcccgaccgcgatcccgagccggatcccgagcctcctcgcaggacaaggaaactaccttccattctgtgcaagtctcagacacctgtaaggaccagcgaggcagacagcaccgactctggcacttctacctctccccctgaaattcacccagtggttcctctgggtatacataaacctgttgctgttaaagtttgcgggcgacgccctgcagtacagtgcattgaggacttgcttcacgatcccgaggaacctttggacttgagccttaaacgccctaggcaataaaccccacctaagtaataaaccccacctaagtaataaaccctgccgcccttggttattgagatgacgcccaatgtttgcttttgaatgacttcatgtgtgtaataaaagtgagtgtgatcataggtctcttgtttgtctgggcggggcttaagggtatataagtctcttggggctaaacttggttacacttgaccccaatggaggcgtgggggtgcttggaggagtttgcggacgtgcgccgtttgctggacgagagctctagcaatacctatactatttggaggtatctgtggggctctactcaggccaagttggtttccagaattaagcaggattacaagtgcgattttgaagagctttttagttcctgcggtgagcttttgcaatccttgaatctgggccatcaggctattttccaggaaaaggttctctcgactttggatttttccactcccgggcgcaccgccgcttgtgtggcttttgtgtcttttgtgcaagataaatggagcgaggagacccacctgagtcacggctacgtactggatttcatggcgatggctctttggagggctcacaacaaatggaagattcagaaggaactgtacggttccgccctacgtcgtccacttctgtcgcgacaggggctgaggtttcccgaccatcggcagcatcagaatctggaagacgagtcggaggagcgagcggaggagaagatcagcttgagagccggcctggaccctcctcaggaggaatgaatctcccgcaggtggttgacctgtttccagaactgagacgggtcctgactatcagggaggatggtcagtttgtgaagaagtttaagagggatcggggtgagggagatgatgaggcggctagcaatttagcttttagtctgatgactcgccaccgaccggaatgtattacctatcagcagattaaggagagttgtgccaacgagctggatcttttgggtcagaagtatagcatagaacagcttaccacttactggcttcagcctggggatgattgggaagaggcgatcagggtgtatgcaaaggtggccctgcggcccgattgcaagtataagattactaagttggttaatattagaaactgctgctatatttctgggaacggggccgaagtggagatagatactcaggacagggtggcttttaggtgttgcatgataaacatgtggcccgggatactggggatggatggggtggtattcatgaatgtgaggtttacgggccccaactttaatggcacggtgttcatgggcaacaccaacttgctcctgcatggtgcgagtttctatgggtttaataacacctgtatagaggcctggaccgatgtaaaggttcgaggttgttccttttatagctgttggaaggcggtggtgtgtcgccctaaaagcaggggttctgtgaaaaaatgcttgtttgaaaggtgcaccttaggcatcctctctgagggcaactccagggtgcgccataatgtggcttcgaactgcggttgcttcatgcaagtgaagggggtgagcgttatcaagcataactcggtgtgtggaaactgcgaggatcgcgcctcccagatgctgacctgctttgatggcaactgtcacctgttgaagaccattcatataagcagccaccccagaaaggcctggcccgtgtttgagcataacatcttgacccgctgctccttgcatctgggggtcaggaggggtatgttcctgccttaccagtgtaactttagccacactaaaatcctgctggaacccgagtgcatgaccaaggtcagcctgaatggtgtgtttgatgtgactctgaaaatctggaaggtgctgaggtatgatgagaccaggaccaggtgccgaccctgcgagtgcggcggcaagcacatgagaaatcagcctgtgatgttggatgtgaccgaggagcttaggcctgaccatctggtgctggcctgcaccagggccgagtttgggtctagcgatgaggataccgattgaggtgggtaaggtgggcgtggctagaagggtggggcgtgtataaattgggggtctaagggtctctctgttttgtcttgcaacagccgccgccatgagcgacaccggcaacagctttgatggaagcatctttagcccctatctgacagtgcgcatgcctcactgggctggagtgcgtcagaatgtgatgggttccaacgtggatggacgccccgttctgccttcaaattcgtctacaatggcctacgcgaccgtgggaggaactccgctggacgccgcgacctccgccgccgcctccgccgccgccgcgaccgcgcgcagcatggctacggacctttacagctctttggtggcgagcggcgcggcctctcgcgcgtctgctcgggatgagaaactgaccgctctgctgcttaaactggaagacttgacccgggagctgggtcaactgacccagcaggtctccagcttgcgtgagagcagccttgcctccccctaatggcccataatataaataaaagccagtctgtttggattaagcaagtgtatgttctttatttaactctccgcgcgcggtaagcccgggaccagcggtctcggtcgtttagggtgcggtggattctttccaacacgtggtacaggtggctctggatgtttagatacatgggcatgagtccatccctggggtggaggtagcaccactgcagagcttcgtgctcgggggtggtgttgtatatgatccagtcgtagcaggagcgctgggcgtggtgctgaaaaatgtccttaagcaagaggcttatagctagggggaggcccttggtgtaagtgtttacaaatctgctcagttgggaggggtgcatccggggggatataatgtgcatcttggactggatttttaggttggctatgttcccacccagatcccttctgggattcatgttgtgcaggaccaccagcacggtatatccagtgcacttgggaaatttatcgtggagcttagacgggaatgcatggaagaacttggagacgcccttgtggcctcccagattttccatacattcgtccatgatgatggcaatgggcccgtgggaagctgcctgagcaaaaatgtttctgggatcgctcacatcgtagttatgttccagggtgaggtcatcataggacatctttacgaatcgggggcggagggtcccggactgggggatgatggtaccctcgggccccggggcgtagttcccctcacagatctgcatctcccaggctttcatttcagagggagggatcatatccacctgcggagcgatgaaaaacacagtttctggcgcaggggagattaactgggatgagagcaggtttctgagcagctgtgactttccacagccggtgggcccatatatcacgcctatcaccggctgcagctggtagttaagagagctgcagctgccgtcctcccggagcaggggggccacctcgttcagcatatccctgacgtggatgttctccctgaccaattccgccagaaggcgctcgccgcccagcgaaagcagctcttgcaaggaagcaaaatttttcagcggttttaggccgtcggccgtgggcatgtttttcagcgtctgggtcagcagttccagcctgtcccacagctcggtgatgtgctctacggcatctcgatccagcagatctcctcgtttcgcgggttggggcggctttcgctgtagggcaccagccgatgggcgtccagcggggccagagtcatgtccttccatgggcgcagggtcctcgtcagggtggtctgggtcacggtgaaggggtgcgctccgggttgggcgctggccagggtgcgcttgaggctggttctgctggtgctgaatcgctgccgctcttcgccctgcgcgtcggccaggtagcatttgaccatggtctcgtagtcgagaccctcggcggcgtgccccttggcgcggagctttcccttggaggtggcgccgcacgaggggcactgcaggctcttcagggcgtagagcttgggagcgagaaacacggactctggggagtaggcgtccgcgccgcaggaagcgcagaccgtctcgcattccaccagccaagtgagctccgggcggtcagggtcaaaaaccaggttgcccccatgctttttgatgcgtttcttacctcggctctccatgaggcggtgtcccttctcggtgacgaagaggctgtccgtgtccccgtagaccgacttcaggggcctgtcttccagcggagtgcctctgtcctcctcgtagagaaactctgaccactctgagacgaaggcccgcgtccaggccaggacgaaggaggccacgtgggaggggtagcggtcgttgtccactagcgggtccaccttctccagggtgtgcaggcacatgtccccctcctccgcgtccagaaaagtgattggcttgtaggtgtaggacacgtgaccgggggttcccgacgggggggtataaaagggggtgggcgccctttcatcttcactctcttccgcatcgctgtctgcgagggccagctgctggggtaagtattccctctcgaaggcgggcatgacctcagcgctcaggttgtcagtttctaaaaatgaggaggatttgatgttcacctgtccggaggtgatacctttgagggtacctgggtccatctggtcagaaaacactatttttttgttgtcaagcttggtggcgaacgacccgtagagggcgttggagagcagcttggcgatggagcgcagggtctggtttttgtcgcggtcggctcgctccttggccgcgatgttgagttgcacgtactcgcgggccacgcacttccactcggggaagacggtggtgcgctcgtctgggattaggcgcaccctccagcctcggttgtgcagggtgaccatgtcgacgctggtggcgacctcgccgcgcaggcgctcgttggtccagcagaggcggccgcccttgcgcgagcagaaggggggtagggggtccagctggtcctcgtttggggggtccgcgtcgatggtgaagaccccggggagcaagcgcgggtcaaagtagtcgatcttgcaagcttgcatgtccagagcccgctgccattcgcgggcggcgagcgcgcgctcgtaggggttgaggggcgggccccagggcatggggtgggtgagcgcggaggcgtacatgccgcagatgtcatacacgtacaggggttccctgaggatgccgaggtaggtggggtagcagcgccccccgcggatgctggcgcgcacgtagtcatagagctcgtgggagggggccagcatgttgggcccgaggttggtgcgctgggggcgctcggcgcggaaggcgatctgcctgaagatggcatgggagttggaggagatggtgggccgctggaagacgttgaagcttgcttcttgcaagcccaccgagtccctgacgaaggaggcgtaggactcgcgcagcttgtgcaccagctcggcggtgacctggacgtcgagcgcgcagtagtcgagggtctcgcggatgatgtcatacttatcctcccccttctttttccacagctcgcggttgaggacgaactcttcgcggtctttccagtactcttggaggggaaacccgtccgtgtccgaacggtaagagcctagcatgtagaactggttgacggcctggtaggggcaacagcccttctccacgggcagcgcgtaggcctgcgccgccttgcggagggaggtgtgggtgagggcgaaagtgtccctgaccatgactttgaggtattgatgtttgaagtctgtgtcatcgcagccgccctgttcccacagggtgtagtccgtgcgctttttggagcgcgggttgggcagggagaaggtgaggtcattgaagaggatcttccccgctcgaggcatgaagtttctggtgatgcgaaagggccctgggaccgaggagcggttgttgatgacctgggcggccaggacgatctcgtcaaagccgtttatgttgtggcccacgatgtagagctccaaaaagcggggctggcccttgatggaggggagctttttgagttcctcgtaggtgagctcctcgggcgattccaggccgtgctcctccagggcccagtcttgcaagtgagggttggccgccaggaaggatcgccagaggtcgcgggccatgagggtctgcaggcggtcgcggaaggttctgaactgtcgccccacggccatcttttcgggggtgatgcagtagaaggtgagggggtctttctcccaggggtcccatctgagctctcgggcgaggtcgcgcgcggcggcgaccagagcctcgtcgccccccagtttcatgaccagcatgaagggcacgagctgcttgccaaaggctcccatccaagtgtaggtctctacatcgtaggtgacaaagaggcgctccgtgcgaggatgagagccgatcgggaagaactggatctcccgccaccagttggaggattggctgttgatgtggtgaaagtagaagtcccgtctgcgggccgagcactcgtgctggcttttgtaaaagcgaccgcagtactggcagcgctgcacgggttgtatatcttgcacgaggtgaacctggcgacctctgacgaggaagcgcagcgggaatctaagtcccccgcctggggtcccgtgtggctggtggtcttctactttggttgtctggccgccagcatctgtctcctggagggcgatggtggagcagaccaccacgccgcgagagccgcaggtccagatctcggcgctcggcgggcggagtttgatgacgacatcgcgcacattggagctgtccatggtctccagctcccgcggcggcaggtcagctgggagttcctggaggttcacctcgcagagacgggtcaaggcgcgggcagtgttgagatggtatctgatttcaaggggcgtgttggcggcggagtcgatggcttgcaggaggccgcagccccggggggccacgatggttccccgcggggcgcgaggggaggcggaagctgggggtgtgttcagaagcggtgacgcgggcgggcccccggaggtagggggggttccggccccacaggcatgggcggcaggggcacgtcttcgccgcgcgcgggcaggggctggtgctggctccgaagagcgcttgcgtgcgcgacgacgcgacggttggtgtcctgtatctgacgcctctgagtgaagaccacgggtcccgtgaccttgaacctgaaagagagttcgacagaatcaatctcggcatcgttgacagcggcctggcgcaggatctcctgcacgtcgcccgagttgtcctggtaggcgatctctgccatgaactgctcgatctcttcttcctggagatctcctcgtccggcgcgctccacggtggccgccaggtcgttggagatgcgacccatgagctgtgagaaggcgttgagcccgccctcgttccagacccggctgtagaccacgcccccctcggcgtcgcgagcgcgcatgaccacctgggccaggttgagctccacgtgtcgcgtgaagacggcgtagttgcgcaggcgctggaaaaggtagttcagggtggtggcggtgtgctcggcgacgaagaagtacatgacccagcgccgcaacgtggattcattgatgtcccccaaggcctccaggcgctccatggcctcgtagaagtccacggcgaagttgaaaaactgggagttgcgagcggacacggtcaactcctcctccagaagacggatgagctcggcgacagtgttgcgcacctcgcgctcgaaggccacggggggcgcttcttcctcttccacctcttcttccatgatcgcttcttcttcttcctcagccgggacgggagggggcggcggcggcgggggaggggcgcggcggcggcggcggcgcaccgggaggcggtcgatgaagcgctcgatcatctccccccgcatgcggcgcatggtctcggtgacggcgcggccgttctcccgggggcgcagctcgaagacgccgcctctcatctcgccgcggggcgagcggccgtgaggtagcgagacggcgctgactatgcatcttaacaattgctgtgtaggtacaccgccgagggacctgattgagtccagatccaccggatccgaaaacctttggaggaaagcgtctatccagtcgcagtcgcaaggtaggctgagcaccgtggcgggcgggggcgggtctggagagttcctggcggagatgctgctgatgatgtaattaaagtaggcggtcttgagaaggcggatggtggacaggagcaccatgtctttgggtccggcctgttggatgcggaggcggtcggccatgccccaggcctcgttctgacaccggcgcaggtctttgtagtagtcttgcatgagtctttccaccggcacctcttctccttcctcttctccatctcgccggtggtttctcgcgccgcccatgcgcgtgaccccaaagcccctgagcggctgcagcagggccaggtcggcgaccacgcgctcggccaagatggcctgctgcacctgagtgagggtcctctcgaagtcatccatgtccacgaagcggtggtaggcgcccgtgttgatggtgtaggtgcagttggccatgacggaccagttgacggtctggtgtcccggctgcgagagctccgtgtaccgcaggcgcgagaaggcgcgggaatcgaacacgtagtcgttgcaagtccgcaccagatactggtagcccaccaggaagtgcggcggaggttggcgatagaggggccagcgctgggtggcgggggcgccgggcgccaggtcttccagcatgaggcggtggtatccgtagatgtacctggacatccaggtgatgccggcggcggtggtggtggcgcgcgcgtagtcgcggacccggttccagatgtttcgcaggggcgagaagtgttccatggtcggcacgctctggccggtgaggcgcgcgcagtcgttgacgctctatacacacacaaaaacgaaagcgtttacagggctttcgttctgtagcctggaggaaagtaaatgggttgggttgcggtgtgccccggttcgagaccaagctgagctcggccggctgaagccgcagctaacgtggtattggcagtcccgtctcgacccaggccctgtatcctccaggatacggtcgagagcccttttgctttcttggccaagcgcccgtggcgcgatctgggatagatggtcgcgatgagaggacaaaagcggctcgcttccgtagtctggagaaacaatcgccagggttgcgttgcggcgtaccccggttcgagcccctatggcggcttgaatcggccggaaccgcggctaacgagggccgtggcagccccgtcctcaggaccccgccagccgacttctccagttacgggagcgagccccttttgttttttattttttagatgcatcccgtgctgcggcagatgcgcccctcgccccggcccgatcagcagcagcaacagcaggcatgcagacccccctctcccctttccgccccggtcaccacggccgcggcggccgtgtcgggcgcggggggcgcgctggagtcagatgagccaccgcggcggcgacctaggcagtatctggacttggaagagggcgagggactggcgcggctgggggcgaactctccagagcgccacccgcgggtgcagttgaaaagggacgcgcgcgaggcgtacctgccgcggcagaacctgtttcgcgaccgcgggggcgaggagcccgaggagatgcgagactgcaggttccaagcggggcgcgagctgcggcgcgggctggacagacagcgcctgctgcgcgaggaggactttgagcccgacacgcagacgggcatcagccccgcgcgcgcgcacgtagccgcggccgacctggtgaccgcctacgagcagacggtaaaccaggagcgcaacttccaaaagagcttcaacaaccacgtgcgcacgctggtggcgcgcgaggaggtgaccctgggtctcatgcatctgtgggacctggtggaggcgatcgtgcagaaccccagcagcaagcccctgaccgcgcagctgttcctggtggtgcagcacagcagggacaacgaggccttcagggaggcgctgctgaacatcaccgagccggaggggcgctggctcctggacctgataaacatcctgcagagcatagtggtgcaggagcgcagcctgagcctggccgagaaggtggcggccatcaactactctatgctgagcctgggcaagttctacgcccgcaagatctacaagaccccctacgtgcccatagacaaggaggtgaagatagacagcttctacatgcgcatggcgctgaaggtgctgaccctgagcgacgacctgggagtgtaccgcaacgagcgcatccacaaggccgtgagcgccagccggcggcgcgagctgagcgaccgcgagctgatgcacagtctgcagcgcgcgctgaccggcgcgggcgagggcgacagggaggtcgagtcctacttcgacatgggggccgacctgcactggcagccgagccgccgcgccctggaggcggcgggggcgtacggcggccccctggcggccgatgaccaggaagaggaggactatgagctagaggagggcgagtacctggaggactgacctggctggtggtgttttggtatagatgcaagatccgaacgtggcggacccggcggtccgggcggcgctgcaaagccagccgtccggcattaactcctctgacgactgggccgcggccatgggtcgcatcatggccctgaccgcgcgcaaccccgaggctttcaggcagcagcctcaggccaaccggctggcggccatcttggaagcggtagtgcccgcgcgctccaaccccacccacgagaaggtgctggccatagtcaacgcgctggcggagagcagggccatccgcgcggacgaggccggactggtgtacgatgcgctgctgcagcgggtggcgcggtacaacagcggcaacgtgcagaccaacctggaccgcctggtgacggacgtgcgcgaggccgtggcgcagcgcgagcgcttgcatcaggacggtaacctgggctcgctggtggcgctaaacgccttcctcagcacccagccggccaacgtaccgcgggggcaggaggactacaccaactttttgagcgcgctgcggctgatggtgaccgaggtccctcagagcgaggtgtaccagtcggggcccgactacttcttccagaccagcagacagggcttgcaaaccgtgaacctgagccaggctttcaagaacctgcgggggctgtggggagtgaaggcgcccaccggcgaccgggctacggtgtccagcctgctaacccccaactcgcgcctgctgctgctgctgatcgcgcccttcacggacagcgggagcgtctcgcgggagacctatctgggccacctgctgacgctgtaccgcgaggccatcgggcaggcgcaggtggacgagcacaccttccaagagatcaccagcgtgagccacgcgctggggcaggaggacacgggcagcctgcaggcgaccctgaactacctgctgaccaacaggcggcagaagattcccacgctgcacagcctgacccaggaggaggagcgcatcttgcgctacgtgcagcagagcgtgagcctgaacctgatgcgcgacggcgtgacgcccagcgtggcgctggacatgaccgcgcgcaacatggaaccgggcatgtacgcctcccaccggccgtttatcaaccgcctgatggactacttgcatcgggcggcggccgtgaaccccgagtacttcactaatgccattctgaatccccactggatgccccctccgggtttctacaacggggactttgaggtgcccgaggtcaacgacgggttcctctgggatgacatggatgacagtgtgttctcacccaacccgctgcgcgccgcgtctctgcgattgaaggagggctctgacagggaaggaccgaggagtctggcctcctccctggctctgggagcggtgggcgccacgggcgcggcggcgcggggcagtagccccttccccagcctggcagactctctgaacagcgggcgggtgagcaggccccgcttgctaggcgaggaggagtatctgaacaactccctgctgcagcccgcgagggacaagaacgctcagcggcagcagtttcccaacaatgggatagagagcctggtggacaagatgtccagatggaagacgtatgcgcaggagtacaaggagtgggaggaccgccagccgcggcccttgccgccccctaggcagcgctggcagcggcgcgcgtccaaccgccgctggaggcaggggcccgaggacgatgatgactctgcagatgacagcagcgtgttggacctgggcgggagcgggaaccccttttcgcacctgcgcccacgcctgggcaagatgttttaaaagaaaaaaaaaataaaactcaccaaggccatggcgacgagcgttggttttttgttcccttccttagtatgcggcgcgcggcgatgttcgaggaggggcctcccccctcttacgagagcgcgatggggatttctcctgcggcgcccctgcagcctccctacgtgcctcctcggtacctgcaacctacaggggggagaaatagcatctgttactctgagctgcagcccctgtacgataccaccagactgtacctggtggacaacaagtccgcggacgtggcctccctgaactaccagaacgaccacagcgattttttgaccacggtgatccaaaacaacgacttcaccccaaccgaggccagcacccagaccataaacctggataacaggtcgaactggggcggcgacctgaagaccatcttgcacaccaacatgcccaacgtgaacgagttcatgttcaccaactcttttaaggcgcgggtgatggtggcgcgcgagcagggggaggcgaagtacgagtgggtggacttcacgctgcccgagggcaactactcagagaccatgactctcgacctgatgaacaatgcgatcgtggaacactatctgaaagtgggcaggcagaacggggtgaaggaaagcgatatcggggtcaagtttgacaccagaaacttccgtctgggctgggaccccgtgaccgggctggtcatgccgggggtctacaccaacgaggcctttcatcccgacatagtgcttctgcccggctgtggggtggacttcacccagagccggctgagcaacctgctgggcattcgcaagcggcagcctttccaggagggtttcaagatcacctatgaggatctgaaggggggcaacattcccgcgctccttgatctggacgcctacgaggagagcttgaaacccgaggagagcgctggcgacagcggcgagagtggcgaggagcaagccggcggcggtggcggcgcgtcggtagaaaacgaaagtacgcccgcagtggcggcggacgctgcggaggtcgagccggaggccatgcagcaggacgcagaggagggcgcacaggagggcgcgcagaaggacatgaacgatggggagatcaggggagacacattcgccacccggggcgaagaaaaagaggcagaggcggcggcggcggcgacggcggaggccgaaaccgaggttgaggcagaggcagagcccgagaccgaagttatggaagacatgaatgatggagaacgtaggggcgacacgttcgccacccggggcgaagagaaggcggcggaggcagaagccgcggctgaggaggcggctgcggctgcggccaagactgaggctgcggctaaggctgaggtcgaagccaatgttgcggttgaggctcaggctgaggaggaggcggcggctgaagcagttaaggaaaaggcccaggcagagcaggaagagaaaaaacctgtcattcaacctctaaaagaagatagcaaaaagcgcagttacaacgtcatcgagggcagcacctttacccagtaccgcagctggtacctggcgtacaactacggcgacccggtcaagggggtgcgctcgtggaccctgctctgcacgccggacgtcacctgcggctccgagcagatgtactggtcgctgccgaacatgatgcaagacccggtgaccttccgctccacgcggcaggttagcaacttcccggtggtgggcgccgaactgctgcccgtgcactccaagagtttttacaacgagcaggccgtctactcccagctgatccgccaggccacctctctgacccacgtgttcaatcgctttcccgagaaccagattttggcgcgcccgccggcccccaccatcaccaccgtgagtgaaaacgttcctgccctcacagatcacgggacgctaccgctgcgcaacagcatctcaggagtccagcgagtgaccattactgacgccagacgccggacctgcccctacgtttacaaggccttgggcatagtctcgccgcgcgtcctctccagtcgcactttttaaaacacatctacccacacgttccaaaatcatgtccgtactcatctcacccagcaacaacaccggctgggggctgcgcgcgcccagcaagatgtttggaggggcgaggaagcgctccgaccagcaccctgtgcgcgtgcgcggccactaccgcgcgccctggggagcgcacaagcgcgggcgcacagggcgcaccactgtggacgacgtcattgactccgtagtggagcaagcgcgccactacacacccggcgcgccgaccgcccccgccgtgtccaccgtggaccaggcgatcgaaagcgtggtacagggcgcgcggcactatgccaaccttaaaagtcgccgccgccgcgtggcccgccgccatcgccggagaccccgggccaccgccgccgcgcgccttactaaggctctgctcaggcgcgccaggcgaactggccaccgggccgccatgagggccgcacggcgggctgccgctgccgcaagcgtcgtggccccgcgggcacgaaggcgcgcggccgctgccgccgccgccgccatttccagcttggcctcgacgcggcgcggtaacatatactgggtgcgcgactcggtaaccggcacgcgggtacccgtgcgctttcgccccccgcggaattagcacaagacaacatacacactgagtctcctgctgttgtgtatcccagcggcgaccgtcagcagcggcgacatgtccaagcgcaaaattaaagaagagatgctccaggtcatcgcgccggagatctatgggcccccgaagaaggaggaggatgattacaagccccgcaagctaaagcgggtcaaaaagaaaaagaaagatgatgatgacgaggcggtggagtttgtccgccgcatggcacccaggcgccccgtgcagtggaagggccggcgcgtgcagcgcgttttgcgccccggcaccgcggtggtcttcacgcccggcgagcgctccacgcgcactttcaagcgggtgtacgatgaggtgtacggcgacgaggacctgttggagcaggccaaccagcgctttggggagtttgcatatgggaaacggccccgcgagagtctaaaagaggacctgctggcgctaccgctggacgagggcaatcccaccccgagtctgaagccggtaaccctgcaacaggtgctgcctttgagcgcgcccagcgagcataagcgagggttgaagcgcgaaggcggggacctggcgcccaccgtgcagttgatggtgcccaagcggcagaagctggaggacgtgctggagaaaatgaaagtagagcccgggatccagcccgagatcaaggtccgccccatcaagcaggtggcgcccggcgtgggagtccagaccgtggacgttaggattcccacggaggagatggaaacccaaaccgccactccctcttcggcggccagcgccaccaccggcaccgcttcggtagaggtgcagacggacccctggctacccgccaccgctgttgccgccgccgccccccgttcgcgcgggcgcaagagaaattatccagcggccagcgcgctcatgccccagtacgcactgcatccatccatcgtgcccacccccggctaccgcgggtactcgtaccgcccgcgcagatcagccggcactcgcggccgccgccgccgtgcgaccacaaccagccgccgccgtcgccgccgccgccagccagtgctgacccccgtgtctgtaaggaaggtggctcgctcggggagcacgctggtggtgcccagagcgcgctaccaccccagcatcgtttaaagccggtctctgtatggttcttgcagatatggccctcacttgtcgcctccgcttcccggtgccgggataccgaggaagaactcaccgccgcagaggcatggcgggcagcggtctccgcggcggccgtcgccatcgccggcgcgcaaaaagcaggcgcatgcgcggcggtgtgctgcctctgctaatcccgctaatcgccgcggcgatcggtgccgtacccgggatcgcctccgtggccctgcaggcgtcccagaaacgttgactcttgcaaccttgcaagcttgcattttttggaggaaaaataaaaaaaagtctagactctcacgctcgcttggtcctgtgactattttgtagaaaaaaagatggaagacatcaactttgcgtcgctggccccgcgtcacggctcgcgcccgttcatgggagactggacagatatcggcaccagcaatatgagcggtggcgccttcagctggggcagtctgtggagcggccttaaaaattttggttccaccattaagaactatggcaacaaagcgtggaacagcagcacgggccagatgctgagagacaagttgaaagagcagaacttccaggagaaggtggcgcagggcctggcctctggcatcagcggggtggtggacatagctaaccaggccgtgcagaaaaagataaacagtcatctggacccccgtcctcaggtggaggaaatgcctccagcgatggagacggtgtctcccgagggcaaaggcgaaaagcgcccgcggcccgacagagaagagaccctggtgtcacacaccgaggagccgccctcttacgaggaggcagtcaaggccggcctgcccaccactcgccccatagcccccatggccaccggtgtggtgggccacaggcaacacactcccgcaacactagatctgcccccgccgtccgagccgccgcgccagccaaaggcggcgacggtgcccgctccctccacttccgccgccaacagagtgcccctgcgccgcgccgcgagcggcccccgggcctcgcgagttagcggcaactggcagagcacactgaacagcatcgtgggcctgggagtgaggagtgtgaagcgccgccgttgctactgaatgagcaagctagctaacgtgttgtatgtgtgtatgcgtcctatgtcgccgccagaggagctgttgagccgccggcgccgtctgcactccagcgaatttcaagatggcgaccccatcgatgatgcctcagtggtcgtacatgcacatctcgggccaggacgcttcggagtacctgagccccgggctggtgcagttcgcccgcgccacagacacctacttcaacatgagtaacaagttcaggaaccccactgtggcgcccacccacgatgtgaccacggaccggtcgcagcgcctgacgctgcggttcatccccgtggatcgggaggacaccgcctactcttacaaggcgcggttcacgctggccgtgggcgacaaccgcgtgctggacatggcctccacttactttgacatcaggggggtgctggacaggggccccaccttcaagccctactcgggtactgcctacaactccctggcccccaagggcgctcccaattcttgcgagtgggaacaagatgaaccagctcaggcagcaatagctgaagatgaagaagaacttgaagaagaacaagctcaggacgaacaggcgcccactaagaaaacccatgtatacgcccaggcacctctttctggtgaaaaaattactaaggatggtttgcaaataggtgtggatgccacacaggcgggagataaccctatatatgctgataaaacattccaacccgaacctcagataggtgagtctcagtggaacgaggctgatgccacagtagcaggaggcagagtcttaaaaaagaccacccctatgagaccttgctatggatcctatgccaaacctactaatgccaatggcggtcaagggatcatggtggccaatgatcagggagcgcttgaatctaaagttgagatgcaatttttctccaccacaacgtctcttaatgtaagggaaggtgaaaacaatcttcagccaaaagtagtgctatacagcgaagatgttaacttggaatcccctgacactcatttgtcttacaaacctaaaaaggatgacaccaactctaaaatcatgttgggtcagcaagccatgcccaacagacccaacctcattgcttttagggacaactttattggacttatgtactacaacagcacaggcaacatgggagtgctggcaggacaggcctcccagctaaacgctgtggtagacttgcaagacagaaacacagagctgtcataccaactgatgcttgattccattggagacagatcaagatacttttccatgtggaaccaggcagtggacagctatgacccagatgtcagaatcattgaaaaccatggggttgaagatgagctgcccaactattgctttcccctgggcggtattggaattacagacacataccagtgcataaaaccaaccgcagctgctaataacactacatggtctaaggatgaagaatttagtgatcgcaatgaaataggggtgggaaacaacttcgccatggagatcaacatccaggccaacctctggaggaacttcctctatgcgaacgtggggctctacctgccagacaagctcaagtacaaccccaccaacgtggacatctctgacaaccccaacacctatgactacatgaacaagcgtgtggtggctcccggcctggtggactgctttgtcaatgtgggagccaggtggtccctggactacatggacaacgtcaaccccttcaaccaccaccgcaatgcgggtctgcgctaccgctccatgatcctgggcaacgggcgctacgtgcccttccacattcaggtgccccagaagttctttgccatcaagaacctcctcctcctgccgggctcctacacttacgagtggaacttcaggaaggatgtcaacatggtcctgcagagctctctgggcaatgaccttagggtggacggggccagcatcaagtttgacagcgtcaccctctatgctaccttcttccccatggctcacaacaccgcctccacgctcgaggccatgctgaggaacgacaccaacgaccagtccttcaatgactacctctctggggccaacatgctctaccccatccccgccaaggccaccaacgtgcccatctccattccctctcgcaactgggccgccttcagaggctgggcctttacccgccttaagaccaaggaaaccccctccctgggctcgggttttgacccctactttgtctactcgggatccatcccctacctggatggcaccttctacctcaaccacacttttaagaagatatccatcatgtatgactcctccgtcagctggccgggcaatgaccgcctgctcacccccaatgagttcgaggtcaagcgcgccgtggacggcgagggctacaacgtggcccagtgcaacatgaccaaggactggttcctggtgcagatgctggccaactacaacataggctaccagggcttctacatcccagagagctacaaggacaggatgtactccttcttcagaaatttccaacccatgagcaggcaggtggtggacgagaccaaatacaaggactatcaggccattggcatcactcaccagcacaacaactcgggattcgtgggctacctggctcccaccatgcgcgaggggcaggcctaccccgccaacttcccctacccgttgataggcaaaaccgcggtcgacagcgtcacccagaaaaagttcctctgcgaccgcaccctctggcgcatccccttctctagcaacttcatgtccatgggtgcgctcacggacctgggccagaacctgctctatgccaactccgcccatgcgctggacatgacttttgaggtggaccccatggacgagcccacccttctctatattgtgtttgaagtgttcgacgtggtcagagtgcaccagccgcaccgcggtgtcatcgagaccgtgtacctgcgcacgcccttctcggccggcaacgccaccacctaaggagacagcgccgccgcctgcatgacgggttccaccgagcaagagctcagggccatcgccagagacctgggatgcggaccctattttttgggcacctatgacaaacgcttcccgggcttcatctcccgagacaagctcgcctgcgccatcgtcaacacggccgcgcgcgagaccgggggcgtgcactggctggcctttggctgggacccgcgctccaaaacctgctacctcttcgacccctttggcttctccgatcagcgcctcagacagatctatgagtttgagtacgaggggctgctgcgccgcagcgcgcttgcctcctcgcccgaccgctgcatcacccttgagaagtccaccgagaccgtgcaggggccccactcggccgcctgcggtctcttctgctgcatgtttttgcacgcctttgtgcgctggccccagagtcccatggatcgcaaccccaccatgaacttgctcaagggagtgcccaacgccatgctccagagcccccaggtccagcccaccctgcgccacaaccaggaacagctctaccgcttcctggagcgccactccccctacttccgcagtcacagcgcgcacatccggggggccacctctttctgccacttgcaagaaaacatgcaagacggaaaatgatgtacagctcgctttttaataaatgtaaagactgtgcactttatttatacacgggctctttctggttatttattcaacaccgccgtcgccatctagaaatcgaaagggttctgccgcgcgtcgccgtgcgccacgggcagagacacgttgcgatactggaagcggctcgcccacttaaactcgggcaccaccatgcggggcagtggttcctcggggaagttctcgccccacagggtgcgggtcagctgcagcgcgctcaggaggtcgggagccgagatcttgaagtcgcagttggggccggaaccctgcgcgcgcgagttgcggtacacggggttgcagcactggaacaccagcagggccggattatgcacgctggccagcaggctctcgtcgctgatcatgtcgctgtccagatcctccgcgttgctcagggcgaacggggtcatcttgcagacctgcctgcccaggaaaggcggcagcccgggcttgccgttgcagtcgcagcgcaggggcatcagcaggtgcccgcggcccgactgcgcctgcgggtacagcgcgcgcatgaaggcttcgatctgcctgaaagccacctgcgtcttggctccctccgaaaagaacatcccacaggacttgctggagaactggttcgcgggacagctggcatcgtgcaggcagcagcgcgcgtcggtgttggcgatctgcaccacgttgcgaccccaccggttcttcactatcttggccttggaagcctgctccttcagcgcgcgctggccgttctcgctggtcacatccatctctatcacctgctccttgttgatcatgtttgtaccgtgcagacacttcaggtcgccctccgtctgggtgcagcggtgctcccacagcgcgcaaccggtgggctcccaatttttgtgggtcacccccgcgtaggcctgcaggtaggcctgcaagaagcgccccatcatggccacaaaggtcttctggctcgtaaaggtcagctgcaggccgcgatgctcttcgttcagccaggtcttgcagatggcggccagcgcctcggtctgctcgggcagcatcctaaaatttgtcttcaggtcgttatccacgtggtacttgtccatcatggcgcgcgccgcctccatgcccttctcccaggcggacaccatgggcaggcttagggggtttatcacttccaccggcgaggacaccgtactttcgatttcttcttcctccccctcttcccggcgcgcgcccacgctgctgcgcgctctcaccgcctgcaccaaggggtcgtcttcaggcaagcgccgcaccgagcgcttgccgcccttgacctgcttaatcagcaccggcgggttgctgaagcccaccatggtcagcgccgcctgctcttcttcgtcttcgctgtctaccactatctctggggaagggcttctccgctctgcggcggcgcgcttcttttttttcttgggagcggccgtgatggagtccgccacggcgacggaggtcgagggcgtggggctgggggtgcgcggtaccagggcctcgtcgccctcggactcttcctctgactccaggcggcggcggagtcgcttctttgggggcgcgcgcgtcagcggcggcggagacggggacggggacggggacgggacgccctccacagggggtggtcttcgcgcagacccgcggccgcgctcgggggtcttctcgagctggtcttggtcccgactggccattgtatcctcctcctcctaggcagagagacataaggagtctatcatgcaagtcgagaaggaggagagcttaaccaccccctctgagaccgccgatgcgcccgccgtcgccgtcgcccccgctgccgccgacgcgcccgccacaccgagcgacacccccgcggacccccccgccgacgcacccctgttcgaggaagcggccgtggagcaggacccgggctttgtctcggcagaggaggatttgcgagaggaggaggataaggagaagaagccctcagtgccaaaagatgataaagagcaagacgagcacgacgcagatgcacaccagggtgaagtcgggcggggggacggagggcatgacggcgccgactacctagacgaagggaacgacgtgctcttgaagcacctgcatcgtcagtgcgccattgtttgcgacgctctgcaggagcgcagcgaagtgcccctcagcgtggcggaggtcagccacgcctacgagctcagcctcttctccccccgggtgcccccccgccgccgcgaaaacggcacatgcgagcccaacccgcgcctcaacttctaccccgcctttgtggtacccgaggtcctggccacctatcacatcttctttcaaaattgcaagatccccctctcgtgccgcgccaaccgtagccgcgccgataagatgctggccctgcgccagggcgaccacatacctgatatcgccgctttggaagatgtaccaaagatcttcgagggtctgggtcgcaacgagaagcgggcagcaaactctctgcaacaggaaaacagcgaaaatgagagtcacaccggggtactggtggagctcgagggcgacaacgcccgcctggcggtggtcaagcgcagcatcgaggtcacccactttgcctaccccgcgctaaacctgccccccaaagtcatgaacgcggccatggacgggctgatcatgcgccgcggccggcccctcgctccagatgcaaacttgcatgaggagaccgaggacggccagcccgtggtcagcgacgagcagctggcgcgctggctggagaccgcggaccccgccgaactggaggagcggcgcaagatgatgatggccgtggtgctggtcaccgtagagctggagtgtctgcagcgcttcttcggcgaccccgagatgcagagaaaggtcgaggagaccctgcactacaccttccgccagggctacgtgcgccaggcttgcaagatctccaacgtggagctcagcaacctggtgtcctacctgggcatcttgcatgagaaccgcctcgggcagagcgtgctgcactccaccctgcgcggggaggcgcgccgcgactacgtgcgcgactgcgtttacctcttcctctgctacacctggcagacggccatgggggtctggcagcagtgcctggaggagcgcaacctcaaggagctggagaagctcctgcagcgcgcgctcaaagatctctggacgggctacaacgagcgctcggtggccgccgcgctggccgacctcatcttccccgagcgcctgctcaaaaccctccagcaggggctgcccgacttcaccagccaaagcatgttgcaaaacttcaggaactttatcctggagcgttctggcatcctacccgccacctgctgcgccctgcccagcgactttgtccccctcgtgtaccgcgagtgccccccgccgctgtggggtcactgctacctgttccaactggccaactacctgtcctaccacgcggacctcatggaggactccagcggcgaggggctcatggagtgccactgccgctgcaacctctgcacgccccaccgctccctggtctgcaacacccaactgctcagcgagagtcagattatcggtaccttcgagctacagggtccgtcctcctcagacgagaagtccgcggctccggggctaaaactcactccggggctgtggacttccgcctacctgcgcaaatttgtacctgaagactaccacgcccacgagatcaggttttacgaagaccaatcccgcccgcccaaggcggagctgaccgcctgcgtcatcacccagggcgagatcctaggccaattgcaagccatccaaaaagcccgccaagactttttgctgaagaagggtcggggggtgtatctggacccccagtcgggtgaggagctcaacccggttcccccgctgccgccgccgcgggaccttgcttcccaggataagcatcgccatggctcccagaaagaagcagcagcggccgccactgccgccaccccacatgctggaggaagaggaggaatactgggacagtcaggcagaggaggtttcggacgaggaggagccggagacggagatggaagagtgggaggaggacagcttagacgaggaggcttccgaagccgaagaggcagacgcaacaccgtcaccctcggccgcagccccctcgcaggcgcccccgaagtccgctcccagcatcagcagcaacagcagcgctataacctccgctcctccaccgccgcgacccacggccgaccgcagacccaaccgtagatgggacaccaccggaaccggggccggtaagtcctccgggagaggcaagcaagcgcagcgccaaggctaccgctcgtggcgcgctcacaagaacgccatagtcgcttgcttgcaagactgcggggggaacatctccttcgcccgccgcttcctgctcttccaccacggtgtggccttcccccgtaacgtcctgcattactaccgtcatctctacagcccctactgcggcggcagtgagccagagacggtcggcggcggcggcggcgcccgtttcggcgcctaggaagacccagggcaagacttcagccaagaaactcgcggcggccgcggcgaacgcggtcgcgggggccctgcgcctgacggtgaacgaacccctgtcgacccgcgaactgaggaaccgaatcttccccactctctatgccatcttccagcagagcagagggcaggatcaggaactgaaagtaaaaaacaggtctctgcgctccctcacccgcagctgtctgtatcacaagagcgaagaccagcttcggcgcacgctggaggacgctgaggcactcttcagcaaatactgcgcgctcactcttaaggactagctccgcgcccttctcgaatttaggcgggaacgcctacgtcatcgcagcgccgccgtcatgagcaaggacattcccacgccatacatgtggagctatcagccgcagatgggactcgcggcgggcgcctcccaagactactccacccgcatgaactggctcagtgccggcccacacatgatctcacaggttaatgatatccgcacccatcgaaaccaaatattggtggagcaggcggcaattaccaccacgccccgcaataatcccaaccccagggagtggcccgcgtccctggtgtatcaggaaattcccggccccaccaccgtactacttccgcgtgattcccaggccgaagtccaaatgactaactcaggggcacagctcgcgggcggctgtcgtcacagggtgcggcctcctcgccagggtataactcacctggagatccgaggcagaggtattcagctcaacgacgagtcggtgagctcctcgctcggtctcagacctgacgggaccttccagatagccggagccggccgatcttccttcacgccccgccaggcgtacctgactctgcaaagctcgtcctcggcgccgcgctcgggcggcatcgggactctccagttcgtgcaggagtttgtgccctcggtctacttcaaccccttctcgggctctcccggtcgctacccggaccagttcatctcgaactttgacgccgcgagggactcggtggacggctacgactgaatgtcgggtggacccggtgcagagcaacttcgcctgaagcacctcgaccactgccgccgccctcagtgctttgcccgctgtcagaccggtgagttccagtacttttccctgcccgactcgcacccggacggcccggcgcacggggtgcgctttttcatcccgagtcaggtgcgctctaccctaatcagggagtttaccgcccgtcccctactggcggagttggaaaaggggccttctatcctaaccattgcctgcatctgctctaaccctggattgcaccaagatctttgctgtcatttgtgtgctgagtataataaaggctgagatcagaatctactcgggctcctgtcgccatcctgtcaacgccaccgtccaagcccggcccgatcagcccgaggtgaacctcacctgcggtctgcaccggcgcctgaggaaatacctagcttggtactacaacagcactccctttgtggtttacaacagctttgaccaggacggggtctcactgagggataacctctcgaacctgagctactccatcaggaagaacagcaccctcgagctacttcctccttacctgcccgggacttaccagtgtgtcaccggtccctgcacccacacccacctgttgatcgtaaacgactctcttccgagaacagacctcaataactcctcttcgcagttccccagaacaggaggtgagctcaggaaaccccgggtaaagaagggtggacgagagttaacacttgtggggtttctggtgtatgtgacgctggtggtggctcttttgattaaggcttttccttccatgtctgaactctccctcttcttttatgaacaactcgactagtgctaacgggaccctacccaacgaatcgggattgaatatcggtaaccaggttgcagtttcacttttgattaccttcatagtcctcttcctgctagtgctgtcgcttctgtgcctgcggatcgggggctgctgcatccacgtttatatctggtgctggctgtttagaaggttcggagaccatcgcaggtagaataaacatgctgctgcttaccctctttgtcctggcgctggccgccagctgccaagccttttccgaggctgactttatagagccccagtgtaatgtgacttttaaagcccatgcacagcgttgtcatactataatcaaatgtgccaccgaacacgatgaataccttatccagtataaagataaatcacacaaagtggcacttgttgacatctggaaacccgaagaccctttggaatacaatgtgaccgttttccagggtgacctcttcaaaatttacaattacactttcccatttgaccagatgtgtgactttgtcatgtacatggaaaagcagcacaagctgtggcctccgactccccagggctgtgtggaaaatccaggctctttctgcatgatctctctctgtgtaactgtgctggcactaatactcacgcttttgtatatcagatttaaatcaaggcaaagcttcattgatgaaaagaaaatgccttaatcgctttcacgcttgattgctaacaccgggtttttatccgcagaatgattggaatcaccctactaatcacctccctccttgcgattgcccatgggttggaacgaatcgaagtccctgtgggggccaatgttaccctggtggggcctgtcggcaatgctacattaatgtgggaaaaatatactaaaaatcaatgggtctcttactgcactaacaaaaatagccacaagcccagagccatctgcgatgggcaaaatctaaccttgattgatgttcaattgctggatgcgggctactattatgggcagctgggtacaatgattaattactggagaccccacagagattacatgctccacgtagtaaagggtccccttagcagcccacccactaccacctctactacccccactaccaccactactcccaccaccagcactgccgcccagcctcctcatagcagaacaaccacttttatcaattccaagtcccactccccccacattgccggcgggccctccgcctcagactccgaaaccaccgagatctgcttctgcaaatgctctgacgccattgcccaggatttggaagatcacgaggaagatgagcatgacttcgcagatgcatgccaggcatcagagccagaagcgctgccggtggccctcaaacagtatgcagacccccacaccacccccgaccttcctccaccttcccagaagccaagtttcctgggggaaaatgaaactctgcctctctccatactcgctctgacatctgttgctatgttgaccgctctgctggtgcttctatgctctatatgctacctgatctgctgcagaaagaaaaaatctcacggccatgctcaccagcccctcatgcacttcccttaccctccagagctgggcgaccacaaactttaagtctgcagtaactatctgcccatcccttgtcagtcgacagcgatgagccccactaatctaacggcctctggacttacaacatcgtctcttaatgagaccaccgctcctcaagacctgtacgatggtgtctccgcgctggttaaccagtgggatcacctgggcatatggtggctcctcataggagcagtgaccctgtgcctaatcctggtctggatcatctgctgcatcaaaagcagaagacccaggcggcggcccatctacaggccctttgtcatcacacctgaagatgatgatgacaccacttccaggctgcagaggctaaagcagctactcttctcttttacagcatggtaaattgaatcatgcctcgcattttcatctacttgtctctccttccactttttctgggctcttctacattggccgctgtgtcccacatcgaggtagactgcctcacgcccttcacagtctacctgcttttcggctttgtcatctgcacctttgtctgcagcgttatcactgtagtgatctgcttcatacagtgcatcgactacgtctgcgtgcgggtggcttactttagacaccacccccagtatcgcaacagggacatagcggctctcctaagacttgtttaaaatcatggccaaattaactgtgattggtcttctgatcatctgctgcgtcctagccgcgattgggactcaagctcctaccaccaccagcgctcccagaaagagacatgtatcctgcagcttcaagcgtccctggaatataccccaatgctttactgatgaacctgaaatctctttggcttggtacttcagcgtcaccgcccttcttatcttctgcagtacggttattgcccttgccatctacccttcccttgacctgggctggaatgctgtcaactctatggaatatcccaccttcccagaaccagacctgccagacctggttgttctaaacgcgtttcctcctcctgctcccgttcaaaatcagtttcgccctccgtcccccacgcccactgaggtcagctactttaatctaacaggcggagatgactgaaaacctagacctagaaatggacggtctctgcagcgagcaacgcacactagagaggcgccggcaaaaagagctcgagcgtcttaaacaagagctccaagacgcggtggccatacaccagtgcaaaaaaggtgtcttctgtctggtaaaacaggccacgctcacctatgaaaaaacaggtgacacccaccgcctaggatacaagctgcccacacagcgccaaaagttcgccctcatgataggcgaacaacccatcaccgtgacccagcactccgtggagacagaaggctgcatacatgctccctgtaggggcgctgactgcctctacaccttgatcaaaaccctctgcggtctcagagaccttatccctttcaattaatcataactgtaatcaataaaaaatcacttacttgaaatctgatagcaagcctctgtccaattttttcagcaacacttccttcccctcctcccaactctggtactctaggcgcctcctagctgcaaacttcctccacagtctgaagggaatgtcagattcctcctcctgtccctccgcacccacgatcttcatgttgttgcagatgaaacgcgcgagatcgtctgacgagaccttcaaccccgtgtacccctacgataccgagatcgctccgacttctgtccctttccttacccctccctttgtgtcatccgcaggaatgcaagaaaatccagctggggtgctgtccctgcacttgtcagagccccttaccacccacaatggggccctgactctaaaaatggggggcggcctgaccctggacaaggaagggaatctcacttcccaaaacatcaccagtgtcgatccccctctcaaaaaaagcaagaacaacatcagccttcagaccgccgcacccctcgccgtcagctccggggccctaacactttttgccactccccccctagcggtcagtggtgacaaccttactgtgcagtctcaggcccctctcactttggaagactcaaaactaactctggccaccaaaggacccctaactgtgtccgaaggcaaacttgtcctagaaac

SEQ ID NO: 24 - DNA, 32728 bp, synthetic construct
  misc_feature 1..32728: note = Adenovirus vector nucleotide sequences
  source 1..32728: mol_type = other DNA
ataatataccttattttggattgtggccaatatgataatgaggtgggcggggagaggcggggcgggtgacgtaggacgcgcgagtagggttgggaggtgtggcggaagtgtggcatttgcaagtgggaggagctcacatgcaagcttccgtcgcggaaaatgtgacgtttttgatgagcgccgcctacctccggaagtgccaattttcgcgcgcttttcaccggatatcgtagtaattttgggcgggaccatgtaagatttggccattttcgcgcgaaaagtgaaacggggaagtgaaaactgaataatagggcgttagtcatagcgcgtaatatttaccgagggccgagggactttgaccgattacgtggaggactcgcccaggtgttttttacgtgaatttccgcgttccgggtcaaagtctccgtttttattgtcaccgtcatttgacgcggagggtatttaaacccgctgcgctcctcaagaggccactcttgagtgccagcgagaagagttttctcctctgctccgcttcggtgatcgaaaaatgagacacatagcctgcactccgggtcttttgtccggtcgggcggcggccgagcttttggacgctttgatcaatgatgtcctaagcgatgattttccgtctactacccactttagcccacctactcttcacgaactgtacgatctggatgtactggtggatgtgaacgatcccaacgaggaggcggtttctgcgttttttcccgagtctgcgctgttggccgctcaggagggatttgacctacacactccgccgcctattttagagtctccgctgccggagcccagtggtataccttatatgcctgaactgcttcccgaagtggtagacctgacctgccacgagcctggctttccgcccagcgacgatgagggtgagccttttgttttagactttgctgagatacctgggcacggttgcaggtcttgtgcatatcatcagagggttaccggagaccccgaggttaagtgttcgctgtgctatatgaggatgacctcttcctttatctacagtaagtttttgtctaggtgggcttttgggtaggtgggttttgtgtcagaacaggtgtaaacgttgcttgtgttttttgtacctgtaggtccggtgtccgagccagacccggagcccgaccgcgatcccgagccggatcccgagcctcctcgcaggacaaggaaactaccttccattctgtgcaagtctcagacacctgtaaggaccagcgaggcagacagcaccgactctggcacttctacctctccccctgaaattcacccagtggttcctctgggtatacataaacctgttgctgttaaagtttgcgggcgacgccctgcagtacagtgcattgaggacttgcttcacgatcccgaggaacctttggacttgagccttaaacgccctaggcaataaaccccacctaagtaataaaccccacctaagtaataaaccctgccgcccttggttattgagatgacgcccaatgtttgcttttgaatgacttcatgtgtgtaataaaagtgagtgtgatcataggtctcttgtttgtctgggcggggcttaagggtatataagtctcttggggctaaacttggttacacttgaccccaatggaggcgtgggggtgcttggaggagtttgcggacgtgcgccgtttgctggacgagagctctagcaatacctatactatttggaggtatctgtggggctctactcaggccaagttggtttccagaattaagcaggattacaagtgcgattttgaagagctttttagttcctgcggtgagcttttgcaatccttgaatctgggccatcaggctattttccaggaaaaggttctctcgactttggatttttccactcccgggcgcaccgccgcttgtgtggcttttgtgtcttttgtgcaagataaatggagcgaggagacccacctgagtcacggctacgtactggatttcatggcgatggctctttggagggctcacaacaaatggaagattcagaaggaactgtacggttccgccctacgtcgtccacttctgtcgcgacaggggctgaggtttcccgaccatcggcagcatcagaatctggaagacgagtcggaggagcgagcggaggagaagatcagcttgagagccggcctggaccctcctcaggaggaatgaatctcccgcaggtggttgacctgtttccagaactgagacgggtcctgactatcagggaggatggtcagtttgtgaagaagtttaagagggatcggggtgagggagatgatgaggcggctagcaatttagcttttagtctgatgactcgccaccgaccggaatgtattacctatcagcagattaaggagagttgtgccaacgagctggatcttttgggtcagaagtatagcatagaacagcttaccacttactggcttcagcctggggatgattgggaagaggcgatcagggtgtatgcaaaggtggccctgcggcccgattgcaagtataagattactaagttggttaatattagaaactgctgctatatttctgggaacggggccgaagtggagatagatactcaggacagggtggcttttaggtgttgcatgataaacatgtggcccgggatactggggatggatggggtggtattcatgaatgtgaggtttacgggccccaactttaatggcacggtgttcatgggcaacaccaacttgctcctgcatggtgcgagtttctatgggtttaataacacctgtatagaggcctggaccgatgtaaaggttcgaggttgttccttttatagctgttggaaggcggtggtgtgtcgccctaaaagcaggggttctgtgaaaaaatgcttgtttgaaaggtgcaccttaggcatcctctctgagggcaactccagggtgcgccataatgtggcttcgaactgcggttgcttcatgcaagtgaagggggtgagcgttatcaagcataactcggtgtgtggaaactgcgaggatcgcgcctcccagatgctgacctgctttgatggcaactgtcacctgttgaagaccattcatataagcagccaccccagaaaggcctggcccgtgtttgagcataacatcttgacccgctgctccttgcatctgggggtcaggaggggtatgttcctgccttaccagtgtaactttagccacactaaaatcctgctggaacccgagtgcatgaccaaggtcagcctgaatggtgtgtttgatgtgactctgaaaatctggaaggtgctgaggtatgatgagaccaggaccaggtgccgaccctgcgagtgcggcggcaagcacatgagaaatcagcctgtgatgttggatgtgaccgaggagcttaggcctgaccatctggtgctggcctgcaccagggccgagtttgggtctagcgatgaggataccgattgaggtgggtaaggtgggcgtggctagaagggtggggcgtgtataaattgggggtctaagggtctctctgttttgtcttgcaacagccgccgccatgagcgacaccggcaacagctttgatggaagcatctttagcccctatctgacagtgcgcatgcctcactgggctggagtgcgtcagaatgtgatgggttccaacgtggatggacgccccgttctgccttcaaattcgtctacaatggcctacgcgaccgtgggaggaactccgctggacgccgcgacctccgccgccgcctccgccgccgccgcgaccgcgcgcagcatggctacggacctttacagctctttggtggcgagcggcgcggcctctcgcgcgtctgctcgggatgagaaactgaccgctctgctgcttaaactggaagacttgacccgggagctgggtcaactgacccagcaggtctccagcttgcgtgagagcagccttgcctccccctaatggcccataatataaataaaagccagtctgtttggattaagcaagtgtatgttctttatttaactctccgcgcgcggtaagcccgggaccagcggtctcggtcgtttagggtgcggtggattctttccaacacgtggtacaggtggctctggatgtttagatacatgggcatgagtccatccctggggtggaggtagcaccactgcagagcttcgtgctcgggggtggtgttgtatatgatccagtcgtagcaggagcgctgggcgtggtgctgaaaaatgtccttaagcaagaggcttatagctagggggaggcccttggtgtaagtgtttacaaatctgctcagttgggaggggtgcatccggggggatataatgtgcatcttggactggatttttaggttggctatgttcccacccagatcccttctgggattcatgttgtgcaggaccaccagcacggtatatccagtgcacttgggaaatttatcgtggagcttagacgggaatgcatggaagaacttggagacgcccttgtggcctcccagattttccatacattcgtccatgatgatggcaatgggcccgtgggaagctgcctgagcaaaaatgtttctgggatcgctcacatcgtagttatgttccagggtgaggtcatcataggacatctttacgaatcgggggcggagggtcccggactgggggatgatggtaccctcgggccccggggcgtagttcccctcacagatctgcatctcccaggctttcatttcagagggagggatcatatccacctgcggagcgatgaaaaacacagtttctggcgcaggggagattaactgggatgagagcaggtttctgagcagctgtgactttccacagccggtgggcccatatatcacgcctatcaccggctgcagctggtagttaagagagctgcagctgccgtcctcccggagcaggggggccacctcgttcagcatatccctgacgtggatgttctccctgaccaattccgccagaaggcgctcgccgcccagcgaaagcagctcttgcaaggaagcaaaatttttcagcggttttaggccgtcggccgtgggcatgtttttcagcgtctgggtcagcagttccagcctgtcccacagctcggtgatgtgctctacggcatctcgatccagcagatctcctcgtttcgcgggttggggcggctttcgctgtagggcaccagccgatgggcgtccagcggggccagagtcatgtccttccatgggcgcagggtcctcgtcagggtggtctgggtcacggtgaaggggtgcgctccgggttgggcgctggccagggtgcgcttgaggctggttctgctggtgctgaatcgctgccgctcttcgccctgcgcgtcggccaggtagcatttgaccatggtctcgtagtcgagaccctcggcggcgtgccccttggcgcggagctttcccttggaggtggcgccgcacgaggggcactgcaggctcttcagggcgtagagcttgggagcgagaaacacggactctggggagtaggcgtccgcgccgcaggaagcgcagaccgtctcgcattccaccagccaagtgagctccgggcggtcagggtcaaaaaccaggttgcccccatgctttttgatgcgtttcttacctcggctctccatgaggcggtgtcccttctcggtgacgaagaggctgtccgtgtccccgtagaccgacttcaggggcctgtcttccagcggagtgcctctgtcctcctcgtagagaaactctgaccactctgagacgaaggcccgcgtccaggccaggacgaaggaggccacgtgggaggggtagcggtcgttgtccactagcgggtccaccttctccagggtgtgcaggcacatgtccccctcctccgcgtccagaaaagtgattggcttgtaggtgtaggacacgtgaccgggggttcccgacgggggggtataaaagggggtgggcgccctttcatcttcactctcttccgcatcgctgtctgcgagggccagctgctggggtaagtattccctctcgaaggcgggcatgacctcagcgctcaggttgtcagtttctaaaaatgaggaggatttgatgttcacctgtccggaggtgatacctttgagggtacctgggtccatctggtcagaaaacactatttttttgttgtcaagcttggtggcgaacgacccgtagagggcgttggagagcagcttggcgatggagcgcagggtctggtttttgtcgcggtcggctcgctccttggccgcgatgttgagttgcacgtactcgcgggccacgcacttccactcggggaagacggtggtgcgctcgtctgggattaggcgcaccctccagcctcggttgtgcagggtgaccatgtcgacgctggtggcgacctcgccgcgcaggcgctcgttggtccagcagaggcggccgcccttgcgcgagcagaaggggggtagggggtccagctggtcctcgtttggggggtccgcgtcgatggtgaagaccccggggagcaagcgcgggtcaaagtagtcgatcttgcaagcttgcatgtccagagcccgctgccattcgcgggcggcgagcgcgcgctcgtaggggttgaggggcgggccccagggcatggggtgggtgagcgcggaggcgtacatgccgcagatgtcatacacgtacaggggttccctgaggatgccgaggtaggtggggtagcagcgccccccgcggatgctggcgcgcacgtagtcatagagctcgtgggagggggccagcatgttgggcccgaggttggtgcgctgggggcgctcggcgcggaaggcgatctgcctgaagatggcatgggagttggaggagatggtgggccgctggaagacgttgaagcttgcttcttgcaagcccaccgagtccctgacgaaggaggcgtaggactcgcgcagcttgtgcaccagctcggcggtgacctggacgtcgagcgcgcagtagtcgagggtctcgcggatgatgtcatacttatcctcccccttctttttccacagctcgcggttgaggacgaactcttcgcggtctttccagtactcttggaggggaaacccgtccgtgtccgaacggtaagagcctagcatgtagaactggttgacggcctggtaggggcaacagcccttctccacgggcagcgcgtaggcctgcgccgccttgcggagggaggtgtgggtgagggcgaaagtgtccctgaccatgactttgaggtattgatgtttgaagtctgtgtcatcgcagccgccctgttcccacagggtgtagtccgtgcgctttttggagcgcgggttgggcagggagaaggtgaggtcattgaagaggatcttccccgctcgaggcatgaagtttctggtgatgcgaaagggccctgggaccgaggagcggttgttgatgacctgggcggccaggacgatctcgtcaaagccgtttatgttgtggcccacgatgtagagctccaaaaagcggggctggcccttgatggaggggagctttttgagttcctcgtaggtgagctcctcgggcgattccaggccgtgctcctccagggcccagtcttgcaagtgagggttggccgccaggaaggatcgccagaggtcgcgggccatgagggtctgcaggcggtcgcggaaggttctgaactgtcgccccacggccatcttttcgggggtgatgcagtagaaggtgagggggtctttctcccaggggtcccatctgagctctcgggcgaggtcgcgcgcggcggcgaccagagcctcgtcgccccccagtttcatgaccagcatgaagggcacgagctgcttgccaaaggctcccatccaagtgtaggtctctacatcgtaggtgacaaagaggcgctccgtgcgaggatgagagccgatcgggaagaactggatctcccgccaccagttggaggattggctgttgatgtggtgaaagtagaagtcccgtctgcgggccgagcactcgtgctggcttttgtaaaagcgaccgcagtactggcagcgctgcacgggttgtatatcttgcacgaggtgaacctggcgacctctgacgaggaagcgcagcgggaatctaagtcccccgcctggggtcccgtgtggctggtggtcttctactttggttgtctggccgccagcatctgtctcctggagggcgatggtggagcagaccaccacgccgcgagagccgcaggtccagatctcggcgctcggcgggcggagtttgatgacgacatcgcgcacattggagctgtccatggtctccagctcccgcggcggcaggtcagctgggagttcctggaggttcacctcgcagagacgggtcaaggcgcgggcagtgttgagatggtatctgatttcaaggggcgtgttggcggcggagtcgatggcttgcaggaggccgcagccccggggggccacgatggttccccgcggggcgcgaggggaggcggaagctgggggtgtgttcagaagcggtgacgcgggcgggcccccggaggtagggggggttccggccccacaggcatgggcggcaggggcacgtcttcgccgcgcgcgggcaggggctggtgctggctccgaagagcgcttgcgtgcgcgacgacgcgacggttggtgtcctgtatctgacgcctctgagtgaagaccacgggtcccgtgaccttgaacctgaaagagagttcgacagaatcaatctcggcatcgttgacagcggcctggcgcaggatctcctgcacgtcgcccgagttgtcctggtaggcgatctctgccatgaactgctcgatctcttcttcctggagatctcctcgtccggcgcgctccacggtggccgccaggtcgttggagatgcgacccatgagctgtgagaaggcgttgagcccgccctcgttccagacccggctgtagaccacgcccccctcggcgtcgcgagcgcgcatgaccacctgggccaggttgagctccacgtgtcgcgtgaagacggcgtagttgcgcaggcgctggaaaaggtagttcagggtggtggcggtgtgctcggcgacgaagaagtacatgacccagcgccgcaacgtggattcattgatgtcccccaaggcctccaggcgctccatggcctcgtagaagtccacggcgaagttgaaaaactgggagttgcgagcggacacggtcaactcctcctccagaagacggatgagctcggcgacagtgttgcgcacctcgcgctcgaaggccacggggggcgcttcttcctcttccacctcttcttccatgatcgcttcttcttcttcctcagccgggacgggagggggcggcggcggcgggggaggggcgcggcggcggcggcggcgcaccgggaggcggtcgatgaagcgctcgatcatctccccccgcatgcggcgcatggtctcggtgacggcgcggccgttctcccgggggcgcagctcgaagacgccgcctctcatctcgccgcggggcgagcggccgtgaggtagcgagacggcgctgactatgcatcttaacaattgctgtgtaggtacaccgccgagggacctgattgagtccagatccaccggatccgaaaacctttggaggaaagcgtctatccagtcgcagtcgcaaggtaggctgagcaccgtggcgggcgggggcgggtctggagagttcctggcggagatgctgctgatgatgtaattaaagtaggcggtcttgagaaggcggatggtggacaggagcaccatgtctttgggtccggcctgttggatgcggaggcggtcggccatgccccaggcctcgttctgacaccggcgcaggtctttgtagtagtcttgcatgagtctttccaccggcacctcttctccttcctcttctccatctcgccggtggtttctcgcgccgcccatgcgcgtgaccccaaagcccctgagcggctgcagcagggccaggtcggcgaccacgcgctcggccaagatggcctgctgcacctgagtgagggtcctctcgaagtcatccatgtccacgaagcggtggtaggcgcccgtgttgatggtgtaggtgcagttggccatgacggaccagttgacggtctggtgtcccggctgcgagagctccgtgtaccgcaggcgcgagaaggcgcgggaatcgaacacgtagtcgttgcaagtccgcaccagatactggtagcccaccaggaagtgcggcggaggttggcgatagaggggccagcgctgggtggcgggggcgccgggcgccaggtcttccagcatgaggcggtggtatccgtagatgtacctggacatccaggtgatgccggcggcggtggtggtggcgcgcgcgtagtcgcggacccggttccagatgtttcgcaggggcgagaagtgttccatggtcggcacgctctggccggtgaggcgcgcgcagtcgttgacgctctatacacacacaaaaacgaaagcgtttacagggctttcgttctgtagcctggaggaaagtaaatgggttgggttgcggtgtgccccggttcgagaccaagctgagctcggccggctgaagccgcagctaacgtggtattggcagtcccgtctcgacccaggccctgtatcctccaggatacggtcgagagcccttttgctttcttggccaagcgcccgtggcgcgatctgggatagatggtcgcgatgagaggacaaaagcggctcgcttccgtagtctggagaaacaatcgccagggttgcgttgcggcgtaccccggttcgagcccctatggcggcttgaatcggccggaaccgcggctaacgagggccgtggcagccccgtcctcaggaccccgccagccgacttctccagttacgggagcgagccccttttgttttttattttttagatgcatcccgtgctgcggcagatgcgcccctcgccccggcccgatcagcagcagcaacagcaggcatgcagacccccctctcccctttccgccccggtcaccacggccgcggcggccgtgtcgggcgcggggggcgcgctggagtcagatgagccaccgcggcggcgacctaggcagtatctggacttggaagagggcgagggactggcgcggctgggggcgaactctccagagcgccacccgcgggtgcagttgaaaagggacgcgcgcgaggcgtacctgccgcggcagaacctgtttcgcgaccgcgggggcgaggagcccgaggagatgcgagactgcaggttccaagcggggcgcgagctgcggcgcgggctggacagacagcgcctgctgcgcgaggaggactttgagcccgacacgcagacgggcatcagccccgcgcgcgcgcacgtagccgcggccgacctggtgaccgcctacgagcagacggtaaaccaggagcgcaacttccaaaagagcttcaacaaccacgtgcgcacgctggtggcgcgcgaggaggtgaccctgggtctcatgcatctgtgggacctggtggaggcgatcgtgcagaaccccagcagcaagcccctgaccgcgcagctgttcctggtggtgcagcacagcagggacaacgaggccttcagggaggcgctgctgaacatcaccgagccggaggggcgctggctcctggacctgataaacatcctgcagagcatagtggtgcaggagcgcagcctgagcctggccgagaaggtggcggccatcaactactctatgctgagcctgggcaagttctacgcccgcaagatctacaagaccccctacgtgcccatagacaaggaggtgaagatagacagcttctacatgcgcatggcgctgaaggtgctgaccctgagcgacgacctgggagtgtaccgcaacgagcgcatccacaaggccgtgagcgccagccggcggcgcgagctgagcgaccgcgagctgatgcacagtctgcagcgcgcgctgaccggcgcgggcgagggcgacagggaggtcgagtcctacttcgacatgggggccgacctgcactggcagccgagccgccgcgccctggaggcggcgggggcgtacggcggccccctggcggccgatgaccaggaagaggaggactatgagctagaggagggcgagtacctggaggactgacctggctggtggtgttttggtatagatgcaagatccgaacgtggcggacccggcggtccgggcggcgctgcaaagccagccgtccggcattaactcctctgacgactgggccgcggccatgggtcgcatcatggccctgaccgcgcgcaaccccgaggctttcaggcagcagcctcaggccaaccggctggcggccatcttggaagcggtagtgcccgcgcgctccaaccccacccacgagaaggtgctggccatagtcaacgcgctggcggagagcagggccatccgcgcggacgaggccggactggtgtacgatgcgctgctgcagcgggtggcgcggtacaacagcggcaacgtgcagaccaacctggaccgcctggtgacggacgtgcgcgaggccgtggcgcagcgcgagcgcttgcatcaggacggtaacctgggctcgctggtggcgctaaacgccttcctcagcacccagccggccaacgtaccgcgggggcaggaggactacaccaactttttgagcgcgctgcggctgatggtgaccgaggtccctcagagcgaggtgtaccagtcggggcccgactacttcttccagaccagcagacagggcttgcaaaccgtgaacctgagccaggctttcaagaacctgcgggggctgtggggagtgaaggcgcccaccggcgaccgggctacggtgtccagcctgctaacccccaactcgcgcctgctgctgctgctgatcgcgcccttcacggacagcgggagcgtctcgcgggagacctatctgggccacctgctgacgctgtaccgcgaggccatcgggcaggcgcaggtggacgagcacaccttccaagagatcaccagcgtgagccacgcgctggggcaggaggacacgggcagcctgcaggcgaccctgaactacctgctgaccaacaggcggcagaagattcccacgctgcacagcctgacccaggaggaggagcgcatcttgcgctacgtgcagcagagcgtgagcctgaacctgatgcgcgacggcgtgacgcccagcgtggcgctggacatgaccgcgcgcaacatggaaccgggcatgtacgcctcccaccggccgtttatcaaccgcctgatggactacttgcatcgggcggcggccgtgaaccccgagtacttcactaatgccattctgaatccccactggatgccccctccgggtttctacaacggggactttgaggtgcccgaggtcaacgacgggttcctctgggatgacatggatgacagtgtgttctcacccaacccgctgcgcgccgcgtctctgcgattgaaggagggctctgacagggaaggaccgaggagtctggcctcctccctggctctgggagcggtgggcgccacgggcgcggcggcgcggggcagtagccccttccccagcctggcagactctctgaacagcgggcgggtgagcaggccccgcttgctaggcgaggaggagtatctgaacaactccctgctgcagcccgcgagggacaagaacgctcagcggcagcagtttcccaacaatgggatagagagcctggtggacaagatgtccagatggaagacgtatgcgcaggagtacaaggagtgggaggaccgccagccgcggcccttgccgccccctaggcagcgctggcagcggcgcgcgtccaaccgccgctggaggcaggggcccgaggacgatgatgactctgcagatgacagcagcgtgttggacctgggcgggagcgggaaccccttttcgcacctgcgcccacgcctgggcaagatgttttaaaagaaaaaaaaaaataaaactcaccaaggccatggcgacgagcgttggttttttgttcccttccttagtatgcggcgcgcggcgatgttcgaggaggggcctcccccctcttacgagagcgcgatggggatttctcctgcggcgcccctgcagcctccctacgtgcctcctcggtacctgcaacctacaggggggagaaatagcatctgttactctgagctgcagcccctgtacgataccaccagactgtacctggtggacaacaagtccgcggacgtggcctccctgaactaccagaacgaccacagcgattttttgaccacggtgatccaaaacaacgacttcaccccaaccgaggccagcacccagaccataaacctggataacaggtcgaactggggcggcgacctgaagaccatcttgcacaccaacatgcccaacgtgaacgagttcatgttcaccaactcttttaaggcgcgggtgatggtggcgcgcgagcagggggaggcgaagtacgagtgggtggacttcacgctgcccgagggcaactactcagagaccatgactctcgacctgatgaacaatgcgatcgtggaacactatctgaaagtgggcaggcagaacggggtgaaggaaagcgatatcggggtcaagtttgacaccagaaacttccgtctgggctgggaccccgtgaccgggctggtcatgccgggggtctacaccaacgaggcctttcatcccgacatagtgcttctgcccggctgtggggtggacttcacccagagccggctgagcaacctgctgggcattcgcaagcggcagcctttccaggagggtttcaagatcacctatgaggatctgaaggggggcaacattcccgcgctccttgatctggacgcctacgaggagagcttgaaacccgaggagagcgctggcgacagcggcgagagtggcgaggagcaagccggcggcggtggcggcgcgtcggtagaaaacgaaagtacgcccgcagtggcggcggacgctgcggaggtcgagccggaggccatgcagcaggacgcagaggagggcgcacaggagggcgcgcagaaggacatgaacgatggggagatcaggggagacacattcgccacccggggcgaagaaaaagaggcagaggcggcggcggcggcgacggcggaggccgaaaccgaggttgaggcagaggcagagcccgagaccgaagttatggaagacatgaatgatggagaacgtaggggcgacacgttcgccacccggggcgaagagaaggcggcggaggcagaagccgcggctgaggaggcggctgcggctgcggccaagactgaggctgcggctaaggctgaggtcgaagccaatgttgcggttgaggctcaggctgaggaggaggcggcggctgaagcagttaaggaaaaggcccaggcagagcaggaagagaaaaaacctgtcattcaacctctaaaagaagatagcaaaaagcgcagttacaacgtcatcgagggcagcacctttacccagtaccgcagctggtacctggcgtacaactacggcgacccggtcaagggggtgcgctcgtggaccctgctctgcacgccggacgtcacctgcggctccgagcagatgtactggtcgctgccgaacatgatgcaagacccggtgaccttccgctccacgcggcaggttagcaacttcccggtggtgggcgccgaactgctgcccgtgcactccaagagtttttacaacgagcaggccgtctactcccagctgatccgccaggccacctctctgacccacgtgttcaatcgctttcccgagaaccagattttggcgcgcccgccggcccccaccatcaccaccgtgagtgaaaacgttcctgccctcacagatcacgggacgctaccgctgcgcaacagcatctcaggagtccagcgagtgaccattactgacgccagacgccggacctgcccctacgtttacaaggccttgggcatagtctcgccgcgcgtcctctccagtcgcactttttaaaacacatctacccacacgttccaaaatcatgtccgtactcatctcacccagcaacaacaccggctgggggctgcgcgcgcccagcaagatgtttggaggggcgaggaagcgctccgaccagcaccctgtgcgcgtgcgcggccactaccgcgcgccctggggagcgcacaagcgcgggcgcacagggcgcaccactgtggacgacgtcattgactccgtagtggagcaagcgcgccactacacacccggcgcgccgaccgcccccgccgtgtccaccgtggaccaggcgatcgaaagcgtggtacagggcgcgcggcactatgccaaccttaaaagtcgccgccgccgcgtggcccgccgccatcgccggagaccccgggccaccgccgccgcgcgccttactaaggctctgctcaggcgcgccaggcgaactggccaccgggccgccatgagggccgcacggcgggctgccgctgccgcaagcgtcgtggccccgcgggcacgaaggcgcgcggccgctgccgccgccgccgccatttccagcttggcctcgacgcggcgcggtaacatatactgggtgcgcgactcggtaaccggcacgcgggtacccgtgcgctttcgccccccgcggaattagcacaagacaacatacacactgagtctcctgctgttgtgtatcccagcggcgaccgtcagcagcggcgacatgtccaagcgcaaaattaaagaagagatgctccaggtcatcgcgccggagatctatgggcccccgaagaaggaggaggatgattacaagccccgcaagctaaagcgggtcaaaaagaaaaagaaagatgatgatgacgaggcggtggagtttgtccgccgcatggcacccaggcgccccgtgcagtggaagggccggcgcgtgcagcgcgttttgcgccccggcaccgcggtggtcttcacgcccggcgagcgctccacgcgcactttcaagcgggtgtacgatgaggtgtacggcgacgaggacctgttggagcaggccaaccagcgctttggggagtttgcatatgggaaacggccccgcgagagtctaaaagaggacctgctggcgctaccgctggacgagggcaatcccaccccgagtctgaagccggtaaccctgcaacaggtgctgcctttgagcgcgcccagcgagcataagcgagggttgaagcgcgaaggcggggacctggcgcccaccgtgcagttgatggtgcccaagcggcagaagctggaggacgtgctggagaaaatgaaagtagagcccgggatccagcccgagatcaaggtccgccccatcaagcaggtggcgcccggcgtgggagtccagaccgtggacgttaggattcccacggaggagatggaaacccaaaccgccactccctcttcggcggccagcgccaccaccggcaccgcttcggtagaggtgcagacggacccctggctacccgccaccgctgttgccgccgccgccccccgttcgcgcgggcgcaagagaaattatccagcggccagcgcgctcatgccccagtacgcactgcatccatccatcgtgcccacccccggctaccgcgggtactcgtaccgcccgcgcagatcagccggcactcgcggccgccgccgccgtgcgaccacaaccagccgccgccgtcgccgccgccgccagccagtgctgacccccgtgtctgtaaggaaggtggctcgctcggggagcacgctggtggtgcccagagcgcgctaccaccccagcatcgtttaaagccggtctctgtatggttcttgcagatatggccctcacttgtcgcctccgcttcccggtgccgggataccgaggaagaactcaccgccgcagaggcatggcgggcagcggtctccgcggcggccgtcgccatcgccggcgcgcaaaaagcaggcgcatgcgcggcggtgtgctgcctctgctaatcccgctaatcgccgcggcgatcggtgccgtacccgggatcgcctccgtggccctgcaggcgtcccagaaacgttgactcttgcaaccttgcaagcttgcattttttggaggaaaaaataaaaaaaaagtctagactctcacgctcgcttggtcctgtgactattttgtagaaaaaaagatggaagacatcaactttgcgtcgctggccccgcgtcacggctcgcgcccgttcatgggagactggacagatatcggcaccagcaatatgagcggtggcgccttcagctggggcagtctgtggagcggccttaaaaattttggttccaccattaagaactatggcaacaaagcgtggaacagcagcacgggccagatgctgagagacaagttgaaagagcagaacttccaggagaaggtggcgcagggcctggcctctggcatcagcggggtggtggacatagctaaccaggccgtgcagaaaaagataaacagtcatctggacccccgtcctcaggtggaggaaatgcctccagcgatggagacggtgtctcccgagggcaaaggcgaaaagcgcccgcggcccgacagagaagagaccctggtgtcacacaccgaggagccgccctcttacgaggaggcagtcaaggccggcctgcccaccactcgccccatagcccccatggccaccggtgtggtgggccacaggcaacacactcccgcaacactagatctgcccccgccgtccgagccgccgcgccagccaaaggcggcgacggtgcccgctccctccacttccgccgccaacagagtgcccctgcgccgcgccgcgagcggcccccgggcctcgcgagttagcggcaactggcagagcacactgaacagcatcgtgggcctgggagtgaggagtgtgaagcgccgccgttgctactgaatgagcaagctagctaacgtgttgtatgtgtgtatgcgtcctatgtcgccgccagaggagctgttgagccgccggcgccgtctgcactccagcgaatttcaagatggcgaccccatcgatgatgcctcagtggtcgtacatgcacatctcgggccaggacgcttcggagtacctgagccccgggctggtgcagttcgcccgcgccacagacacctacttcaacatgagtaacaagttcaggaaccccactgtggcgcccacccacgatgtgaccacggaccggtcgcagcgcctgacgctgcggttcatccccgtggatcgggaggacaccgcctactcttacaaggcgcggttcacgctggccgtgggcgacaaccgcgtgctggacatggcctccacttactttgacatcaggggggtgctggacaggggccccaccttcaagccctactcgggtactgcctacaactccctggcccccaagggcgctcccaattcttgcgagtgggaacaagatgaaccagctcaggcagcaatagctgaagatgaagaagaacttgaagaagaacaagctcaggacgaacaggcgcccactaagaaaacccatgtatacgcccaggcacctctttctggtgaaaaaattactaaggatggtttgcaaataggtgtggatgccacacaggcgggagataaccctatatatgctgataaaacattccaacccgaacctcagataggtgagtctcagtggaacgaggctgatgccacagtagcaggaggcagagtcttaaaaaagaccacccctatgagaccttgctatggatcctatgccaaacctactaatgccaatggcggtcaagggatcatggtggccaatgatcagggagcgcttgaatctaaagttgagatgcaatttttctccaccacaacgtctcttaatgtaagggaaggtgaaaacaatcttcagccaaaagtagtgctatacagcgaagatgttaacttggaatcccctgacactcatttgtcttacaaacctaaaaaggatgacaccaactctaaaatcatgttgggtcagcaagccatgcccaacagacccaacctcattgcttttagggacaactttattggacttatgtactacaacagcacaggcaacatgggagtgctggcaggacaggcctcccagctaaacgctgtggtagacttgcaagacagaaacacagagctgtcataccaactgatgcttgattccattggagacagatcaagatacttttccatgtggaaccaggcagtggacagctatgacccagatgtcagaatcattgaaaaccatggggttgaagatgagctgcccaactattgctttcccctgggcggtattggaattacagacacataccagtgcataaaaccaaccgcagctgctaataacactacatggtctaaggatgaagaatttagtgatcgcaatgaaataggggtgggaaacaacttcgccatggagatcaacatccaggccaacctctggaggaacttcctctatgcgaacgtggggctctacctgccagacaagctcaagtacaaccccaccaacgtggacatctctgacaaccccaacacctatgactacatgaacaagcgtgtggtggctcccggcctggtggactgctttgtcaatgtgggagccaggtggtccctggactacatggacaacgtcaaccccttcaaccaccaccgcaatgcgggtctgcgctaccgctccatgatcctgggcaacgggcgctacgtgcccttccacattcaggtgccccagaagttctttgccatcaagaacctcctcctcctgccgggctcctacacttacgagtggaacttcaggaaggatgtcaacatggtcctgcagagctctctgggcaatgaccttagggtggacggggccagcatcaagtttgacagcgtcaccctctatgctaccttcttccccatggctcacaacaccgcctccacgctcgaggccatgctgaggaacgacaccaacgaccagtccttcaatgactacctctctggggccaacatgctctaccccatccccgccaaggccaccaacgtgcccatctccattccctctcgcaactgggccgccttcagaggctgggcctttacccgccttaagaccaaggaaaccccctccctgggctcgggttttgacccctactttgtctactcgggatccatcccctacctggatggcaccttctacctcaaccacacttttaagaagatatccatcatgtatgactcctccgtcagctggccgggcaatgaccgcctgctcacccccaatgagttcgaggtcaagcgcgccgtggacggcgagggctacaacgtggcccagtgcaacatgaccaaggactggttcctggtgcagatgctggccaactacaacataggctaccagggcttctacatcccagagagctacaaggacaggatgtactccttcttcagaaatttccaacccatgagcaggcaggtggtggacgagaccaaatacaaggactatcaggccattggcatcactcaccagcacaacaactcgggattcgtgggctacctggctcccaccatgcgcgaggggcaggcctaccccgccaacttcccctacccgttgataggcaaaaccgcggtcgacagcgtcacccagaaaaagttcctctgcgaccgcaccctctggcgcatccccttctctagcaacttcatgtccatgggtgcgctcacggacctgggccagaacctgctctatgccaactccgcccatgcgctggacatgacttttgaggtggaccccatggacgagcccacccttctctatattgtgtttgaagtgttcgacgtggtcagagtgcaccagccgcaccgcggtgtcatcgagaccgtgtacctgcgcacgcccttctcggccggcaacgccaccacctaaggagacagcgccgccgcctgcatgacgggttccaccgagcaagagctcagggccatcgccagagacctgggatgcggaccctattttttgggcacctatgacaaacgcttcccgggcttcatctcccgagacaagctcgcctgcgccatcgtcaacacggccgcgcgcgagaccgggggcgtgcactggctggcctttggctgggacccgcgctccaaaacctgctacctcttcgacccctttggcttctccgatcagcgcctcagacagatctatgagtttgagtacgaggggctgctgcgccgcagcgcgcttgcctcctcgcccgaccgctgcatcacccttgagaagtccaccgagaccgtgcaggggccccactcggccgcctgcggtctcttctgctgcatgtttttgcacgcctttgtgcgctggccccagagtcccatggatcgcaaccccaccatgaacttgctcaagggagtgcccaacgccatgctccagagcccccaggtccagcccaccctgcgccacaaccaggaacagctctaccgcttcctggagcgccactccccctacttccgcagtcacagcgcgcacatccggggggccacctctttctgccacttgcaagaaaacatgcaagacggaaaatgatgtacagctcgctttttaataaatgtaaagactgtgcactttatttatacacgggctctttctggttatttattcaacaccgccgtcgccatctagaaatcgaaagggttctgccgcgcgtcgccgtgcgccacgggcagagacacgttgcgatactggaagcggctcgcccacttaaactcgggcaccaccatgcggggcagtggttcctcggggaagttctcgccccacagggtgcgggtcagctgcagcgcgctcaggaggtcgggagccgagatcttgaagtcgcagttggggccggaaccctgcgcgcgcgagttgcggtacacggggttgcagcactggaacaccagcagggccggattatgcacgctggccagcaggctctcgtcgctgatcatgtcgctgtccagatcctccgcgttgctcagggcgaacggggtcatcttgcagacctgcctgcccaggaaaggcggcagcccgggcttgccgttgcagtcgcagcgcaggggcatcagcaggtgcccgcggcccgactgcgcctgcgggtacagcgcgcgcatgaaggcttcgatctgcctgaaagccacctgcgtcttggctccctccgaaaagaacatcccacaggacttgctggagaactggttcgcgggacagctggcatcgtgcaggcagcagcgcgcgtcggtgttggcgatctgcaccacgttgcgaccccaccggttcttcactatcttggccttggaagcctgctccttcagcgcgcgctggccgttctcgctggtcacatccatctctatcacctgctccttgttgatcatgtttgtaccgtgcagacacttcaggtcgccctccgtctgggtgcagcggtgctcccacagcgcgcaaccggtgggctcccaatttttgtgggtcacccccgcgtaggcctgcaggtaggcctgcaagaagcgccccatcatggccacaaaggtcttctggctcgtaaaggtcagctgcaggccgcgatgctcttcgttcagccaggtcttgcagatggcggccagcgcctcggtctgctcgggcagcatcctaaaatttgtcttcaggtcgttatccacgtggtacttgtccatcatggcgcgcgccgcctccatgcccttctcccaggcggacaccatgggcaggcttagggggtttatcacttccaccggcgaggacaccgtactttcgatttcttcttcctccccctcttcccggcgcgcgcccacgctgctgcgcgctctcaccgcctgcaccaaggggtcgtcttcaggcaagcgccgcaccgagcgcttgccgcccttgacctgcttaatcagcaccggcgggttgctgaagcccaccatggtcagcgccgcctgctcttcttcgtcttcgctgtctaccactatctctggggaagggcttctccgctctgcggcggcgcgcttcttttttttcttgggagcggccgtgatggagtccgccacggcgacggaggtcgagggcgtggggctgggggtgcgcggtaccagggcctcgtcgccctcggactcttcctctgactccaggcggcggcggagtcgcttctttgggggcgcgcgcgtcagcggcggcggagacggggacggggacggggacgggacgccctccacagggggtggtcttcgcgcagacccgcggccgcgctcgggggtcttctcgagctggtcttggtcccgactggccattgtatcctcctcctcctaggcagagagacataaggagtctatcatgcaagtcgagaaggaggagagcttaaccaccccctctgagaccgccgatgcgcccgccgtcgccgtcgcccccgctgccgccgacgcgcccgccacaccgagcgacacccccgcggacccccccgccgacgcacccctgttcgaggaagcggccgtggagcaggacccgggctttgtctcggcagaggaggatttgcgagaggaggaggataaggagaagaagccctcagtgccaaaagatgataaagagcaagacgagcacgacgcagatgcacaccagggtgaagtcgggcggggggacggagggcatgacggcgccgactacctagacgaagggaacgacgtgctcttgaagcacctgcatcgtcagtgcgccattgtttgcgacgctctgcaggagcgcagcgaagtgcccctcagcgtggcggaggtcagccacgcctacgagctcagcctcttctccccccgggtgcccccccgccgccgcgaaaacggcacatgcgagcccaacccgcgcctcaacttctaccccgcctttgtggtacccgaggtcctggccacctatcacatcttctttcaaaattgcaagatccccctctcgtgccgcgccaaccgtagccgcgccgataagatgctggccctgcgccagggcgaccacatacctgatatcgccgctttggaagatgtaccaaagatcttcgagggtctgggtcgcaacgagaagcgggcagcaaactctctgcaacaggaaaacagcgaaaatgagagtcacaccggggtactggtggagctcgagggcgacaacgcccgcctggcggtggtcaagcgcagcatcgaggtcacccactttgcctaccccgcgctaaacctgccccccaaagtcatgaacgcggccatggacgggctgatcatgcgccgcggccggcccctcgctccagatgcaaacttgcatgaggagaccgaggacggccagcccgtggtcagcgacgagcagctggcgcgctggctggagaccgcggaccccgccgaactggaggagcggcgcaagatgatgatggccgtggtgctggtcaccgtagagctggagtgtctgcagcgcttcttcggcgaccccgagatgcagagaaaggtcgaggagaccctgcactacaccttccgccagggctacgtgcgccaggcttgcaagatctccaacgtggagctcagcaacctggtgtcctacctgggcatcttgcatgagaaccgcctcgggcagagcgtgctgcactccaccctgcgcggggaggcgcgccgcgactacgtgcgcgactgcgtttacctcttcctctgctacacctggcagacggccatgggggtctggcagcagtgcctggaggagcgcaacctcaaggagctggagaagctcctgcagcgcgcgctcaaagatctctggacgggctacaacgagcgctcggtggccgccgcgctggccgacctcatcttccccgagcgcctgctcaaaaccctccagcaggggctgcccgacttcaccagccaaagcatgttgcaaaacttcaggaactttatcctggagcgttctggcatcctacccgccacctgctgcgccctgcccagcgactttgtccccctcgtgtaccgcgagtgccccccgccgctgtggggtcactgctacctgttccaactggccaactacctgtcctaccacgcggacctcatggaggactccagcggcgaggggctcatggagtgccactgccgctgcaacctctgcacgccccaccgctccctggtctgcaacacccaactgctcagcgagagtcagattatcggtaccttcgagctacagggtccgtcctcctcagacgagaagtccgcggctccggggctaaaactcactccggggctgtggacttccgcctacctgcgcaaatttgtacctgaagactaccacgcccacgagatcaggttttacgaagaccaatcccgcccgcccaaggcggagctgaccgcctgcgtcatcacccagggcgagatcctaggccaattgcaagccatccaaaaagcccgccaagactttttgctgaagaagggtcggggggtgtatctggacccccagtcgggtgaggagctcaacccggttcccccgctgccgccgccgcgggaccttgcttcccaggataagcatcgccatggctcccagaaagaagcagcagcggccgccactgccgccaccccacatgctggaggaagaggaggaatactgggacagtcaggcagaggaggtttcggacgaggaggagccggagacggagatggaagagtgggaggaggacagcttagacgaggaggcttccgaagccgaagaggcagacgcaacaccgtcaccctcggccgcagccccctcgcaggcgcccccgaagtccgctcccagcatcagcagcaacagcagcgctataacctccgctcctccaccgccgcgacccacggccgaccgcagacccaaccgtagatgggacaccaccggaaccggggccggtaagtcctccgggagaggcaagcaagcgcagcgccaaggctaccgctcgtggcgcgctcacaagaacgccatagtcgcttgcttgcaagactgcggggggaacatctccttcgcccgccgcttcctgctcttccaccacggtgtggccttcccccgtaacgtcctgcattactaccgtcatctctacagcccctactgcggcggcagtgagccagagacggtcggcggcggcggcggcgcccgtttcggcgcctaggaagacccagggcaagacttcagccaagaaactcgcggcggccgcggcgaacgcggtcgcgggggccctgcgcctgacggtgaacgaacccctgtcgacccgcgaactgaggaaccgaatcttccccactctctatgccatcttccagcagagcagagggcaggatcaggaactgaaagtaaaaaacaggtctctgcgctccctcacccgcagctgtctgtatcacaagagcgaagaccagcttcggcgcacgctggaggacgctgaggcactcttcagcaaatactgcgcgctcactcttaaggactagctccgcgcccttctcgaatttaggcgggaacgcctacgtcatcgcagcgccgccgtcatgagcaaggacattcccacgccatacatgtggagctatcagccgcagatgggactcgcggcgggcgcctcccaagactactccacccgcatgaactggctcagtgccggcccacacatgatctcacaggttaatgatatccgcacccatcgaaaccaaatattggtggagcaggcggcaattaccaccacgccccgcaataatcccaaccccagggagtggcccgcgtccctggtgtatcaggaaattcccggccccaccaccgtactacttccgcgtgattcccaggccgaagtccaaatgactaactcaggggcacagctcgcgggcggctgtcgtcacagggtgcggcctcctcgccagggtataactcacctggagatccgaggcagaggtattcagctcaacgacgagtcggtgagctcctcgctcggtctcagacctgacgggaccttccagatagccggagccggccgatcttccttcacgccccgccaggcgtacctgactctgcaaagctcgtcctcggcgccgcgctcgggcggcatcgggactctccagttcgtgcaggagtttgtgccctcggtctacttcaaccccttctcgggctctcccggtcgctacccggaccagttcatctcgaactttgacgccgcgagggactcggtggacggctacgactgaatgtcgggtggacccggtgcagagcaacttcgcctgaagcacctcgaccactgccgccgccctcagtgctttgcccgctgtcagaccggtgagttccagtacttttccctgcccgactcgcacccggacggcccggcgcacggggtgcgctttttcatcccgagtcaggtgcgctctaccctaatcagggagtttaccgcccgtcccctactggcggagttggaaaaggggccttctatcctaaccattgcctgcatctgctctaaccctggattgcaccaagatctttgctgtcatttgtgtgctgagtataataaaggctgagatcagaatctactcgggctcctgtcgccatcctgtcaacgccaccgtccaagcccggcccgatcagcccgaggtgaacctcacctgcggtctgcaccggcgcctgaggaaatacctagcttggtactacaacagcactccctttgtggtttacaacagctttgaccaggacggggtctcactgagggataacctctcgaacctgagctactccatcaggaagaacagcaccctcgagctacttcctccttacctgcccgggacttaccagtgtgtcaccggtccctgcacccacacccacctgttgatcgtaaacgactctcttccgagaacagacctcaataactcctcttcgcagttccccagaacaggaggtgagctcaggaaaccccgggtaaagaagggtggacgagagttaacacttgtggggtttctggtgtatgtgacgctggtggtggctcttttgattaaggcttttccttccatgtctgaactctccctcttcttttatgaacaactcgactagtgctaacgggaccctacccaacgaatcgggattgaatatcggtaaccaggttgcagtttcacttttgattaccttcatagtcctcttcctgctagtgctgtcgcttctgtgcctgcggatcgggggctgctgcatccacgtttatatctggtgctggctgtttagaaggttcggagaccatcgcaggtagaataaacatgctgctgcttaccctctttgtcctggcgctggccgccagctgccaagccttttccgaggctgactttatagagccccagtgtaatgtgacttttaaagcccatgcacagcgttgtcatactataatcaaatgtgccaccgaacacgatgaataccttatccagtataaagataaatcacacaaagtggcacttgttgacatctggaaacccgaagaccctttggaatacaatgtgaccgttttccagggtgacctcttcaaaatttacaattacactttcccatttgaccagatgtgtgactttgtcatgtacatggaaaagcagcacaagctgtggcctccgactccccagggctgtgtggaaaatccaggctctttctgcatgatctctctctgtgtaactgtgctggcactaatactcacgcttttgtatatcagatttaaatcaaggcaaagcttcattgatgaaaagaaaatgccttaatcgctttcacgcttgattgctaacaccgggtttttatccgcagaatgattggaatcaccctactaatcacctccctccttgcgattgcccatgggttggaacgaatcgaagtccctgtgggggccaatgttaccctggtggggcctgtcggcaatgctacattaatgtgggaaaaatatactaaaaatcaatgggtctcttactgcactaacaaaaatagccacaagcccagagccatctgcgatgggcaaaatctaaccttgattgatgttcaattgctggatgcgggctactattatgggcagctgggtacaatgattaattactggagaccccacagagattacatgctccacgtagtaaagggtccccttagcagcccacccactaccacctctactacccccactaccaccactactcccaccaccagcactgccgcccagcctcctcatagcagaacaaccacttttatcaattccaagtcccactccccccacattgccggcgggccctccgcctcagactccgaaaccaccgagatctgcttctgcaaatgctctgacgccattgcccaggatttggaagatcacgaggaagatgagcatgacttcgcagatgcatgccaggcatcagagccagaagcgctgccggtggccctcaaacagtatgcagacccccacaccacccccgaccttcctccaccttcccagaagccaagtttcctgggggaaaatgaaactctgcctctctccatactcgctctgacatctgttgctatgttgaccgctctgctggtgcttctatgctctatatgctacctgatctgctgcagaaagaaaaaatctcacggccatgctcaccagcccctcatgcacttcccttaccctccagagctgggcgaccacaaactttaagtctgcagtaactatctgcccatcccttgtcagtcgacagcgatgagccccactaatctaacggcctctggacttacaacatcgtctcttaatgagaccaccgctcctcaagacctgtacgatggtgtctccgcgctggttaaccagtgggatcacctgggcatatggtggctcctcataggagcagtgaccctgtgcctaatcctggtctggatcatctgctgcatcaaaagcagaagacccaggcggcggcccatctacaggccctttgtcatcacacctgaagatgatgatgacaccacttccaggctgcagaggctaaagcagctactcttctcttttacagcatggtaaattgaatcatgcctcgcattttcatctacttgtctctccttccactttttctgggctcttctacattggccgctgtgtcccacatcgaggtagactgcctcacgcccttcacagtctacctgcttttcggctttgtcatctgcacctttgtctgcagcgttatcactgtagtgatctgcttcatacagtgcatcgactacgtctgcgtgcgggtggcttactttagacaccacccccagtatcgcaacagggacatagcggctctcctaagacttgtttaaaatcatggccaaattaactgtgattggtcttctgatcatctgctgcgtcctagccgcgattgggactcaagctcctaccaccaccagcgctcccagaaagagacatgtatcctgcagcttcaagcgtccctggaatataccccaatgctttactgatgaacctgaaatctctttggcttggtacttcagcgtcaccgcccttcttatcttctgcagtacggttattgcccttgccatctacccttcccttgacctgggctggaatgctgtcaactctatggaatatcccaccttcccagaaccagacctgccagacctggttgttctaaacgcgtttcctcctcctgctcccgttcaaaatcagtttcgccctccgtcccccacgcccactgaggtcagctactttaatctaacaggcggagatgactgaaaacctagacctagaaatggacggtctctgcagcgagcaacgcacactagagaggcgccggcaaaaagagctcgagcgtcttaaacaagagctccaagacgcggtggccatacaccagtgcaaaaaaggtgtcttctgtctggtaaaacaggccacgctcacctatgaaaaaacaggtgacacccaccgcctaggatacaagctgcccacacagcgccaaaagttcgccctcatgataggcgaacaacccatcaccgtgacccagcactccgtggagacagaaggctgcatacatgctccctgtaggggcgctgactgcctctacaccttgatcaaaaccctctgcggtctcagagaccttatccctttcaattaatcataactgtaatcaataaaaaatcacttacttgaaatctgatagcaagcctctgtccaattttttcagcaacacttccttcccctcctcccaactctggtactctaggcgcctcctagctgcaaacttcctccacagtctgaagggaatgtcagattcctcctcctgtccctccgcacccacgatcttcatgttgttgcagatgaaacgcgcgagatcgtctgacgagaccttcaaccccgtgtacccctacgataccgagatcgctccgacttctgtccctttccttacccctccctttgtgtcatccgcaggaatgcaagaaaatccagctggggtgctgtccctgcacttgtcagagccccttaccacccacaatggggccctgactctaaaaatggggggcggcctgaccctggacaaggaagggaatctcacttcccaaaacatcaccagtgtcgatccccctctcaaaaaaagcaagaacaacatcagccttcagaccgccgcacccctcgccgtcagctccggggccctaacactttttgccactccccccctagcggtcagtggtgacaaccttactgtgcagtctcaggcccctctcactttggaagactcaaaactaactctggccaccaaaggacccctaactgtgtccgaaggcaaacttgtcctaga

SEQ ID NO: 25 - DNA, 32728 bp, synthetic construct
  misc_feature 1..32728: note = Adenovirus vector nucleotide sequences
  source 1..32728: mol_type = other DNA
catcatcaataatataccttattttggattgtggccaatatgataatgaggtgggcggggagaggcggggcgggtgacgtaggacgcgcgagtagggttgggaggtgtggcggaagtgtggcatttgcaagtgggaggagctcacatgcaagcttccgtcgcggaaaatgtgacgtttttgatgagcgccgcctacctccggaagtgccaattttcgcgcgcttttcaccggatatcgtagtaattttgggcgggaccatgtaagatttggccattttcgcgcgaaaagtgaaacggggaagtgaaaactgaataatagggcgttagtcatagtgcgtaatatttaccgagggccgagggactttgaccgattacgtggaggactcgcccaggtgttttttacgtgaatttccgcgttccgggtcaaagtctccgttttattgtcaccgtcatttgacgcttaggcctgaccatctggtgctggcctgcaccagggccgagtttgggtctagcgatgaggataccgattgaggtgggtaaggtgggcgtggctagaagggtggggcgtgtataaattgggggtctaagggtctctctgttttgtcttgcaacagccgccgccatgagcgacaccggcaacagctttgatggaagcatctttagcccctatctgacagtgcgcatgcctcactgggctggagtgcgtcagaatgtgatgggttccaacgtggatggacgccccgttctgccttcaaattcgtctacaatggcctacgcgaccgtgggaggaactccgctggacgccgcgacctccgccgccgcctccgccgccgccgcgaccgcgcgcagcatggctacggacctttacagctctttggtggcgagcggcgcggcctctcgcgcgtctgctcgggatgagaaactgaccgctctgctgcttaaactggaagacttgacccgggagctgggtcaactgacccagcaggtctccagcttgcgtgagagcagccttgcctccccctaatggcccataatataaataaaagccagtctgtttggattaagcaagtgtatgttctttatttaactctccgcgcgcggtaagcccgggaccagcggtctcggtcgtttagggtgcggtggattctttccaacacgtggtacaggtggctctggatgtttagatacatgggcatgagtccatccctggggtggaggtagcaccactgcagagcttcgtgctcgggggtggtgttgtatatgatccagtcgtagcaggagcgctgggcgtggtgctgaaaaatgtccttaagcaagaggcttatagctagggggaggcccttggtgtaagtgtttacaaatctgctcagttgggaggggtgcatccggggggatataatgtgcatcttggactggatttttaggttggctatgttcccacccagatcccttctgggattcatgttgtgcaggaccaccagcacggtatatccagtgcacttgggaaatttatcgtggagcttagacgggaatgcatggaagaacttggagacgcccttgtggcctcccagattttccatacattcgtccatgatgatggcaatgggcccgtgggaagctgcctgagcaaaaatgtttctgggatcgctcacatcgtagttatgttccagggtgaggtcatcataggacatctttacgaatcgggggcggagggtcccggactgggggatgatggtaccctcgggccccggggcgtagttcccctcacagatctgcatctcccaggctttcatttcagagggagggatcatatccacctgcggagcgatgaaaaacacagtttctggcgcaggggagattaactgggatgagagcaggtttctgagcagctgtgactttccacagccggtgggcccatatatcacgcctatcaccggctgcagctggtagttaagagagctgcagctgccgtcctcccggagcaggggggccacctcgttcagcatatccctgacgtggatgttctccctgaccaattccgccagaaggcgctcgccgcccagcgaaagcagctcttgcaaggaagcaaaatttttcagcggttttaggccgtcggccgtgggcatgtttttcagcgtctgggtcagcagttccagcctgtcccacagctcggtgatgtgctctacggcatctcgatccagcagatctcctcgtttcgcgggttggggcggctttcgctgtagggcaccagccgatgggcgtccagcggggccagagtcatgtccttccatgggcgcagggtcctcgtcagggtggtctgggtcacggtgaaggggtgcgctccgggttgggcgctggccagggtgcgcttgaggctggttctgctggtgctgaatcgctgccgctcttcgccctgcgcgtcggccaggtagcatttgaccatggtctcgtagtcgagaccctcggcggcgtgccccttggcgcggagctttcccttggaggtggcgccgcacgaggggcactgcaggctcttcagggcgtagagcttgggagcgagaaacacggactctggggagtaggcgtccgcgccgcaggaagcgcagaccgtctcgcattccaccagccaagtgagctccgggcggtcagggtcaaaaaccaggttgcccccatgctttttgatgcgtttcttacctcggctctccatgaggcggtgtcccttctcggtgacgaagaggctgtccgtgtccccgtagaccgacttcaggggcctgtcttccagcggagtgcctctgtcctcctcgtagagaaactctgaccactctgagacgaaggcccgcgtccaggccaggacgaaggaggccacgtgggaggggtagcggtcgttgtccactagcgggtccaccttctccagggtgtgcaggcacatgtccccctcctccgcgtccagaaaagtgattggcttgtaggtgtaggacacgtgaccgggggttcccgacgggggggtataaaagggggtgggcgccctttcatcttcactctcttccgcatcgctgtctgcgagggccagctgctggggtaagtattccctctcgaaggcgggcatgacctcagcgctcaggttgtcagtttctaaaaatgaggaggatttgatgttcacctgtccggaggtgatacctttgagggtacctgggtccatctggtcagaaaacactatttttttgttgtcaagcttggtggcgaacgacccgtagagggcgttggagagcagcttggcgatggagcgcagggtctggtttttgtcgcggtcggctcgctccttggccgcgatgttgagttgcacgtactcgcgggccacgcacttccactcggggaagacggtggtgcgctcgtctgggattaggcgcaccctccagcctcggttgtgcagggtgaccatgtcgacgctggtggcgacctcgccgcgcaggcgctcgttggtccagcagaggcggccgcccttgcgcgagcagaaggggggtagggggtccagctggtcctcgtttggggggtccgcgtcgatggtgaagaccccggggagcaagcgcgggtcaaagtagtcgatcttgcaagcttgcatgtccagagcccgctgccattcgcgggcggcgagcgcgcgctcgtaggggttgaggggcgggccccagggcatggggtgggtgagcgcggaggcgtacatgccgcagatgtcatacacgtacaggggttccctgaggatgccgaggtaggtggggtagcagcgccccccgcggatgctggcgcgcacgtagtcatagagctcgtgggagggggccagcatgttgggcccgaggttggtgcgctgggggcgctcggcgcggaaggcgatctgcctgaagatggcatgggagttggaggagatggtgggccgctggaagacgttgaagcttgcttcttgcaagcccaccgagtccctgacgaaggaggcgtaggactcgcgcagcttgtgcaccagctcggcggtgacctggacgtcgagcgcgcagtagtcgagggtctcgcggatgatgtcatacttatcctcccccttctttttccacagctcgcggttgaggacgaactcttcgcggtctttccagtactcttggaggggaaacccgtccgtgtccgaacggtaagagcctagcatgtagaactggttgacggcctggtaggggcaacagcccttctccacgggcagcgcgtaggcctgcgccgccttgcggagggaggtgtgggtgagggcgaaagtgtccctgaccatgactttgaggtattgatgtttgaagtctgtgtcatcgcagccgccctgttcccacagggtgtagtccgtgcgctttttggagcgcgggttgggcagggagaaggtgaggtcattgaagaggatcttccccgctcgaggcatgaagtttctggtgatgcgaaagggccctgggaccgaggagcggttgttgatgacctgggcggccaggacgatctcgtcaaagccgtttatgttgtggcccacgatgtagagctccaaaaagcggggctggcccttgatggaggggagctttttgagttcctcgtaggtgagctcctcgggcgattccaggccgtgctcctccagggcccagtcttgcaagtgagggttggccgccaggaaggatcgccagaggtcgcgggccatgagggtctgcaggcggtcgcggaaggttctgaactgtcgccccacggccatcttttcgggggtgatgcagtagaaggtgagggggtctttctcccaggggtcccatctgagctctcgggcgaggtcgcgcgcggcggcgaccagagcctcgtcgccccccagtttcatgaccagcatgaagggcacgagctgcttgccaaaggctcccatccaagtgtaggtctctacatcgtaggtgacaaagaggcgctccgtgcgaggatgagagccgatcgggaagaactggatctcccgccaccagttggaggattggctgttgatgtggtgaaagtagaagtcccgtctgcgggccgagcactcgtgctggcttttgtaaaagcgaccgcagtactggcagcgctgcacgggttgtatatcttgcacgaggtgaacctggcgacctctgacgaggaagcgcagcgggaatctaagtcccccgcctggggtcccgtgtggctggtggtcttctactttggttgtctggccgccagcatctgtctcctggagggcgatggtggagcagaccaccacgccgcgagagccgcaggtccagatctcggcgctcggcgggcggagtttgatgacgacatcgcgcacattggagctgtccatggtctccagctcccgcggcggcaggtcagctgggagttcctggaggttcacctcgcagagacgggtcaaggcgcgggcagtgttgagatggtatctgatttcaaggggcgtgttggcggcggagtcgatggcttgcaggaggccgcagccccggggggccacgatggttccccgcggggcgcgaggggaggcggaagctgggggtgtgttcagaagcggtgacgcgggcgggcccccggaggtagggggggttccggccccacaggcatgggcggcaggggcacgtcttcgccgcgcgcgggcaggggctggtgctggctccgaagagcgcttgcgtgcgcgacgacgcgacggttggtgtcctgtatctgacgcctctgagtgaagaccacgggtcccgtgaccttgaacctgaaagagagttcgacagaatcaatctcggcatcgttgacagcggcctggcgcaggatctcctgcacgtcgcccgagttgtcctggtaggcgatctctgccatgaactgctcgatctcttcttcctggagatctcctcgtccggcgcgctccacggtggccgccaggtcgttggagatgcgacccatgagctgtgagaaggcgttgagcccgccctcgttccagacccggctgtagaccacgcccccctcggcgtcgcgagcgcgcatgaccacctgggccaggttgagctccacgtgtcgcgtgaagacggcgtagttgcgcaggcgctggaaaaggtagttcagggtggtggcggtgtgctcggcgacgaagaagtacatgacccagcgccgcaacgtggattcattgatgtcccccaaggcctccaggcgctccatggcctcgtagaagtccacggcgaagttgaaaaactgggagttgcgagcggacacggtcaactcctcctccagaagacggatgagctcggcgacagtgttgcgcacctcgcgctcgaaggccacggggggcgcttcttcctcttccacctcttcttccatgatcgcttcttcttcttcctcagccgggacgggagggggcggcggcggcgggggaggggcgcggcggcggcggcggcgcaccgggaggcggtcgatgaagcgctcgatcatctccccccgcatgcggcgcatggtctcggtgacggcgcggccgttctcccgggggcgcagctcgaagacgccgcctctcatctcgccgcggggcgagcggccgtgaggtagcgagacggcgctgactatgcatcttaacaattgctgtgtaggtacaccgccgagggacctgattgagtccagatccaccggatccgaaaacctttggaggaaagcgtctatccagtcgcagtcgcaaggtaggctgagcaccgtggcgggcgggggcgggtctggagagttcctggcggagatgctgctgatgatgtaattaaagtaggcggtcttgagaaggcggatggtggacaggagcaccatgtctttgggtccggcctgttggatgcggaggcggtcggccatgccccaggcctcgttctgacaccggcgcaggtctttgtagtagtcttgcatgagtctttccaccggcacctcttctccttcctcttctccatctcgccggtggtttctcgcgccgcccatgcgcgtgaccccaaagcccctgagcggctgcagcagggccaggtcggcgaccacgcgctcggccaagatggcctgctgcacctgagtgagggtcctctcgaagtcatccatgtccacgaagcggtggtaggcgcccgtgttgatggtgtaggtgcagttggccatgacggaccagttgacggtctggtgtcccggctgcgagagctccgtgtaccgcaggcgcgagaaggcgcgggaatcgaacacgtagtcgttgcaagtccgcaccagatactggtagcccaccaggaagtgcggcggaggttggcgatagaggggccagcgctgggtggcgggggcgccgggcgccaggtcttccagcatgaggcggtggtatccgtagatgtacctggacatccaggtgatgccggcggcggtggtggtggcgcgcgcgtagtcgcggacccggttccagatgtttcgcaggggcgagaagtgttccatggtcggcacgctctggccggtgaggcgcgcgcagtcgttgacgctctatacacacacaaaaacgaaagcgtttacagggctttcgttctgtagcctggaggaaagtaaatgggttgggttgcggtgtgccccggttcgagaccaagctgagctcggccggctgaagccgcagctaacgtggtattggcagtcccgtctcgacccaggccctgtatcctccaggatacggtcgagagcccttttgctttcttggccaagcgcccgtggcgcgatctgggatagatggtcgcgatgagaggacaaaagcggctcgcttccgtagtctggagaaacaatcgccagggttgcgttgcggcgtaccccggttcgagcccctatggcggcttgaatcggccggaaccgcggctaacgagggccgtggcagccccgtcctcaggaccccgccagccgacttctccagttacgggagcgagccccttttgttttttattttttagatgcatcccgtgctgcggcagatgcgcccctcgccccggcccgatcagcagcagcaacagcaggcatgcagacccccctctcccctttccgccccggtcaccacggccgcggcggccgtgtcgggcgcggggggcgcgctggagtcagatgagccaccgcggcggcgacctaggcagtatctggacttggaagagggcgagggactggcgcggctgggggcgaactctccagagcgccacccgcgggtgcagttgaaaagggacgcgcgcgaggcgtacctgccgcggcagaacctgtttcgcgaccgcgggggcgaggagcccgaggagatgcgagactgcaggttccaagcggggcgcgagctgcggcgcgggctggacagacagcgcctgctgcgcgaggaggactttgagcccgacacgcagacgggcatcagccccgcgcgcgcgcacgtagccgcggccgacctggtgaccgcctacgagcagacggtaaaccaggagcgcaacttccaaaagagcttcaacaaccacgtgcgcacgctggtggcgcgcgaggaggtgaccctgggtctcatgcatctgtgggacctggtggaggcgatcgtgcagaaccccagcagcaagcccctgaccgcgcagctgttcctggtggtgcagcacagcagggacaacgaggccttcagggaggcgctgctgaacatcaccgagccggaggggcgctggctcctggacctgataaacatcctgcagagcatagtggtgcaggagcgcagcctgagcctggccgagaaggtggcggccatcaactactctatgctgagcctgggcaagttctacgcccgcaagatctacaagaccccctacgtgcccatagacaaggaggtgaagatagacagcttctacatgcgcatggcgctgaaggtgctgaccctgagcgacgacctgggagtgtaccgcaacgagcgcatccacaaggccgtgagcgccagccggcggcgcgagctgagcgaccgcgagctgatgcacagtctgcagcgcgcgctgaccggcgcgggcgagggcgacagggaggtcgagtcctacttcgacatgggggccgacctgcactggcagccgagccgccgcgccctggaggcggcgggggcgtacggcggccccctggcggccgatgaccaggaagaggaggactatgagctagaggagggcgagtacctggaggactgacctggctggtggtgttttggtatagatgcaagatccgaacgtggcggacccggcggtccgggcggcgctgcaaagccagccgtccggcattaactcctctgacgactgggccgcggccatgggtcgcatcatggccctgaccgcgcgcaaccccgaggctttcaggcagcagcctcaggccaaccggctggcggccatcttggaagcggtagtgcccgcgcgctccaaccccacccacgagaaggtgctggccatagtcaacgcgctggcggagagcagggccatccgcgcggacgaggccggactggtgtacgatgcgctgctgcagcgggtggcgcggtacaacagcggcaacgtgcagaccaacctggaccgcctggtgacggacgtgcgcgaggccgtggcgcagcgcgagcgcttgcatcaggacggtaacctgggctcgctggtggcgctaaacgccttcctcagcacccagccggccaacgtaccgcgggggcaggaggactacaccaactttttgagcgcgctgcggctgatggtgaccgaggtccctcagagcgaggtgtaccagtcggggcccgactacttcttccagaccagcagacagggcttgcaaaccgtgaacctgagccaggctttcaagaacctgcgggggctgtggggagtgaaggcgcccaccggcgaccgggctacggtgtccagcctgctaacccccaactcgcgcctgctgctgctgctgatcgcgcccttcacggacagcgggagcgtctcgcgggagacctatctgggccacctgctgacgctgtaccgcgaggccatcgggcaggcgcaggtggacgagcacaccttccaagagatcaccagcgtgagccacgcgctggggcaggaggacacgggcagcctgcaggcgaccctgaactacctgctgaccaacaggcggcagaagattcccacgctgcacagcctgacccaggaggaggagcgcatcttgcgctacgtgcagcagagcgtgagcctgaacctgatgcgcgacggcgtgacgcccagcgtggcgctggacatgaccgcgcgcaacatggaaccgggcatgtacgcctcccaccggccgtttatcaaccgcctgatggactacttgcatcgggcggcggccgtgaaccccgagtacttcactaatgccattctgaatccccactggatgccccctccgggtttctacaacggggactttgaggtgcccgaggtcaacgacgggttcctctgggatgacatggatgacagtgtgttctcacccaacccgctgcgcgccgcgtctctgcgattgaaggagggctctgacagggaaggaccgaggagtctggcctcctccctggctctgggagcggtgggcgccacgggcgcggcggcgcggggcagtagccccttccccagcctggcagactctctgaacagcgggcgggtgagcaggccccgcttgctaggcgaggaggagtatctgaacaactccctgctgcagcccgcgagggacaagaacgctcagcggcagcagtttcccaacaatgggatagagagcctggtggacaagatgtccagatggaagacgtatgcgcaggagtacaaggagtgggaggaccgccagccgcggcccttgccgccccctaggcagcgctggcagcggcgcgcgtccaaccgccgctggaggcaggggcccgaggacgatgatgactctgcagatgacagcagcgtgttggacctgggcgggagcgggaaccccttttcgcacctgcgcccacgcctgggcaagatgttttaaaagaaaaaaaaaataaaactcaccaaggccatggcgacgagcgttggttttttgttcccttccttagtatgcggcgcgcggcgatgttcgaggaggggcctcccccctcttacgagagcgcgatggggatttctcctgcggcgcccctgcagcctccctacgtgcctcctcggtacctgcaacctacaggggggagaaatagcatctgttactctgagctgcagcccctgtacgataccaccagactgtacctggtggacaacaagtccgcggacgtggcctccctgaactaccagaacgaccacagcgattttttgaccacggtgatccaaaacaacgacttcaccccaaccgaggccagcacccagaccataaacctggataacaggtcgaactggggcggcgacctgaagaccatcttgcacaccaacatgcccaacgtgaacgagttcatgttcaccaactcttttaaggcgcgggtgatggtggcgcgcgagcagggggaggcgaagtacgagtgggtggacttcacgctgcccgagggcaactactcagagaccatgactctcgacctgatgaacaatgcgatcgtggaacactatctgaaagtgggcaggcagaacggggtgaaggaaagcgatatcggggtcaagtttgacaccagaaacttccgtctgggctgggaccccgtgaccgggctggtcatgccgggggtctacaccaacgaggcctttcatcccgacatagtgcttctgcccggctgtggggtggacttcacccagagccggctgagcaacctgctgggcattcgcaagcggcagcctttccaggagggtttcaagatcacctatgaggatctgaaggggggcaacattcccgcgctccttgatctggacgcctacgaggagagcttgaaacccgaggagagcgctggcgacagcggcgagagtggcgaggagcaagccggcggcggtggcggcgcgtcggtagaaaacgaaagtacgcccgcagtggcggcggacgctgcggaggtcgagccggaggccatgcagcaggacgcagaggagggcgcacaggagggcgcgcagaaggacatgaacgatggggagatcaggggagacacattcgccacccggggcgaagaaaaagaggcagaggcggcggcggcggcgacggcggaggccgaaaccgaggttgaggcagaggcagagcccgagaccgaagttatggaagacatgaatgatggagaacgtaggggcgacacgttcgccacccggggcgaagagaaggcggcggaggcagaagccgcggctgaggaggcggctgcggctgcggccaagactgaggctgcggctaaggctgaggtcgaagccaatgttgcggttgaggctcaggctgaggaggaggcggcggctgaagcagttaaggaaaaggcccaggcagagcaggaagagaaaaaacctgtcattcaacctctaaaagaagatagcaaaaagcgcagttacaacgtcatcgagggcagcacctttacccagtaccgcagctggtacctggcgtacaactacggcgacccggtcaagggggtgcgctcgtggaccctgctctgcacgccggacgtcacctgcggctccgagcagatgtactggtcgctgccgaacatgatgcaagacccggtgaccttccgctccacgcggcaggttagcaacttcccggtggtgggcgccgaactgctgcccgtgcactccaagagtttttacaacgagcaggccgtctactcccagctgatccgccaggccacctctctgacccacgtgttcaatcgctttcccgagaaccagattttggcgcgcccgccggcccccaccatcaccaccgtgagtgaaaacgttcctgccctcacagatcacgggacgctaccgctgcgcaacagcatctcaggagtccagcgagtgaccattactgacgccagacgccggacctgcccctacgtttacaaggccttgggcatagtctcgccgcgcgtcctctccagtcgcactttttaaaacacatctacccacacgttccaaaatcatgtccgtactcatctcacccagcaacaacaccggctgggggctgcgcgcgcccagcaagatgtttggaggggcgaggaagcgctccgaccagcaccctgtgcgcgtgcgcggccactaccgcgcgccctggggagcgcacaagcgcgggcgcacagggcgcaccactgtggacgacgtcattgactccgtagtggagcaagcgcgccactacacacccggcgcgccgaccgcccccgccgtgtccaccgtggaccaggcgatcgaaagcgtggtacagggcgcgcggcactatgccaaccttaaaagtcgccgccgccgcgtggcccgccgccatcgccggagaccccgggccaccgccgccgcgcgccttactaaggctctgctcaggcgcgccaggcgaactggccaccgggccgccatgagggccgcacggcgggctgccgctgccgcaagcgtcgtggccccgcgggcacgaaggcgcgcggccgctgccgccgccgccgccatttccagcttggcctcgacgcggcgcggtaacatatactgggtgcgcgactcggtaaccggcacgcgggtacccgtgcgctttcgccccccgcggaattagcacaagacaacatacacactgagtctcctgctgttgtgtatcccagcggcgaccgtcagcagcggcgacatgtccaagcgcaaaattaaagaagagatgctccaggtcatcgcgccggagatctatgggcccccgaagaaggaggaggatgattacaagccccgcaagctaaagcgggtcaaaaagaaaaagaaagatgatgatgacgaggcggtggagtttgtccgccgcatggcacccaggcgccccgtgcagtggaagggccggcgcgtgcagcgcgttttgcgccccggcaccgcggtggtcttcacgcccggcgagcgctccacgcgcactttcaagcgggtgtacgatgaggtgtacggcgacgaggacctgttggagcaggccaaccagcgctttggggagtttgcatatgggaaacggccccgcgagagtctaaaagaggacctgctggcgctaccgctggacgagggcaatcccaccccgagtctgaagccggtaaccctgcaacaggtgctgcctttgagcgcgcccagcgagcataagcgagggttgaagcgcgaaggcggggacctggcgcccaccgtgcagttgatggtgcccaagcggcagaagctggaggacgtgctggagaaaatgaaagtagagcccgggatccagcccgagatcaaggtccgccccatcaagcaggtggcgcccggcgtgggagtccagaccgtggacgttaggattcccacggaggagatggaaacccaaaccgccactccctcttcggcggccagcgccaccaccggcaccgcttcggtagaggtgcagacggacccctggctacccgccaccgctgttgccgccgccgccccccgttcgcgcgggcgcaagagaaattatccagcggccagcgcgctcatgccccagtacgcactgcatccatccatcgtgcccacccccggctaccgcgggtactcgtaccgcccgcgcagatcagccggcactcgcggccgccgccgccgtgcgaccacaaccagccgccgccgtcgccgccgccgccagccagtgctgacccccgtgtctgtaaggaaggtggctcgctcggggagcacgctggtggtgcccagagcgcgctaccaccccagcatcgtttaaagccggtctctgtatggttcttgcagatatggccctcacttgtcgcctccgcttcccggtgccgggataccgaggaagaactcaccgccgcagaggcatggcgggcagcggtctccgcggcggccgtcgccatcgccggcgcgcaaaaagcaggcgcatgcgcggcggtgtgctgcctctgctaatcccgctaatcgccgcggcgatcggtgccgtacccgggatcgcctccgtggccctgcaggcgtcccagaaacgttgactcttgcaaccttgcaagcttgcattttttggaggaaaaataaaaaaaagtctagactctcacgctcgcttggtcctgtgactattttgtagaaaaaaagatggaagacatcaactttgcgtcgctggccccgcgtcacggctcgcgcccgttcatgggagactggacagatatcggcaccagcaatatgagcggtggcgccttcagctggggcagtctgtggagcggccttaaaaattttggttccaccattaagaactatggcaacaaagcgtggaacagcagcacgggccagatgctgagagacaagttgaaagagcagaacttccaggagaaggtggcgcagggcctggcctctggcatcagcggggtggtggacatagctaaccaggccgtgcagaaaaagataaacagtcatctggacccccgtcctcaggtggaggaaatgcctccagcgatggagacggtgtctcccgagggcaaaggcgaaaagcgcccgcggcccgacagagaagagaccctggtgtcacacaccgaggagccgccctcttacgaggaggcagtcaaggccggcctgcccaccactcgccccatagcccccatggccaccggtgtggtgggccacaggcaacacactcccgcaacactagatctgcccccgccgtccgagccgccgcgccagccaaaggcggcgacggtgcccgctccctccacttccgccgccaacagagtgcccctgcgccgcgccgcgagcggcccccgggcctcgcgagttagcggcaactggcagagcacactgaacagcatcgtgggcctgggagtgaggagtgtgaagcgccgccgttgctactgaatgagcaagctagctaacgtgttgtatgtgtgtatgcgtcctatgtcgccgccagaggagctgttgagccgccggcgccgtctgcactccagcgaatttcaagatggcgaccccatcgatgatgcctcagtggtcgtacatgcacatctcgggccaggacgcttcggagtacctgagccccgggctggtgcagttcgcccgcgccacagacacctacttcaacatgagtaacaagttcaggaaccccactgtggcgcccacccacgatgtgaccacggaccggtcgcagcgcctgacgctgcggttcatccccgtggatcgggaggacaccgcctactcttacaaggcgcggttcacgctggccgtgggcgacaaccgcgtgctggacatggcctccacttactttgacatcaggggggtgctggacaggggccccaccttcaagccctactcgggtactgcctacaactccctggcccccaagggcgctcccaattcttgcgagtgggaacaagatgaaccagctcaggcagcaatagctgaagatgaagaagaacttgaagaagaacaagctcaggacgaacaggcgcccactaagaaaacccatgtatacgcccaggcacctctttctggtgaaaaaattactaaggatggtttgcaaataggtgtggatgccacacaggcgggagataaccctatatatgctgataaaacattccaacccgaacctcagataggtgagtctcagtggaacgaggctgatgccacagtagcaggaggcagagtcttaaaaaagaccacccctatgagaccttgctatggatcctatgccaaacctactaatgccaatggcggtcaagggatcatggtggccaatgatcagggagcgcttgaatctaaagttgagatgcaatttttctccaccacaacgtctcttaatgtaagggaaggtgaaaacaatcttcagccaaaagtagtgctatacagcgaagatgttaacttggaatcccctgacactcatttgtcttacaaacctaaaaaggatgacaccaactctaaaatcatgttgggtcagcaagccatgcccaacagacccaacctcattgcttttagggacaactttattggacttatgtactacaacagcacaggcaacatgggagtgctggcaggacaggcctcccagctaaacgctgtggtagacttgcaagacagaaacacagagctgtcataccaactgatgcttgattccattggagacagatcaagatacttttccatgtggaaccaggcagtggacagctatgacccagatgtcagaatcattgaaaaccatggggttgaagatgagctgcccaactattgctttcccctgggcggtattggaattacagacacataccagtgcataaaaccaaccgcagctgctaataacactacatggtctaaggatgaagaatttagtgatcgcaatgaaataggggtgggaaacaacttcgccatggagatcaacatccaggccaacctctggaggaacttcctctatgcgaacgtggggctctacctgccagacaagctcaagtacaaccccaccaacgtggacatctctgacaaccccaacacctatgactacatgaacaagcgtgtggtggctcccggcctggtggactgctttgtcaatgtgggagccaggtggtccctggactacatggacaacgtcaaccccttcaaccaccaccgcaatgcgggtctgcgctaccgctccatgatcctgggcaacgggcgctacgtgcccttccacattcaggtgccccagaagttctttgccatcaagaacctcctcctcctgccgggctcctacacttacgagtggaacttcaggaaggatgtcaacatggtcctgcagagctctctgggcaatgaccttagggtggacggggccagcatcaagtttgacagcgtcaccctctatgctaccttcttccccatggctcacaacaccgcctccacgctcgaggccatgctgaggaacgacaccaacgaccagtccttcaatgactacctctctggggccaacatgctctaccccatccccgccaaggccaccaacgtgcccatctccattccctctcgcaactgggccgccttcagaggctgggcctttacccgccttaagaccaaggaaaccccctccctgggctcgggttttgacccctactttgtctactcgggatccatcccctacctggatggcaccttctacctcaaccacacttttaagaagatatccatcatgtatgactcctccgtcagctggccgggcaatgaccgcctgctcacccccaatgagttcgaggtcaagcgcgccgtggacggcgagggctacaacgtggcccagtgcaacatgaccaaggactggttcctggtgcagatgctggccaactacaacataggctaccagggcttctacatcccagagagctacaaggacaggatgtactccttcttcagaaatttccaacccatgagcaggcaggtggtggacgagaccaaatacaaggactatcaggccattggcatcactcaccagcacaacaactcgggattcgtgggctacctggctcccaccatgcgcgaggggcaggcctaccccgccaacttcccctacccgttgataggcaaaaccgcggtcgacagcgtcacccagaaaaagttcctctgcgaccgcaccctctggcgcatccccttctctagcaacttcatgtccatgggtgcgctcacggacctgggccagaacctgctctatgccaactccgcccatgcgctggacatgacttttgaggtggaccccatggacgagcccacccttctctatattgtgtttgaagtgttcgacgtggtcagagtgcaccagccgcaccgcggtgtcatcgagaccgtgtacctgcgcacgcccttctcggccggcaacgccaccacctaaggagacagcgccgccgcctgcatgacgggttccaccgagcaagagctcagggccatcgccagagacctgggatgcggaccctattttttgggcacctatgacaaacgcttcccgggcttcatctcccgagacaagctcgcctgcgccatcgtcaacacggccgcgcgcgagaccgggggcgtgcactggctggcctttggctgggacccgcgctccaaaacctgctacctcttcgacccctttggcttctccgatcagcgcctcagacagatctatgagtttgagtacgaggggctgctgcgccgcagcgcgcttgcctcctcgcccgaccgctgcatcacccttgagaagtccaccgagaccgtgcaggggccccactcggccgcctgcggtctcttctgctgcatgtttttgcacgcctttgtgcgctggccccagagtcccatggatcgcaaccccaccatgaacttgctcaagggagtgcccaacgccatgctccagagcccccaggtccagcccaccctgcgccacaaccaggaacagctctaccgcttcctggagcgccactccccctacttccgcagtcacagcgcgcacatccggggggccacctctttctgccacttgcaagaaaacatgcaagacggaaaatgatgtacagctcgctttttaataaatgtaaagactgtgcactttatttatacacgggctctttctggttatttattcaacaccgccgtcgccatctagaaatcgaaagggttctgccgcgcgtcgccgtgcgccacgggcagagacacgttgcgatactggaagcggctcgcccacttaaactcgggcaccaccatgcggggcagtggttcctcggggaagttctcgccccacagggtgcgggtcagctgcagcgcgctcaggaggtcgggagccgagatcttgaagtcgcagttggggccggaaccctgcgcgcgcgagttgcggtacacggggttgcagcactggaacaccagcagggccggattatgcacgctggccagcaggctctcgtcgctgatcatgtcgctgtccagatcctccgcgttgctcagggcgaacggggtcatcttgcagacctgcctgcccaggaaaggcggcagcccgggcttgccgttgcagtcgcagcgcaggggcatcagcaggtgcccgcggcccgactgcgcctgcgggtacagcgcgcgcatgaaggcttcgatctgcctgaaagccacctgcgtcttggctccctccgaaaagaacatcccacaggacttgctggagaactggttcgcgggacagctggcatcgtgcaggcagcagcgcgcgtcggtgttggcgatctgcaccacgttgcgaccccaccggttcttcactatcttggccttggaagcctgctccttcagcgcgcgctggccgttctcgctggtcacatccatctctatcacctgctccttgttgatcatgtttgtaccgtgcagacacttcaggtcgccctccgtctgggtgcagcggtgctcccacagcgcgcaaccggtgggctcccaatttttgtgggtcacccccgcgtaggcctgcaggtaggcctgcaagaagcgccccatcatggccacaaaggtcttctggctcgtaaaggtcagctgcaggccgcgatgctcttcgttcagccaggtcttgcagatggcggccagcgcctcggtctgctcgggcagcatcctaaaatttgtcttcaggtcgttatccacgtggtacttgtccatcatggcgcgcgccgcctccatgcccttctcccaggcggacaccatgggcaggcttagggggtttatcacttccaccggcgaggacaccgtactttcgatttcttcttcctccccctcttcccggcgcgcgcccacgctgctgcgcgctctcaccgcctgcaccaaggggtcgtcttcaggcaagcgccgcaccgagcgcttgccgcccttgacctgcttaatcagcaccggcgggttgctgaagcccaccatggtcagcgccgcctgctcttcttcgtcttcgctgtctaccactatctctggggaagggcttctccgctctgcggcggcgcgcttcttttttttcttgggagcggccgtgatggagtccgccacggcgacggaggtcgagggcgtggggctgggggtgcgcggtaccagggcctcgtcgccctcggactcttcctctgactccaggcggcggcggagtcgcttctttgggggcgcgcgcgtcagcggcggcggagacggggacggggacggggacgggacgccctccacagggggtggtcttcgcgcagacccgcggccgcgctcgggggtcttctcgagctggtcttggtcccgactggccattgtatcctcctcctcctaggcagagagacataaggagtctatcatgcaagtcgagaaggaggagagcttaaccaccccctctgagaccgccgatgcgcccgccgtcgccgtcgcccccgctgccgccgacgcgcccgccacaccgagcgacacccccgcggacccccccgccgacgcacccctgttcgaggaagcggccgtggagcaggacccgggctttgtctcggcagaggaggatttgcgagaggaggaggataaggagaagaagccctcagtgccaaaagatgataaagagcaagacgagcacgacgcagatgcacaccagggtgaagtcgggcggggggacggagggcatgacggcgccgactacctagacgaagggaacgacgtgctcttgaagcacctgcatcgtcagtgcgccattgtttgcgacgctctgcaggagcgcagcgaagtgcccctcagcgtggcggaggtcagccacgcctacgagctcagcctcttctccccccgggtgcccccccgccgccgcgaaaacggcacatgcgagcccaacccgcgcctcaacttctaccccgcctttgtggtacccgaggtcctggccacctatcacatcttctttcaaaattgcaagatccccctctcgtgccgcgccaaccgtagccgcgccgataagatgctggccctgcgccagggcgaccacatacctgatatcgccgctttggaagatgtaccaaagatcttcgagggtctgggtcgcaacgagaagcgggcagcaaactctctgcaacaggaaaacagcgaaaatgagagtcacaccggggtactggtggagctcgagggcgacaacgcccgcctggcggtggtcaagcgcagcatcgaggtcacccactttgcctaccccgcgctaaacctgccccccaaagtcatgaacgcggccatggacgggctgatcatgcgccgcggccggcccctcgctccagatgcaaacttgcatgaggagaccgaggacggccagcccgtggtcagcgacgagcagctggcgcgctggctggagaccgcggaccccgccgaactggaggagcggcgcaagatgatgatggccgtggtgctggtcaccgtagagctggagtgtctgcagcgcttcttcggcgaccccgagatgcagagaaaggtcgaggagaccctgcactacaccttccgccagggctacgtgcgccaggcttgcaagatctccaacgtggagctcagcaacctggtgtcctacctgggcatcttgcatgagaaccgcctcgggcagagcgtgctgcactccaccctgcgcggggaggcgcgccgcgactacgtgcgcgactgcgtttacctcttcctctgctacacctggcagacggccatgggggtctggcagcagtgcctggaggagcgcaacctcaaggagctggagaagctcctgcagcgcgcgctcaaagatctctggacgggctacaacgagcgctcggtggccgccgcgctggccgacctcatcttccccgagcgcctgctcaaaaccctccagcaggggctgcccgacttcaccagccaaagcatgttgcaaaacttcaggaactttatcctggagcgttctggcatcctacccgccacctgctgcgccctgcccagcgactttgtccccctcgtgtaccgcgagtgccccccgccgctgtggggtcactgctacctgttccaactggccaactacctgtcctaccacgcggacctcatggaggactccagcggcgaggggctcatggagtgccactgccgctgcaacctctgcacgccccaccgctccctggtctgcaacacccaactgctcagcgagagtcagattatcggtaccttcgagctacagggtccgtcctcctcagacgagaagtccgcggctccggggctaaaactcactccggggctgtggacttccgcctacctgcgcaaatttgtacctgaagactaccacgcccacgagatcaggttttacgaagaccaatcccgcccgcccaaggcggagctgaccgcctgcgtcatcacccagggcgagatcctaggccaattgcaagccatccaaaaagcccgccaagactttttgctgaagaagggtcggggggtgtatctggacccccagtcgggtgaggagctcaacccggttcccccgctgccgccgccgcgggaccttgcttcccaggataagcatcgccatggctcccagaaagaagcagcagcggccgccactgccgccaccccacatgctggaggaagaggaggaatactgggacagtcaggcagaggaggtttcggacgaggaggagccggagacggagatggaagagtgggaggaggacagcttagacgaggaggcttccgaagccgaagaggcagacgcaacaccgtcaccctcggccgcagccccctcgcaggcgcccccgaagtccgctcccagcatcagcagcaacagcagcgctataacctccgctcctccaccgccgcgacccacggccgaccgcagacccaaccgtagatgggacaccaccggaaccggggccggtaagtcctccgggagaggcaagcaagcgcagcgccaaggctaccgctcgtggcgcgctcacaagaacgccatagtcgcttgcttgcaagactgcggggggaacatctccttcgcccgccgcttcctgctcttccaccacggtgtggccttcccccgtaacgtcctgcattactaccgtcatctctacagcccctactgcggcggcagtgagccagagacggtcggcggcggcggcggcgcccgtttcggcgcctaggaagacccagggcaagacttcagccaagaaactcgcggcggccgcggcgaacgcggtcgcgggggccctgcgcctgacggtgaacgaacccctgtcgacccgcgaactgaggaaccgaatcttccccactctctatgccatcttccagcagagcagagggcaggatcaggaactgaaagtaaaaaacaggtctctgcgctccctcacccgcagctgtctgtatcacaagagcgaagaccagcttcggcgcacgctggaggacgctgaggcactcttcagcaaatactgcgcgctcactcttaaggactagctccgcgcccttctcgaatttaggcgggaacgcctacgtcatcgcagcgccgccgtcatgagcaaggacattcccacgccatacatgtggagctatcagccgcagatgggactcgcggcgggcgcctcccaagactactccacccgcatgaactggctcagtgccggcccacacatgatctcacaggttaatgatatccgcacccatcgaaaccaaatattggtggagcaggcggcaattaccaccacgccccgcaataatcccaaccccagggagtggcccgcgtccctggtgtatcaggaaattcccggccccaccaccgtactacttccgcgtgattcccaggccgaagtccaaatgactaactcaggggcacagctcgcgggcggctgtcgtcacagggtgcggcctcctcgccagggtataactcacctggagatccgaggcagaggtattcagctcaacgacgagtcggtgagctcctcgctcggtctcagacctgacgggaccttccagatagccggagccggccgatcttccttcacgccccgccaggcgtacctgactctgcaaagctcgtcctcggcgccgcgctcgggcggcatcgggactctccagttcgtgcaggagtttgtgccctcggtctacttcaaccccttctcgggctctcccggtcgctacccggaccagttcatctcgaactttgacgccgcgagggactcggtggacggctacgactgaatgtcgggtggacccggtgcagagcaacttcgcctgaagcacctcgaccactgccgccgccctcagtgctttgcccgctgtcagaccggtgagttccagtacttttccctgcccgactcgcacccggacggcccggcgcacggggtgcgctttttcatcccgagtcaggtgcgctctaccctaatcagggagtttaccgcccgtcccctactggcggagttggaaaaggggccttctatcctaaccattgcctgcatctgctctaaccctggattgcaccaagatctttgctgtcatttgtgtgctgagtataataaaggctgagatcagaatctactcgggctcctgtcgccatcctgtcaacgccaccgtccaagcccggcccgatcagcccgaggtgaacctcacctgcggtctgcaccggcgcctgaggaaatacctagcttggtactacaacagcactccctttgtggtttacaacagctttgaccaggacggggtctcactgagggataacctctcgaacctgagctactccatcaggaagaacagcaccctcgagctacttcctccttacctgcccgggacttaccagtgtgtcaccggtccctgcacccacacccacctgttgatcgtaaacgactctcttccgagaacagacctcaataactcctcttcgcagttccccagaacaggaggtgagctcaggaaaccccgggtaaagaagggtggacgagagttaacacttgtggggtttctggtgtatgtgacgctggtggtggctcttttgattaaggcttttccttccatgtctgaactctccctcttcttttatgaacaactcgactagtgctaacgggaccctacccaacgaatcgggattgaatatcggtaaccaggttgcagtttcacttttgattaccttcatagtcctcttcctgctagtgctgtcgcttctgtgcctgcggatcgggggctgctgcatccacgtttatatctggtgctggctgtttagaaggttcggagaccatcgcaggtagaataaacatgctgctgcttaccctctttgtcctggcgctggccgccagctgccaagccttttccgaggctgactttatagagccccagtgtaatgtgacttttaaagcccatgcacagcgttgtcatactataatcaaatgtgccaccgaacacgatgaataccttatccagtataaagataaatcacacaaagtggcacttgttgacatctggaaacccgaagaccctttggaatacaatgtgaccgttttccagggtgacctcttcaaaatttacaattacactttcccatttgaccagatgtgtgactttgtcatgtacatggaaaagcagcacaagctgtggcctccgactccccagggctgtgtggaaaatccaggctctttctgcatgatctctctctgtgtaactgtgctggcactaatactcacgcttttgtatatcagatttaaatcaaggcaaagcttcattgatgaaaagaaaatgccttaatcgctttcacgcttgattgctaacaccgggtttttatccgcagaatgattggaatcaccctactaatcacctccctccttgcgattgcccatgggttggaacgaatcgaagtccctgtgggggccaatgttaccctggtggggcctgtcggcaatgctacattaatgtgggaaaaatatactaaaaatcaatgggtctcttactgcactaacaaaaatagccacaagcccagagccatctgcgatgggcaaaatctaaccttgattgatgttcaattgctggatgcgggctactattatgggcagctgggtacaatgattaattactggagaccccacagagattacatgctccacgtagtaaagggtccccttagcagcccacccactaccacctctactacccccactaccaccactactcccaccaccagcactgccgcccagcctcctcatagcagaacaaccacttttatcaattccaagtcccactccccccacattgccggcgggccctccgcctcagactccgaaaccaccgagatctgcttctgcaaatgctctgacgccattgcccaggatttggaagatcacgaggaagatgagcatgacttcgcagatgcatgccaggcatcagagccagaagcgctgccggtggccctcaaacagtatgcagacccccacaccacccccgaccttcctccaccttcccagaagccaagtttcctgggggaaaatgaaactctgcctctctccatactcgctctgacatctgttgctatgttgaccgctctgctggtgcttctatgctctatatgctacctgatctgctgcagaaagaaaaaatctcacggccatgctcaccagcccctcatgcacttcccttaccctccagagctgggcgaccacaaactttaagtctgcagtaactatctgcccatcccttgtcagtcgacagcgatgagccccactaatctaacggcctctggacttacaacatcgtctcttaatgagaccaccgctcctcaagacctgtacgatggtgtctccgcgctggttaaccagtgggatcacctgggcatatggtggctcctcataggagcagtgaccctgtgcctaatcctggtctggatcatctgctgcatcaaaagcagaagacccaggcggcggcccatctacaggccctttgtcatcacacctgaagatgatgatgacaccacttccaggctgcagaggctaaagcagctactcttctcttttacagcatggtaaattgaatcatgcctcgcattttcatctacttgtctctccttccactttttctgggctcttctacattggccgctgtgtcccacatcgaggtagactgcctcacgcccttcacagtctacctgcttttcggctttgtcatctgcacctttgtctgcagcgttatcactgtagtgatctgcttcatacagtgcatcgactacgtctgcgtgcgggtggcttactttagacaccacccccagtatcgcaacagggacatagcggctctcctaagacttgtttaaaatcatggccaaattaactgtgattggtcttctgatcatctgctgcgtcctagccgcgattgggactcaagctcctaccaccaccagcgctcccagaaagagacatgtatcctgcagcttcaagcgtccctggaatataccccaatgctttactgatgaacctgaaatctctttggcttggtacttcagcgtcaccgcccttcttatcttctgcagtacggttattgcccttgccatctacccttcccttgacctgggctggaatgctgtcaactctatggaatatcccaccttcccagaaccagacctgccagacctggttgttctaaacgcgtttcctcctcctgctcccgttcaaaatcagtttcgccctccgtcccccacgcccactgaggtcagctactttaatctaacaggcggagatgactgaaaacctagacctagaaatggacggtctctgcagcgagcaacgcacactagagaggcgccggcaaaaagagctcgagcgtcttaaacaagagctccaagacgcggtggccatacaccagtgcaaaaaaggtgtcttctgtctggtaaaacaggccacgctcacctatgaaaaaacaggtgacacccaccgcctaggatacaagctgcccacacagcgccaaaagttcgccctcatgataggcgaacaacccatcaccgtgacccagcactccgtggagacagaaggctgcatacatgctccctgtaggggcgctgactgcctctacaccttgatcaaaaccctctgcggtctcagagaccttatccctttcaattaatcataactgtaatcaataaaaaatcacttacttgaaatctgatagcaagcctctgtccaattttttcagcaacacttccttcccctcctcccaactctggtactctaggcgcctcctagctgcaaacttcctccacagtctgaagggaatgtcagattcctcctcctgtccctccgcacccacgatcttcatgttgttgcagatgaaacgcgcgagatcgtctgacgagaccttcaaccccgtgtacccctacgataccgagatcgctccgacttctgtccctttccttacccctccctttgtgtcatccgcaggaatgcaagaaaatccagctggggtgctgtccctgcacttgtcagagccccttaccacccacaatggggccctgactctaaaaatggggggcggcctgaccctggacaaggaagggaatctcacttcccaaaacatcaccagtgtcgatccccctctcaaaaaaagcaagaacaacatcagccttcagaccgccgcacccctcgccgtcagctccggggccctaacactttttgccactccccccctagcggtcagtggtgacaaccttactgtgcagtctcaggcccctctcactttggaagactcaaaactaactctggccaccaaaggacccctaactgtgtccgaaggcaaacttgtcctagaaacagaggctcccctgcatgcaagtgacagcagcagcctgggccttagcgttacggccccacttagcattaacaatgacagcctaggactagatctgcaggcacccattgtctctcaaaatggaaaactggctctaaatgtagcaggccccctagctgtggccaatggcattaatgctttgacagtaggcacaggcaaaggtattggtctaaatgaaaccagcactcacttgcaagcaaagttggtcgcccccctaggctttgataccaatggcaacattaagctaagcgttgcaggaggcatgagactaaataatgacacacttatactagatgtaaactacccatttgaagctcaaggccaactaagtctaagagtgggccagggtccgctgtatgtagattctagcagccataacctgaccattagatgccttagaggattatacataacatcgtctaataaccaaaccggtctagaggccaacataaaactaacaaaaggccttgtctatgatggaaatgccatagcagtcaatgttggtcaaggattgcaatacagcactactgccacatcggaaggtgtgtatcctatacagtctaagataggtttgggaatggaatatgataccaacggagccatgatgacaaaactaggctctggactaagctttgacaattcaggagccattgtagtgggaaacaaaaatgatgacaggcttactctgtggactacaccagacccatctcctaactgtagaatttattctgaaaaagatactaaactaaccttggtgctgactaagtgtggcagccaaatcctaggcacagtatctgcccttgctgtcagaggcagccttgcgcccatcactaatgcatccagcatagtccaaatatttctaagatttgatgaaaatggactattgatgagcaactcatcgctagacggtgattactggaattacagaaatggggactccactaatagcacaccatatacaaatgcagtaggctttatgcctaatctagcagcctatcctaaaggtcaggctacagctgcaaaaagcagtattgtaagccaggtatacatggatggtgacactactaaacctataacactaaaaataaacttcaatggcattgatgaaacaacagaaaatacccctgttagtaaatattccatgacattctcatggagctggcccaccgcaagctacataggccacacttttgcaacaaactcttttactttctcctacatcgcccaagaataaagaaagcacagagatgcttgttttgatttcaaaattgtgtgcttttatttattttcagcttacagtatttccagtagtcattcgaataaagcttaatcaaactgcatgagaacccttccacatagcttaaattagcaccagtgcaaatggagaaaagcctcgaggtcgttgcgcggccgggatcggtgatcaccgatccagacatgataagatacattgatgagtttggacaaaccacaactagaatgcagtgaaaaaaatgctttatttgtgaaatttgtgatgctattgctttatttgtaaccattataagctgcaataaacaagttcccggatcgcgatccggcccgaggctgtagccgacgatggtgcgccaggagagttgttgattcattgtttgcctccctgctgcggtttttcaccgaagttcatgccagtccagcgtttttgcagcagaaaagccgccgacttcggtttgcggtcgcgagtgaagatccctttcttgttaccgccaacgcgcaatatgccttgcgaggtcgcaaaatcggcgaaattccatacctgttcaccgacgacggcgctgacgcgatcaaagacgcggtgatacatatccagccatgcacactgatactcttcactccacatgtcggtgtacattgagtgcagcccggctaacgtatccacgccgtattcggtgatgataatcggctgatgcagtttctcctgccaggccagaagttctttttccagtaccttctctgccgtttccaaatcgccgctttggacataccatccgtaataacggttcaggcacagcacatcaaagagatcgctgatggtatcggtgtgagcgtcgcagaacattacattgacgcaggtgatcggacgcgtcgggtcgagtttacgcgttgcttccgccagtggcgcgaaatattcccgtgcaccttgcggacgggtatccggttcgttggcaatactccacatcaccacgcttgggtggtttttgtcacgcgctatcagctctttaatcgcctgtaagtgcgcttgctgagtttccccgttgactgcctcttcgctgtacagttctttcggcttgttgcccgcttcgaaaccaatgcctaaagagaggttaaagccgacagcagcagtttcatcaatcaccacgatgccatgttcatctgcccagtcgagcatctcttcagcgtaagggtaatgcgaggtacggtaggagttggccccaatccagtccattaatgcgtggtcgtgcaccatcagcacgttatcgaatcctttgccacgcaagtccgcatcttcatgacgaccaaagccagtaaagtagaacggtttgtggttaatcaggaactgttcgcccttcactgccactgaccggatgccgacgcgaagcgggtagatatcacactctgtctggcttttggctgtgacgcacagttcatagagataaccttcacccggttgccagaggtgcggattcaccacttgcaaagtcccgctagtgccttgtccagttgcaaccacctgttgatccgcatcacgcagttcaacgctgacatcaccattggccaccacctgccagtcaacagacgcgtggttacagtcttgcgcgacatgcgtcaccacggtgatatcgtccacccaggtgttcggcgtggtg

SEQ ID NO: 26 - DNA, 114 bp, synthetic construct
  misc_feature 1..114: note = IL-2 core promoter
  source 1..114: mol_type = other DNA
acattttgacacccccataatatttttccagaattaacagtataaattgcatctcttgttcaagagttccctatcactctctttaatcactactcacagtaacctcaactcctg

SEQ ID NO: 27 - DNA, 55 bp, synthetic construct
  misc_feature 1..55: note = IL-2 minimal promoter
  source 1..55: mol_type = other DNA
tcaagagttccctatcactctctttaatcactactcacagtaacctcaactcctg

SEQ ID NO: 28 - DNA, 380 bp, synthetic construct
  misc_feature 1..380: note = IL-2 enhancer and promoter variant
  source 1..380: mol_type = other DNA
tgatatcttttctgagttacttttgtatccccacccccttaaagaaaggaggaaaaactgtttcatacagaaggcgttaattgcatgaattagagctatcacctaagtgtgggctaatgtaacaaagagggatttcacctacatccattcagtcagtctttgggggtttaaagaaattccaaagagtcatcagaagaggaaaaatgaaggtaatgttttttcagactggtaaagtctttgaaaatatgtgtaatatgtaaaacattttgacacccccataatatttttccagaattaacagtataaattgcatctcttgttcaagagttccctatcactctctttaatcactactcacagtaacctcaactcctgccaca

SEQ ID NO: 29 - DNA, 373 bp, synthetic construct
  misc_feature 1..373: note = L-2 enhancer and promoter variant
  source 1..373: mol_type = other DNA
ttttctgagttacttttgtatccccacccccttaaagaaaggaggaaaaactgtttcatacagaaggcgttaattgcatgaattagagctatcacctaagtgtgggctaatgtaacaaagagggatttcacctacatccattcagtcagtctttgggggtttaaagaaattccaaagagtcatcagaagaggaaaaatgaaggtaatgttttttcagactggtaaagtctttgaaaatatgtgtaatatgtaaaacattttgacacccccataatatttttccagaattaacagtataaattgcatctcttgttcaagagttccctatcactctctttaatcactactcacagtaacctcaactcctgccaca

SEQ ID NO: 30 - DNA, 161 bp, synthetic construct
  misc_feature 1..161: note = (NF-?B)1-IL2 promoter variant
  source 1..161: mol_type = other DNA
aattggtcccatcgaagagggatttcacctacataattggtcccgggacattttgacacccccataatatttttccagaattaacagtataaattgcatctcttgttcaagagttccctatcactctctttaatcactactcacagtaacctcaactcctg

SEQ ID NO: 31 - DNA, 201 bp, synthetic construct
  misc_feature 1..201: note = (NF-?B)3-IL2 promoter variant
  source 1..201: mol_type = other DNA
aattggtcccatcgaagagggatttcacctacataagagggatttcacctacataagagggatttcacctacataattggtcccgggacattttgacacccccataatatttttccagaattaacagtataaattgcatctcttgttcaagagttccctatcactctctttaatcactactcacagtaacctcaactcctg

SEQ ID NO: 32 - DNA, 268 bp, synthetic construct
  misc_feature 1..268: note = (NF-?B)6-IL2 promoter variant
  source 1..268: mol_type = other DNA
aattggtcccatcgaagagggatttcacctacataagagggatttcacctacataagagggatttcacctacataattggtaagagggatttcacctacataagagggatttcacctacataagagggatttcacctacataattggtcccgggacattttgacacccccataatatttttccagaattaacagtataaattgcatctcttgttcaagagttccctatcactctctttaatcactactcacagtaacctcaactcctg

SEQ ID NO: 33 - DNA, 177 bp, synthetic construct
  misc_feature 1..177: note = 1X NFAT response elements-IL2 promoter variant
  source 1..177: mol_type = other DNA
aattggtcccatcgaattaggaggaaaaactgtttcatacagaaggcgtcaattggtcccgggacattttgacacccccataatatttttccagaattaacagtataaattgcatctcttgttcaagagttccctatcactctctttaatcactactcacagtaacctcaactcctg

SEQ ID NO: 34 - DNA, 256 bp, synthetic construct
  misc_feature 1..256: note = 3X NFAT response elements-IL2 promoter variant
  source 1..256: mol_type = other DNA
tgatatcaattggtcccatcgaattaggaggaaaaactgtttcatacagaaggcgtcaattaggaggaaaaactgtttcatacagaaggcgtcaattaggaggaaaaactgtttcatacagaaggcgtcaattggtcccgggacattttgacacccccataatatttttccagaattaacagtataaattgcatctcttgttcaagagttccctatcactctctttaatcactactcacagtaacctcaactcctg

SEQ ID NO: 35 - DNA, 249 bp, synthetic construct
  misc_feature 1..249: note = 3X NFAT response elements-IL2 promoter variant
  source 1..249: mol_type = other DNA
aattggtcccatcgaattaggaggaaaaactgtttcatacagaaggcgtcaattaggaggaaaaactgtttcatacagaaggcgtcaattaggaggaaaaactgtttcatacagaaggcgtcaattggtcccgggacattttgacacccccataatatttttccagaattaacagtataaattgcatctcttgttcaagagttccctatcactctctttaatcactactcacagtaacctcaactcctg

SEQ ID NO: 36 - DNA, 358 bp, synthetic construct
  misc_feature 1..358: note = 6X NFAT response elements-IL2 promoter variant
  source 1..358: mol_type = other DNA
gaattaggaggaaaaactgtttcatacagaaggcgtcaattaggaggaaaaactgtttcatacagaaggcgtcaattaggaggaaaaactgtttcatacagaaggcgtcaattggtcccatcgaattaggaggaaaaactgtttcatacagaaggcgtcaattaggaggaaaaactgtttcatacagaaggcgtcaattaggaggaaaaactgtttcatacagaaggcgtcaattggtcccgggacattttgacacccccataatatttttccagaattaacagtataaattgcatctcttgttcaagagttccctatcactctccttaatcactactcacagtaacctcaactcctg

SEQ ID NO: 37 - DNA, 374 bp, synthetic construct
  misc_feature 1..374: note = 6X NFAT response elements-IL2 promoter variant
  source 1..374: mol_type = other DNA
tgatatcgaattaggaggaaaaactgtttcatacagaaggcgtcaattaggaggaaaaactgtttcatacagaaggcgtcaattaggaggaaaaactgtttcatacagaaggcgtcaattggtcccatcgaattaggaggaaaaactgtttcatacagaaggcgtcaattaggaggaaaaactgtttcatacagaaggcgtcaattaggaggaaaaactgtttcatacagaaggcgtcaattggtcccgggacattttgacacccccataatatttttccagaattaacagtataaattgcatctcttgttcaagagttccctatcactctctttaatcactactcacagtaacctcaactcctgaattccatg

SEQ ID NO: 38 - DNA, 358 bp, synthetic construct
  misc_feature 1..358: note = 6X NFAT response elements-IL2 promoter variant
  source 1..358: mol_type = other DNA
gaattaggaggaaaaactgtttcatacagaaggcgtcaattaggaggaaaaactgtttcatacagaaggcgtcaattaggaggaaaaactgtttcatacagaaggcgtcaattggtcccatcgaattaggaggaaaaactgtttcatacagaaggcgtcaattaggaggaaaaactgtttcatacagaaggcgtcaattaggaggaaaaactgtttcatacagaaggcgtcaattggtcccgggacattttgacacccccataatatttttccagaattaacagtataaattgcatctcttgttcaagagttccctatcactctctttaatcactactcacagtaacctcaactcctg

SEQ ID NO: 39 - DNA, 365 bp, synthetic construct
  misc_feature 1..365: note = 6X NFAT response elements-IL2 promoter variant
  source 1..365: mol_type = other DNA
tgatatcgaattaggaggaaaaactgtttcatacagaaggcgtcaattaggaggaaaaactgtttcatacagaaggcgtcaattaggaggaaaaactgtttcatacagaaggcgtcaattggtcccatcgaattaggaggaaaaactgtttcatacagaaggcgtcaattaggaggaaaaactgtttcatacagaaggcgtcaattaggaggaaaaactgtttcatacagaaggcgtcaattggtcccgggacattttgacacccccataatatttttccagaattaacagtataaattgcatctcttgttcaagagttccctatcactctctttaatcactactcacagtaacctcaactcctg

SEQ ID NO: 40 - DNA, 244 bp, synthetic construct
  misc_feature 1..244: note = human EEF1A1 promoter variant
  source 1..244: mol_type = other DNA
gagcgtgcgtgaggctccggtgcccgtcagtgggcagagcgcacatcgcccacagtccccgagaagttggggggagggggtcggcgattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacag

SEQ ID NO: 41 - DNA, 236 bp, synthetic construct
  misc_feature 1..236: note = human EEF1A1 promoter variant
  source 1..236: mol_type = other DNA
gcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacaca

SEQ ID NO: 42 - DNA, 1266 bp, synthetic construct
  misc_feature 1..1266: note = human EEF1A1 promoter and enhancer
  source 1..1266: mol_type = other DNA
gagctttgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgag

SEQ ID NO: 43 - DNA, 571 bp, synthetic construct
  misc_feature 1..571: note = human UBC promoter
  source 1..571: mol_type = other DNA
ggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctgggtacgtgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacg

SEQ ID NO: 44 - DNA, 81 bp, synthetic construct
  misc_feature 1..81: note = synthetic minimal promoter 1
  source 1..81: mol_type = other DNA
aggtctatataagcagagctcgtttagtgaaccctcattctggagacggatcccgagccgagtgttttgacctccatagaa

SEQ ID NO: 45 - protein, 151 aa, synthetic construct
  REGION 1..151: note = HPV antigen design 1 HPV16 E6 amino acids
MFQDPQERPRKLPQLCTALQTTIHDIILECVYCKQQLLRREVYDFAFRDGCIVYRDGNPYAVCDKCLKFYSKISEYRHYCYSLYGTTLEQQYNKPLCDLLIRCINCQKPLCPEEKQRHLDKKQRFHNIRGRWTGRCMSCCRSSRTRRAAAA

SEQ ID NO: 46 - protein, 98 aa, synthetic construct
  REGION 1..98: note = HPV antigen design 1 HPV16 E7 amino acids
MPGDTPTLHEYMLDLQPETTDLYGYEQLNDSSEEEDEIDGPAGQAAPDRAHYNIVTFCCKCDSTLRRCVQSTHVDIRTLEDLLMGTLGIVCPICSQKP

SEQ ID NO: 47 - protein, 58 aa, synthetic construct
  REGION 1..58: note = HPV antigen design 1 HPV16 E5 amino acids
MTNLDTASTTLLACFLLCFCVLLCVCLLIRPLLLSVSTYTRCFIVYIIFVYIPLFLIH

SEQ ID NO: 48 - protein, 120 aa, synthetic construct
  REGION 1..120: note = HPV antigen design 1 HPV18 E6 amino acids
MARFEDPTRRPYKLPDLCTALNTSLQDIEITCVYCKTVLELTEVFEFAFKDGFVVYRDSIPHAACHKCIDFYSRIRELRHYSDSVYGDTLEKLTNTGLYNLLIRCLRCQKPLNPAEKLRH

SEQ ID NO: 49 - protein, 66 aa, synthetic construct
  REGION 1..66: note = HPV antigen design 1 HPV18 E7 amino acids
EIDGVNHQHLPARRAAPQRHTMLCMCCKCEARIERVVESSADDLRAFQQLFLNTLSFVCPWCASQQ

SEQ ID NO: 50 - protein, 53 aa, synthetic construct
  REGION 1..53: note = HPV antigen design 1 HPV18 E5 amino acids
MLSLIFLFCFCVCMYVCCHVPLLPSVVVITSPATAFTVYLLPMLLLHIHAILS

SEQ ID NO: 51 - protein, 547 aa, synthetic construct
  REGION 1..547: note = HPV antigen design 1 amino acids
MMFQDPQERPRKLPQLCTALQTTIHDIILECVYCKQQLLRREVYDFAFRDGCIVYRDGNPYAVCDKCLKFYSKISEYRHYCYSLYGTTLEQQYNKPLCDLLIRCINCQKPLCPEEKQRHLDKKQRFHNIRGRWTGRCMSCCRSSRTRRAAAAMPGDTPTLHEYMLDLQPETTDLYGYEQLNDSSEEEDEIDGPAGQAAPDRAHYNIVTFCCKCDSTLRRCVQSTHVDIRTLEDLLMGTLGIVCPICSQKPMTNLDTASTTLLACFLLCFCVLLCVCLLIRPLLLSVSTYTRCFIVYIIFVYIPLFLIHMARFEDPTRRPYKLPDLCTALNTSLQDIEITCVYCKTVLELTEVFEFAFKDGFVVYRDSIPHAACHKCIDFYSRIRELRHYSDSVYGDTLEKLTNTGLYNLLIRCLRCQKPLNPAEKLRHEIDGVNHQHLPARRAAPQRHTMLCMCCKCEARIERVVESSADDLRAFQQLFLNTLSFVCPWCASQQMLSLIFLFCFCVCMYVCCHVPLLPSVVVITSPATAFTVYLLPMLLLHIHAILS

SEQ ID NO: 52 - protein, 563 aa, synthetic construct
  REGION 1..563: note = HPV antigen design 2 amino acids
MQTDRTGETALHLAARYSRSDHLDKKQRFHNIADARWTGRCMSCCDNMTTLEQQYNKPLCDLLPLHAAVSADISEYRHYCYRNRATDLVYDFAFRDLMHDTIHDIILECVPLILAARLAVKLPQLCTELSHADVFAFRDLCIVYDDLLCIVYRDGNPYAVCDALHWAAAVNNVLCVQSTHVDIGANKRTLEDLLMGTNNRTLGIVCPIPLFLAAREGSYELLMGTLGIVANRTLHEYMLDLDHMAHYNIVTFCCPRDYMLDLQPETTIVRLLDLEVSQTSKLTRQTDRTGETALHLAARYSRSDCDSTLRLCVADAKLTNTGLYNLDNMKCIDFYSRIPLHAAVSADFAFKDLFVVRNRATDLNLLIRCLRCMHDKLPDLCTELPLILAARLAVELTEVFEFASHADVSLQDIEITCVDDLKTVLELTEVLHWAAAVNNVDDLRAFQQLFLNTLSGANKFQQLFLNTLNNRQLFLNTLSFVPLFLAAREGSYELFLNTLSFVCPWCASANRTLQDIVLHLDHMSEEENDEIDGVNHQHLPARRPRDYIIFVYIPLIVRLLDLE

SEQ ID NO: 53 - protein, 605 aa, synthetic construct
  REGION 1..605: note = HPV antigen design 3 amino acids
MKLPQLCTEVMFQDPQERPRKLPQLCTALQTTIHDIILECVYCKQQLLRREVYDFAFRDGCIVYRDGNPYAVCDKCLKFYSKISEYRHYCYSLYGTTLEQQYNKPLCDLLIRCINCQKPLCPEEKQRHLDKKQRFHNIRGRWTGRCMSCCRSSRTRRAAAAQLYNKPLCDVMPGDTPTLHEYMLDLQPETTDLYGYEQLNDSSEEEDEIDGPAGQAAPDRAHYNIVTFCCKCDSTLRRCVQSTHVDIRTLEDLLMGTLGIVCPICSQKPRTLEDLLMGVMTNLDTASTTLLACFLLCFCVLLCVCLLIRPLLLSVSTYTRCFIVYIIFVYIPLFLIHKLPDLCTEVMARFEDPTRRPYKLPDLCTALNTSLQDIEITCVYCKTVLELTEVFEFAFKDGFVVYRDSIPHAACHKCIDFYSRIRELRHYSDSVYGDTLEKLTNTGLYNLLIRCLRCQKPLNPAEKLRHKLTNTGLYNVEIDGVNHQHLPARRAAPQRHTMLCMCCKCEARIERVVESSADDLRAFQQLFLNTLSFVCPWCASQQRAFQQLFLNVMLSLIFLFCFCVCMYVCCHVPLLPSVVVITSPATAFTVYLLPMLLLHIHAILS

SEQ ID NO: 54 - protein, 572 aa, synthetic construct
  REGION 1..572: note = HPV antigen design 4 amino acids
MQTDRELTEVFEFAARYSRSDYIIFVYIPLADAKLPQLCTEVDNMLLMGTLGIVPLHAAVSADTLHEYMLDLRNRATDLSEEENDEIDGVNHQHLPARRMHDNLLIRCLRCPLILAARLAVLFLNTLSFVCPWCASSHADVKCIDFYSRIDDLKLPDLCTELALHWAAAVNNVQLFLNTLSFVGANKFQQLFLNTLNNRVYDFAFRDLPLFLAAREGSYEKLTNTGLYNLANRLCIVYRDGNPYAVCDDHMYMLDLQPETTPRDFAFRDLCIVYIVRLLDLEVSQTSKLTRQTDRTLGIVCPIAARYSRSDRTLEDLLMGVADATIHDIILECVDNMHLDKKQRFHNIPLHAAVSADTTLEQQYNKPLCDLLRNRATDLCDSTLRLCVMHDRWTGRCMSCCPLILAARLAVSLQDIEITCVSHADVLCVQSTHVDIDDLAHYNIVTFCCLHWAAAVNNVDDLRAFQQLFLNTLSGANKISEYRHYCYNNRKLPQLCTELPLFLAAREGSYEKTVLELTEVANRRTLEDLLMGTDHMFAFKDLFVVPRDQLYNKPLCDVIVRTLQDIVLHLLE

SEQ ID NO: 55 - protein, 436 aa, synthetic construct
  REGION 1..436: note = HPV antigen design 5 amino acids
MKKELTEVFEFAKKYIIFVYIPLKKKLPQLCTEVKKLLMGTLGIVKKTLHEYMLDLKKSEEENDEIDGVNHQHLPARRKKNLLIRCLRCKKLFLNTLSFVCPWCASKKKCIDFYSRIKKKLPDLCTELKKQLFLNTLSFVKKFQQLFLNTLKKVYDFAFRDLKKKLTNTGLYNLKKLCIVYRDGNPYAVCDKKYMLDLQPETTKKFAFRDLCIVYKKTLGIVCPIKKRTLEDLLMGVKKTIHDIILECVKKHLDKKQRFHNIKKTTLEQQYNKPLCDLLKKCDSTLRLCVKKRWTGRCMSCCKKSLQDIEITCVKKLCVQSTHVDIKKAHYNIVTFCCKKDDLRAFQQLFLNTLSKKISEYRHYCYKKKLPQLCTELKKKTVLELTEVKKRTLEDLLMGTKKFAFKDLFVVKKQLYNKPLCDVKKTLQDIVLHLKK

SEQ ID NO: 56 - DNA, 6380 bp, synthetic construct
  misc_feature 1..6380: note = HPV antigen design 1 full nucleotide sequences of gorilla adenovirus shuttle plasmid
  source 1..6380: mol_type = other DNA
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctgggtcaagtcttccagtttaagcagcagagcggtcagtttctcatcccgagcagacgcgcgagaggccgcgccgctcgccaccaaagagctgtaaaggtccgtagccatgctgcgcgcggtcgcggcggcggcggaggcggcggcggaggtcgcggcgtccagcggagttcctcccacggtcgcgtaggccattgtagacgaatttgaaggcagaacggggcgtccatccacgttggaacccatcacattctgacgcactccagcccagtgaggcatgcgcactgtcagataggggctaaagatgcttccatcaaagctgttgccggtgtcgctcatggcggcggctgttgcaagacaaaacagagagacccttagacccccaatttatacacgccccacccttctagccacgcccaccttacccacctcaatcggtatcctcatcgctagacccaaactcggccctggtgcaggccagcaccagatggtcaggcctctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcacttgaggtgtggcaggcttgagatctggccatacacttgagtgacaatgacatccactttgcctttctctccacaggtgtccactcccaggtccaactgcagccggatccggtaccgccaccatgatgttccaggatcctcaagaacggcctcggaaactacctcagctgtgtacagccttacagacaacaatacatgatataatcctggagtgcgtctactgtaaacagcaactgttgcgtcgcgaggtctatgatttcgccttcagggatggttgtattgtatacagagacggcaatccgtatgcagtgtgtgataagtgtctcaagttctatagcaaaatcagtgaataccggcactattgctatagcctgtatggaactacactagagcagcagtataacaagccactctgtgacctgctaattagatgtatcaactgccagaagcctctgtgccccgaagagaagcagcggcaccttgacaagaagcagcggtttcataatatcagagggaggtggaccgggcggtgtatgtcctgctgccgcagcagcaggacccgtcgggccgcggcggcaatgccaggggacacgcccacactccacgagtacatgctcgaccttcagccggaaaccaccgatctctacggctatgaacagttgaacgacagctccgaagaggaagatgagatcgacggcccagcaggacaggcggcccctgacagagcacactacaatattgttaccttctgctgcaagtgcgacagcacgctgcgccgatgcgtacaatctacgcatgtggacattaggacccttgaggatctgcttatggggacactcggaattgtttgtcccatatgcagtcagaaacctatgaccaacctagacaccgcgagcacgactcttctggcttgcttcctgctatgtttttgcgtgctgctttgtgtttgcctactcattcggcccctgctgctgtctgtgagcacgtacacgaggtgcttcattgtctatattatcttcgtttacataccgctgttcctgattcacatggcaagatttgaagatcccacgcggcggccatacaaactccctgatctctgcacggcactgaacacttcgttgcaggacatagaaatcacttgcgtgtactgcaagactgtgctagagctgaccgaggtgtttgagtttgccttcaaggacggttttgtggtgtatcgggatagcatcccccatgccgcctgccataaatgtattgatttctattcgagaattagagaactcaggcattattccgactccgtatacggcgacaccctagaaaaactgaccaacaccggcttgtataacctcctcatccgctgcctccgatgccagaagccacttaacccagccgagaaattacgccacgagatcgacggggtcaaccatcagcacctgccggccagacgagccgcccctcagcgccataccatgctgtgcatgtgttgcaagtgcgaagccaggatcgaaagggtggtggaatctagcgctgacgacttaagggcattccaacaactctttcttaacaccctgagcttcgtgtgtccttggtgtgcttcccaacagatgcttagtctgatttttctgttttgcttttgcgtgtgtatgtatgtctgctgccacgtgcctctcctaccatctgtagtggtgattacatcccccgcgactgccttcaccgtgtacctgttgccaatgctcctccttcacatacacgccatcctgagctaagcggccgctctcgagtctagctagtctagactagctagaaagatccgggaacttgtttattgcagcttataatggttacaaataaagcaatagcatcacaaatttcacaaataaagcatttttttcactgcattctagttgtggtttgtccaaactcatcaatgtatcttatcatgtctggatcggtgatcaccgatcccgatctgtcaaatgacggtgacaataaaacggagactttgacccggaacgcggaaattcacgtaaaaaacacctgggcgagtcctccacgtaatcggtcaaagtccctcggccctcggtaaatattacgcactatgactaacgccctattattcagttttcacttccccgtttcacttttcgcgcgaaaatggccaaatcttacatggtcccgcccaaaattactacgatatccggtgaaaagcgcgcgaaaattggcacttccggaggtaggcggcgctcatcaaaaacgtcacattttccgcgacggaagcttgcatgtgagctcctcccacttgcaaatgccacacttccgccacacctcccaaccctactcgcgcgtcctacgtcacccgccccgcctctccccgcccacctcattatcatattggccacaatccaaaataaggtatattattgatgatggtttaaacgcccaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 57 - DNA, 6428 bp, synthetic construct
  misc_feature 1..6428: note = HPV antigen design 2 full nucleotide sequences of gorilla adenovirus shuttle plasmid
  source 1..6428: mol_type = other DNA
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctgggtcaagtcttccagtttaagcagcagagcggtcagtttctcatcccgagcagacgcgcgagaggccgcgccgctcgccaccaaagagctgtaaaggtccgtagccatgctgcgcgcggtcgcggcggcggcggaggcggcggcggaggtcgcggcgtccagcggagttcctcccacggtcgcgtaggccattgtagacgaatttgaaggcagaacggggcgtccatccacgttggaacccatcacattctgacgcactccagcccagtgaggcatgcgcactgtcagataggggctaaagatgcttccatcaaagctgttgccggtgtcgctcatggcggcggctgttgcaagacaaaacagagagacccttagacccccaatttatacacgccccacccttctagccacgcccaccttacccacctcaatcggtatcctcatcgctagacccaaactcggccctggtgcaggccagcaccagatggtcaggcctctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcacttgaggtgtggcaggcttgagatctggccatacacttgagtgacaatgacatccactttgcctttctctccacaggtgtccactcccaggtccaactgcagccggatccggtaccgccaccatgcagactgatagaaccggcgaaaccgctctacaccttgccgcccgttatagtcgaagcgaccacctcgataagaaacagcgattccacaacatcgcagacgcaagatggaccgggcggtgtatgtcctgctgtgacaatatgacaacactggaacagcagtataacaagcccctgtgtgacctgttgcccctgcacgccgccgtgtcagccgacatatccgagtatcggcattactgctaccggaaccgtgccaccgacctcgtgtacgactttgcatttagagacctgatgcacgataccatccatgacatcatcctggaatgtgtcccactgatcctggctgctcggcttgcagtgaagctaccccagctatgcactgagctatcacacgcagacgtgttcgccttcagggatctgtgcattgtttacgatgacctgttgtgtattgtgtacagagatggcaacccttatgcagtgtgtgacgccctgcattgggctgccgccgtgaataatgtcctttgtgtccagagcacccacgtcgatataggcgctaataagcggacactggaggacctgttgatggggactaataatagaaccctgggaatcgtgtgccccattccattattcctggccgcccgcgagggatcttatgaactcctgatgggcacactaggcatcgtggccaataggacattgcacgagtatatgctagacttagaccacatggctcactataacattgtcacattttgttgcccccgcgattacatgcttgatttacagcccgaaacaaccatcgtgagactgctcgatctggaggtatctcagacaagtaagctcacaagacaaacagacagaaccggcgagactgctttgcatctggctgcacgatattccaggtccgactgtgatagcaccctgcggttatgtgtggcagacgcaaaacttaccaataccggcctatataacctcgacaacatgaagtgcatcgacttctacagcagaatccccctccatgccgccgtgagcgccgattttgccttcaaggacctgttcgtggtgagaaaccgagccaccgacctgaacctgttgattcggtgtttacgctgtatgcacgacaagctgcccgatttgtgtactgagctgcctctcattctggccgcacggctcgcagttgaactaaccgaagtgttcgagttcgcctcccacgccgacgtgtccctacaggacatcgagatcacatgcgtagacgatctgaaaaccgttctcgaactcacagaagtgcttcattgggccgccgcagtcaacaacgtggacgatctccgggcattccagcaactgttcctgaatacactgtctggcgctaacaagtttcagcagttatttttgaacacacttaataaccgccagctattcttgaataccctgtcattcgtgccgctgttcttggctgcgcgcgaggggtcctacgagctgtttctgaatacgctctccttcgtgtgtccctggtgcgcctccgctaataggaccctgcaagacatcgtacttcacctggatcacatgtcagaggaggagaacgatgagatcgacggtgtcaaccaccagcacctccccgcccggaggccccgagattacatcatttttgtatatattcccttaatcgtaagattactcgacctggagtaagcggccgctctcgagtctagctagtctagactagctagaaagatccgggaacttgtttattgcagcttataatggttacaaataaagcaatagcatcacaaatttcacaaataaagcatttttttcactgcattctagttgtggtttgtccaaactcatcaatgtatcttatcatgtctggatcggtgatcaccgatcccgatctgtcaaatgacggtgacaataaaacggagactttgacccggaacgcggaaattcacgtaaaaaacacctgggcgagtcctccacgtaatcggtcaaagtccctcggccctcggtaaatattacgcactatgactaacgccctattattcagttttcacttccccgtttcacttttcgcgcgaaaatggccaaatcttacatggtcccgcccaaaattactacgatatccggtgaaaagcgcgcgaaaattggcacttccggaggtaggcggcgctcatcaaaaacgtcacattttccgcgacggaagcttgcatgtgagctcctcccacttgcaaatgccacacttccgccacacctcccaaccctactcgcgcgtcctacgtcacccgccccgcctctccccgcccacctcattatcatattggccacaatccaaaataaggtatattattgatgatggtttaaacgcccaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 58 - DNA, 6554 bp, synthetic construct
  misc_feature 1..6554: note = HPV antigen design 3 full nucleotide sequences of gorilla adenovirus shuttle plasmid
  source 1..6554: mol_type = other DNA
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctgggtcaagtcttccagtttaagcagcagagcggtcagtttctcatcccgagcagacgcgcgagaggccgcgccgctcgccaccaaagagctgtaaaggtccgtagccatgctgcgcgcggtcgcggcggcggcggaggcggcggcggaggtcgcggcgtccagcggagttcctcccacggtcgcgtaggccattgtagacgaatttgaaggcagaacggggcgtccatccacgttggaacccatcacattctgacgcactccagcccagtgaggcatgcgcactgtcagataggggctaaagatgcttccatcaaagctgttgccggtgtcgctcatggcggcggctgttgcaagacaaaacagagagacccttagacccccaatttatacacgccccacccttctagccacgcccaccttacccacctcaatcggtatcctcatcgctagacccaaactcggccctggtgcaggccagcaccagatggtcaggcctctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcacttgaggtgtggcaggcttgagatctggccatacacttgagtgacaatgacatccactttgcctttctctccacaggtgtccactcccaggtccaactgcagccggatccggtaccgccaccatgaagctaccgcagctctgtacagaagtgatgttccaggatcctcaagaacggcctcggaaactacctcagctgtgtacagccttacagacaacaatacatgatataatcctggagtgcgtctactgtaaacagcaactgttgcgtcgcgaggtctatgatttcgccttcagggatggttgtattgtatacagagacggcaatccgtatgcagtgtgtgataagtgtctcaagttctatagcaaaatcagtgaataccggcactattgctatagcctgtatggaactacactagagcagcagtataacaagccactctgtgacctgctaattagatgtatcaactgccagaagcctctgtgccccgaagagaagcagcggcaccttgacaagaagcagcggtttcataatatcagagggaggtggaccgggcggtgtatgtcctgctgccgcagcagcaggacccgtcgggccgcggcggcacagctgtacaacaaaccactgtgtgacgtgatgccaggggacacgcccacactccacgagtacatgctcgaccttcagccggaaaccaccgatctctacggctatgaacagttgaacgacagctccgaagaggaagatgagatcgacggcccagcaggacaggcggcccctgacagagcacactacaatattgttaccttctgctgcaagtgcgacagcacgctgcgccgatgcgtacaatctacgcatgtggacattaggacccttgaggatctgcttatggggacactcggaattgtttgtcccatatgcagtcagaaacctcggacactggaagatttgctcatgggcgttatgaccaacctagacaccgcgagcacgactcttctggcttgcttcctgctatgtttttgcgtgctgctttgtgtttgcctactcattcggcccctgctgctgtctgtgagcacgtacacgaggtgcttcattgtctatattatcttcgtttacataccgctgttcctgattcacaagctgcccgatttgtgcacagaggtgatggcaagatttgaagatcccacgcggcggccatacaaactccctgatctctgcacggcactgaacacttcgttgcaggacatagaaatcacttgcgtgtactgcaagactgtgctagagctgaccgaggtgtttgagtttgccttcaaggacggttttgtggtgtatcgggatagcatcccccatgccgcctgccataaatgtattgatttctattcgagaattagagaactcaggcattattccgactccgtatacggcgacaccctagaaaaactgaccaacaccggcttgtataacctcctcatccgctgcctccgatgccagaagccacttaacccagccgagaaattacgccacaagctgacgaacaccggtttgtacaatgtggagatcgacggggtcaaccatcagcacctgccggccagacgagccgcccctcagcgccataccatgctgtgcatgtgttgcaagtgcgaagccaggatcgaaagggtggtggaatctagcgctgacgacttaagggcattccaacaactctttcttaacaccctgagcttcgtgtgtccttggtgtgcttcccaacagcgcgccttccaacaattgttcctcaatgtaatgcttagtctgatttttctgttttgcttttgcgtgtgtatgtatgtctgctgccacgtgcctctcctaccatctgtagtggtgattacatcccccgcgactgccttcaccgtgtacctgttgccaatgctcctccttcacatacacgccatcctgagctaagcggccgctctcgagtctagctagtctagactagctagaaagatccgggaacttgtttattgcagcttataatggttacaaataaagcaatagcatcacaaatttcacaaataaagcatttttttcactgcattctagttgtggtttgtccaaactcatcaatgtatcttatcatgtctggatcggtgatcaccgatcccgatctgtcaaatgacggtgacaataaaacggagactttgacccggaacgcggaaattcacgtaaaaaacacctgggcgagtcctccacgtaatcggtcaaagtccctcggccctcggtaaatattacgcactatgactaacgccctattattcagttttcacttccccgtttcacttttcgcgcgaaaatggccaaatcttacatggtcccgcccaaaattactacgatatccggtgaaaagcgcgcgaaaattggcacttccggaggtaggcggcgctcatcaaaaacgtcacattttccgcgacggaagcttgcatgtgagctcctcccacttgcaaatgccacacttccgccacacctcccaaccctactcgcgcgtcctacgtcacccgccccgcctctccccgcccacctcattatcatattggccacaatccaaaataaggtatattattgatgatggtttaaacgcccaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 59 - DNA, 6455 bp, synthetic construct
  misc_feature 1..6455: note = HPV antigen design 4 full nucleotide sequences of gorilla adenovirus shuttle plasmid
  source 1..6455: mol_type = other DNA
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctgggtcaagtcttccagtttaagcagcagagcggtcagtttctcatcccgagcagacgcgcgagaggccgcgccgctcgccaccaaagagctgtaaaggtccgtagccatgctgcgcgcggtcgcggcggcggcggaggcggcggcggaggtcgcggcgtccagcggagttcctcccacggtcgcgtaggccattgtagacgaatttgaaggcagaacggggcgtccatccacgttggaacccatcacattctgacgcactccagcccagtgaggcatgcgcactgtcagataggggctaaagatgcttccatcaaagctgttgccggtgtcgctcatggcggcggctgttgcaagacaaaacagagagacccttagacccccaatttatacacgccccacccttctagccacgcccaccttacccacctcaatcggtatcctcatcgctagacccaaactcggccctggtgcaggccagcaccagatggtcaggcctctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcacttgaggtgtggcaggcttgagatctggccatacacttgagtgacaatgacatccactttgcctttctctccacaggtgtccactcccaggtccaactgcagccggatccggtaccgccaccatgcaaaccgacagagagcttaccgaggttttcgagtttgcagcccggtactcccggagcgactacattatttttgtctatatccctctcgcagacgcaaagctgcctcaactgtgtacagaggttgataatatgctactgatgggcactctcggcatcgtgccgctccacgccgccgtgagcgccgacaccctgcatgaatacatgctggacctgagaaatcgggcaaccgacttatccgaggaggagaacgacgaaattgatggagttaatcaccagcacctaccagcaagacgaatgcacgacaacctactgattagatgcttgaggtgtccccttattttggcagccagactcgccgttctgttcctgaacaccctgagctttgtatgcccctggtgtgccagctcgcacgcagacgtgaaatgtattgacttctattccagaatcgacgacctaaaactgcctgatctctgtacagagcttgcgttgcactgggcagccgccgtgaacaacgtgcagttgtttctgaacaccttatccttcgtgggcgctaacaaattccagcaacttttcttgaacaccctaaacaacagagtttatgactttgctttcagagacctccccctgttccttgctgccagagagggatcatacgagaaactgacaaatacgggcctctacaatttggccaacagactgtgcatcgtgtacagagatggcaatccctacgccgtttgcgacgaccacatgtacatgctcgacctccagccggagacaactcccagagacttcgcttttcgggacctgtgtatcgtgtatattgtaagactcctggacctggaagtgagccaaacaagtaagctaaccagacaaacagacagaacacttgggatcgtgtgtcctattgcagcccggtatagtaggtcagaccggactctggaggacctgttaatgggggtggcggacgcaacaatccacgacattatcttggagtgtgttgacaatatgcacctggacaagaaacagagattccacaacatacccctgcacgcagccgtcagcgcggatactactctggagcagcaatataacaagcccctgtgcgacctcctgcggaatcgggcaactgatctctgtgacagtacccttcgtttgtgcgttatgcacgacaggtggactgggagatgtatgtcttgctgtcctctcattctggccgcaagactggccgttagtctgcaagacatcgagatcacctgtgtcagtcacgccgatgtgctatgcgtgcagagcacccacgttgatattgatgacctagcacactacaacatcgttactttttgctgtctacactgggccgcagccgtgaataacgtggacgacctacgcgctttccaacaactgttccttaacaccctgagcggcgcaaacaagatcagcgagtaccgccactactgttataataaccggaagctgccacagctatgcacagagttgccactgttcctggcagcaagagagggatcttatgaaaagaccgtgctggagctgacagaggtggctaatcgccgcacactggaggacctgttgatgggaaccgatcacatgttcgcctttaaggaccttttcgttgttcccagagaccagctttacaacaaacctctatgcgacgtaatagtgaggaccttgcaggatattgtgctccatctgctggaataagcggccgctctcgagtctagctagtctagactagctagaaagatccgggaacttgtttattgcagcttataatggttacaaataaagcaatagcatcacaaatttcacaaataaagcatttttttcactgcattctagttgtggtttgtccaaactcatcaatgtatcttatcatgtctggatcggtgatcaccgatcccgatctgtcaaatgacggtgacaataaaacggagactttgacccggaacgcggaaattcacgtaaaaaacacctgggcgagtcctccacgtaatcggtcaaagtccctcggccctcggtaaatattacgcactatgactaacgccctattattcagttttcacttccccgtttcacttttcgcgcgaaaatggccaaatcttacatggtcccgcccaaaattactacgatatccggtgaaaagcgcgcgaaaattggcacttccggaggtaggcggcgctcatcaaaaacgtcacattttccgcgacggaagcttgcatgtgagctcctcccacttgcaaatgccacacttccgccacacctcccaaccctactcgcgcgtcctacgtcacccgccccgcctctccccgcccacctcattatcatattggccacaatccaaaataaggtatattattgatgatggtttaaacgcccaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 60 - DNA, 6047 bp, synthetic construct
  misc_feature 1..6047: note = HPV antigen design 5 full nucleotide sequences of gorilla adenovirus shuttle plasmid
  source 1..6047: mol_type = other DNA
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctgggtcaagtcttccagtttaagcagcagagcggtcagtttctcatcccgagcagacgcgcgagaggccgcgccgctcgccaccaaagagctgtaaaggtccgtagccatgctgcgcgcggtcgcggcggcggcggaggcggcggcggaggtcgcggcgtccagcggagttcctcccacggtcgcgtaggccattgtagacgaatttgaaggcagaacggggcgtccatccacgttggaacccatcacattctgacgcactccagcccagtgaggcatgcgcactgtcagataggggctaaagatgcttccatcaaagctgttgccggtgtcgctcatggcggcggctgttgcaagacaaaacagagagacccttagacccccaatttatacacgccccacccttctagccacgcccaccttacccacctcaatcggtatcctcatcgctagacccaaactcggccctggtgcaggccagcaccagatggtcaggcctctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcacttgaggtgtggcaggcttgagatctggccatacacttgagtgacaatgacatccactttgcctttctctccacaggtgtccactcccaggtccaactgcagccggatccggtaccgccaccatgaagaaagaattgaccgaagtatttgaatttgccaagaagtacatcatctttgtttacatacccctgaagaagaaattaccccagctgtgtacagaggtgaagaagttattgatgggaacactcggaattgtgaaaaagaccctgcacgaatatatgctagatttgaagaagtctgaggaggagaacgacgagatcgacggggtgaaccaccagcacctccccgccaggagaaagaagaatcttctcatacggtgtctgcggtgcaaaaagctgttcctgaacaccctgtcgttcgtatgtccctggtgcgcttccaagaagaagtgcatcgacttttactctcggatcaagaagaagctgcccgatctgtgtactgagctgaagaagcaactgttcctgaacacgctgtcgtttgtgaagaagtttcagcagctgttcctgaacactctaaagaaggtgtacgattttgctttcagagacctgaaaaagaagttgacaaatacggggctgtataatctgaagaaactctgtatcgtttacagggacggtaacccttacgctgtgtgtgacaaaaagtacatgctagacctccagcccgaaacgaccaagaagttcgcgttcagagacctgtgtatcgtctacaagaaaacactaggcatcgtgtgtcccattaagaaacggactcttgaggacctattgatgggtgtgaaaaagacaattcatgacattatcttggagtgtgtgaagaaacatctcgataagaaacaaagattccataacatcaaaaagacaaccctggaacagcagtataacaagcctctttgcgatctgctaaagaaatgtgactccaccttgaggctgtgcgtaaagaagcgatggaccggaagatgtatgtcttgttgtaaaaagagtctccaagatattgagattacttgcgtgaaaaagttgtgcgttcagagtacacatgttgatattaagaaagcccattataatatcgtgacattttgctgtaagaaggatgacctgagagcttttcagcagctgtttctgaatacactgagcaagaagatcagcgagtaccgacactattgttacaagaaaaagctgccccagctgtgtaccgaattgaagaagaaaaccgtcctagagcttaccgaagtgaagaagcgtaccctggaggacctgttaatgggcacgaagaagtttgcattcaaggacctgtttgtcgtgaaaaagcagctgtacaataagcccctgtgcgacgttaaaaagacactccaagacatcgtcttacacctgaaaaagtaagcggccgctctcgagtctagctagtctagactagctagaaagatccgggaacttgtttattgcagcttataatggttacaaataaagcaatagcatcacaaatttcacaaataaagcatttttttcactgcattctagttgtggtttgtccaaactcatcaatgtatcttatcatgtctggatcggtgatcaccgatcccgatctgtcaaatgacggtgacaataaaacggagactttgacccggaacgcggaaattcacgtaaaaaacacctgggcgagtcctccacgtaatcggtcaaagtccctcggccctcggtaaatattacgcactatgactaacgccctattattcagttttcacttccccgtttcacttttcgcgcgaaaatggccaaatcttacatggtcccgcccaaaattactacgatatccggtgaaaagcgcgcgaaaattggcacttccggaggtaggcggcgctcatcaaaaacgtcacattttccgcgacggaagcttgcatgtgagctcctcccacttgcaaatgccacacttccgccacacctcccaaccctactcgcgcgtcctacgtcacccgccccgcctctccccgcccacctcattatcatattggccacaatccaaaataaggtatattattgatgatggtttaaacgcccaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 61 - DNA, 10410 bp, synthetic construct
  misc_feature 1..10410: note = GCAd-RTS-IL12 design 1
  source 1..10410: mol_type = other DNA
ttcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctgggtcaagtcttccagtttaagcagcagagcggtcagtttctcatcccgagcagacgcgcgagaggccgcgccgctcgccaccaaagagctgtaaaggtccgtagccatgctgcgcgcggtcgcggcggcggcggaggcggcggcggaggtcgcggcgtccagcggagttcctcccacggtcgcgtaggccattgtagacgaatttgaaggcagaacggggcgtccatccacgttggaacccatcacattctgacgcactccagcccagtgaggcatgcgcactgtcagataggggctaaagatgcttccatcaaagctgttgccggtgtcgctcatggcggcggctgttgcaagacaaaacagagagacccttagacccccaatttatacacgccccacccttctagccacgcccaccttacccacctcaatcggtatcctcatcgctagacccaaactcggccctggtgcaggccagcaccagatggtcaggcctgcaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgataatgcaggtcggagtactgtcctccgagcggagtactgtcctccgagcggagtactgtcctccgagcggagtactgtcctccgagcggagtactgtcctccgagcggagtactgtcctccgagcggagactcttcgaaggaagaggggcggggtcgatcgaccccgcccctcttccttcgaaggaagaggggcggggtcgaagacctagagggtatataatgggtgccttagctggtgtgtgagctcatcttcctgtagatcacgcgtgccaccatgggtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttaggttgggcgagctcgaattcattgatcccccgggctgcaggaattcgatatcaagctcgggatccgaattccgccccccccccccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatgggtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaacgtacgtcgacatcgagaacttgtttattgcagcttataatggttacaaataaagcaatagcatcacaaatttcacaaataaagcatttttttcactgcattctagttgtggtttgtccaaactcatcaatgtatcttatcatgtctgggcgcgccggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctgggtacgtgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgagctagcgccgccaccatgggccctaaaaagaagcgtaaagtcgcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtggggaattcgagatgcctgtggacaggatcctggaggcagagcttgctgtggaacagaagagtgaccagggcgttgagggtcctgggggaaccgggggtagcggcagcagcccaaatgaccctgtgactaacatctgtcaggcagctgacaaacagctattcacgcttgttgagtgggcgaagaggatcccacacttttcctccttgcctctggatgatcaggtcatattgctgcgggcaggctggaatgaactcctcattgcctccttttcacaccgatccattgatgttcgagatggcatcctccttgccacaggtcttcacgtgcaccgcaactcagcccattcagcaggagtaggagccatctttgatcgggtgctgacagagctagtgtccaaaatgcgtgacatgaggatggacaagacagagcttggctgcctgagggcaatcattctgtttaatccagaggtgaggggtttgaaatccgcccaggaagttgaacttctacgtgaaaaagtatatgccgctttggaagaatatactagaacaacacatcccgatgaaccaggaagatttgcaaaacttttgcttcgtctgccttctttacgttccataggccttaagtgtttggagcatttgtttttctttcgccttattggagatgttccaattgatacgttcctgatggagatgcttgaatcaccttctgattcataatctagcctagcccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatctctaggcgccaccatgaagctactgtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgagaagaccttgacatgattttgaaaatggattctttacaggatataaaagcattgttaacaggattatttgtacaagataatgtgaataaagatgccgtcacagatagattggcttcagtggagactgatatgcctctaacattgagacagcatagaataagtgcgacatcatcatcggaagagagtagtaacaaaggtcaaagacagttgactgtatcgccggaattcccggggatccggcctgagtgcgtagtacccgagactcagtgcgccatgaagcggaaagagaagaaagcacagaaggagaaggacaaactgcctgtcagcacgacgacggtggacgaccacatgccgcccattatgcagtgtgaacctccacctcctgaagcagcaaggattcacgaagtggtcccaaggtttctctccgacaagctgttggtgacaaaccggcagaaaaacatcccccagttgacagccaaccagcagttccttatcgccaggctcatctggtaccaggacgggtacgagcagccttctgatgaagatttgaagaggattacgcagacgtggcagcaagcggacgatgaaaacgaagagtcggacactcccttccgccagatcacagagatgactatcctcacggtccaacttatcgtggagttcgcgaagggattgccagggttcgccaagatctcgcagcctgatcaaattacgctgcttaaggcttgctcaagtgaggtaatgatgctccgagtcgcgcgacgatacgatgcggcctcagacagtattctgttcgcgaacaaccaagcgtacactcgcgacaactaccgcaaggctggcatggccgaggtcatcgaggatctactgcacttctgccggtgcatgtactctatggcgttggacaacatccattacgcgctgctcacggctgtcgtcatcttttctgaccggccagggttggagcagccgcaactggtggaagagatccagcggtactacctgaatacgctccgcatctatatcctgaaccagctgagcgggtcggcgcgttcgtccgtcatatacggcaagatcctctcaatcctctctgagctacgcacgctcggcatgcaaaactccaacatgtgcatctccctcaagctcaagaacagaaagctgccgcctttcctcgaggagatctgggatgtggcggacatgtcgcacacccaaccgccgcctatcctcgagtcccccacgaatctctaggcggcctctagagcggccgccaccgcggggagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcaccggtcaaatgacggtgacaataaaacggagactttgacccggaacgcggaaattcacgtaaaaaacacctgggcgagtcctccacgtaatcggtcaaagtccctcggccctcggtaaatattacgcactatgactaacgccctattattcagttttcacttccccgtttcacttttcgcgcgaaaatggccaaatcttacatggtcccgcccaaaattactacgatatccggtgaaaagcgcgcgaaaattggcacttccggaggtaggcggcgctcatcaaaaacgtcacattttccgcgacggaagcttgcatgtgagctcctcccacttgcaaatgccacacttccgccacacctcccaaccctactcgcgcgtcctacgtcacccgccccgcctctccccgcccacctcattatcatattggccacaatccaaaataaggtatattattgatgatggtttaaacgcccaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaataca

SEQ ID NO: 62 - DNA, 10040 bp, synthetic construct
  misc_feature 1..10040: note = GCAd-RTS-IL12 design 2
  source 1..10040: mol_type = other DNA
ttcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctgggtcaagtcttccagtttaagcagcagagcggtcagtttctcatcccgagcagacgcgcgagaggccgcgccgctcgccaccaaagagctgtaaaggtccgtagccatgctgcgcgcggtcgcggcggcggcggaggcggcggcggaggtcgcggcgtccagcggagttcctcccacggtcgcgtaggccattgtagacgaatttgaaggcagaacggggcgtccatccacgttggaacccatcacattctgacgcactccagcccagtgaggcatgcgcactgtcagataggggctaaagatgcttccatcaaagctgttgccggtgtcgctcatggcggcggctgttgcaagacaaaacagagagacccttagacccccaatttatacacgccccacccttctagccacgcccaccttacccacctcaatcggtatcctcatcgctagacccaaactcggccctggtgcaggccagcaccagatggtcaggcctgcaggtacgtagccgcaataaaatatctttattttcattacatctgtgtgttggttttttgtgtgaatccatagtactaacatacgctctccatcaaaacaaaacgaaacaaaacaaactagcaaaataggctgtccccagtgcaagtccaggtgccagaacatttctctatccataatgcaggggtaccggaaggaagaggggcggggtcgatcgaccccgcccctcttccttcgaaggaagaggggcggggtccaattgcggagtactgtcctccgagcggagtactgtcctccgagcggagtactgtcctccgagcggagtactgtcctccgagcggagtactgtcctccgagcggagtactgtcctccgagcggagagtccccggggacctagagggtatataatgggtgccttagctggtgtgtgacctcatcttcctgtacgcccctgcaggagatcacgcgtgccaccatgggtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtctcgagggcggcggagagggcagaggaagtcttctaacatgcggtgacgtggaggagaatcccggccctaggatgggtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaaatcgatttatttatcggcataaataatttttttgaagaagtaatactatttttctttttttttgtaaataaatgggttaagggatgtaacattgtttgttgtttggtgggggttggggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctgggtacgtgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgagctagcgccgccaccatgggccctaaaaagaagcgtaaagtcgcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtggggaattcgagatgcctgtggacaggatcctggaggcagagcttgctgtggaacagaagagtgaccagggcgttgagggtcctgggggaaccgggggtagcggcagcagcccaaatgaccctgtgactaacatctgtcaggcagctgacaaacagctattcacgcttgttgagtgggcgaagaggatcccacacttttcctccttgcctctggatgatcaggtcatattgctgcgggcaggctggaatgaactcctcattgcctccttttcacaccgatccattgatgttcgagatggcatcctccttgccacaggtcttcacgtgcaccgcaactcagcccattcagcaggagtaggagccatctttgatcgggtgctgacagagctagtgtccaaaatgcgtgacatgaggatggacaagacagagcttggctgcctgagggcaatcattctgtttaatccagaggtgaggggtttgaaatccgcccaggaagttgaacttctacgtgaaaaagtatatgccgctttggaagaatatactagaacaacacatcccgatgaaccaggaagatttgcaaaacttttgcttcgtctgccttctttacgttccataggccttaagtgtttggagcatttgtttttctttcgccttattggagatgttccaattgatacgttcctgatggagatgcttgaatcaccttctgattcataatctagcctagcccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatctctaggcgccaccatgaagctactgtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgagaagaccttgacatgattttgaaaatggattctttacaggatataaaagcattgttaacaggattatttgtacaagataatgtgaataaagatgccgtcacagatagattggcttcagtggagactgatatgcctctaacattgagacagcatagaataagtgcgacatcatcatcggaagagagtagtaacaaaggtcaaagacagttgactgtatcgccggaattcccggggatccggcctgagtgcgtagtacccgagactcagtgcgccatgaagcggaaagagaagaaagcacagaaggagaaggacaaactgcctgtcagcacgacgacggtggacgaccacatgccgcccattatgcagtgtgaacctccacctcctgaagcagcaaggattcacgaagtggtcccaaggtttctctccgacaagctgttggtgacaaaccggcagaaaaacatcccccagttgacagccaaccagcagttccttatcgccaggctcatctggtaccaggacgggtacgagcagccttctgatgaagatttgaagaggattacgcagacgtggcagcaagcggacgatgaaaacgaagagtcggacactcccttccgccagatcacagagatgactatcctcacggtccaacttatcgtggagttcgcgaagggattgccagggttcgccaagatctcgcagcctgatcaaattacgctgcttaaggcttgctcaagtgaggtaatgatgctccgagtcgcgcgacgatacgatgcggcctcagacagtattctgttcgcgaacaaccaagcgtacactcgcgacaactaccgcaaggctggcatggccgaggtcatcgaggatctactgcacttctgccggtgcatgtactctatggcgttggacaacatccattacgcgctgctcacggctgtcgtcatcttttctgaccggccagggttggagcagccgcaactggtggaagagatccagcggtactacctgaatacgctccgcatctatatcctgaaccagctgagcgggtcggcgcgttcgtccgtcatatacggcaagatcctctcaatcctctctgagctacgcacgctcggcatgcaaaactccaacatgtgcatctccctcaagctcaagaacagaaagctgccgcctttcctcgaggagatctgggatgtggcggacatgtcgcacacccaaccgccgcctatcctcgagtcccccacgaatctctaaatcgattacgctcctctactctttgagacatcactggcctataataaatgggttaatttatgtaacaaaattgccttggcttgttaactttattagacattctgatgtttgcattgtgtaaatactgttgtattggaaaagcgtgccaagatggattattgtaattcagtgtcttttttagtagcgtcacgtgccaaacactgttagtcacagagggcatgagacagcctgtgctggaacagctcagttcatagggctatggagatggggagaaaggggcgcttctgtcagagacaagctgtggtctgggaaggccttagcactaaaagcaccacaatgagaagcaaccgccagaagcagggcccgcaggcctttgttccagctgcaaagagaaaggaaaaagtggggaataagagttggggctgcggagggggtggggagcattgtgcaggttccgtacttgaacagaaagcagggaccaacacaaggaagggcgcgccaccggtcaaatgacggtgacaataaaacggagactttgacccggaacgcggaaattcacgtaaaaaacacctgggcgagtcctccacgtaatcggtcaaagtccctcggccctcggtaaatattacgcactatgactaacgccctattattcagttttcacttccccgtttcacttttcgcgcgaaaatggccaaatcttacatggtcccgcccaaaattactacgatatccggtgaaaagcgcgcgaaaattggcacttccggaggtaggcggcgctcatcaaaaacgtcacattttccgcgacggaagcttgcatgtgagctcctcccacttgcaaatgccacacttccgccacacctcccaaccctactcgcgcgtcctacgtcacccgccccgcctctccccgcccacctcattatcatattggccacaatccaaaataaggtatattattgatgatggtttaaacgcccaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaataca

SEQ ID NO: 63 - DNA, 9949 bp, synthetic construct
  misc_feature 1..9949: note = GCAd-RTS-IL12 design 3
  source 1..9949: mol_type = other DNA
ttcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctgggtcaagtcttccagtttaagcagcagagcggtcagtttctcatcccgagcagacgcgcgagaggccgcgccgctcgccaccaaagagctgtaaaggtccgtagccatgctgcgcgcggtcgcggcggcggcggaggcggcggcggaggtcgcggcgtccagcggagttcctcccacggtcgcgtaggccattgtagacgaatttgaaggcagaacggggcgtccatccacgttggaacccatcacattctgacgcactccagcccagtgaggcatgcgcactgtcagataggggctaaagatgcttccatcaaagctgttgccggtgtcgctcatggcggcggctgttgcaagacaaaacagagagacccttagacccccaatttatacacgccccacccttctagccacgcccaccttacccacctcaatcggtatcctcatcgctagacccaaactcggccctggtgcaggccagcaccagatggtcaggcctgcaggtacgtagccgcaataaaatatctttattttcattacatctgtgtgttggttttttgtgtgaatccatagtactaacatacgctctccatcaaaacaaaacgaaacaaaacaaactagcaaaataggctgtccccagtgcaagtccaggtgccagaacatttctctatccataatgcaggggtaccggaaggaagaggggcggggtcgatcgaccccgcccctcttccttcgaaggaagaggggcggggtccaattgcggagtactgtcctccgagcggagtactgtcctccgagcggagtactgtcctccgagcggagtactgtcctccgagcggagtactgtcctccgagcggagtactgtcctccgagcggagagtccccggggacctagagggtatataatgggtgccttagctggtgtgtgacctcatcttcctgtacgcccctgcaggcagccgctaaatccaaggtaaggtcagaagagctagcgccaccatgtgtcaccagcagttggtcatctcttggttcagcctggtttttctggcatctcccctcgtggccatctgggaactgaagaaagatgtttatgtcgtagaattggattggtatcccgacgcccctggagaaatggtggtcctgacatgtgacacccctgaagaagatggtatcacctggaccttggaccagagcagtgaggtcttaggctctggcaagaccctgaccatccaagtcaaagagtttggagatgctggccagtacacctgtcacaaaggaggcgaggttctaagccattcgctcctgctgcttcacaaaaaggaagatggaatttggtccactgacattctgaaggaccagaaagaacccaagaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactcagcgccgagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagcccctgaagaacagcagacaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgctccggtggcggtggcggcggatctagaaacctccccgtggccactccagacccaggaatgttcccatgccttcaccacagccagaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagctgctgatggaccccaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaatcagagcagtgactattgatagagtgatgagctatctgaatgcttcctaaatcgatttatttatcggcataaataatttttttgaagaagtaatactatttttctttttttttgtaaataaatgggttaagggatgtaacattgtttgttgtttggtgggggttggggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctgggtacgtgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgagctagcgccgccaccatgggccctaaaaagaagcgtaaagtcgcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtggggaattcgagatgcctgtggacaggatcctggaggcagagcttgctgtggaacagaagagtgaccagggcgttgagggtcctgggggaaccgggggtagcggcagcagcccaaatgaccctgtgactaacatctgtcaggcagctgacaaacagctattcacgcttgttgagtgggcgaagaggatcccacacttttcctccttgcctctggatgatcaggtcatattgctgcgggcaggctggaatgaactcctcattgcctccttttcacaccgatccattgatgttcgagatggcatcctccttgccacaggtcttcacgtgcaccgcaactcagcccattcagcaggagtaggagccatctttgatcgggtgctgacagagctagtgtccaaaatgcgtgacatgaggatggacaagacagagcttggctgcctgagggcaatcattctgtttaatccagaggtgaggggtttgaaatccgcccaggaagttgaacttctacgtgaaaaagtatatgccgctttggaagaatatactagaacaacacatcccgatgaaccaggaagatttgcaaaacttttgcttcgtctgccttctttacgttccataggccttaagtgtttggagcatttgtttttctttcgccttattggagatgttccaattgatacgttcctgatggagatgcttgaatcaccttctgattcataatctagcctagcccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatctctaggcgccaccatgaagctactgtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgagaagaccttgacatgattttgaaaatggattctttacaggatataaaagcattgttaacaggattatttgtacaagataatgtgaataaagatgccgtcacagatagattggcttcagtggagactgatatgcctctaacattgagacagcatagaataagtgcgacatcatcatcggaagagagtagtaacaaaggtcaaagacagttgactgtatcgccggaattcccggggatccggcctgagtgcgtagtacccgagactcagtgcgccatgaagcggaaagagaagaaagcacagaaggagaaggacaaactgcctgtcagcacgacgacggtggacgaccacatgccgcccattatgcagtgtgaacctccacctcctgaagcagcaaggattcacgaagtggtcccaaggtttctctccgacaagctgttggtgacaaaccggcagaaaaacatcccccagttgacagccaaccagcagttccttatcgccaggctcatctggtaccaggacgggtacgagcagccttctgatgaagatttgaagaggattacgcagacgtggcagcaagcggacgatgaaaacgaagagtcggacactcccttccgccagatcacagagatgactatcctcacggtccaacttatcgtggagttcgcgaagggattgccagggttcgccaagatctcgcagcctgatcaaattacgctgcttaaggcttgctcaagtgaggtaatgatgctccgagtcgcgcgacgatacgatgcggcctcagacagtattctgttcgcgaacaaccaagcgtacactcgcgacaactaccgcaaggctggcatggccgaggtcatcgaggatctactgcacttctgccggtgcatgtactctatggcgttggacaacatccattacgcgctgctcacggctgtcgtcatcttttctgaccggccagggttggagcagccgcaactggtggaagagatccagcggtactacctgaatacgctccgcatctatatcctgaaccagctgagcgggtcggcgcgttcgtccgtcatatacggcaagatcctctcaatcctctctgagctacgcacgctcggcatgcaaaactccaacatgtgcatctccctcaagctcaagaacagaaagctgccgcctttcctcgaggagatctgggatgtggcggacatgtcgcacacccaaccgccgcctatcctcgagtcccccacgaatctctaaatcgattacgctcctctactctttgagacatcactggcctataataaatgggttaatttatgtaacaaaattgccttggcttgttaactttattagacattctgatgtttgcattgtgtaaatactgttgtattggaaaagcgtgccaagatggattattgtaattcagtgtcttttttagtagcgtcacgtgccaaacactgttagtcacagagggcatgagacagcctgtgctggaacagctcagttcatagggctatggagatggggagaaaggggcgcttctgtcagagacaagctgtggtctgggaaggccttagcactaaaagcaccacaatgagaagcaaccgccagaagcagggcccgcaggcctttgttccagctgcaaagagaaaggaaaaagtggggaataagagttggggctgcggagggggtggggagcattgtgcaggttccgtacttgaacagaaagcagggaccaacacaaggaagggcgcgccaccggtcaaatgacggtgacaataaaacggagactttgacccggaacgcggaaattcacgtaaaaaacacctgggcgagtcctccacgtaatcggtcaaagtccctcggccctcggtaaatattacgcactatgactaacgccctattattcagttttcacttccccgtttcacttttcgcgcgaaaatggccaaatcttacatggtcccgcccaaaattactacgatatccggtgaaaagcgcgcgaaaattggcacttccggaggtaggcggcgctcatcaaaaacgtcacattttccgcgacggaagcttgcatgtgagctcctcccacttgcaaatgccacacttccgccacacctcccaaccctactcgcgcgtcctacgtcacccgccccgcctctccccgcccacctcattatcatattggccacaatccaaaataaggtatattattgatgatggtttaaacgcccaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaataca

SEQ ID NO: 64 - DNA, 54 bp, synthetic construct
  misc_feature 1..54: note = Whitlow Linker
  source 1..54: mol_type = other DNA
ggcagcacctccggcagcggcaagcctggcagcggcgagggcagcaccaagggc

SEQ ID NO: 65 - DNA, 78 bp, synthetic construct
  misc_feature 1..78: note = Linker
  source 1..78: mol_type = other DNA
tctggcggaggatctggaggaggcggatctggaggaggaggcagtggaggcggaggatctggcggaggatctctgcag

SEQ ID NO: 66 - skipped

SEQ ID NO: 67 - DNA, 12 bp, synthetic construct
  misc_feature 1..12: note = SGSG linker
  source 1..12: mol_type = other DNA
agtggcagcggc

SEQ ID NO: 68 - DNA, 45 bp, synthetic construct
  misc_feature 1..45: note = (G4S)3 linker
  source 1..45: mol_type = other DNA
ggtggcggtggctcgggcggtggtgggtcgggtggcggcggatct

SEQ ID NO: 69 - DNA, 12 bp, synthetic construct
  misc_feature 1..12: note = Furin cleavage site/ Furinlink1
  source 1..12: mol_type = other DNA
cgtgcaaagcgt

SEQ ID NO: 70 - DNA, 84 bp, synthetic construct
  misc_feature 1..84: note = Fmdv
  source 1..84: mol_type = other DNA
agagccaagagggcaccggtgaaacagactttgaattttgaccttctgaagttggcaggagacgttgagtccaaccctgggccc

SEQ ID NO: 71 - DNA, 54 bp, synthetic construct
  misc_feature 1..54: note = Thosea asigna virus 2A region (T2A)
  source 1..54: mol_type = other DNA
gagggcagaggaagtctgctaacatgcggtgacgtcgaggagaatcctggacct

SEQ ID NO: 72 - DNA, 75 bp, synthetic construct
  misc_feature 1..75: note = Furin-GSG-T2A
  source 1..75: mol_type = other DNA
agagctaagaggggaagcggagagggcagaggaagtctgctaacatgcggtgacgtcgaggagaatcctggacct

SEQ ID NO: 73 - DNA, 78 bp, synthetic construct
  misc_feature 1..78: note = Furin-SGSG-T2A
  source 1..78: mol_type = other DNA
agggccaagaggagtggcagcggcgagggcagaggaagtcttctaacatgcggtgacgtggaggagaatcccggccct

SEQ ID NO: 74 - DNA, 57 bp, synthetic construct
  misc_feature 1..57: note = Porcine teschovirus-1 2A region (P2A)
  source 1..57: mol_type = other DNA
gcaacgaacttctctctcctaaaacaggctggtgatgtggaggagaatcctggtcca

SEQ ID NO: 75 - DNA, 66 bp, synthetic construct
  misc_feature 1..66: note = GSG-P2A
  source 1..66: mol_type = other DNA
ggaagcggagctactaacttcagcctgctgaagcaggctggagacgtggaggagaaccctggacct

SEQ ID NO: 76 - DNA, 60 bp, synthetic construct
  misc_feature 1..60: note = Equine rhinitis A virus 2A region (E2A)
  source 1..60: mol_type = other DNA
cagtgtactaattatgctctcttgaaattggctggagatgttgagagcaaccctggacct

SEQ ID NO: 77 - DNA, 66 bp, synthetic construct
  misc_feature 1..66: note = Foot-and-mouth disease virus 2A region (F2A)
  source 1..66: mol_type = other DNA
gtcaaacagaccctaaactttgatctgctaaaactggccggggatgtggaaagtaatcccggcccc

SEQ ID NO: 78 - DNA, 93 bp, synthetic construct
  misc_feature 1..93: note = FP2A
  source 1..93: mol_type = other DNA
cgtgcaaagcgtgcaccggtgaaacagggaagcggagctactaacttcagcctgctgaagcaggctggagacgtggaggagaaccctggacct

SEQ ID NO: 79 - DNA, 24 bp, synthetic construct
  misc_feature 1..24: note = Linker-GSG
  source 1..24: mol_type = other DNA
gcaccggtgaaacagggaagcgga

SEQ ID NO: 80 - DNA, 15 bp, synthetic construct
  misc_feature 1..15: note = Linker
  source 1..15: mol_type = other DNA
gcaccggtgaaacag

SEQ ID NO: 81 - protein, 18 aa, synthetic construct
  REGION 1..18: note = Whitlow Linker Amino Acid Sequence
GSTSGSGKPGSGEGSTKG

SEQ ID NO: 82 - protein, 26 aa, synthetic construct
  REGION 1..26: note = Linker Amino Acid Sequence
SGGGSGGGGSGGGGSGGGGSGGGSLQ

SEQ ID NO: 83 - skipped

SEQ ID NO: 84 - protein, 4 aa, synthetic construct
  REGION 1..4: note = SGSG Linker Amino Acid Sequence
SGSG

SEQ ID NO: 85 - protein, 15 aa, synthetic construct
  REGION 1..15: note = (G4S) 3 Linker Amino Acid sequence
GGGGSGGGGSGGGGS

SEQ ID NO: 86 - protein, 4 aa, synthetic construct
  REGION 1..4: note = Linker Polypeptide component
RAKR

SEQ ID NO: 87 - protein, 28 aa, synthetic construct
  REGION 1..28: note = Fmdv Amino Acid Sequence
RAKRAPVKQTLNFDLLKLAGDVESNPGP

SEQ ID NO: 88 - protein, 18 aa, synthetic construct
  REGION 1..18: note = Furin-GSG-T2A Amino Acid Sequence
EGRGSLLTCGDVEENPGP

SEQ ID NO: 89 - protein, 25 aa, synthetic construct
  REGION 1..25: note = Furin-SGSG-T2A Amino Acid Sequence
RAKRGSGEGRGSLLTCGDVEENPGP

SEQ ID NO: 90 - protein, 26 aa, synthetic construct
  REGION 1..26: note = Furin-SGSG-T2A
RAKRSGSGEGRGSLLTCGDVEENPGP

SEQ ID NO: 91 - protein, 19 aa, synthetic construct
  REGION 1..19: note = Porcine teschovirus-1 2A region (P2A)
ATNFSLLKQAGDVEENPGP

SEQ ID NO: 92 - protein, 22 aa, synthetic construct
  REGION 1..22: note = GSG-P2A Amino Acid Sequence
GSGATNFSLLKQAGDVEENPGP

SEQ ID NO: 93 - protein, 20 aa, synthetic construct
  REGION 1..20: note = Equine rhinitis A virus 2A region (E2A) Amino Acid Sequence
QCTNYALLKLAGDVESNPGP

SEQ ID NO: 94 - protein, 22 aa, synthetic construct
  REGION 1..22: note = Foot-and-mouth disease virus 2A region (F2A) Amino Acid Sequence
VKQTLNFDLLKLAGDVESNPGP

SEQ ID NO: 95 - protein, 31 aa, synthetic construct
  REGION 1..31: note = FP2A Amino Acid Sequence
RAKRAPVKQGSGATNFSLLKQAGDVEENPGP

SEQ ID NO: 96 - protein, 8 aa, synthetic construct
  REGION 1..8: note = Linker-GSG Amino Acid
APVKQGSG

SEQ ID NO: 97 - protein, 5 aa, synthetic construct
  REGION 1..5: note = Linker Amino Acid
APVKQ

SEQ ID NO: 98 - protein, 154 aa, synthetic construct
  REGION 1..154: note = HBV HBx domain of HBV design 1
MAARLCCQLDPARDVLCLRPVGAESRGRPFSGPLGALSSSSPPAVPTDHGAHLSLRGLPVCAFSSAGPCALRFTSARRMETTVNAHQFLPKVLHKRTLGLSAMSTTDLEAYFKDCLFKDWEELGEELRLKVFVLGGCRHKLVCAPAPCNFFTSA

SEQ ID NO: 99 - protein, 454 aa, synthetic construct
  REGION 1..454: note = HBV Pol domain of HBV design 1
GPCAEHGEHHIRIPRTPARVTGGVFLVDKNPHNTAESRLVVDFSQFSRGNYRVSWPKFAVPNLQSLTNLLSSNLCWLSLDVSAAFYHLPLHPAAMPHLLVGSSGLSRYVARLSSNSRIINHQHGTLQNLHDSCSRNLYVSLLLLYKTFGWKLHLYSHPIILGFRKIPMGVGLSPFLLAQFTSAICSVVRRAFPHCLAFSGAKSVQHLESLFTAVTNFLLSLGIHLNPNKTKRWGYSLNFMGYVIGSWGSLPQDHIRHKIKECFRKLPVHRPIDWKVCQRIVGLLGFAAPFTQCGYPALMPLYACIQSKQAFTFSPTYKAFLCKQYLNLYPVARQRPGLCQVFADATPTGWGLVMGHQRMRGTFSSRKYTSFPWLLGCAANWILRGTSFVYVPSALNPADDPSRGRLGPCRPLLHLPFRPTTGRTSLYADSPSVPSHLPDRVHFASPLHVAWRPP

SEQ ID NO: 100 - protein, 226 aa, synthetic construct
  REGION 1..226: note = HBV Surface (Env1) domain of HBV domain 1
MENITSGFLGPLLVLQAGFFLLTRILTIPQSLDSWWTSLSFLGGTTVCLGQNSQSPTSNHSPTSCPPTCVGYRWMCLRRFIIFLFILLLCLIFLLVLLDYQGMLPVCPLIPGSSTTSTGPCRTCTTPAQGTSMYPSCCCTKPSDGNCTCIPIPSSWAFGKFLWEWASARFSWLSLLVPFVQWFVGLSPTVWLSVIWMMWYWGPSLYNTLSPFLPLLPIFFYLWVYI

SEQ ID NO: 101 - protein, 180 aa, synthetic construct
  REGION 1..180: note = HBV Core domain of HBV design 1
MQLFHLCLIISCSCPTVQASKLCLGWLWDMDIDPYKEFGASVELLSFLPSDFFPSVRDLLDTATALYRDALESPEHCTPHHTALRHVCLCWGDLMNLATWVGTNLEDQASRDLVVSYVNTNMGLKFRQLLWFHISCLTFGRDLVLEYLVSFGVWIRTPPAYRPSNAPILSTLPETTVVRQ

SEQ ID NO: 102 - protein, 226 aa, synthetic construct; identical to SEQ ID NO: 100
  REGION 1..226: note = SHB(Env) domain

SEQ ID NO: 103 - protein, 180 aa, synthetic construct; identical to SEQ ID NO: 101
  REGION 1..180: note = HBeAg domain

SEQ ID NO: 104 - protein, 154 aa, synthetic construct; identical to SEQ ID NO: 98
  REGION 1..154: note = HBx domain

SEQ ID NO: 105 - protein, 454 aa, synthetic construct; identical to SEQ ID NO: 99
  REGION 1..454: note = Pol domain

SEQ ID NO: 106 - protein, 1014 aa, synthetic construct
  REGION 1..1014: note = HBV design 1
MENITSGFLGPLLVLQAGFFLLTRILTIPQSLDSWWTSLSFLGGTTVCLGQNSQSPTSNHSPTSCPPTCVGYRWMCLRRFIIFLFILLLCLIFLLVLLDYQGMLPVCPLIPGSSTTSTGPCRTCTTPAQGTSMYPSCCCTKPSDGNCTCIPIPSSWAFGKFLWEWASARFSWLSLLVPFVQWFVGLSPTVWLSVIWMMWYWGPSLYNTLSPFLPLLPIFFYLWVYIMQLFHLCLIISCSCPTVQASKLCLGWLWDMDIDPYKEFGASVELLSFLPSDFFPSVRDLLDTATALYRDALESPEHCTPHHTALRHVCLCWGDLMNLATWVGTNLEDQASRDLVVSYVNTNMGLKFRQLLWFHISCLTFGRDLVLEYLVSFGVWIRTPPAYRPSNAPILSTLPETTVVRQMAARLCCQLDPARDVLCLRPVGAESRGRPFSGPLGALSSSSPPAVPTDHGAHLSLRGLPVCAFSSAGPCALRFTSARRMETTVNAHQFLPKVLHKRTLGLSAMSTTDLEAYFKDCLFKDWEELGEELRLKVFVLGGCRHKLVCAPAPCNFFTSAGPCAEHGEHHIRIPRTPARVTGGVFLVDKNPHNTAESRLVVDFSQFSRGNYRVSWPKFAVPNLQSLTNLLSSNLCWLSLDVSAAFYHLPLHPAAMPHLLVGSSGLSRYVARLSSNSRIINHQHGTLQNLHDSCSRNLYVSLLLLYKTFGWKLHLYSHPIILGFRKIPMGVGLSPFLLAQFTSAICSVVRRAFPHCLAFSGAKSVQHLESLFTAVTNFLLSLGIHLNPNKTKRWGYSLNFMGYVIGSWGSLPQDHIRHKIKECFRKLPVHRPIDWKVCQRIVGLLGFAAPFTQCGYPALMPLYACIQSKQAFTFSPTYKAFLCKQYLNLYPVARQRPGLCQVFADATPTGWGLVMGHQRMRGTFSSRKYTSFPWLLGCAANWILRGTSFVYVPSALNPADDPSRGRLGPCRPLLHLPFRPTTGRTSLYADSPSVPSHLPDRVHFASPLHVAWRPP

SEQ ID NO: 107 - protein, 1021 aa, synthetic construct
  REGION 1..1021: note = HBV design 2
SVELLSFLPSDFFPSVRDLLDTATALYRDALESPEHCTPHHTALRHVCLCWGDLMNLATWVGTNLEDQASRDLVVSYVNTNMGLKFRQLLWFHISCLTFGRDLVLEYLVSFGVWIRTPPAYRPSNAPILSTLPETTVVRQRGRTIVLHKRTLGLMGQNLSTSNPLGFFPDHQLDPAFRANTNNPDWDFNPNKDTWPDANKVGAGAFGLGFTPPHGGLLGWSPQAQGIMQTLPANPPPASTNRQSGRQPTPLLPKVLHKRTLMPLSYQHFRKLLLLDNEAGPLEEELPRLADEDLNRRVAEDLNLGNLNVSIPWTHKVGNFTGLYSSSVPVFNPHWKTPSFPNIHLHQDIIKKCEQFVGPLTVNEKRRLKLIMPARFYPNFTKYLPLDKGIKPYYPEHLVNHYFHTRHYLHTLWKAGILYKRVSTHSASFCGSPYSWEQELQHGAESFHQQSSGILSRPSVGSSLQSKHQQSRLGLQSQQGHLARRQQGRSWSIRTRVHPTARRPSGVEPSGSGHNANLASKSASCLYQSTVRTAAYPAVSTSENHSSSGHAVELHNLPPNSARSQSERPVSPCWWLQFRNSKPCSDYCLSHIVNLLEDWGPCHKRTLGLSAMSPPLRTTHPQAMQWNSTTFHQTLQDPRVRGLYLPAGGSSSGTVNPVPTTASPTLSTSSRIGDPALNQFLPKVLHKRSRGNYRVSWPKFAVPNLQSLTNLLSSNLCWLSLDVSAAFYHLPLHPAAMPHLLVGSSGLSRYVARLSSNSRIINHQHGTLQNLHDSCSRNLYVSLLLLYKTFGWKLHLYSHPIILGFRKIPMGVGLSPFLLAQFTSAITVNAHQFLPKQNSQSPTSNHSPTSCPPTCVGYRWMCLRRFIIFLFILLLCLIFLLVLLDYQGMLPVCPLIPGSSTTSTGPCRTCTTPAQGTSMYPSCCCTKPSDGNCTCIPIPSSWAFGKFLWEWASARFSWLSLLVPFVQWFVGLSPTVWLSVIWMMWYWGPSLYNTLSPFLPLLPIFFYLWVYILSAMSTTDL

SEQ ID NO: 108 - protein, 532 aa, synthetic construct
  REGION 1..532: note = HBV design 3
MQTDRTGETALHLAARYSRSDFLPSDFFPSVADAGVWIRTPPADNMEYLVSFGVWPLHAAVSADCWGELMTLRNRATDLGGPNLDNILMHDILRSFIPLLPLILAARLAVSTLPETTVVRRSHADVFLGGPPVCLDDLFLLTRILTIALHWAAAVNNVLTFGRETVLEYGANKWLSLLVPFVNNRFLKQQYMNLPLFLAAREGSYEDLLDTASALYANRFLSKQYMDLDHMTVSTKLCKIPRDLWFHISCLTFIVRLLDLEVSQTSKLTRQTDRTGETALHLAARYSRSDLTTVPAASLLAADAGLSRYVARLDNMKLHLYSHPIPLHAAVSADGLSPTVWLSVRNRATDLFLLSLGIHLMHDSLYADSPSVPLILAARLAVHKRTLGLSAMSHADVTLCIPHVAVDDLLLLKATLCIALHWAAAVNNVQFLPKVLHKRGANKALMPLYACINNRTVNAHQFLPKPLFLAAREGSYELPKVLHKRTLANRVLHKRTLGLDHMLSAMSTTDLPRDLLVPFVQWFVIVRLLDLE

SEQ ID NO: 109 - protein, 368 aa, synthetic construct
  REGION 1..368: note = HBV design 4
KKFLPSDFFPSVKKCWGELMTLKKGVWIRTPPAKKSTLPETTVVRRKKLTFGRETVLEYKKDLLDTASALYKKLWFHISCLTFKKEYLVSFGVWKKGGPNLDNILKKLTTVPAASLLAKKILRSFIPLLKKFLGGPPVCLKKFLLTRILTIKKWLSLLVPFVKKGLSPTVWLSVKKLLVPFVQWFVKKFLKQQYMNLKKFLSKQYMDLKKTVSTKLCKIKKGLSRYVARLKKKLHLYSHPIKKFLLSLGIHLKKSLYADSPSVKKALMPLYACIKKLLLKATLCIKKTLCIPHVAVKKVLHKRTLGLKKLPKVLHKRTLKKHKRTLGLSAMKKQFLPKVLHKRKKTVNAHQFLPKKKLSAMSTTDLKK

SEQ ID NO: 110 - DNA, 20 bp, synthetic construct
  misc_feature 1..20: note = splice primer
  source 1..20: mol_type = other DNA
tgccaagagtgacgtgtcca

SEQ ID NO: 111 - DNA, 21 bp, synthetic construct
  misc_feature 1..21: note = splice probe
  source 1..21: mol_type = other DNA
cccaggtccaactgcagccgg

SEQ ID NO: 112 - DNA, 12 bp, synthetic construct
  misc_feature 1..12: note = Furin linker polypeptide
  source 1..12: mol_type = other DNA
agagctaagagg

SEQ ID NO: 113 - protein, 11 aa, synthetic construct
  REGION 1..11: note = HPV16-E6
HLDKKQRFHNI

SEQ ID NO: 114 - protein, 10 aa, synthetic construct
  REGION 1..10: note = HPV16-E6
RWTGRCMSCC

SEQ ID NO: 115 - protein, 15 aa, synthetic construct
  REGION 1..15: note = HPV16-E6
TTLEQQYNKPLCDLL

SEQ ID NO: 116 - protein, 9 aa, synthetic construct
  REGION 1..9: note = HPV16-E6
ISEYRHYCY

SEQ ID NO: 117 - protein, 9 aa, synthetic construct
  REGION 1..9: note = HPV16-E6
VYDFAFRDL

SEQ ID NO: 118 - protein, 10 aa, synthetic construct
  REGION 1..10: note = HPV16-E6
TIHDIILECV

SEQ ID NO: 119 - protein, 9 aa, synthetic construct
  REGION 1..9: note = HPV16-E6
KLPQLCTEL

SEQ ID NO: 120 - protein, 10 aa, synthetic construct
  REGION 1..10: note = HPV16-E6
FAFRDLCIVY

SEQ ID NO: 121 - protein, 15 aa, synthetic construct
  REGION 1..15: note = HPV16-E6
LCIVYRDGNPYAVCD

SEQ ID NO: 122 - protein, 10 aa, synthetic construct
  REGION 1..10: note = HPV16-E7
LCVQSTHVDI

SEQ ID NO: 123 - protein, 10 aa, synthetic construct
  REGION 1..10: note = HPV16-E7
RTLEDLLMGT

SEQ ID NO: 124 - protein, 8 aa, synthetic construct
  REGION 1..8: note = HPV16-E7
TLGIVCPI

SEQ ID NO: 125 - protein, 9 aa, synthetic construct
  REGION 1..9: note = HPV16-E7
LLMGTLGIV

SEQ ID NO: 126 - protein, 9 aa, synthetic construct
  REGION 1..9: note = HPV16-E7
TLHEYMLDL

SEQ ID NO: 127 - protein, 10 aa, synthetic construct
  REGION 1..10: note = HPV16-E7
AHYNIVTFCC

SEQ ID NO: 128 - protein, 10 aa, synthetic construct
  REGION 1..10: note = HPV16-E7
YMLDLQPETT

SEQ ID NO: 129 - protein, 9 aa, synthetic construct
  REGION 1..9: note = HPV16-E7
CDSTLRLCV

SEQ ID NO: 130 - protein, 9 aa, synthetic construct
  REGION 1..9: note = HPV16-E5
YIIFVYIPL

SEQ ID NO: 131 - protein, 10 aa, synthetic construct
  REGION 1..10: note = HPV16-E5
KLTNTGLYNL

SEQ ID NO: 132 - protein, 9 aa, synthetic construct
  REGION 1..9: note = HPV16-E5
KCIDFYSRI

SEQ ID NO: 133 - protein, 9 aa, synthetic construct
  REGION 1..9: note = HPV16-E5
FAFKDLFVV

SEQ ID NO: 134 - protein, 9 aa, synthetic construct
  REGION 1..9: note = HPV16-E5
NLLIRCLRC

SEQ ID NO: 135 - protein, 9 aa, synthetic construct
  REGION 1..9: note = HPV16-E5
KLPDLCTEL

SEQ ID NO: 136 - protein, 9 aa, synthetic construct
  REGION 1..9: note = HPV16-E5
ELTEVFEFA

SEQ ID NO: 137 - protein, 10 aa, synthetic construct
  REGION 1..10: note = HPV16-E5
SLQDIEITCV

SEQ ID NO: 138 - protein, 9 aa, synthetic construct
  REGION 1..9: note = HPV16-E5
KTVLELTEV

SEQ ID NO: 139 - protein, 15 aa, synthetic construct
  REGION 1..15: note = HPV18-E7
DDLRAFQQLFLNTLS

SEQ ID NO: 140 - protein, 9 aa, synthetic construct
  REGION 1..9: note = HPV18-E7
FQQLFLNTL

SEQ ID NO: 141 - protein, 10 aa, synthetic construct
  REGION 1..10: note = HPV18-E7
QLFLNTLSFV

SEQ ID NO: 142 - protein, 15 aa, synthetic construct
  REGION 1..15: note = HPV18-E7
LFLNTLSFVCPWCAS

SEQ ID NO: 143 - protein, 9 aa, synthetic construct
  REGION 1..9: note = HPV18-E7
TLQDIVLHL

SEQ ID NO: 144 - protein, 20 aa, synthetic construct
  REGION 1..20: note = HPV18-E7
SEEENDEIDGVNHQHLPARR

SEQ ID NO: 145 - protein, 9 aa, synthetic construct
  REGION 1..9: note = Agonist peptides
KLPQLCTEV

SEQ ID NO: 146 - protein, 10 aa, synthetic construct
  REGION 1..10: note = Agonist peptides
QLYNKPLCDV

SEQ ID NO: 147 - protein, 10 aa, synthetic construct
  REGION 1..10: note = Agonist peptides
RTLEDLLMGV

(19) United States
(12) Patent Application Publication
(10) Pub. No.: US 2026/0098063 A1
(43) Pub. Date: Apr. 9, 2026

(54) HUMAN PAPILLOMAVIRUS VACCINES AND USES OF THE SAME

(71) Applicant: PRECIGEN, INC., Germantown, MD (US)

(72) Inventors: Douglas E. BROUGH, Germantown, MD (US); Cheryl G. BOLINGER, Germantown, MD (US); Ramya YARLAGADDA, Germantown, MD (US); Vinodhbabu KURELLA, Germantown, MD (US); Prabakaran PONRAJ, Germantown, MD (US); Simon METENOU, Germantown, MD (US); Kuan-fu DING, Germantown, MD (US)

(21) Appl. No.: 19/261,890

(22) Filed: Jul. 7, 2025

Related U.S. Application Data
(62) Division of application No. 16/978,573, now Pat. No. 12,404,306.
(60) Provisional application No. 62/639,354, filed on Mar. 6, 2018.

(51) Int. Cl. C07K 14/005; A61K 39/12; A61K 39/29; A61K 48/00; C07K 14/02; C12N 7/00; C12N 15/861

(52) U.S. Cl. CPC C07K 14/005; A61K 39/12; A61K 39/292; A61K 48/0066; C07K 14/02; C12N 7/00; C12N 15/861; C07K 2319/33; C12N 2710/20022; C12N 2710/20034

(57) ABSTRACT

Provided herein are engineered human papilloma virus (HPV) molecular vaccine constructs. Vaccine constructs can also include ligand-inducible engineered gene switch systems for modulating expression of heterologous genes, such as a cytokines, in host cells.

### REFERENCE TO SEQUENCE LISTING

The content of the electronically submitted sequence listing (Name: 16978573DIVSL.xml; Size: 438,529 bytes; Date of Creation: Jul. 7, 2025) filed with this application is incorporated herein by reference in its entirety.

### FIELD OF THE DISCLOSURE

The present disclosure relates to improved, broad-spectrum HPV molecular vaccines designed via use of advanced principles in bioinformatics and protein engineering.

### BACKGROUND OF THE DISCLOSURE

Globally, tens of millions of people are currently infected with human papillomavirus (HPV), and millions more become newly infected each year. HPV has been determined to be a precursor of cervical and other cancers, such as head and neck cancers. Tens of thousands of women get cervical cancer each year. Currently, vaccinations can protect against diseases caused by HPV when given to recommended age groups; however, there is a tremendous need for HPV vaccines with broad coverage (against multiple HPV strains) and functionality against HPV-associated cancers.

The present disclosure relates to improved, broad-spectrum HPV molecular vaccines designed via use of advanced principles in bioinformatics and protein engineering. These novel HPV vaccines can be used as a therapeutic vaccine against HPV related diseases.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY OF THE DISCLOSURE

Provided herein is a non-naturally occurring polynucleotide encoding a polypeptide comprising at least one of one or more immune response-inducing human papilloma virus (HPV) polypeptides.

In some embodiments, said non-naturally occurring polynucleotide encodes a polypeptide comprising two or more HPV polypeptides. In some embodiments, said two or more HPV polypeptides comprise one or more HPV-16 immune response-inducing polypeptide sequences. In some embodiments, said HPV-16 peptide comprises at least one of an E5 peptide, an E6 peptide or an E7 peptide. In some embodiments, said HPV-16 peptide comprises an E5 peptide, and said E5 peptide has a sequence as shown in SEQ ID NO: 47. In some embodiments, said HPV-16 peptide comprises an E6 peptide, and said E6 peptide has a sequence as shown in SEQ ID NO: 45. In some embodiments, said HPV-16 peptide comprises an E7 peptide, and said E7 peptide has a sequence as shown in SEQ ID NO: 46. In some embodiments, said one or more HPV peptides comprises an HPV-18 peptide. In some embodiments, said HPV-18 peptide comprises at least one of an E5 peptide, an E6 peptide or an E7 peptide. In some embodiments, said HPV-18 peptide comprises an E5 peptide, and said E5 peptide has a sequence as shown in SEQ ID NO: 50. In some embodiments, said HPV-18 peptide comprises an E6 peptide, and said E6 peptide has a sequence as shown in SEQ ID NO: 48. In some embodiments, said HPV-18 peptide comprises an E7 peptide, and said E7 peptide has a sequence as shown in SEQ ID NO: 49. In some embodiments, said polypeptide has a sequence as shown in SEQ ID NO: 51. In some embodiments, at least one of said one or more HPV peptides is connected to an agonist peptide. In some embodiments, said agonist peptide has a sequence comprising an agonist peptide sequence as shown in Table 2. In some embodiments, said polypeptide has a sequence as shown in SEQ ID NO: 53.

Provided herein is a polynucleotide comprising any of the polynucleotides provided herein, further comprising one or more polynucleotides encoding a gene switch system for inducible control of heterologous gene expression, wherein heterologous gene expression is regulated by said gene switch system; and, wherein said heterologous gene comprises any of the polynucleotide described herein. In some embodiments, said gene switch system is an ecdysone receptor-based (EcR-based) gene switch system. In some embodiments, said one or more HPV polypeptides is for use in a vaccine.

Provided herein is a vector comprising any of the polynucleotides provided herein. In some embodiments, said vector is an adenoviral vector. In some embodiments, said adenoviral vector is a gorilla adenoviral vector.

Provided herein is a method of regulating the expression of a heterologous gene in a cell, the method comprising: introducing into said cell one or more polynucleotides that comprise (i) an repressible or inducible gene switch, and (ii) a heterologous immune response-inducing gene, wherein expression of said heterologous immune response-inducing gene is regulated by said gene switch, wherein said heterologous immune response-inducing gene encodes at least one of one or more HPV polypeptides; and exposing said cell to a compound in an amount sufficient to repress or induce expression of said heterologous immune response-inducing gene.

In some embodiments, said target cell is a mammalian cell in a method of regulating the expression of a heterologous gene in a cell described herein. In some embodiments, said gene switch comprises a ligand binding domain derived from at least one of an ecdysone receptor (EcR), a ubiquitous receptor, an orphan receptor 1, an NER-1, a steroid hormone nuclear receptor 1, a retinoid X receptor interacting protein-15, a liver X receptor β, a steroid hormone receptor like protein, a liver X receptor, a liver X receptor α, a farnesoid X receptor, a receptor interacting protein 14, and a famesol receptor.

Provided herein is an E6 peptide, wherein said E6 peptide comprises an E18A amino acid substitution and at least one of an L50G, E148A, T149A, Q150A and L151A amino acid substitution as compared to a wildtype E6 peptide. In some embodiments, said E6 peptide comprises said E18A amino acid substitution and said L50G, E148A, T149A, Q150A and L151A amino acid substitutions. In some embodiments, said E6 peptide has a sequence as shown in SEQ ID NO: 45. In some embodiments, said E6 peptide is fused to an agonist peptide. In some embodiments, said agonist peptide is fused to at least one of a C-terminus and an N-terminus of said E6 peptide. In some embodiments, said wildtype E6 peptide is from HPV-16.

Provided herein is an E6 peptide, wherein said E6 peptide comprises a deletion as compared to a wildtype E6 peptide, wherein said deletion comprises a C-terminus of said wildtype E6 peptide. In some embodiments, said deletion comprises amino acids from amino acid 121 to a C-terminus of said wildtype E6 peptide. In some embodiments, said E6 peptide comprises at least one of an E18A and L50G substitution as compared to said wildtype E6 peptide. In some embodiments, said wildtype E6 peptide is from HPV-18. In some embodiments, said E6 peptide has a sequence as shown in SEQ ID NO: 48.

Provided herein is an E7 peptide, wherein said E7 peptide comprises a deletion as compared to a wildtype E7 peptide, wherein said deletion comprises an N-terminus of said wildtype E7 peptide. In some embodiments, said deletion comprises amino acids 1-39 of said wildtype E7 peptide. In some embodiments, said E7 peptide comprises at least one of an E55A and L74R substitution as compared to said wildtype E7 peptide. In some embodiments, said wildtype E7 peptide is from HPV-18. In some embodiments, said E7 peptide has a sequence as shown in SEQ ID NO: 49.

Provided herein is an E5 peptide, wherein said E5 peptide comprises a deletion as compared to a wildtype E5 peptide, wherein said deletion comprises amino acids 41-57 of said wildtype E5 peptide. In some embodiments, said E5 peptide has a sequence as shown in SEQ ID NO: 47. In some embodiments, said wildtype E5 peptide is from HPV-16.

Provided herein is an E5 peptide, wherein said E5 peptide comprises a deletion as compared to a wildtype E5 peptide, wherein said deletion comprises at least one of amino acids 27-40 or amino acids 54-57 of said wildtype E5 peptide. In some embodiments, said E5 peptide has a sequence as shown in SEQ ID NO: 50. In some embodiments, said wildtype E5 peptide is from HPV-18.

Provided herein is a polypeptide construct comprising any one of the presently described E5, E6, and E7.

Provided herein is a polypeptide construct, wherein said polypeptide construct comprises an HPV-16 E6 peptide, wherein said HPV-16 E6 peptide comprises an E18A amino acid substitution and at least one of an L50G, E148A, T149A, Q150A and L151A amino acid substitution as compared to a wildtype HPV-16 E6 peptide. In some embodiments, said HPV-16 E6 peptide comprises said E18A amino acid substitution and said L50G, E148A, T149A, Q150A and L151A amino acid substitutions. In some embodiments, said HPV-16 E6 peptide has a sequence as shown in SEQ ID NO: 45. In some embodiments, said polypeptide construct further comprises an HPV-16 E7 peptide, wherein said HPV-16 E7 peptide comprises at least one of an H2P, C24G, E46A and L67R amino acid substitution as compared to a wildtype HPV-16 E7 peptide. In some embodiments, said HPV-16 E7 peptide comprises said H2P, C24G, E46A and L67R amino acid substitutions. In some embodiments, said HPV-16 E7 peptide has a sequence as shown in SEQ ID NO: 46. In some embodiments, said polypeptide construct further comprises an HPV-16 E5 peptide. In some embodiments, said HPV-16 E5 peptide comprises a deletion of one or more amino acids as compared to a wildtype HPV-16 E5 peptide. In some embodiments, said deletion comprises amino acids 41-57 of said wildtype HPV-16 E5 peptide. In some embodiments, said HPV-16 E5 peptide has a sequence as shown in SEQ ID NO: 47.

In some embodiments, said polypeptide construct comprising an HPV-16 E6 peptide, wherein said HPV-16 E6 peptide comprises an E18A amino acid substitution and at least one of an L50G, E148A, T149A, Q150A and L151A amino acid substitution as compared to a wildtype HPV-16 E6 peptide, further comprises an HPV-18 E6 peptide. In some embodiments, said HPV-18 E6 peptide comprises an E18A and L50G substitution as compared to a wildtype HPV-18 E6 peptide. In some embodiments, said HPV-18 E6 peptide comprises a deletion of at least one C-terminus amino acid relative to said wildtype HPV-18 E6 peptide. In some embodiments, said deletion comprises amino acids from amino acid 121 to said C-terminus of said wildtype HPV-18 E6 peptide. In some embodiments, said HPV-18 E6 peptide has a sequence as shown in SEQ ID NO: 48. In some embodiments, said polypeptide construct further comprises an HPV-18 E7 peptide. In some embodiments, said HPV-18 E7 peptide comprises an E55A and L74R substitution as compared to a wildtype HPV-18 E7 peptide. In some embodiments, said HPV-18 E7 peptide comprises a deletion of at least one amino acid from an N-terminus of said HPV-18 E7 peptide. In some embodiments, said deletion comprises amino acids 1-40 of said wildtype HPV-18 E7 peptide. In some embodiments, said HPV-18 E7 peptide has a sequence as shown in SEQ ID NO: 49. In some embodiments, said polypeptide construct further comprises an HPV-18 E5 peptide. In some embodiments, said HPV-18 E5 peptide comprises a deletion of at least one amino acid as compared to a wildtype HPV-18 E5 peptide. In some embodiments, said deletion comprises amino acids 27-40 or 54-57 of said wildtype HPV-18 E5 peptide. In some embodiments, said HPV-18 E5 peptide has a sequence as shown in SEQ ID NO: 50. In some embodiments, said polypeptide construct has a sequence as shown in SEQ ID NO: 51. In some embodiments, said polypeptide construct further comprises at least one agonist peptide. In some embodiments, said at least one agonist peptide has a sequence comprising an agonist peptide sequence as shown in Table 2. In some embodiments, said polypeptide construct has a sequence as shown in SEQ ID NO: 53.

Provided herein is a polypeptide construct comprising an ankyrin-like repeat domain and an HPV peptide. In some embodiments, said ankyrin-like repeat protein is a human ankyrin-like repeat protein. In some embodiments, said HPV peptide is linked to said ankyrin-like repeat protein by a linker. In some embodiments, said HPV peptide comprises at least one of an HPV-16 peptide or an HPV-18 peptide. In some embodiments, said HPV peptide comprises an HPV-16 peptide, and said HPV-16 peptide comprises at least one of an E5 peptide, an E6 peptide or an E7 peptide. In some embodiments, said HPV peptide comprises an HPV-18 peptide, and said HPV-18 peptide comprises at least one of an E6 peptide or an E7 peptide. In some embodiments, said HPV peptide comprises an HPV-16 E5 sequence, an HPV-16 E6 sequence, an HPV-16 E7 sequence, an HPV-18 E6 sequence or an HPV-18 E7 sequence as shown in Table 2. In some embodiments, said polypeptide construct has a sequence as shown in SEQ ID NO: 52. In some embodiments, said polypeptide construct further comprises at least one agonist peptide. In some embodiments, said polypeptide construct comprises three agonist peptides. In some embodiments, said polypeptide construct has a sequence as shown in SEQ ID NO: 54.

Provided herein is a polypeptide construct, wherein said polypeptide construct comprises at least two HPV amino acid sequences as shown in Table 2, wherein said at least two HPV amino acid sequences are connected by a peptide linker, wherein said peptide linker is a KK linker. In some embodiments, said at least two HPV amino acid sequences comprise at least one of an HPV-16 peptide or an HPV-18 peptide as shown in Table 2. In some embodiments, said at least two HPV amino acid sequences comprise an HPV-16 peptide, and said HPV-16 peptide comprises at least one of an HPV-16 E5 peptide, an HPV-16 E6 peptide or an HPV-16 E7 peptide as shown in Table 2. In some embodiments, said at least two HPV amino acid sequences comprise an HPV-18 peptide, and said HPV-18 peptide comprises at least one of an HPV-18 E6 peptide or an HPV-18 E7 peptide as shown in Table 2. In some embodiments, said at least two HPV amino acid sequences comprise each of the amino acid sequences as shown in Table 2. In some embodiments, said each of the amino acid sequences is connected to another of said each of the amino acid sequences by said KK linker. In some embodiments, said polypeptide construct has a sequence as shown in SEQ ID NO: 55. In some embodiments, any of the polypeptide constructs described herein is for use in a vaccine.

Provided herein is a polynucleotide encoding any of the presently described polypeptide constructs. Also provided herein is a vector comprising said polynucleotide. In some embodiments, said vector is an adenoviral vector. In some embodiments, said adenoviral vector is a gorilla adenoviral vector.

Provided herein is a vector, wherein said vector comprises a polynucleotide that encodes at least one HPV peptide, wherein said vector is an adenoviral vector.

Provided herein is a vector, wherein said vector comprises a polynucleotide that encodes at least one HPV peptide, wherein said vector is an adenoviral vector, wherein said adenoviral vector is a gorilla adenoviral vector.

In some embodiments, any of the polypeptide constructs described herein is for use in a vaccine. Also provided herein is a polynucleotide encoding any of the polypeptide constructs presently described. Also provided herein is a vector comprising said polynucleotide. In some embodiments, said vector is an adenoviral vector. In some embodiments, said adenoviral vector is a gorilla adenoviral vector.

### BRIEF DESCRIPTION OF THE DRAWINGS

The features of the present disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 is a schematic diagram of HPV genome. The HPV genomes includes seven early genes (E1 to E7) and two late genes (L1 and L2), and each gene has specific functions. E5, E6, and E7 genes are associated with cancer development.

FIG. 2 is a schematic overall workflow implemented for designing HPV vaccine antigens.

FIG. 3 is schematic diagrams of HPV16 and HPV18 antigenic designs for HPV design 1 and HPV design 3. Consensus sequence information was utilized to select HPV16/HPV18 reference sequences for the design which included all major variants. The vaccine composition comprising different E6, E7 and E5 protein components with domain boundaries and mutation information is shown. These different domains contain most prevalent peptides deduced from IEDB predictions for MHC-I binding. HPV design 3 is similar to HPV design 1 with the addition of enhancer agonist peptides.

FIG. 4A and FIG. 4B show a homology model of HPV design 2 and HPV design 4, respectively. A homology model is used to assess the overall structural feature and to compare the HPV design against native ankyrin repeats. HPV designs are shown in the same orientation and suggest different structural conformations due to shuffled peptides although maintaining the same overall fold.

FIG. 5A shows HPV design 5 (subject) mapped onto HPV design 4 (Query) using protein blast. Strong and weak binders were identified using netMHC. FIG. 5B shows density plots of HPV designs 4 and 5 which were extracted based on the mapped positions. Similar patterns in predicted strong/weak binding peptides were observed. The binding affinity predictions on the matched regions of HPV designs 4 and 5 were similar.

FIG. 6 is a schematic illustration showing short and long primer and probe sets generated for RNA qPCR relative expression assay. Specific primers were designed for each HPV antigen design.

FIG. 7A shows NetMHC 4.0 antigenicity predictions. Predicted strong and weak binding peptide indices are plotted against peptide locations. FIG. 7B shows NetMHC 4.0 antigenicity prediction density plot. First/second order differentials are employed in order to identify peaks. FIG. 7C shows amino acid sequences aligned against consensus sequences in order to determine coverage across HPV subtypes.

FIG. 8 shows a comparison of HPV design1 (MOD-1755822) and HPV design 3 (MOD-1755825).

FIG. 9A shows HPV16 E6 sequence alignment to wildtype HPV16 E6 from UP P03126. FIG. 9B shows HPV16 E7 sequence alignment to wildtype HPV16 E7 from UP P03129. FIG. 9C shows HPV16 E5 sequence alignment to wildtype HPV16 E5 from UP P06927.

FIG. 10A shows HPV18 E6 sequence alignment to wildtype HPV18E6 from UP P06463. FIG. 10B shows HPV18 E7 sequence alignment to wildtype HPV18E7 from UP P06788. FIG. 10C shows HPV18 E5 sequence alignment to wildtype HPV18E5 from UP P06792.

FIG. 11 shows an overview of IL-12 promoting immune response by activating NK cells and T cells.

FIG. 12 shows various structural components of diverse IL-12 ligand-inducible gene switch vector systems.

### DETAILED DESCRIPTION OF THE DISCLOSURE

The following description and examples illustrate embodiments of the present disclosure in detail.

It is to be understood that the present disclosure is not limited to the particular embodiments described herein and as such can vary. Those of skill in the art will recognize that there are variations and modifications of the present disclosure, which are encompassed within its scope.

All terms are intended to be understood as they would be understood by a person skilled in the art. Unless defined otherwise, all technical and scientific terms used herein have the same meaning as commonly understood by one of ordinary skill in the art to which the disclosure pertains.

The section headings used herein are for organizational purposes only and are not to be construed as limiting the subject matter described.

Although various features of the disclosure can be described in the context of a single embodiment, the features can also be provided separately or in any suitable combination. Conversely, although the present disclosure can be described herein in the context of separate embodiments for clarity, the present disclosure can also be implemented in a single embodiment.

The following definitions supplement those in the art and are directed to the current application and are not to be imputed to any related or unrelated case, e.g., to any commonly owned patent or application. Although any methods and materials similar or equivalent to those described herein can be used in the practice for testing of the present disclosure, the preferred materials and methods are described herein. Accordingly, the terminology used herein is for the purpose of describing particular embodiments only, and is not intended to be limiting.

### Definitions

In this application, the use of the singular includes the plural unless specifically stated otherwise. It must be noted that, as used in the specification, the singular forms “a,” “an” and “the” include plural referents unless the context clearly dictates otherwise.

In this application, the use of “or” means “and/or” unless stated otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively, unless the context specifically refers to a disjunctive use.

Furthermore, use of the term “including” as well as other forms, such as “include,” “includes,” and “included,” is not limiting.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the disclosure, and vice versa. Furthermore, compositions of the present disclosure can be used to achieve methods of the present disclosure.

The term “about” in relation to a reference numerical value and its grammatical equivalents as used herein can include the numerical value itself and a range of values plus or minus 10% from that numerical value.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. In another example, the amount “about 10” includes 10 and any amounts from 9 to 11. In yet another example, the term “about” in relation to a reference numerical value can also include a range of values plus or minus 10%, 9%, 8%, 7%, 6%, 5%, 4%, 3%, 2%, or 1% from that value. Alternatively, particularly with respect to biological systems or processes, the term “about” can mean within an order of magnitude, preferably within 5-fold, and more preferably within 2-fold, of a value. Where particular values are described in the application and claims, unless otherwise stated the term “about” meaning within an acceptable error range for the particular value should be assumed.

The term “isolated” and its grammatical equivalents as used herein refer to the removal of a nucleic acid from its natural environment. The term “purified” and its grammatical equivalents as used herein refer to a molecule or composition, whether removed from nature (including genomic DNA and mRNA) or synthesized (including cDNA) and/or amplified under laboratory conditions, that has been increased in purity, wherein “purity” is a relative term, not “absolute purity.” It is to be understood, however, that nucleic acids and proteins can be formulated with diluents or adjuvants and still for practical purposes be isolated. For example, nucleic acids typically are mixed with an acceptable carrier or diluent when used for introduction into cells. The term “substantially purified” and its grammatical equivalents as used herein refer to a nucleic acid sequence, polypeptide, protein or other compound which is essentially free, i.e., is more than about 50% free of, more than about 70% free of, more than about 90% free of, the polynucleotides, proteins, polypeptides and other molecules that the nucleic acid, polypeptide, protein or other compound is naturally associated with.

“Polynucleotide”, “oligonucleotide”, “polynucleotide construct”, “gene”, “gene construct”, “heterologous gene” and their grammatical equivalents as used herein refer to a polymeric form of nucleotides or nucleic acids of any length, either ribonucleotides or deoxyribonucleotides. This term refers only to the primary structure of the molecule. Thus, this term includes double and single stranded DNA, triplex DNA, as well as double and single stranded RNA. It also includes modified, for example, by methylation and/or by capping, and unmodified forms of the polynucleotide. The term is also meant to include molecules that include non-naturally occurring or synthetic nucleotides as well as nucleotide analogs. The nucleic acid sequences and vectors disclosed or contemplated herein can be introduced into a cell by, for example, transfection, transformation, or transduction.

“Transfection,” “transformation,” or “transduction” as used herein refer to the introduction of one or more exogenous polynucleotides into a host cell by using physical or chemical methods. Many transfection techniques are known in the art and include, for example, calcium phosphate DNA co-precipitation (see, e.g., Murray E. J. (ed.), Methods in Molecular Biology, Vol. 7, Gene Transfer and Expression Protocols, Humana Press (1991)); DEAE-dextran; electroporation; cationic liposome-mediated transfection; tungsten particle-facilitated microparticle bombardment (Johnston, Nature, 346:776-777 (1990)); and strontium phosphate DNA co-precipitation (Brash et al., Mol. Cell Biol., 7:2031-2034 (1987)). Phage or viral vectors can be introduced into host cells, after growth of infectious particles in suitable packaging cells, many of which are commercially available.

“Polypeptide”, “peptide” “polypeptide construct” and “peptide construct” and their grammatical equivalents as used herein refer to a polymer of amino acid residues. A “mature protein” is a protein which is full-length and which, optionally, includes glycosylation or other modifications typical for the protein in a given cellular environment. Polypeptides and proteins disclosed herein (including functional portions and functional variants thereof) can comprise synthetic amino acids in place of one or more naturally-occurring amino acids. Such synthetic amino acids are known in the art, and include, for example, aminocyclohexane carboxylic acid, norleucine, α-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, β-phenylserine β-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, α-aminocyclopentane carboxylic acid, α-aminocyclohexane carboxylic acid, α-aminocycloheptane carboxylic acid, α-(2-amino-2-norbornane)-carboxylic acid, α,γ-diaminobutyric acid, α,β-diaminopropionic acid, homophenylalanine, and α-tert-butylglycine. The present disclosure further contemplates that expression of polypeptides described herein in an engineered cell can be associated with post-translational modifications of one or more amino acids of the polypeptide constructs. Non-limiting examples of post-translational modifications include phosphorylation, acylation including acetylation and formylation, glycosylation (including N-linked and O-linked), amidation, hydroxylation, alkylation including methylation and ethylation, ubiquitylation, addition of pyrrolidone carboxylic acid, formation of disulfide bridges, sulfation, myristoylation, palmitoylation, isoprenylation, farnesylation, geranylation, glypiation, lipoylation and iodination.

Nucleic acids and/or nucleic acid sequences are “homologous” when they are derived, naturally or artificially, from a common ancestral nucleic acid or nucleic acid sequence. Proteins and/or protein sequences are “homologous” when their encoding DNAs are derived, naturally or artificially, from a common ancestral nucleic acid or nucleic acid sequence. The homologous molecules can be termed homologs. For example, any naturally occurring proteins, as described herein, can be modified by any available mutagenesis method. When expressed, this mutagenized nucleic acid encodes a polypeptide that is homologous to the protein encoded by the original nucleic acid. Homology is generally inferred from sequence identity between two or more nucleic acids or proteins (or sequences thereof). The precise percentage of identity between sequences that is useful in establishing homology varies with the nucleic acid and protein at issue, but as little as 25% sequence identity is routinely used to establish homology. Higher levels of sequence identity, e.g., 30%, 40%, 50%, 60%, 70%, 80%, 90%, 95% or 99% or more can also be used to establish homology. Methods for determining sequence identity percentages (e.g., BLASTP and BLASTN using default parameters) are described herein and are generally available.

The term “identical” and its grammatical equivalents as used herein or “sequence identity” in the context of two nucleic acid sequences or amino acid sequences of polypeptides refers to the residues in the two sequences which are the same when aligned for maximum correspondence over a specified comparison window. A “comparison window”, as used herein, refers to a segment of at least about 20 contiguous positions, usually about 50 to about 200, more usually about 100 to about 150 in which a sequence can be compared to a reference sequence of the same number of contiguous positions after the two sequences are aligned optimally. Methods of alignment of sequences for comparison are well-known in the art. Optimal alignment of sequences for comparison can be conducted by the local homology algorithm of Smith and Waterman, Adv. Appl. Math., 2:482 (1981); by the alignment algorithm of Needleman and Wunsch, J. Mol. Biol., 48:443 (1970); by the search for similarity method of Pearson and Lipman, Proc. Nat. Acad. Sci U.S.A., 85:2444 (1988); by computerized implementations of these algorithms (including, but not limited to CLUSTAL in the PC/Gene program by Intelligentics, Mountain View Calif., GAP, BESTFIT, BLAST, FASTA, and TFASTA in the Wisconsin Genetics Software Package, Genetics Computer Group (GCG), 575 Science Dr., Madison, Wis., U.S.A.); the CLUSTAL program is well described by Higgins and Sharp, Gene, 73:237-244 (1988) and Higgins and Sharp, CABIOS, 5:151-153 (1989); Corpet et al., Nucleic Acids Res., 16:10881-10890 (1988); Huang et al., Computer Applications in the Biosciences, 8:155-165 (1992); and Pearson et al., Methods in Molecular Biology, 24:307-331 (1994). Alignment is also often performed by inspection and manual alignment. In one class of embodiments, the polypeptides herein are at least 80%, 85%, 90%, 98% 99% or 100% identical to a reference polypeptide, or a fragment thereof, e.g., as measured by BLASTP (or CLUSTAL, or any other available alignment software) using default parameters. Similarly, nucleic acids can also be described with reference to a starting nucleic acid, e.g., they can be 50%, 60%, 70%, 75%, 80%, 85%, 90%, 98%, 99% or 100% identical to a reference nucleic acid or a fragment thereof, e.g., as measured by BLASTN (or CLUSTAL, or any other available alignment software) using default parameters. When one molecule is said to have certain percentage of sequence identity with a larger molecule, it means that when the two molecules are optimally aligned, said percentage of residues in the smaller molecule finds a match residue in the larger molecule in accordance with the order by which the two molecules are optimally aligned.

The term “substantially identical” and its grammatical equivalents as applied to nucleic acid or amino acid sequences mean that a nucleic acid or amino acid sequence comprises a sequence that has at least 90% sequence identity or more, at least 95%, at least 98% and at least 99%, compared to a reference sequence using the programs described above, e.g., BLAST, using standard parameters. For example, the BLASTN program (for nucleotide sequences) uses as defaults a word length (W) of 11, an expectation (E) of 10, M=5, N=−4, and a comparison of both strands. For amino acid sequences, the BLASTP program uses as defaults a word length (W) of 3, an expectation (E) of 10, and the BLOSUM62 scoring matrix (see Henikoff & Henikoff, Proc. Natl. Acad. Sci. USA 89:10915 (1992)). Percentage of sequence identity is determined by comparing two optimally aligned sequences over a comparison window, wherein the portion of the polynucleotide sequence in the comparison window can comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. In embodiments, the substantial identity exists over a region of the sequences that is at least about 50 residues in length, over a region of at least about 100 residues, and in embodiments, the sequences are substantially identical over at least about 150 residues. In embodiments, the sequences are substantially identical over the entire length of the coding regions.

An “expression vector” or “vector” is any genetic element, e.g., a plasmid, chromosome, virus, transposon, behaving either as an autonomous unit of polynucleotide replication within a cell. (i.e. capable of replication under its own control) or being rendered capable of replication by insertion into a host cell chromosome, having attached to it another polynucleotide segment, so as to bring about the replication and/or expression of the attached segment. Suitable vectors include, but are not limited to, plasmids, transposons, bacteriophages and cosmids. Vectors can contain polynucleotide sequences which are necessary to effect ligation or insertion of the vector into a desired host cell and to effect the expression of the attached segment. Such sequences differ depending on the host organism; they include promoter sequences to effect transcription, enhancer sequences to increase transcription, ribosomal binding site sequences and transcription and translation termination sequences. Alternatively, expression vectors can be capable of directly expressing nucleic acid sequence products encoded therein without ligation or integration of the vector into host cell DNA sequences. In some embodiments, the vector is an “episomal expression vector” or “episome,” which is able to replicate in a host cell, and persists as an extrachromosomal segment of DNA within the host cell in the presence of appropriate selective pressure (see, e.g., Conese et al., Gene Therapy, 11:1735-1742 (2004)). Representative commercially available episomal expression vectors include, but are not limited to, episomal plasmids that utilize Epstein Barr Nuclear Antigen 1 (EBNA1) and the Epstein Barr Virus (EBV) origin of replication (oriP). The vectors pREP4, pCEP4, PREP7, and pcDNA3.1 from Invitrogen (Carlsbad, Calif.) and pBK-CMV from Stratagene (La Jolla, Calif.) represent non-limiting examples of an episomal vector that uses T-antigen and the SV40 origin of replication in lieu of EBNA1 and oriP. Vector also can comprise a selectable marker gene.

The term “adenovirus,” as used herein, refers to an adenovirus that retains the ability to participate in the adenovirus life cycle and has not been physically inactivated by, for example, disruption (e.g., sonication), denaturing (e.g., using heat or solvents), or cross-linkage (e.g., via formalin cross-linking). The “adenovirus life cycle” includes (1) virus binding and entry into cells, (2) transcription of the adenoviral genome and translation of adenovirus proteins, (3) replication of the adenoviral genome, and (4) viral particle assembly (see, e.g., Fields Virology, 5^{th} ed., Knipe et al. (eds.), Lippincott Williams & Wilkins, Philadelphia, PA (2006)). The term “adenoviral vector,” as used herein, refers to an adenovirus in which the adenoviral genome has been manipulated to accommodate a nucleic acid sequence that is non-native with respect to the adenoviral genome. Typically, an adenoviral vector is generated by introducing one or more mutations (e.g., a deletion, insertion, or substitution) into the adenoviral genome of the adenovirus so as to accommodate the insertion of a non-native nucleic acid sequence, for example, for gene transfer, into the adenovirus.

The term “selectable marker gene” as used herein refers to a nucleic acid sequence that allows cells expressing the nucleic acid sequence to be specifically selected for or against, in the presence of a corresponding selective agent. Suitable selectable marker genes are known in the art and described in, e.g., International Patent Application Publications WO 1992/08796 and WO 1994/28143; Wigler et al., Proc. Natl. Acad. Sci. USA, 77:3567 (1980); O'Hare et al., Proc. Natl. Acad. Sci. USA, 78:1527 (1981); Mulligan & Berg, Proc. Natl. Acad. Sci. USA, 78:2072 (1981); Colberre-Garapin et al., J. Mol. Biol., 150:1 (1981); Santerre et al., Gene, 30:147 (1984); Kent et al., Science, 237:901-903 (1987); Wigler et al., Cell, 11:223 (1977); Szybalska & Szybalski, Proc. Natl. Acad. Sci. USA, 48:2026 (1962); Lowy et al., Cell, 22:817 (1980); and U.S. Pat. Nos. 5,122,464 and 5,770,359.

The term “coding sequence” as used herein refers to a segment of a polynucleotide that codes for protein. The region or sequence is bounded nearer the 5′ end by a start codon and nearer the 3′ end with a stop codon. Coding sequences can also be referred to as open reading frames.

The term “operably linked” as used herein refers to refers to the physical and/or functional linkage of a DNA segment to another DNA segment in such a way as to allow the segments to function in their intended manners. A DNA sequence encoding a gene product is operably linked to a regulatory sequence when it is linked to the regulatory sequence, such as, for example, promoters, enhancers and/or silencers, in a manner which allows modulation of transcription of the DNA sequence, directly or indirectly. For example, a DNA sequence is operably linked to a promoter when it is ligated to the promoter downstream with respect to the transcription initiation site of the promoter, in the correct reading frame with respect to the transcription initiation site and allows transcription elongation to proceed through the DNA sequence. An enhancer or silencer is operably linked to a DNA sequence coding for a gene product when it is ligated to the DNA sequence in such a manner as to increase or decrease, respectively, the transcription of the DNA sequence. Enhancers and silencers can be located upstream, downstream or embedded within the coding regions of the DNA sequence. A DNA for a signal sequence is operably linked to DNA coding for a polypeptide if the signal sequence is expressed as a pre-protein that participates in the secretion of the polypeptide. Linkage of DNA sequences to regulatory sequences is typically accomplished by ligation at suitable restriction sites or via adapters or linkers inserted in the sequence using restriction endonucleases known to one of skill in the art.

The terms “induce”, “induction” and its grammatical equivalents as used herein refer to an increase in nucleic acid sequence transcription, promoter activity and/or expression brought about by a transcriptional regulator, relative to some basal level of transcription.

The term “transcriptional regulator” refers to a biochemical element that acts to prevent or inhibit the transcription of a promoter-driven DNA sequence under certain environmental conditions (e.g., a repressor or nuclear inhibitory protein), or to permit or stimulate the transcription of the promoter-driven DNA sequence under certain environmental conditions (e.g., an inducer or an enhancer).

The term “enhancer” as used herein, refers to a DNA sequence that increases transcription of, for example, a nucleic acid sequence to which it is operably linked. Enhancers can be located many kilobases away from the coding region of the nucleic acid sequence and can mediate the binding of regulatory factors, patterns of DNA methylation, or changes in DNA structure. A large number of enhancers from a variety of different sources are well known in the art and are available as or within cloned polynucleotides (from, e.g., depositories such as the ATCC as well as other commercial or individual sources). A number of polynucleotides comprising promoters (such as the commonly-used CMV promoter) also comprise enhancer sequences. Enhancers can be located upstream, within, or downstream of coding sequences. The term “Ig enhancers” refers to enhancer elements derived from enhancer regions mapped within the immunoglobulin (lg) locus (such enhancers include for example, the heavy chain (mu) 5′ enhancers, light chain (kappa) 5′ enhancers, kappa and mu intronic enhancers, and 3′ enhancers (see generally Paul W. E. (ed), Fundamental Immunology, 3rd Edition, Raven Press, New York (1993), pages 353-363; and U.S. Pat. No. 5,885,827).

The term “promoter” refers to a region of a polynucleotide that initiates transcription of a coding sequence. Promoters are located near the transcription start sites of genes, on the same strand and upstream on the DNA (towards the 5′ region of the sense strand). Some promoters are constitutive as they are active in all circumstances in the cell, while others are regulated becoming active in response to specific stimuli, e.g., an inducible promoter. The term “promoter activity” and its grammatical equivalents as used herein refer to the extent of expression of nucleotide sequence that is operably linked to the promoter whose activity is being measured. Promoter activity can be measured directly by determining the amount of RNA transcript produced, for example by Northern blot analysis or indirectly by determining the amount of product coded for by the linked nucleic acid sequence, such as a reporter nucleic acid sequence linked to the promoter.

“Inducible promoter” as used herein refers to a promoter which is induced into activity by the presence or absence of transcriptional regulators, e.g., biotic or abiotic factors. Inducible promoters are useful because the expression of genes operably linked to them can be turned on or off at certain stages of development of an organism or in a particular tissue. Non-limiting examples of inducible promoters include alcohol-regulated promoters, tetracycline-regulated promoters, steroid-regulated promoters, metal-regulated promoters, pathogenesis-regulated promoters, temperature-regulated promoters and light-regulated promoters. The inducible promoter can be part of a gene switch or genetic switch. The inducible promoter can be a gene switch ligand inducible promoter. In some cases, an inducible promoter can be a small molecule ligand-inducible two polypeptide ecdysone receptor-based gene switch. In some cases, a gene switch can be selected from ecdysone-based receptor components as described in, but without limitation to, any of the systems described in: International Patent Applications WO 2001/070816; WO 2002/029075; WO 2002/066613; WO 2002/066614; WO 2002/066612; WO 2002/066615; WO 2003/027266; WO 2003/027289; WO 2005/108617; WO 2009/045370; WO 2009/048560; WO 2010/042189; WO 2010/042189; WO 2011/119773; and WO 2012/122025; and U.S. Pat. Nos. 7,091,038; 7,776,587; 7,807,417; 8,202,718; 8,105,825; 8,168,426; 7,531,326; 8,236,556; 8,598,409; 8,715,959; 7,601,508; 7,829,676; 7,919,269; 8,030,067; 7,563,879; 8,021,878; 8,497,093; 7,935,510; 8,076,454; 9,402,919; 9,493,540; 9,249,207; and 9,492,482, each of which is incorporated by reference in its entirety).

The term “gene switch” or “genetic switch” refers to the combination of a response element associated with a promoter, and for instance, an EcR based system, which, in the presence of one or more ligands, modulates the expression of a gene into which the response element and promoter are incorporated. Tightly regulated inducible gene expression systems or gene switches are useful for various applications such as gene therapy, large scale production of proteins in cells, cell based high throughput screening assays, functional genomics and regulation of traits in transgenic plants and animals. Such inducible gene expression systems can include ligand inducible heterologous gene expression systems.

“Sleeping Beauty (SB) Transposon System” refers a synthetic DNA transposon system for to introducing DNA sequences into the chromosomes of vertebrates. Some exemplary embodiments of the system are described, for example, in U.S. Pat. Nos. 6,489,458, 8,227,432, 9,228,180 and WO/2016/145146. The Sleeping Beauty transposon system is composed of a Sleeping Beauty (SB) transposase and a SB transposon. In embodiments, the Sleeping Beauty transposon system can include the SB11 transposon system, the SB100X transposon system, or the SB110 transposon system.

“Transposon” or “transposable element” (TE) is a vector DNA sequence that can change its position within the genome, sometimes creating or reversing mutations and altering the cell's genome size. Transposition often results in duplication of the TE. Class I TEs are copied in two stages: first they are transcribed from DNA to RNA, and the RNA produced is then reverse transcribed to DNA. This copied DNA is then inserted at a new position into the genome. The reverse transcription step is catalyzed by a reverse transcriptase, which can be encoded by the TE itself. The characteristics of retrotransposons are similar to retroviruses, such as HIV. The cut-and-paste transposition mechanism of class II TEs does not involve an RNA intermediate. The transpositions are catalyzed by several transposase enzymes. Some transposases non-specifically bind to any target site in DNA, whereas others bind to specific DNA sequence targets. The transposase makes a staggered cut at the target site resulting in single-strand 5′ or 3′ DNA overhangs (sticky ends). This step cuts out the DNA transposon, which is then ligated into a new target site; this process involves activity of a DNA polymerase that fills in gaps and of a DNA ligase that closes the sugar-phosphate backbone. This results in duplication of the target site. The insertion sites of DNA transposons can be identified by short direct repeats which can be created by the staggered cut in the target DNA and filling in by DNA polymerase, followed by a series of inverted repeats important for the TE excision by transposase. Cut-and-paste TEs can be duplicated if their transposition takes place during S phase of the cell cycle when a donor site has already been replicated, but a target site has not yet been replicated. Transposition can be classified as either autonomous or non-autonomous in both Class I and Class II TEs. Autonomous TEs can move by themselves while non-autonomous TEs require the presence of another TE to move. This is often because non-autonomous TEs lack transposase (for class II) or reverse transcriptase (for class I).

“Transposase” refers an enzyme that binds to the end of a transposon and catalyzes the movement of the transposon to another part of the genome by a cut and paste mechanism or a replicative transposition mechanism.

“T cell” or “T lymphocyte” as used herein is a type of lymphocyte that plays a central role in cell-mediated immunity. They can be distinguished from other lymphocytes, such as B cells and natural killer cells (NK cells), by the presence of a T-cell receptor (TCR) on the cell surface.

“T helper cells” (T_{H} cells) assist other white blood cells in immunologic processes, including maturation of B cells into plasma cells and memory B cells, and activation of cytotoxic T cells and macrophages. These cells are also known as CD4+ T cells because they express the CD4 glycoprotein on their surfaces. Helper T cells become activated when they are presented with peptide antigens by MHC class II molecules, which are expressed on the surface of antigen-presenting cells (APCs). Once activated, they divide rapidly and secrete small proteins called cytokines that regulate or assist in the active immune response. These cells can differentiate into one of several subtypes, including T_{H}1, T_{H}2, T_{H}3, T_{H}9, T_{H}17, T_{H}22 or T_{FH} (T follicular helper cells), which secrete different cytokines to facilitate different types of immune responses. Signaling from the APCs directs T cells into particular subtypes.

“Cytotoxic T cells” (TC cells, or CTLs) or “cytotoxic T lymphocytes” destroy virus-infected cells and tumor cells, and are also implicated in transplant rejection. These cells are also known as CD8+ T cells since they express the CD8 glycoprotein at their surfaces. These cells recognize their targets by binding to antigen associated with MHC class I molecules, which are present on the surface of all nucleated cells. Through IL-10, adenosine, and other molecules secreted by regulatory T cells, the CD8+ cells can be inactivated to an anergic state, which prevents autoimmune diseases.

“Memory T cells” are a subset of antigen-specific T cells that persist long-term after an infection has resolved. They quickly expand to large numbers of effector T cells upon re-exposure to their cognate antigen, thus providing the immune system with memory against past infections. Memory T cells comprise three subtypes: central memory T cells (T_{CM} cells) and two types of effector memory T cells (T_{EM} cells and T_{EMRA} cells). Memory cells can be either CD4+ or CD8+. Memory T cells typically express the cell surface proteins CD45RO, CD45RA and/or CCR7.

“Regulatory T cells” (Treg cells), formerly known as suppressor T cells, play a role in the maintenance of immunological tolerance. Their major role is to shut down T cell-mediated immunity toward the end of an immune reaction and to suppress autoreactive T cells that escaped the process of negative selection in the thymus.

“Natural killer T cells” (NKT cells—not to be confused with natural killer cells of the innate immune system) bridge the adaptive immune system with the innate immune system. Unlike conventional T cells that recognize peptide antigens presented by major histocompatibility complex (MHC) molecules, NKT cells recognize glycolipid antigen presented by a molecule called CD1d. Once activated, these cells can perform functions ascribed to both T helper (T_{H}) and cytotoxic T (TC) cells (i.e., cytokine production and release of cytolytic/cell killing molecules). They are also able to recognize and eliminate some tumor cells and cells infected with herpes viruses.

“Adoptive T cell transfer” refers to the isolation and ex vivo expansion of tumor specific T cells to achieve greater number of T cells than what could be obtained by vaccination alone or the patient's natural tumor response. The tumor specific T cells are then infused into patients with cancer in an attempt to give their immune system the ability to overwhelm remaining tumor via T cells which can attack and kill cancer. There are many forms of adoptive T cell therapy being used for cancer treatment; culturing tumor infiltrating lymphocytes or TIL, isolating and expanding one particular T cell or clone, and even using T cells that have been engineered to potently recognize and attack tumors.

“Antibody” as used herein refers to monoclonal or polyclonal antibodies. The term “monoclonal antibodies,” as used herein, refers to antibodies that are produced by a single clone of B-cells and bind to the same epitope. In contrast, “polyclonal antibodies” refer to a population of antibodies that are produced by different B-cells and bind to different epitopes of the same antigen. A whole antibody typically consists of four polypeptides: two identical copies of a heavy (H) chain polypeptide and two identical copies of a light (L) chain polypeptide. Each of the heavy chains contains one N-terminal variable (VH) region and three C-terminal constant (CH1, CH2 and CH3) regions, and each light chain contains one N-terminal variable (VL) region and one C-terminal constant (CL) region. The variable regions of each pair of light and heavy chains form the antigen binding site of an antibody. The VH and VL regions have a similar general structure, with each region comprising four framework regions, whose sequences are relatively conserved. The framework regions are connected by three complementarity determining regions (CDRs). The three CDRs, known as CDR1, CDR2, and CDR3, form the “hypervariable region” of an antibody, which is responsible for antigen binding.

“Antibody like molecules” can be for example proteins that are members of the Ig-superfamily which are able to selectively bind a partner. MHC molecules and T cell receptors are such molecules. In one embodiment, the antibody-like molecule is an TCR. In one embodiment, the TCR has been modified to increase its MHC binding affinity.

The terms “fragment of an antibody,” “antibody fragment,” “functional fragment of an antibody,” “antigen-binding portion” or its grammatical equivalents are used interchangeably herein to mean one or more fragments or portions of an antibody that retain the ability to specifically bind to an antigen (see, generally, Holliger et al., Nat. Biotech., 23(9):1126-1129 (2005)). The antibody fragment desirably comprises, for example, one or more CDRs, the variable region (or portions thereof), the constant region (or portions thereof), or combinations thereof. Non-limiting examples of antibody fragments include (i) a Fab fragment, which is a monovalent fragment consisting of the VL, VH, CL, and CH1 domains; (ii) a F(ab′)2 fragment, which is a bivalent fragment comprising two Fab fragments linked by a disulfide bridge at the stalk region; (iii) a Fv fragment consisting of the VL and VH domains of a single arm of an antibody; (iv) a single chain Fv (scFv), which is a monovalent molecule consisting of the two domains of the Fv fragment (i.e., VL and VH) joined by a synthetic linker which enables the two domains to be synthesized as a single polypeptide chain (see, e.g., Bird et al., Science, 242:423-426 (1988); Huston et al., Proc. Natl. Acad. Sci. USA, 85:5879-5883 (1988); and Osbourn et al., Nat. Biotechnol., 16:778 (1998)) and (v) a diabody, which is a dimer of polypeptide chains, wherein each polypeptide chain comprises a VH connected to a VL by a peptide linker that is too short to allow pairing between the VH and VL on the same polypeptide chain, thereby driving the pairing between the complementary domains on different VH-VL polypeptide chains to generate a dimeric molecule having two functional antigen binding sites. Antibody fragments are known in the art and are described in more detail in, e.g., U.S. Pat. No. 8,603,950.

“Antigen recognition moiety” or “antigen recognition domain” refers to a molecule or portion of a molecule that specifically binds to an antigen. In one embodiment, the antigen recognition moiety is an antibody, antibody like molecule or fragment thereof and the antigen is a tumor antigen.

The term “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz, G. E. and Schirmer, R. H., Principles of Protein Structure, Springer-Verlag, New York (1979)). According to such analyses, groups of amino acids can be defined where amino acids within a group exchange preferentially with each other, and therefore resemble each other most in their impact on the overall protein structure (Schulz, G. E. and Schirmer, R. H., supra). Examples of conservative mutations include amino acid substitutions of amino acids within the sub-groups above, for example, lysine for arginine and vice versa such that a positive charge can be maintained; glutamic acid for aspartic acid and vice versa such that a negative charge can be maintained; serine for threonine such that a free —OH can be maintained; and glutamine for asparagine such that a free —NH₂ can be maintained. Alternatively or additionally, the functional variants can comprise the amino acid sequence of the reference protein with at least one non-conservative amino acid substitution.

The term “non-conservative mutations” involve amino acid substitutions between different groups, for example, lysine for tryptophan, or phenylalanine for serine, etc. In this case, it is preferable for the non-conservative amino acid substitution to not interfere with, or inhibit the biological activity of, the functional variant. The non-conservative amino acid substitution can enhance the biological activity of the functional variant, such that the biological activity of the functional variant is increased as compared to the homologous parent protein.

The term “ankyrin” refers to a family of adaptor proteins that mediate the attachment of integral membrane proteins to the spectrin-actin based membrane cytoskeleton. Ankyrins have binding sites for the beta subunit of spectrin and at least 12 families of integral membrane proteins. This linkage is required to maintain the integrity of the plasma membranes and to anchor specific ion channels, ion exchangers and ion transporters in the plasma membrane. Ankyrins contain four functional domains: an N-terminal domain that contains 24 tandem ankyrin repeats, a central domain that binds to spectrin, a death domain that binds to proteins involved in apoptosis, and a C-terminal regulatory domain that is highly variable between different ankyrin proteins. The 24 tandem ankyrin repeats are responsible for the recognition of a wide range of membrane proteins. These 24 repeats contain 3 structurally distinct binding sites ranging from repeat 1-14. These binding sites are quasi-independent of each other and can be used in combination. The interactions the sites use to bind to membrane proteins are non-specific and consist of: hydrogen bonding, hydrophobic interactions and electrostatic interactions. These non-specific interactions gives ankyrin the property to recognize a large range of proteins as the sequence doesn't have to be conserved just the properties of the amino acids. The quasi-independence means that if a binding site is not used, it won't have a large effect on the overall binding. These two properties in combination give rise to large repertoire of proteins ankyrin can recognize. Ankyrins are encoded by three genes (ANK1, ANK2 and ANK3) in mammals. Each gene in turn produces multiple proteins through alternative splicing.

The term “proliferative disease” as referred to herein refers to a unifying concept in which excessive proliferation of cells and/or turnover of cellular matrix contributes significantly to the pathogenesis of the disease, including cancer.

“Patient” or “subject” as used herein refers to a mammalian subject diagnosed with or suspected of having or developing a proliferative disorder such as cancer. In some embodiments, the term “patient” refers to a mammalian subject with a higher than average likelihood of developing a proliferative disorder such as cancer. Exemplary patients can be humans, apes, dogs, pigs, cattle, cats, horses, goats, sheep, rodents and other mammalians that can benefit from the therapies disclosed herein. Exemplary human patients can be male and/or female. “Patient in need thereof” or “subject in need thereof” is referred to herein as a patient diagnosed with or suspected of having a disease or disorder, for instance, but not restricted to human papilloma virus (HPV) infection.

“Administering” is referred to herein as providing one or more compositions described herein to a patient or a subject. By way of example and not limitation, composition administration, e.g., injection, can be performed by intravenous (i.v.) injection, sub-cutaneous (s.c.) injection, intradermal (i.d.) injection, intraperitoneal (i.p.) injection, or intramuscular (i.m.) injection. One or more such routes can be employed. Parenteral administration can be, for example, by bolus injection or by gradual perfusion over time. Alternatively, or concurrently, administration can be by the oral route. Additionally, administration can also be by surgical deposition of a bolus or pellet of cells, or positioning of a medical device. In an embodiment, a composition of the present disclosure can comprise engineered cells or host cells expressing nucleic acid sequences described herein, or a vector comprising at least one nucleic acid sequence described herein, in an amount that is effective to treat or prevent proliferative disorders. A pharmaceutical composition can comprise a target cell population as described herein, in combination with one or more pharmaceutically or physiologically acceptable carriers, diluents or excipients. Such compositions can comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

As used herein, the term “treatment”, “treating”, or its grammatical equivalents refers to obtaining a desired pharmacologic and/or physiologic effect. In embodiments, the effect is therapeutic, i.e., the effect partially or completely cures a disease and/or adverse symptom attributable to the disease. To this end, the inventive method comprises administering a therapeutically effective amount of the composition comprising the host cells expressing the inventive nucleic acid sequence, or a vector comprising the inventive nucleic acid sequences.

The term “therapeutically effective amount”, therapeutic amount”, “immunologically effective amount”, “anti-tumor effective amount”, “tumor-inhibiting effective amount” or its grammatical equivalents refers to an amount effective, at dosages and for periods of time necessary, to achieve a desired therapeutic result. The therapeutically effective amount can vary according to factors such as the disease state, age, sex, and weight of the individual, and the ability of a composition described herein to elicit a desired response in one or more subjects. The precise amount of the compositions of the present disclosure to be administered can be determined by a physician with consideration of individual differences in age, weight, tumor size, extent of infection or metastasis, and condition of the patient (subject).

Alternatively, the pharmacologic and/or physiologic effect of administration of one or more compositions described herein to a patient or a subject of can be “prophylactic,” i.e., the effect completely or partially prevents a disease or symptom thereof. A “prophylactically effective amount” refers to an amount effective, at dosages and for periods of time necessary, to achieve a desired prophylactic result (e.g., prevention of disease onset).

### HPV Molecular Vaccine

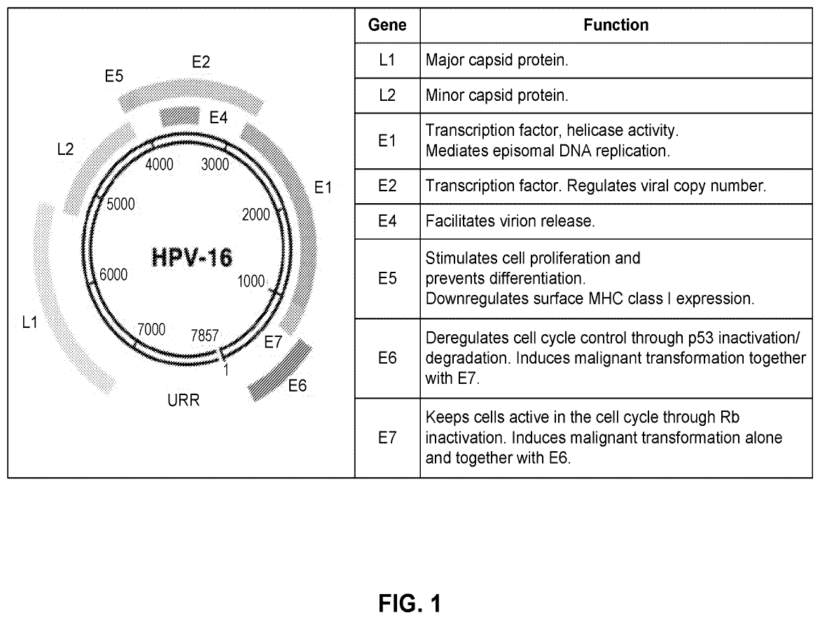

Human papillomavirus (HPV) is a group of more than 200 related viruses. Each HPV virus in this large group is given a number which is called its HPV type (or serotype). HPV is a small, non-enveloped deoxyribonucleic acid (DNA) virus that infects skin or mucosal cells. The circular, double-stranded viral genome is approximately 8-kb in length. The genome encodes for seven early proteins (E1 to E7) responsible for virus replication and two late proteins (L1 and L2), which are the viral structural proteins. As depicted in FIG. 1, each gene has specific functions. At least 13 of more than 200 known HPV types can cause cancer of the cervix and are associated with other anogenital cancers and cancers of the head and neck. The two most common “high-risk” serotypes (HPV-16 and HPV-18) cause approximately 70% of all cervical cancers. In HPV-16 and HPV-18, two primary oncoproteins, E6 and E7, are constitutively expressed by HPV-associated tumors and are critical for the induction and maintenance of cellular transformation in HPV-infected cells. Recent evidence also suggests the E5 protein also impacts viral transformation. HPV types 16, 18, 31, 33, 35, 39, 45, 51, 52, 56, 58, 59, 68, 73, and 82 are considered carcinogenic. Two “low-risk” HPV 6 and 11 are known to cause genital warts, a common benign condition of the external genitalia that causes significant morbidity. HPV is highly transmissible, with peak incidence soon after the onset of sexual activity, and most persons acquire infection at some time in their lives.

Prophylactic vaccines composed of virus-like particles, only induce immunity to the capsid structure and not to the non-structural proteins responsible for cell transformation. Initial HPV studies in animal models showed that inoculation with species-specific papillomaviruses induced an immune response that conferred protection against homologous virus challenge. However, native papillomaviruses are not good substrates for vaccine development as they cannot be grown easily in tissue culture. Subsequent studies were initiated on the production of viral particles from expression of the structural proteins in heterologous expression systems, such as yeast or baculovirus vectors. Results showed that expression of L1 alone led to the production of virus-like particles (VLPs) which morphologically resemble the authentic HPV virions but contain no viral DNA. These VLPs are produced by self-assembly of the L1 protein when expressed in a heterologous cell substrate. In animal studies, VLPs were shown to protect against high dose experimental infection by homologous virus. HPV VLPs are highly immunogenic in mice or rabbits, and the resulting antibodies have been shown to be neutralizing and type restricted when tested in a pseudovirion neutralization assay. Immunization with denatured particles does not result in the production of neutralizing antibodies, or protect from experimental virus challenge, indicating that neutralizing epitopes are conformation dependent.

Provided herein are compositions, kits, and systems comprising methods of making HPV recombinant vaccines. The HPV recombinant vaccines (e.g., HPV designs 1-5) in the present disclosure are engineered through protein engineering of E5, E6, and E7. These vaccines include a greater protein level sequence consensus amongst different HPV-16 and HPV-18 isolates. Moreover, they include genetically modified mutations to avoid oncogenic activities, improve their expression, and trigger broader immune response. Also provided herein are multi-epitope recombinant antigens containing immunogenic epitopes of the E5, E6, and E7 proteins. Along with the present HPV vaccine designs, certain enhancer agonist peptides previously shown to activate HPV-specific T cells are included. In some embodiments, the agonist peptides comprise one or more peptide sequences as shown in Table 2.

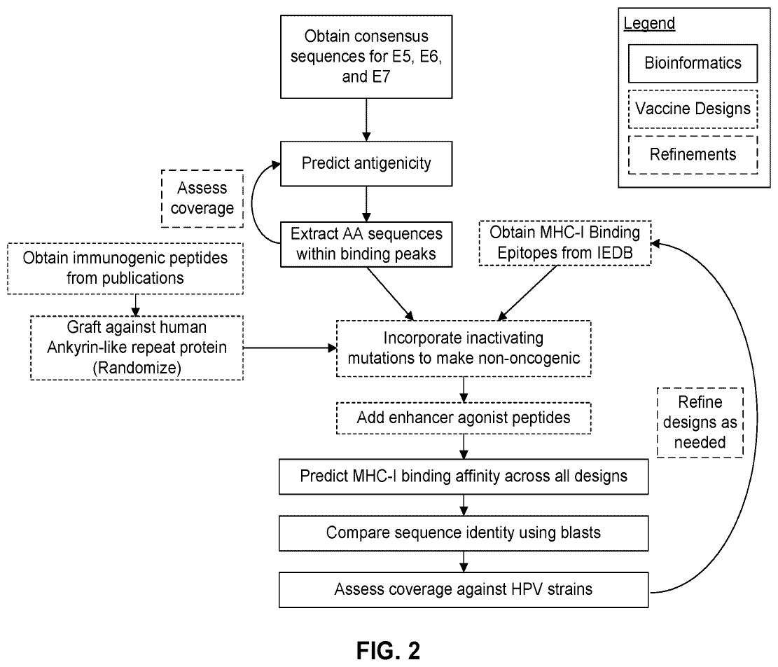

Each HPV vaccine antigen design was inspired via inventor-selected combinatorial use of bioinformatics analysis and in silico protein engineering methods (e.g., selection of antigenic sequences based on consensus sequences, antigenicity predictions, and T cell epitope mapping, targeted to induce MHC-I binding and cytokine production following T cell activation). The overall workflow of the presently disclosed HPV vaccine designs is shown in FIG. 2 and is further detailed in Example 2.

The naturally existing sequence variations on numerous HPV strains present a significant hurdle to the development of effective, broad spectrum HPV vaccines. As a solution to this problem, the present vaccine design approach utilizes advanced bioinformatics and protein engineering approaches to select and design antigen sequences with broad coverage of T cell epitopes, novel mutations, and enhancer agonist peptides. Drawing on information available of extended coverage of antigen regions with CTL-specific epitopes and in silico prediction results, the designed HPV vaccine antigens are targeted to induce robust HPV-16 and HPV-18 specific responses and provide therapeutic benefit to persons at risk of HPV-derived cancers.

The present disclosure provides five HPV antigen designs (HPV designs 1-5) constructed in a multi-deleted gorilla adenovector (GC46). Detailed methods for each of the HPV antigen designs are elaborated in Examples 1-3. For RNA qPCR relative expression assay, 5′-TGCCAAGAGTGACGTGTCCA-3′ (SEQ ID NO: 110) was used as a splice primer, and 5′-CCCAGGTCCAACTGCAGCCGG-3′ (SEQ ID NO: 111) was used as a splice probe. Specific primers designed for each antigen were used as reverse primers (FIG. 6).

### Delivery System

### Gorilla Adenovirus Shuttle Vector

Certain aspects of the present disclosure are directed to a vector comprising a polynucleotide encoding a polypeptide comprising one or more immune response-inducing HPV polypeptides described herein. In certain embodiments, the vector is a viral vector. In particular embodiments, the vector is an adenoviral vector. Adenoviruses are generally associated with benign pathologies in humans, and the genomes of adenoviruses isolated from a variety of species, including humans, have been extensively studied. Adenovirus is a medium-sized (90-100 nm), non-enveloped icosahedral virus containing approximately 36 kb of double-stranded DNA. The adenovirus capsid mediates the key interactions of the early stages of the infection of a cell by the virus, and is required for packaging adenovirus genomes at the end of the adenovirus life cycle. The capsid comprises 252 capsomeres, which includes 240 hexons, 12 penton base proteins, and 12 fibers (Ginsberg et al., Virology, 28:782-83 (1966)). The hexon comprises three identical proteins, namely polypeptide II (Roberts et al., Science, 232:1148-51 (1986)). The penton base comprises five identical proteins and the fiber comprises three identical proteins. Proteins IIIa, VI, and IX are present in the adenoviral coat and are believed to stabilize the viral capsid (Stewart et al., Cell, 67:145-54 (1991), and Stewart et al., EMBO J., 12(7): 2589-99 (1993)). The expression of the capsid proteins, with the exception of pIX, is dependent on the adenovirus polymerase protein. Therefore, major components of an adenovirus particle are expressed from the genome only when the polymerase protein gene is present and expressed.

Several features of adenoviruses make them ideal vehicles for transferring genetic material to cells for therapeutic applications (i.e., “gene therapy”), or for use as antigen delivery systems for vaccine applications. For example, adenoviruses can be produced in high titers (e.g., about 1013 particle units (pu)), and can transfer genetic material to non-replicating and replicating cells. The adenoviral genome can be manipulated to carry a large amount of exogenous DNA (up to about 8 kb), and the adenoviral capsid can potentiate the transfer of even longer sequences (Curiel et al., Hum. Gene Ther., 3:147-154 (1992)). Additionally, adenoviruses generally do not integrate into the host cell chromosome, but rather are maintained as a linear episome, thereby minimizing the likelihood that a recombinant adenovirus will interfere with normal cell function.

In some embodiments, the adenovirus described herein is isolated from a gorilla. There are four widely recognized gorilla subspecies within the two species of Eastern Gorilla (Gorilla beringei) and Western Gorilla (Gorilla gorilla). The Western Gorilla species includes the subspecies Western Lowland Gorilla (Gorilla gorilla gorilla) and Cross River Gorilla (Gorilla gorilla diehli). The Eastern Gorilla species includes the subspecies Mountain Gorilla (Gorilla beringei beringei) and Eastern Lowland Gorilla (Gorilla beringei graueri) (see, e.g., Wilson and Reeder, eds., Mammalian Species of the World, 3^{rd} ed., Johns Hopkins University Press, Baltimore, Maryland (2005)). In some embodiments, the adenovirus of the present disclosure is isolated from Mountain Gorilla (Gorilla beringei beringei).

Various Gorilla adenoviruses or adenoviral vectors are described in International Patent Application Publications WO 2013/052832; WO 2013/052811; and WO 2013 052799, each of which is herein incorporated by reference in its entirety.

The genomes of several such adenoviruses have been analyzed, and it has been determined that the adenovirus can have the nucleic acid sequence of, for example, SEQ ID NO: 21, SEQ ID NO: 22, SEQ ID NO: 23, SEQ ID NO: 24, or SEQ ID NO: 25, each of which includes a number of sub-sequences that serve to uniquely define the adenovirus, namely the nucleic acid sequences SEQ ID NOs: 1-10, and amino acid sequences SEQ ID NOs: 11-20. SEQ ID NOs: 6-10 encode the amino acid sequences of SEQ ID NOs: 16-20, respectively. SEQ ID NOs: 1-5 are a subset of the nucleic acid sequences of SEQ ID NOS: 6-10, respectively. SEQ ID NOs: 11-15 are a subset of the amino acid sequences of SEQ ID NOs: 16-20, respectively.

The adenovirus can be modified in the same manner as previously known adenoviruses to be used as an adenoviral vector, e.g., a gene delivery vehicle. The adenovirus and adenoviral vector can be replication-competent, conditionally replication-competent, or replication-deficient.

A replication-competent adenovirus or adenoviral vector can replicate in typical host cells, i.e., cells typically capable of being infected by an adenovirus. A replication-competent adenovirus or adenoviral vector can have one or more mutations as compared to the wild-type adenovirus (e.g., one or more deletions, insertions, and/or substitutions) in the adenoviral genome that do not inhibit viral replication in host cells. For example, the adenovirus or adenoviral vector can have a partial or entire deletion of the adenoviral early region known as the E3 region, which is not essential for propagation of the adenovirus or adenoviral genome.

A conditionally-replicating adenovirus or adenoviral vector is an adenovirus or adenoviral vector that has been engineered to replicate under pre-determined conditions. For example, replication-essential gene functions, e.g., gene functions encoded by the adenoviral early regions, can be operably linked to an inducible, repressible, or tissue-specific transcription control sequence, e.g., promoter. In such an embodiment, replication requires the presence or absence of specific factors that interact with the transcription control sequence. Conditionally-replicating adenoviral vectors are further described in U.S. Pat. No. 5,998,205.

A replication-deficient adenovirus or adenoviral vector is an adenovirus or adenoviral vector that requires complementation of one or more gene functions or regions of the adenoviral genome that are required for replication, as a result of, for example, a deficiency in one or more replication-essential gene function or regions, such that the adenovirus or adenoviral vector does not replicate in typical host cells, especially those in a human to be infected by the adenovirus or adenoviral vector.

A deficiency in a gene function or genomic region, as used herein, is defined as a disruption (e.g., deletion) of sufficient genetic material of the adenoviral genome to obliterate or impair the function of the gene (e.g., such that the function of the gene product is reduced by at least about 2-fold, 5-fold, 10-fold, 20-fold, 30-fold, or 50-fold) whose nucleic acid sequence was disrupted (e.g., deleted) in whole or in part. Deletion of an entire gene region often is not required for disruption of a replication-essential gene function. However, for the purpose of providing sufficient space in the adenoviral genome for one or more transgenes, removal of a majority of one or more gene regions can be desirable. While deletion of genetic material is preferred, mutation of genetic material by addition or substitution also is appropriate for disrupting gene function. Replication-essential gene functions are those gene functions that are required for adenovirus replication (e.g., propagation) and are encoded by, for example, the adenoviral early regions (e.g., the E1, E2, and E4 regions), late regions (e.g., the L1, L2, L3, L4, and L5 regions), genes involved in viral packaging (e.g., the IVa2 gene), and virus-associated RNAs (e.g., VA-RNA-1 and/or VA-RNA-2).

Whether the adenovirus or adenoviral vector is replication-competent or replication-deficient, the adenovirus or adenoviral vector retains at least a portion of the adenoviral genome. The adenovirus or adenoviral vector can comprise any portion of the adenoviral genome, including protein coding and non-protein coding regions. Desirably, the adenovirus or adenoviral vector comprises at least one nucleic acid sequence that encodes an adenovirus protein. The adenovirus or adenoviral vector can comprise a nucleic acid sequence that encodes any suitable adenovirus protein, such as, for example, a protein encoded by any one of the early region genes (i.e., E1A, E1B, E2A, E2B, E3, and/or E4 regions), or a protein encoded by any one of the late region genes, which encode the virus structural proteins (i.e., L1, L2, L3, L4, and L5 regions).

The adenovirus or adenoviral vector desirably comprises one or more nucleic acid sequences that encode the pIX protein, the DNA polymerase protein, the penton protein, the hexon protein, and/or the fiber protein. The adenovirus or adenoviral vector can comprise a full-length nucleic acid sequence that encodes a full-length amino acid sequence of an adenovirus protein. Alternatively, the adenovirus or adenoviral vector can comprise a portion of a full-length nucleic acid sequence that encodes a portion of a full-length amino acid sequence of an adenovirus protein.

A “portion” of a nucleic acid sequence comprises at least ten nucleotides (e.g., about 10 to about 5000 nucleotides). Preferably, a “portion” of a nucleic acid sequence comprises 10 or more (e.g., 15 or more, 20 or more, 25 or more, 30 or more, 35 or more, 40 or more, 45 or more, 50 or more, or 100 or more) nucleotides, but less than 5,000 (e.g., 4900 or less, 4000 or less, 3000 or less, 2000 or less, 1000 or less, 800 or less, 500 or less, 300 or less, or 100 or less) nucleotides. Preferably, a portion of a nucleic acid sequence is about 10 to about 3500 nucleotides (e.g., about 10, 20, 30, 50, 100, 300, 500, 700, 1000, 1500, 2000, 2500, or 3000 nucleotides), about 10 to about 1000 nucleotides (e.g., about 25, 55, 125, 325, 525, 725, or 925 nucleotides), or about 10 to about 500 nucleotides (e.g., about 15, 30, 40, 50, 60, 70, 80, 90, 150, 175, 250, 275, 350, 375, 450, 475, 480, 490, 495, or 499 nucleotides), or a range defined by any two of the foregoing values. More preferably, a “portion” of a nucleic acid sequence comprises no more than about 3200 nucleotides (e.g., about 10 to about 3200 nucleotides, about 10 to about 3000 nucleotides, or about 30 to about 500 nucleotides, or a range defined by any two of the foregoing values).

A “portion” of an amino acid sequence comprises at least three amino acids (e.g., about 3 to about 1,200 amino acids). Preferably, a “portion” of an amino acid sequence comprises 3 or more (e.g., 5 or more, 10 or more, 15 or more, 20 or more, 25 or more, 30 or more, 40 or more, or 50 or more) amino acids, but less than 1,200 (e.g., 1,000 or less, 800 or less, 700 or less, 600 or less, 500 or less, 400 or less, 300 or less, 200 or less, or 100 or less) amino acids. Preferably, a portion of an amino acid sequence is about 3 to about 500 amino acids (e.g., about 10, 100, 200, 300, 400, or 500 amino acids), about 3 to about 300 amino acids (e.g., about 20, 50, 75, 95, 150, 175, or 200 amino acids), or about 3 to about 100 amino acids (e.g., about 15, 25, 35, 40, 45, 60, 65, 70, 80, 85, 90, 95, or 99 amino acids), or a range defined by any two of the foregoing values. More preferably, a “portion” of an amino acid sequence comprises no more than about 500 amino acids (e.g., about 3 to about 400 amino acids, about 10 to about 250 amino acids, or about 50 to about 100 amino acids, or a range defined by any two of the foregoing values).

The adenovirus pIX protein is present in the adenovirus capsid, has been shown to strengthen hexon nonamer interactions, and is essential for the packaging of full-length genomes (see, e.g., Boulanger et al., J. Gen. Virol., 44:783-800 (1979); Horwitz M. S., “Adenoviridae and their replication” in Virology, 2^{nd} ed., B. N. Fields et al. (eds.), Raven Press, Ltd., New York, pp. 1679-1721 (1990), Ghosh-Choudhury et al., EMBO J., 6:1733-1739 (1987), and van Oostrum et al, J. Virol., 56:439-448 (1985)). In addition to its contribution to adenovirus structure, pIX also has been shown to exhibit transcriptional properties, such as stimulation of adenovirus major late promoter (MLP) activity (see, e.g., Lutz et al., J. Virol., 71(7): 5102-5109 (1997)). Nucleic acid sequences that encode all or a portion of an adenovirus pIX protein include, for example, SEQ ID NO: 6 and SEQ ID NO: 1. Amino acid sequences that comprise a full-length pIX protein, or a portion thereof, include, for example, SEQ ID NO: 16 and SEQ ID NO: 11.

The adenovirus DNA polymerase protein is essential for viral DNA replication both in vitro and in vivo. The polymerase co-purifies in a complex with the precursor (pTP) of the terminal protein (TP), which is covalently attached to the 5′ ends of adenovirus DNA (Field et al., J. Biol. Chem., 259:9487-9495 (1984)). Both the adenovirus DNA polymerase and pTP are encoded by the E2 region. The polymerase protein is required for the expression of all the structural proteins except for pIX. Without the gene sequence for polymerase protein, polymerase protein is not produced. As a result, the viral genome is not replicated, the Major Late Promoter is not activated, and the capsid proteins are not expressed. Nucleic acid sequences that encode all or a portion of an adenovirus DNA polymerase protein include, for example, SEQ ID NO: 7 and SEQ ID NO: 2. Amino acid sequences that comprise a full-length adenovirus DNA polymerase, or a portion thereof, include, for example, SEQ ID NO: 17 and SEQ ID NO: 12.

The adenovirus hexon protein is the largest and most abundant protein in the adenovirus capsid. The hexon protein is essential for virus capsid assembly, determination of the icosahedral symmetry of the capsid (which in turn defines the limits on capsid volume and DNA packaging size), and integrity of the capsid. In addition, hexon is a primary target for modification in order to reduce neutralization of adenoviral vectors (see, e.g., Gall et al., J. Virol., 72:10260-264 (1998), and Rux et al., J. Virol., 77(17): 9553-9566 (2003)). The major structural features of the hexon protein are shared by adenoviruses across serotypes, but the hexon protein differs in size and immunological properties between serotypes (Jornvall et al., J. Biol. Chem., 256(12): 6181-6186 (1981)). A comparison of 15 adenovirus hexon proteins revealed that the predominant antigenic and serotype-specific regions of the hexon appear to be in loops 1 and 2 (i.e., L1 or l1, and LII or l2, respectively), within which are seven discrete hypervariable regions (HVR1 to HVR7) varying in length and sequence between adenoviral serotypes (Crawford-Miksza et al., J. Virol., 70(3): 1836-1844 (1996)). Nucleic acid sequences that encode all or a portion of an adenovirus hexon protein include, for example, SEQ ID NO: 9 and SEQ ID NO: 4. Amino acid sequences that comprise a full-length adenovirus hexon protein, or a portion thereof, include, for example, SEQ ID NO: 19 and SEQ ID NO: 14.

The adenovirus fiber protein is a homotrimer of the adenoviral polypeptide IV that has three domains: the tail, shaft, and knob. (Devaux et al., J. Molec. Biol., 215:567-88 (1990), Yeh et al., Virus Res., 33:179-98 (1991)). The fiber protein mediates primary viral binding to receptors on the cell surface via the knob and the shaft domains (Henry et al., J. Virol., 68(8): 5239-46 (1994)). The amino acid sequences for trimerization are located in the knob, which appears necessary for the amino terminus of the fiber (the tail) to properly associate with the penton base (Novelli et al., Virology, 185:365-76 (1991)). In addition to recognizing cell receptors and binding the penton base, the fiber contributes to serotype identity. Fiber proteins from different adenoviral serotypes differ considerably (see, e.g., Green et al., EMBO J., 2:1357-65 (1983), Chroboczek et al., Virology, 186:280-85 (1992), and Signas et al., J. Virol., 53:672-78 (1985)). Thus, the fiber protein has multiple functions key to the life cycle of adenovirus. Nucleic acid sequences that encode all or a portion of an adenovirus fiber protein include, for example, SEQ ID NO: 10 and SEQ ID NO: 5. Amino acid sequences that comprise a full-length adenovirus fiber protein, or a portion thereof, include, for example, SEQ ID NO: 20 and SEQ ID NO: 15.

The adenovirus penton base protein is located at the vertices of the icosahedral capsid and comprises five identical monomers. The penton base protein provides a structure for bridging the hexon proteins on multiple facets of the icosahedral capsid, and provides the essential interface for the fiber protein to be incorporated in the capsid. Each monomer of the penton base contains an RGD tripeptide motif (Neumann et al., Gene, 69:153-157 (1988)). The RGD tripeptide mediates binding to av integrins and adenoviruses that have point mutations in the RGD sequence of the penton base are restricted in their ability to infect cells (Bai et al., J. Virol., 67:5198-5205 (1993)). Thus, the penton base protein is essential for the architecture of the capsid and for maximum efficiency of virus-cell interaction. Nucleic acid sequences that encode all or a portion of an adenovirus penton base protein include, for example, SEQ ID NO: 8 and SEQ ID NO: 3. Amino acid sequences that comprise a full-length adenovirus penton base protein, or a portion thereof, include, for example, SEQ ID NO: 18 and SEQ ID NO: 13.

Nucleic acid or amino acid sequence “identity,” as described herein, can be determined by comparing a nucleic acid or amino acid sequence of interest to a reference nucleic acid or amino acid sequence. The numbers of nucleotides or amino acid residues that have been changed and/or modified (such as, e.g., by point mutations, insertions, or deletions) in the reference sequence so as to result in the sequence of interest are counted. The total number of such changes is subtracted from the total length of the sequence of interest, and the difference is divided by the length of the sequence of interest and expressed as a percentage. A number of mathematical algorithms for obtaining the optimal alignment and calculating identity between two or more sequences are known and incorporated into a number of available software programs. Examples of such programs include CLUSTAL-W, T-Coffee, and ALIGN (for alignment of nucleic acid and amino acid sequences), BLAST programs (e.g., BLAST 2.1, BL2SEQ, and later versions thereof) and FASTA programs (e.g., FASTA3x, FAS™, and SSEARCH) (for sequence alignment and sequence similarity searches). Sequence alignment algorithms also are disclosed in, for example, Altschul et al., J. Molecular Biol., 215(3): 403-410 (1990), Beigert et al., Proc. Natl. Acad. Sci. USA, 106(10): 3770-3775 (2009), Durbin et al., eds., Biological Sequence Analysis: Probabilistic Models of Proteins and Nucleic Acids, Cambridge University Press, Cambridge, UK (2009), Soding, Bioinformatics, 21(7): 951-960 (2005), Altschul et al., Nucleic Acids Res., 25(17): 3389-3402 (1997), and Gusfield, Algorithms on Strings, Trees and Sequences, Cambridge University Press, Cambridge UK (1997)).

The adenovirus or adenoviral vector can comprise one, two, three, four, or all five of the aforementioned sequences alone or in any combination. In this respect, the adenovirus or adenoviral vector can comprise any combination of any two of the aforementioned sequences, any combination of any three of the aforementioned sequences, any combination of any four of the aforementioned sequences, or all five of the aforementioned sequences.

As discussed herein, the adenovirus or adenoviral vector can be replication-competent, conditionally-replicating, or replication-deficient. Preferably, the adenovirus or adenoviral vector is replication-deficient, such that the replication-deficient adenovirus or adenoviral vector requires complementation of at least one replication-essential gene function of one or more regions of the adenoviral genome for propagation (e.g., to form adenoviral vector particles).

The replication-deficient adenovirus or adenoviral vector can be modified in any suitable manner to cause the deficiencies in the one or more replication-essential gene functions in one or more regions of the adenoviral genome for propagation. The complementation of the deficiencies in the one or more replication-essential gene functions of one or more regions of the adenoviral genome refers to the use of exogenous means to provide the deficient replication-essential gene functions. Such complementation can be effected in any suitable manner, for example, by using complementing cells and/or exogenous DNA (e.g., helper adenovirus) encoding the disrupted replication-essential gene functions.

The adenovirus or adenoviral vector can be deficient in one or more replication-essential gene functions of only the early regions (i.e., E1-E4 regions) of the adenoviral genome, only the late regions (i.e., L1-L5 regions) of the adenoviral genome, both the early and late regions of the adenoviral genome, or all adenoviral genes (i.e., a high capacity adenovector (HC-Ad). See Morsy et al., Proc. Natl. Acad. Sci. USA, 95:965-976 (1998); Chen et al., Proc. Natl. Acad. Sci. USA, 94:1645-1650 (1997); and Kochanek et al., Hum. Gene Ther., 10:2451-2459 (1999). Examples of replication-deficient adenoviral vectors are disclosed in U.S. Pat. Nos. 5,837,511; 5,851,806; 5,994,106; 6,127,175; 6,482,616; and 7,195,896, and International Patent Application Publications WO 1994/028152, WO 1995/002697, WO 1995/016772, WO 1995/034671, WO 1996/022378, WO 1997/012986, WO 1997/021826, and WO 2003/022311.

The early regions of the adenoviral genome include the E1, E2, E3, and E4 regions. The E1 region comprises the E1A and E1B subregions, and one or more deficiencies in replication-essential gene functions in the E1 region can include one or more deficiencies in replication-essential gene functions in either or both of the E1A and E1B subregions, thereby requiring complementation of the E1A subregion and/or the E1B subregion of the adenoviral genome for the adenovirus or adenoviral vector to propagate (e.g., to form adenoviral vector particles). The E2 region comprises the E2A and E2B subregions, and one or more deficiencies in replication-essential gene functions in the E2 region can include one or more deficiencies in replication-essential gene functions in either or both of the E2A and E2B subregions, thereby requiring complementation of the E2A subregion and/or the E2B subregion of the adenoviral genome for the adenovirus or adenoviral vector to propagate (e.g., to form adenoviral vector particles).

The E3 region does not include any replication-essential gene functions, such that a deletion of the E3 region in part or in whole does not require complementation of any gene functions in the E3 region for the adenovirus or adenoviral vector to propagate (e.g., to form adenoviral vector particles). In the context of the present disclosure, the E3 region is defined as the region that initiates with the open reading frame that encodes a protein with high homology to the 12.5K protein from the E3 region of human adenovirus 5 (NCBI reference sequence AP_000218) and ends with the open reading frame that encodes a protein with high homology to the 14.7K protein from the E3 region of human adenovirus 5 (NCBI reference sequence AP_000224.1). The E3 region can be deleted in whole or in part, or retained in whole or in part. The size of the deletion can be tailored so as to retain an adenovirus or adenoviral vector whose genome closely matches the optimum genome packaging size. A larger deletion will accommodate the insertion of larger heterologous nucleic acid sequences in the adenovirus or adenoviral genome. In one embodiment of the present disclosure, the L4 polyadenylation signal sequences, which reside in the E3 region, are retained.

The E4 region comprises multiple open reading frames (ORFs). An adenovirus or adenoviral vector with a deletion of all of the open reading frames of the E4 region except ORF6, and in some cases ORF3, does not require complementation of any gene functions in the E4 region for the adenovirus or adenoviral vector to propagate (e.g., to form adenoviral vector particles). Conversely, an adenovirus or adenoviral vector with a disruption or deletion of ORF6, and in some cases ORF3, of the E4 region (e.g., with a deficiency in a replication-essential gene function based in ORF6 and/or ORF3 of the E4 region), with or without a disruption or deletion of any of the other open reading frames of the E4 region or the native E4 promoter, polyadenylation sequence, and/or the right-side inverted terminal repeat (ITR), requires complementation of the E4 region (specifically, of ORF6 and/or ORF3 of the E4 region) for the adenovirus or adenoviral vector to propagate (e.g., to form adenoviral vector particles). The late regions of the adenoviral genome include the L1, L2, L3, L4, and L5 regions. The adenovirus or adenoviral vector also can have a mutation in the major late promoter (MLP), as discussed in International Patent Application Publication WO 2000/000628, which can render the adenovirus or adenoviral vector replication-deficient if desired.

The one or more regions of the adenoviral genome that contain one or more deficiencies in replication-essential gene functions desirably are one or more early regions of the adenoviral genome, i.e., the E1, E2, and/or E4 regions, optionally with the deletion in part or in whole of the E3 region.

The replication-deficient adenovirus or adenoviral vector also can have one or more mutations as compared to the wild-type adenovirus (e.g., one or more deletions, insertions, and/or substitutions) in the adenoviral genome that do not inhibit viral replication in host cells. Thus, in addition to one or more deficiencies in replication-essential gene functions, the adenovirus or adenoviral vector can be deficient in other respects that are not replication-essential. For example, the adenovirus or adenoviral vector can have a partial or entire deletion of the adenoviral early region known as the E3 region, which is not essential for propagation of the adenovirus or adenoviral genome.

In one embodiment, the adenovirus or adenoviral vector is replication-deficient and requires, at most, complementation of the E1 region or the E4 region of the adenoviral genome, for propagation (e.g., to form adenoviral vector particles). Thus, the replication-deficient adenovirus or adenoviral vector requires complementation of at least one replication-essential gene function of the E1A subregion and/or the E1B region of the adenoviral genome (denoted an E1-deficient adenoviral vector) or the E4 region of the adenoviral genome (denoted an E4-deficient adenoviral vector) for propagation (e.g., to form adenoviral vector particles). The adenovirus or adenoviral vector can be deficient in at least one replication-essential gene function (desirably all replication-essential gene functions) of the E1 region of the adenoviral genome and at least one gene function of the nonessential E3 region of the adenoviral genome (denoted an E1/E3-deficient adenoviral vector). The adenovirus or adenoviral vector can be deficient in at least one replication-essential gene function (desirably all replication-essential gene functions) of the E4 region of the adenoviral genome and at least one gene function of the nonessential E3 region of the adenoviral genome (denoted an E3/E4-deficient adenoviral vector).

In one embodiment, the adenovirus or adenoviral vector is replication-deficient and requires, at most, complementation of the E2 region, preferably the E2A subregion, of the adenoviral genome, for propagation (e.g., to form adenoviral vector particles). Thus, the replication-deficient adenovirus or adenoviral vector requires complementation of at least one replication-essential gene function of the E2A subregion of the adenoviral genome (denoted an E2A-deficient adenoviral vector) for propagation (e.g., to form adenoviral vector particles). The adenovirus or adenoviral vector can be deficient in at least one replication-essential gene function (desirably all replication-essential gene functions) of the E2A region of the adenoviral genome and at least one gene function of the nonessential E3 region of the adenoviral genome (denoted an E2A/E3-deficient adenoviral vector).

In one embodiment, the adenovirus or adenoviral vector is replication-deficient and requires, at most, complementation of the E1 and E4 regions of the adenoviral genome for propagation (e.g., to form adenoviral vector particles). Thus, the replication-deficient adenovirus or adenoviral vector requires complementation of at least one replication-essential gene function of both the E1 and E4 regions of the adenoviral genome (denoted an E1/E4-deficient adenoviral vector) for propagation (e.g., to form adenoviral vector particles). The adenovirus or adenoviral vector can be deficient in at least one replication-essential gene function (desirably all replication-essential gene functions) of the E1 region of the adenoviral genome, at least one replication-essential gene function of the E4 region of the adenoviral genome, and at least one gene function of the nonessential E3 region of the adenoviral genome (denoted an E1/E3/E4-deficient adenoviral vector). The adenovirus or adenoviral vector preferably requires, at most, complementation of the E1 region of the adenoviral genome for propagation, and does not require complementation of any other deficiency of the adenoviral genome for propagation. More preferably, the adenovirus or adenoviral vector requires, at most, complementation of the E1 and E4 regions of the adenoviral genome for propagation, and does not require complementation of any other deficiency of the adenoviral genome for propagation.

The adenovirus or adenoviral vector, when deficient in multiple replication-essential gene functions of the adenoviral genome (e.g., an E1/E4-deficient adenoviral vector), can include a spacer sequence to provide viral growth in a complementing cell line similar to that achieved by adenoviruses or adenoviral vectors deficient in a single replication-essential gene function (e.g., an E1-deficient adenoviral vector). The spacer sequence can contain any nucleotide sequence or sequences which are of a desired length, such as sequences at least about 15 base pairs (e.g., between about 15 nucleotides and about 12,000 nucleotides), preferably about 100 nucleotides to about 10,000 nucleotides, more preferably about 500 nucleotides to about 8,000 nucleotides, even more preferably about 1,500 nucleotides to about 6,000 nucleotides, and most preferably about 2,000 to about 3,000 nucleotides in length, or a range defined by any two of the foregoing values. The spacer sequence can be coding or non-coding and native or non-native with respect to the adenoviral genome, but does not restore the replication-essential function to the deficient region. The spacer also can contain an expression cassette. More preferably, the spacer comprises a polyadenylation sequence and/or a gene that is non-native with respect to the adenovirus or adenoviral vector. The use of a spacer in an adenoviral vector is further described in, for example, U.S. Pat. No. 5,851,806 and International Patent Application Publication WO 1997/021826.

By removing all or part of the adenoviral genome, for example, the E1, E3, and E4 regions of the adenoviral genome, the resulting adenovirus or adenoviral vector is able to accept inserts of exogenous nucleic acid sequences while retaining the ability to be packaged into adenoviral capsids. An exogenous nucleic acid sequence can be inserted at any position in the adenoviral genome so long as insertion in the position allows for the formation of adenovirus or the adenoviral vector particle. The exogenous nucleic acid sequence preferably is positioned in the E1 region, the E3 region, or the E4 region of the adenoviral genome.

The replication-deficient adenovirus or adenoviral vector of the present disclosure can be produced in complementing cell lines that provide gene functions not present in the replication-deficient adenovirus or adenoviral vector, but required for viral propagation, at appropriate levels in order to generate high titers of viral vector stock. Such complementing cell lines are known and include, but are not limited to, 293 cells (described in, e.g., Graham et al., J. Gen. Virol., 36:59-72 (1977)), PER.C6 cells (described in, e.g., International Patent Application Publication WO 1997/000326, and U.S. Pat. Nos. 5,994,128 and 6,033,908), and 293-ORF6 cells (described in, e.g., International Patent Application Publication WO 95/34671 and Brough et al., J. Virol., 71:9206-9213 (1997)). Other suitable complementing cell lines to produce the replication-deficient adenovirus or adenoviral vector of the present disclosure include complementing cells that have been generated to propagate adenoviral vectors encoding transgenes whose expression inhibits viral growth in host cells (see, e.g., U.S. Patent Application Publication No. 2008/0233650). Additional suitable complementing cells are described in, for example, U.S. Pat. Nos. 6,677,156 and 6,682,929, and International Patent Application Publication WO 2003/020879. In some instances, the cellular genome need not comprise nucleic acid sequences, the gene products of which complement for all of the deficiencies of a replication-deficient adenoviral vector. One or more replication-essential gene functions lacking in a replication-deficient adenoviral vector can be supplied by a helper virus, e.g., an adenoviral vector that supplies in trans one or more essential gene functions required for replication of the replication-deficient adenovirus or adenoviral vector. Alternatively, the inventive adenovirus or adenoviral vector can comprise a non-native replication-essential gene that complements for the one or more replication-essential gene functions lacking in the inventive replication-deficient adenovirus or adenoviral vector. For example, an E1/E4-deficient adenoviral vector can be engineered to contain a nucleic acid sequence encoding E4 ORF 6 that is obtained or derived from a different adenovirus (e.g., an adenovirus of a different serotype than the inventive adenovirus or adenoviral vector, or an adenovirus of a different species than the inventive adenovirus or adenoviral vector).

The adenovirus or adenoviral vector can further comprise a transgene. The term “transgene” is defined herein as a non-native nucleic acid sequence that is operably linked to appropriate regulatory elements (e.g., a promoter), such that the non-native nucleic acid sequence can be expressed to produce a protein (e.g., peptide or polypeptide). The regulatory elements (e.g., promoter) can be native or non-native to the adenovirus or adenoviral vector.

A “non-native” nucleic acid sequence is any nucleic acid sequence (e.g., DNA, RNA, or cDNA sequence) that is not a naturally occurring nucleic acid sequence of an adenovirus in a naturally occurring position. Thus, the non-native nucleic acid sequence can be naturally found in an adenovirus, but located at a non-native position within the adenoviral genome and/or operably linked to a non-native promoter. The terms “non-native nucleic acid sequence,” “heterologous nucleic acid sequence,” and “exogenous nucleic acid sequence” are synonymous and can be used interchangeably in the context of the present disclosure. The non-native nucleic acid sequence preferably is DNA and preferably encodes a protein (i.e., one or more nucleic acid sequences encoding one or more proteins).

The non-native nucleic acid sequence can encode a therapeutic protein that can be used to prophylactically or therapeutically treat a mammal for a disease. Examples of suitable therapeutic proteins include cytokines, toxins, tumor suppressor proteins, growth factors, hormones, receptors, mitogens, immunoglobulins, neuropeptides, neurotransmitters, and enzymes. Alternatively, the non-native nucleic acid sequence can encode an antigen of a pathogen (e.g., a bacterium or a virus), and the adenovirus or adenoviral vector can be used as a vaccine.

### Viral Based Delivery System

The present disclosure also provides delivery systems, such as viral-based systems, in which a nucleic acid described herein is inserted. Representative viral expression vectors include, but are not limited to, adeno-associated viral vectors, adenovirus-based vectors, lentivirus-based vectors, retroviral vectors, and herpes virus-based vectors. In an embodiment, the viral vector is a lentivirus vector. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity. In an additional embodiment, the viral vector is an adeno-associated viral vector. In a further embodiment, the viral vector is a retroviral vector. In general, and in embodiments, a suitable vector contains an origin of replication functional in at least one organism, a promoter sequence, convenient restriction endonuclease sites, and one or more selectable markers.

Additional suitable vectors include integrating expression vectors, which can randomly integrate into the host cell's DNA, or can include a recombination site to enable the specific recombination between the expression vector and the host cell's chromosome. Such integrating expression vectors can utilize the endogenous expression control sequences of the host cell's chromosomes to effect expression of the desired protein. Examples of vectors that integrate in a site specific manner include, for example, components of the flp-in system from Invitrogen (Carlsbad, Calif.) (e.g., pcDNATM5/FRT), or the cre-lox system, such as can be found in the pExchange-6 Core Vectors from Stratagene (La Jolla, Calif.). Examples of vectors that randomly integrate into host cell chromosomes include, for example, pcDNA3.1 (when introduced in the absence of T-antigen) from Invitrogen (Carlsbad, Calif.), and pCI or pFN10A (ACT) FLEXITM from Promega (Madison, Wis.). Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto.

However, other constitutive promoter sequences can also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the present disclosure should not be limited to the use of constitutive promoters. Inducible promoters are also contemplated as part of the present disclosure. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

Reporter genes can be used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes can include genes encoding luciferase, beta-galactosidase, chloramphenicol acetyl transferase, secreted alkaline phosphatase, or the green fluorescent protein gene (e.g., Ui-Tei et al., FEBS Letters 479:79-82 (2000)). Suitable expression systems are well known and can be prepared using known techniques or obtained commercially. In general, the construct with the minimal 5′ flanking region showing the highest level of expression of reporter gene is identified as the promoter. Such promoter regions can be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (Molecular Cloning: A Laboratory Manual, Cold Spring Harbor Laboratory, New York (2001)). In embodiments, a method for the introduction of a polynucleotide into a host cell is calcium phosphate transfection or polyethylenimine (PEI) Transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

### Non-Viral Based Delivery System

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

The use of lipid formulations is contemplated for the introduction of the nucleic acids into a host cell (in vitro, ex vivo or in vivo). In another aspect, the nucleic acid can be associated with a lipid. The nucleic acid associated with a lipid can be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they can be present in a bilayer structure, as micelles, or with a “collapsed” structure. They can also simply be interspersed in a solution, possibly forming aggregates that are not uniform in size or shape. Lipids are fatty substances which can be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids can be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., Glycobiology 5:505-10 (1991)). However, compositions that have different structures in solution than the normal vesicular structure are also encompassed. For example, the lipids can assume a micellar structure or merely exist as nonuniform aggregates of lipid molecules. Also contemplated are lipofectamine-nucleic acid complexes.

In some instances, polynucleotides encoding polypeptides can also be introduced into cells using non-viral based delivery systems, such as the “Sleeping Beauty (SB) Transposon System,” which refers a synthetic DNA transposon system for introducing DNA sequences into the chromosomes of vertebrates. Some exemplary embodiments of the system are described, for example, in U.S. Pat. Nos. 6,489,458 and 8,227,432. The Sleeping Beauty transposon system is composed of a Sleeping Beauty (SB) transposase and a SB transposon. In embodiments, the Sleeping Beauty transposon system can include the SB11 transposon system, the SB100X transposon system, or the SB110 transposon system.

DNA transposons translocate from one DNA site to another in a simple, cut-and-paste manner. Transposition is a precise process in which a defined DNA segment is excised from one DNA molecule and moved to another site in the same or different DNA molecule or genome. As do other Tc1/mariner-type transposases, SB transposase inserts a transposon into a TA dinucleotide base pair in a recipient DNA sequence. The insertion site can be elsewhere in the same DNA molecule, or in another DNA molecule (or chromosome). In mammalian genomes, including humans, there are approximately 200 million TA sites. The TA insertion site is duplicated in the process of transposon integration. This duplication of the TA sequence is a hallmark of transposition and used to ascertain the mechanism in some experiments. The transposase can be encoded either within the transposon or the transposase can be supplied by another source, for instance a DNA or mRNA source, in which case the transposon becomes a non-autonomous element. Non-autonomous transposons are most useful as genetic tools because after insertion they cannot independently continue to excise and re-insert. SB transposons envisaged to be used as non-viral vectors for introduction of genes into genomes of vertebrate animals and for gene therapy.

Regardless of the method used to introduce exogenous nucleic acids into a host cell or otherwise expose a cell to the inhibitor of the present disclosure, in order to confirm the presence of the recombinant DNA sequence in the host cell, a variety of assays can be performed. Such assays include, for example, molecular assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the present disclosure.

In embodiments, a modified effector cell described herein and other genetic elements are delivered to a cell using the SB11 transposon system, the SB100X transposon system, the SB110 transposon system, the piggyBac transposon system (see, e.g., Wilson et al, “PiggyBac Transposon-mediated Gene Transfer in Human Cells,” Molecular Therapy 15:139-145 (2007), incorporated herein by reference in its entirety) and/or the piggyBac transposon system (see, e.g., Mitra et al., “Functional characterization of piggyBac from the bat Myotis lucifugus unveils an active mammalian DNA transposon,” Proc. Natl. Acad. Sci USA 110:234-239 (2013). Additional transposases and transposon systems are provided in U.S. Pat. Nos. 6,489,458; 6,613,752, 7,148,203; 7,985,739; 8,227,432; 9,228,180; U.S. Patent Pub. No. 2011/0117072; Mates et al., Nat Genet, 41(6):753-61 (2009). doi: 10.1038/ng.343. Epub 2009 May 3, Gene Ther., 18(9):849-56 (2011). doi: 10.1038/gt.2011.40. Epub 2011 Mar. 31 and in Ivics et al., Cell, 91(4):501-10, (1997), each of which is incorporated herein by reference in their entirety.

Additional suitable non-viral systems can include integrating expression vectors, which can randomly integrate into the host cell's DNA, or can include a recombination site to enable the specific recombination between the expression vector and the host cell's chromosome. Targeted integration of transgenes into predefined genetic loci is a desirable goal for many applications. First, a first recombination site for a site-specific recombinase is inserted at a genomic site, either at a random or at a predetermined location. Subsequently, the cells are transfected with a plasmid carrying the gene or DNA of interest and the second recombination site and a source for recombinase (expression plasmid, RNA, protein, or virus-expressing recombinase). Recombination between the first and second recombination sites leads to integration of plasmid DNA.

Such integrating expression vectors can utilize the endogenous expression control sequences of the host cell's chromosomes to effect expression of the desired protein. In some embodiments, targeted integration is promoted by the presence of sequences on the donor polynucleotide that are homologous to sequences flanking the integration site. For example, targeted integration using the donor polynucleotides described herein can be achieved following conventional transfection techniques, e.g. techniques used to create gene knockouts or knockins by homologous recombination. In other embodiments, targeted integration is promoted both by the presence of sequences on the donor polynucleotide that are homologous to sequences flanking the integration site, and by contacting the cells with donor polynucleotide in the presence of a site-specific recombinase. By a site-specific recombinase, or simply a recombinase, it is meant is a polypeptide that catalyzes conservative site-specific recombination between its compatible recombination sites. As used herein, a site-specific recombinase includes native polypeptides as well as derivatives, variants and/or fragments that retain activity, and native polynucleotides, derivatives, variants, and/or fragments that encode a recombinase that retains activity.

Also provided herein is a system for integrating heterologous genes in a host cell, said system comprising one or more gene expression cassettes. In some instances, the system includes a first gene expression cassette comprising a first polynucleotide encoding a first polypeptide construct. In other instances, the system can include a second gene expression cassette comprising a second polynucleotide encoding a second polypeptide construct. In yet other instances, the system can include a third gene expression cassette. In one embodiment, one of the gene expression cassettes can comprise a gene switch polynucleotide encoding one or more of: (i) a transactivation domain; (ii) nuclear receptor ligand binding domain; (iii) a DNA-binding domain; and (iv) ecdysone receptor binding domain. In another embodiment, the system further includes recombinant attachment sites; and a serine recombinase; such that upon contacting said host cell with at least said first gene expression cassette, in the presence of said serine recombinase, said heterologous genes are integrated in said host cell.

In some instances, the system further comprises a ligand; such that upon contacting said host cell, in the presence of said ligand, said heterologous gene are expressed in said host cell. In one instance, the system also includes recombinant attachment sites. In some instances, one recombination attachment site is a phage genomic recombination attachment site (attP) or a bacterial genomic recombination attachment site (attB). In one instance, the host cell is an eukaryotic cell. In another instance, the host cell is a human cell. In further instances, the host cell is a T cell or NK cell.

### Promoters

“Promoter” refers to a region of a polynucleotide that initiates transcription of a coding sequence. Promoters are located near the transcription start sites of genes, on the same strand and upstream on the DNA (towards the 5′ region of the sense strand). Some promoters are constitutive as they are active in all circumstances in the cell, while others are regulated becoming active in response to specific stimuli, e.g., an inducible promoter. Yet other promoters are tissue specific or activated promoters, including but not limited to T-cell specific promoters.

The term “promoter activity” and its grammatical equivalents as used herein refer to the extent of expression of nucleotide sequence that is operably linked to the promoter whose activity is being measured. Promoter activity can be measured directly by determining the amount of RNA transcript produced, for example by Northern blot analysis or indirectly by determining the amount of product coded for by the linked nucleic acid sequence, such as a reporter nucleic acid sequence linked to the promoter.

“Inducible promoter” as used herein refers to a promoter which is induced into activity by the presence or absence of transcriptional regulators, e.g., biotic or abiotic factors. Inducible promoters are useful because the expression of genes operably linked to them can be turned on or off at certain stages of development of an organism or in a particular tissue. Examples of inducible promoters are alcohol-regulated promoters, tetracycline-regulated promoters, steroid-regulated promoters, metal-regulated promoters, pathogenesis-regulated promoters, temperature-regulated promoters and light-regulated promoters. In one embodiment, the inducible promoter is part of a genetic switch. The inducible promoter can be a gene switch ligand inducible promoter. In some cases, an inducible promoter can be a small molecule ligand-inducible two polypeptide ecdysone receptor-based gene switch, such as RHEOSWITCH® gene switch. In some cases, a gene switch can be selected from ecdysone-based receptor components as described in, but without limitation to, any of the systems described in: PCT/US2001/009050 (WO 2001/070816); U.S. Pat. Nos. 7,091,038; 7,776,587; 7,807,417; 8,202,718; PCT/US2001/030608 (WO 2002/029075); U.S. Pat. Nos. 8,105,825; 8,168,426; PCT/1J52002/005235 (WO 2002/066613); U.S. application Ser. No. 10/468,200 (U.S. Pub. No. 20120167239); PCT/US2002/005706 (WO 2002/066614); U.S. Pat. Nos. 7,531,326; 8,236,556; 8,598,409; PCT/US2002/005090 (WO 2002/066612); U.S. Pat. No. 8,715,959 (U.S. Pub. No. 20060100416); PCT/US2002/005234 (WO 2003/027266); U.S. Pat. Nos. 7,601,508; 7,829,676; 7,919,269; 8,030,067; PCT/US2002/005708 (WO 2002/066615); U.S. application Ser. No. 10/468,192 (U.S. Pub. No. 20110212528); PCT/US2002/005026 (WO 2003/027289); U.S. Pat. Nos. 7,563,879; 8,021,878; 8,497,093; PCT/US2005/015089 (WO 2005/108617); U.S. Pat. Nos. 7,935,510; 8,076,454; PCT/US2008/011270 (WO 2009/045370); U.S. application Ser. No. 12/241,018 (U.S. Pub. No. 20090136465); PCT/US2008/011563 (WO 2009/048560); U.S. application Ser. No. 12/247,738 (U.S. Pub. No. 20090123441); PCT/US2009/005510 (WO 2010/042189); U.S. application Ser. No. 13/123,129 (U.S. Pub. No. 20110268766); PCT/US2011/029682 (WO 2011/119773); U.S. application Ser. No. 13/636,473 (U.S. Pub. No. 20130195800); PCT/US2012/027515 (WO 2012/122025); and, U.S. Pat. No. 9,402,919 each of which is incorporated by reference in its entirety).

Provided herein are methods comprising administering to a subject at least one non-viral vector comprising a polynucleotide encoding a polypeptide sequence described herein comprising at least two functional proteins or portions thereof; at least one promoter; and at least one engineered recombination site; wherein said at least one promoter drives expression of said at least two functional proteins. In some cases, at least one promoter can be constitutive. In some cases, at least one promoter can be tissue-specific. In some cases, at least one promoter can be inducible. In some cases, an inducible promoter is a small molecule ligand-inducible two polypeptide ecdysone receptor-based gene switch. In other cases, a combination of promoters wherein at least one promoter can be inducible and at least one promoter can be activation specific can be utilized.

An inducible promoter utilizes a ligand for dose-regulated control of expression of said at least two genes. In some cases, a ligand can be selected from a group consisting of ecdysteroid, 9-cis-retinoic acid, synthetic analogs of retinoic acid, N,N′-diacylhydrazines, oxadiazolines, dibenzoylalkyl cyanohydrazines, N-alkyl-N,N′-diaroylhydrazines, N-acyl-N-alkylcarbonylhydrazines, N-aroyl-N-alkyl-N′-aroylhydrazines, arnidoketones, 3,5-di-tert-butyl-4-hydroxy-N-isobutyl-benzamide, 8-O-acetylharpagide, oxysterols, 22(R) hydroxycholesterol, 24 (S) hydroxycholesterol, 25-epoxycholesterol, T0901317, 5-alpha-6-alpha-epoxycholesterol-3-sulfate (ECHS), 7-ketocholesterol-3-sulfate, framesol, bile acids, 1,1-biphosphonate esters, juvenile hormone III, RG-115819 (3,5-Dimethyl-benzoic acid N-(1-ethyl-2,2-dimethyl-propyl)-N′-(2-methyl-3-methoxy-benzoyl)-hydrazide-), RG-115932 ((R)-3,5-Dimethyl-benzoic acid N-(1-tert-butyl-butyl)-N′-(2-ethyl-3-methoxy-benzoyl)-hydrazide), and RG-115830 (3,5-Dimethyl-benzoic acid N-(1-tert-butyl-butyl)-N′-(2-ethyl-3-methoxy-benzoyl)-hydrazide), and any combination thereof.

In some embodiments, a promoter is an inducible promoter. In some embodiments, a promoter is a non-inducible promoter. In some cases, a promoter can be a tissue-specific promoter. Herein “tissue-specific” refers to regulated expression of a gene in a subset of tissues or cell types. In some cases, a tissue-specific promoter can be regulated spatially such that the promoter drives expression only in certain tissues or cell types of an organism. In other cases, a tissue-specific promoter can be regulated temporally such that the promoter drives expression in a cell type or tissue differently across time, including during development of an organism. In some cases, a tissue-specific promoter is regulated both spatially and temporally. In certain embodiments, a tissue-specific promoter is activated in certain cell types either constitutively or intermittently at particular times or stages of the cell type. For example, a tissue-specific promoter can be a promoter that is activated when a specific cell such as a T cell or a NK cell is activated. T cells can be activated in a variety of ways, for example, when presented with peptide antigens by MHC class II molecules.

In one case, at least one promoter is an engineered promoter or variants thereof. As described herein, the promoter can incorporate minimal promoter sequences from IL-2 and one or more of the following: nuclear factor of activated T-cells (NFAT) response element(s); NFIL2D response element, NFkB/TCF response element, NF_AT/NFIL2B response element or NFIL2A/OCT response element. Examples of response elements are described in Mattila et al., EMBO J. 9(13):4425-33 (1990); incorporated herein in its entirety.

In some embodiments, at least one promoter comprises IL-2 core promoter (SEQ ID NO: 26). In one embodiment, at least one promoter comprises IL-2 minimal promoter (SEQ ID NO: 27). In another embodiment, at least one promoter comprises IL-2 enhancer and promoter variant (SEQ ID NOS: 26-28). In yet another embodiment, at least one promoter comprises NF-κB binding site (SEQ ID NOS: 30-32). In some embodiments, at least one promoter comprises (NF-κB)₁-IL2 promoter variant (SEQ ID NO: 30). In some embodiments, at least one promoter comprises (NF-κB)₃-IL2 promoter variant (SEQ ID NO: 31). In some embodiments, at least one promoter comprises (NF-κB)₆-IL2 promoter variant (SEQ ID NO: 32). In one embodiment, at least one promoter comprises 1× nuclear factor of activated T-cells (NFAT) response elements-IL2 promoter variant (SEQ ID NO: 33). In another embodiment, at least one promoter comprises 3×NFAT response element (SEQ ID NOS: 34-35). In yet another embodiment, at least one promoter comprises 6×NFAT response elements-IL2 promoter variant (SEQ ID NOS: 36-39). In some embodiments, at least one promoter comprises human EF1A1 promoter variant (SEQ ID NOS: 40-41). In some embodiment, at least one promoter comprises human EF1A1 promoter and enhancer (SEQ ID NO: 42). In some embodiments, at least one promoter comprises human UBC promoter (SEQ ID NO: 43). In some embodiments, at least one promoter comprises 6 site GAL4-inducible proximal factor binding element (PFB). In some embodiment, at least one promoter comprises synthetic minimal promoter 1 (inducible promoter) (SEQ ID NO: 44).

Use of gene switch for ligand inducible control of IL-12 expression described herein can improve the safety profile of IL-12 by for example allowing for regulated expression and improving therapeutic index. However, a condition for ligand dose dependent expression of IL-12 using gene switch(es) is the presence or absence of activator ligand (e.g. veledimex). In certain embodiments, an additional conditional control for induction of IL-12 expression is contemplated. Gene switch components under the control of T cell activated specific promoters are provided. This results in conditional expression (e.g., T cell activation) of gene switch components necessary for veledimex controlled expression of transgene(s) under control of a gene switch. In some embodiments, this results in preferential expression of cytokines such as IL-12 or IL-15 by tumor specific T cells when veledimex is present and T cells are activated. This can lead to increased localized levels of gene switch controlled transgene expression.

For example, T cell activation specific expression of gene switch components can be controlled by promoter comprising Nuclear Factor of Activated T-cells (NFAT) response element(s). NFAT transcription factors are key modulators of effector T-cell states. NFATs are early transcriptional checkpoint progressively driving exhaustion. NFATs are quickly activated in T cells following TCR stimulation and form a protein complex with AP-1 induced by appropriate co-stimulation signaling and regulate effector genes and T-cell functions. NFAT response element(s) can be fused with other minimal promoter sequences (e.g. IL2 minimal promoter) to drive expression of transgenes in response to T cell activation.

Other examples of activation specific promoters include but are not limited to interleukin-2 (IL2) promoter and Programmed Death (PD)-1 (CD279) promoter. Gene switch components can also be conditionally expressed upon immune cell activation by fusing binding sites for other nuclear factors like NF-κB of proinflammatory signaling pathway to minimal promoter sequence (e.g. IL2).

In certain embodiments, the promoter can be any one or more of: IL-2 core promoter, IL-2 minimal promoter, IL-2 enhancer and promoter variant, (NF-κB)₁-IL2 promoter variant, (NF-κB)₃-IL2 promoter variant, (NF-κB)₆-IL2 promoter variant, 1×NFAT response elements-IL2 promoter variant, 3×NFAT response elements-IL2 promoter variant, 6×NFAT response elements-IL2 promoter variant, human EEF1A1 promoter variant, human EEF1A1 promoter and enhancer, human UBC promoter and synthetic minimal promoter 1. In certain embodiments, the promoter nucleotides can comprise SEQ ID NOs: 26-44.

### Gene Switch

Provided herein are gene switch polypeptides, polynucleotides encoding ligand-inducible gene switch polypeptides, and methods and systems incorporating these polypeptides and/or polynucleotides. In certain aspects, the present disclosure is directed to a polynucleotide comprising one or more polynucleotides encoding a gene switch system for inducible control of heterologous gene expression, wherein the heterologous gene expression is regulated by said gene switch system; and, wherein said heterologous gene comprises a polynucleotide encoding a polypeptide comprising one or more immune response-inducing human papilloma virus (HPV) polypeptides, disclosed herein.

The term “gene switch” refers to the combination of a response element associated with a promoter, and for instance, an EcR based system which, in the presence of one or more ligands, modulates the expression of a gene into which the response element and promoter are incorporated. Tightly regulated inducible gene expression systems or gene switches are useful for various applications such as gene therapy, large scale production of proteins in cells, cell based high throughput screening assays, functional genomics and regulation of traits in transgenic plants and animals. Such inducible gene expression systems can include ligand inducible heterologous gene expression systems.

An early version of EcR-based gene switch used Drosophila melanogaster EcR (DmEcR) and Mus musculus RXR (MmRXR) polypeptides and showed that these receptors in the presence of steroid, ponasteroneA, transactivate reporter genes in mammalian cell lines and transgenic mice (Christopherson et al., Proc. Natl. Acad. Sci. USA 89(14):6314-18 (1992); No et al., Proc. Natl. Acad. Sci. USA 93(8):3346-51 (1996)). Later, Suhr et al. (Proc. Natl. Acad. Sci. USA 95(14):7999-8004 (1998)) showed that non-steroidal ecdysone agonist, tebufenozide, induced high level of transactivation of reporter genes in mammalian cells through Bombyx mori EcR (BmEcR) in the absence of exogenous heterodimer partner.

International Patent Applications No. PCT/US97/05330 (WO 97/38117) and PCT/US99/08381 (WO99/58155) disclose methods for modulating the expression of an exogenous gene in which a DNA construct comprising the exogenous gene and an ecdysone response element is activated by a second DNA construct comprising an ecdysone receptor that, in the presence of a ligand therefor, and optionally in the presence of a receptor capable of acting as a silent partner, binds to the ecdysone response element to induce gene expression. In this example, the ecdysone receptor was isolated from Drosophila melanogaster. Typically, such systems require the presence of the silent partner, preferably retinoid X receptor (RXR), in order to provide optimum activation. In mammalian cells, insect ecdysone receptor (EcR) is capable of heterodimerizing with mammalian retinoid X receptor (RXR) and, thereby, be used to regulate expression of target genes or heterologous genes in a ligand dependent manner. International Patent Application No. PCT/US98/14215 (WO 99/02683) discloses that the ecdysone receptor isolated from the silk moth Bombyx mori is functional in mammalian systems without the need for an exogenous dimer partner.

U.S. Pat. No. 6,265,173 discloses that various members of the steroid/thyroid superfamily of receptors can combine with Drosophila melanogaster ultraspiracle receptor (USP) or fragments thereof comprising at least the dimerization domain of USP for use in a gene expression system. U.S. Pat. No. 5,880,333 discloses a Drosophila melanogaster EcR and ultraspiracle (USP) heterodimer system used in plants in which the transactivation domain and the DNA binding domain are positioned on two different hybrid proteins. In each of these cases, the transactivation domain and the DNA binding domain (either as native EcR as in International Patent Application No. PCT/US98/14215 or as modified EcR as in International Patent Application No. PCT/US97/05330) were incorporated into a single molecule and the other heterodimeric partners, either USP or RXR, were used in their native state.

International Patent Application No. PCT/US01/0905 discloses an ecdysone receptor-based inducible gene expression system in which the transactivation and DNA binding domains are separated from each other by placing them on two different proteins results in greatly reduced background activity in the absence of a ligand and significantly increased activity over background in the presence of a ligand. This two-hybrid system is a significantly improved inducible gene expression modulation system compared to the two systems disclosed in applications PCT/US97/05330 and PCT/US98/14215. The two-hybrid system is believed to exploit the ability of a pair of interacting proteins to bring the transcription activation domain into a more favorable position relative to the DNA binding domain such that when the DNA binding domain binds to the DNA binding site on the gene, the transactivation domain more effectively activates the promoter (see, for example, U.S. Pat. No. 5,283,173). The two-hybrid gene expression system comprises two gene expression cassettes; the first encoding a DNA binding domain fused to a nuclear receptor polypeptide, and the second encoding a transactivation domain fused to a different nuclear receptor polypeptide. In the presence of ligand, it is believed that a conformational change is induced which promotes interaction of the first polypeptide with the second polypeptide thereby resulting in dimerization of the DNA binding domain and the transactivation domain. Since the DNA binding and transactivation domains reside on two different molecules, the background activity in the absence of ligand is greatly reduced.

Another surprising discovery was that certain modifications of the two-hybrid system could also provide improved sensitivity to non-steroidal ligands for example, diacylhydrazines, when compared to steroidal ligands for example, ponasterone A (“PonA”) or muristerone A (“MurA”). That is, when compared to steroids, the non-steroidal ligands provided higher gene transcription activity at a lower ligand concentration. Furthermore, the two-hybrid system avoids some side effects due to overexpression of RXR that can occur when unmodified RXR is used as a switching partner. In a preferred two-hybrid system, native DNA binding and transactivation domains of EcR or RXR are eliminated and as a result, these hybrid molecules have less chance of interacting with other steroid hormone receptors present in the cell, thereby resulting in reduced side effects.

The ecdysone receptor (EcR) is a member of the nuclear receptor superfamily and is classified into subfamily 1, group H (referred to herein as “Group H nuclear receptors”). The members of each group share 40-60% amino acid identity in the E (ligand binding) domain (Laudet et al., A Unified Nomenclature System for the Nuclear Receptor Subfamily, 1999; Cell 97:161-163). In addition to the ecdysone receptor, other members of this nuclear receptor subfamily 1, group H include: ubiquitous receptor (UR), Orphan receptor 1 (OR-1), steroid hormone nuclear receptor 1 (NER-1), RXR interacting protein-15 (RIP-15), liver x receptor β (LXRβ), steroid hormone receptor like protein (RLD-1), liver x receptor (LXR), liver x receptor α (LXRα), farnesoid x receptor (FXR), receptor interacting protein 14 (RIP-14), and farnesol receptor (HRR-1).

In some cases, an inducible promoter can be a small molecule ligand-inducible two polypeptide ecdysone receptor-based gene switch, such as Intrexon Corporation's RHEOSWITCH® gene switch. In some cases, a gene switch can be selected from ecdysone-based receptor components as described in, but without limitation to, any of the systems described in: PCT/US2001/009050 (WO 2001/070816); U.S. Pat. Nos. 7,091,038; 7,776,587; 7,807,417; 8,202,718; PCT/US2001/030608 (WO 2002/029075); U.S. Pat. Nos. 8,105,825; 8,168,426; PCT/1J52002/005235 (WO 2002/066613); U.S. application Ser. No. 10/468,200 (U.S. Pub. No. 20120167239); PCT/US2002/005706 (WO 2002/066614); U.S. Pat. Nos. 7,531,326; 8,236,556; 8,598,409; PCT/US2002/005090 (WO 2002/066612); U.S. Pat. No. 8,715,959 (U.S. Pub. No. 20060100416); PCT/US2002/005234 (WO 2003/027266); U.S. Pat. Nos. 7,601,508; 7,829,676; 7,919,269; 8,030,067; PCT/US2002/005708 (WO 2002/066615); U.S. application Ser. No. 10/468,192 (U.S. Pub. No. 20110212528); PCT/US2002/005026 (WO 2003/027289); U.S. Pat. Nos. 7,563,879; 8,021,878; 8,497,093; PCT/US2005/015089 (WO 2005/108617); U.S. Pat. Nos. 7,935,510; 8,076,454; PCT/US2008/011270 (WO 2009/045370); U.S. application Ser. No. 12/241,018 (U.S. Pub. No. 20090136465); PCT/US2008/011563 (WO 2009/048560); U.S. application Ser. No. 12/247,738 (U.S. Pub. No. 20090123441); PCT/US2009/005510 (WO 2010/042189); U.S. application Ser. No. 13/123,129 (U.S. Pub. No. 20110268766); PCT/US2011/029682 (WO 2011/119773); U.S. application Ser. No. 13/636,473 (U.S. Pub. No. 20130195800); PCT/US2012/027515 (WO 2012/122025); and, U.S. Pat. No. 9,402,919 each of which is incorporated by reference in its entirety.

Provided are systems for modulating the expression of a heterologous gene and an interleukin in a host cell, comprising polynucleotides expressing gene-switch polypeptides disclosed herein.

In some embodiments are systems for modulating the expression of a heterologous gene and a cytokine in a host cell, comprising a first gene expression cassette comprising a first polynucleotide encoding a first polypeptide; a second gene expression cassette comprising a second polynucleotide encoding a second polypeptide; and a ligand; wherein said first and second polypeptides comprise one or more of: (i) a transactivation domain; (ii) a DNA-binding domain; and (iii) a ligand binding domain; (iv) said heterologous gene; and (vi) said cytokine such that upon contacting said host cell with said first gene expression cassette and said second gene expression cassette in the presence of said ligand, said heterologous gene and said cytokine are expressed in said host cell. In some cases, the heterologous gene comprises an antigen binding polypeptide described herein. In some cases, the cytokine comprises at least one chemokine, interferon, interleukin, lymphokine, tumor necrosis factor, or variant or combination thereof. In some cases, the cytokine is an interleukin. In some cases the interleukin is at least one of IL12, IL2, IL15, IL21, and functional variants and fragments thereof. In some embodiments, the cytokines can be membrane bound or secreted. In other embodiments, the cytokines can be intracellular. The interleukin can comprise membrane bound IL-15 (mbIL-15) or a fusion of IL-15 and IL-15Rα. In some embodiments, a mbIL-15 is a membrane-bound chimeric IL-15 which can be co-expressed with a modified effector cell described herein. In some embodiments, the mbIL-15 comprises a full-length IL-15 (e.g., a native IL-15 polypeptide) or fragment or variant thereof, fused in frame with a full length IL-15Rα, functional fragment or variant thereof. In some cases, the IL-15 is indirectly linked to the IL-15Rα through a linker. In some instances, the mbIL-15 is as described in Hurton et al., “Tethered IL-15 augments antitumor activity and promotes a stem-cell memory subset in tumor-specific T cells,” Proc. Natl. Acad. Sci. USA 113(48):E7788-E7797 (2016). In another aspect, the interleukin can comprise IL-12. In some embodiments, the IL-12 is a single chain IL-12 (scIL-12), protease sensitive IL-12, destabilized IL-12, membrane bound IL-12, intercalated IL-12. In some instances, the IL-12 variants are as described in WO2015/095249, WO2016/048903, WO2017/062953, all of which is incorporated by reference in their entireties.

Provided herein are polynucleotides encoding gene switch polypeptides, wherein said gene switch polypeptides comprise: a) a first gene switch polypeptide comprising a DNA-binding domain fused to a nuclear receptor ligand binding domain, and b) a second gene switch polypeptide comprising a transactivation domain fused to a nuclear receptor ligand binding domain, wherein the first gene switch polypeptide and the second gene switch polypeptide are connected by a linker. In some cases, the linker can be a linker described herein, for instance GSG linker, furinlink, a 2A linker such as F/T2A, T2A, p2A, GSG-p2A, variants and derivatives thereof. In other instances, the linker can be an IRES.

In some cases, the DNA binding domain (DBD) comprises a DBD described herein, for instance at least one of GAL4 (GAL4 DBD), a LexA DBD, a transcription factor DBD, a steroid/thyroid hormone nuclear receptor superfamily member DBD, a bacterial LacZ DBD, and a yeast DBD. The transactivation domain can comprise a transactivation domain described herein, for instance one of a VP16 transactivation domain, a p53 transactivation domain and a B42 acidic activator transactivation domain. The Nuclear receptor ligand binding domain can comprise at least one of a ecdysone receptor (EcR), a ubiquitous receptor, an orphan receptor 1, a NER-1, a steroid hormone nuclear receptor 1, a retinoid X receptor interacting protein-15, a liver X receptor β, a steroid hormone receptor like protein, a liver X receptor, a liver X receptor α, a farnesoid X receptor, a receptor interacting protein 14, and a famesol receptor.

In some cases, the gene switch polypeptides connected by a polypeptide linker or ribosome-skipping sequence exhibit improved dose-dependent ligand-inducible control of gene expression compared to a ligand-inducible gene switch wherein the gene switch polypeptides are connected by non-coding sequences, such as an IRES. In some cases, the gene switch polypeptides connected by a 2A linker can exhibit improved dose-dependent ligand-inducible control of heterologous gene expression compared to a gene switch wherein said gene switch polypeptides are separated by an IRES.

In some embodiments, the gene switch comprises a VP16 transactivation domain. In one embodiment, the gene switch comprises at least one of an ecdysone receptor (EcR), a ubiquitous receptor, an orphan receptor 1, a NER-1, a steroid hormone nuclear receptor 1, a retinoid X receptor interacting protein-15, a liver X receptor β, a steroid hormone receptor like protein, a liver X receptor, a liver X receptor α, a farnesoid X receptor, a receptor interacting protein 14, and a famesol receptor. In another embodiment, a DNA-binding domain (DBD) of the gene switch comprises at least one of GAL4 (GAL4 DBD), a LexA DBD, a transcription factor DBD, a steroid/thyroid hormone nuclear receptor superfamily member DBD, a bacterial LacZ DBD, and a yeast DBD. In yet another case, the gene switch further comprises at least one of ultraspiracle protein (USP), retinoid receptor X (RXR), functional fragments and variants thereof wherein said functional fragments and variants are capable of binding to an EcR.

The polypeptides and polynucleotides as described herein can be expressed in an engineered cell. Herein an engineered cell is a cell which has been modified from its natural or endogenous state. An example of an engineered cell is a cell described herein which has been modified (e.g., by transfection of a polynucleotide into the cell) to encode for example, gene switch polypeptides, gene of interest (GOI), cell tags, heterologous genes and any other polypeptides and polynucleotides described herein.

### Ligands

In some embodiments, a ligand used for inducible gene switch regulation can be selected from any of, but without limitation to, following: N-[(1R)-1-(1,1-dimethylethyl)butyl]-N′-(2-ethyl-3-methoxybenzoyl)-3,5-dimethylbenzohydrazide (also referred to as veledimex), (2S,3R,5R,9R,10R,13R,14S,17R)-17-[(2S,3R)-3,6-dihydroxy-6-methylheptan-2-yl]-2,3,14-trihydroxy-10,13-dimethyl-2,3,4,5,9,11,12,15,16,17-decahydro-1H-cyclopenta[a]phenanthren-6-one; N′-(3,5-Dimethylbenzoyl)-N′-[(3R)-2,2-dimethyl-3-hexanyl]-2-ethyl-3-methoxybenzohydrazide; 5-Methyl-2,3-dihydro-benzo[1,4]dioxine-6-carboxylic acid N′-(3,5-dimethyl-benzoyl)-N′-(1-ethyl-2,2-dimethyl-propyl)-hydrazide; 5-Methyl-2,3-dihydro-benzo[1,4]dioxine-6-carboxylic acid N′-(3,5-dimethoxy-4-methyl-benzoyl)-N′-(1-ethyl-2,2-dimethyl-propyl)-hydrazide; 5-Methyl-2,3-dihydro-benzo[1,4]dioxine-6-carboxylic acid N′-(1-tert-butyl-butyl)-N′-(3,5-dimethyl-benzoyl)-hydrazide; 5-Methyl-2,3-dihydro-benzo[1,4]dioxine-6-carboxylic acid N′-(1-tert-butyl-butyl)-N′-(3,5-dimethoxy-4-methyl-benzoyl)-hydrazide; 5-Ethyl-2,3-dihydro-benzo[1,4]dioxine-6-carboxylic acid N′-(3,5-dimethyl-benzoyl)-N′-(1-ethyl-2,2-dimethyl-propyl)-hydrazide; 5-Ethyl-2,3-dihydro-benzo[1,4]dioxine-6-carboxylic acid N′-(3,5-dimethoxy-4-methyl-benzoyl)-N′-(1-ethyl-2,2-dimethyl-propyl)-hydrazide; 5-Ethyl-2,3-dihydro-benzo[1,4]dioxine-6-carboxylic acid N′-(1-tert-butyl-butyl)-N′-(3,5-dimethyl-benzoyl)-hydrazide; 5-Ethyl-2,3-dihydro-benzo[1,4]dioxine-6-carboxylic acid N′-(1-tert-butyl-butyl)-N′-(3,5-dimethoxy-4-methyl-benzoyl)-hydrazide; 3,5-Dimethyl-benzoic acid N-(1-ethyl-2,2-dimethyl-propyl)-N′-(3-methoxy-2-methyl-benzoyl)-hydrazide; 3,5-Dimethoxy-4-methyl-benzoic acid N-(1-ethyl-2,2-dimethyl-propyl)-N′-(3-methoxy-2-methyl-benzoyl)-hydrazide; 3,5-Dimethyl-benzoic acid N-(1-tert-butyl-butyl)-N′-(3-methoxy-2-methyl-benzoyl)-hydrazide; 3,5-Dimethoxy-4-methyl-benzoic acid N-(1-tert-butyl-butyl)-N′-(3-methoxy-2-methyl-benzoyl)-hydrazide; 3,5-Dimethyl-benzoic acid N-(1-ethyl-2,2-dimethyl-propyl)-N′-(2-ethyl-3-methoxy-benzoyl)-hydrazide; 3,5-Dimethoxy-4-methyl-benzoic acid N-(1-ethyl-2,2-dimethyl-propyl)-N′-(2-ethyl-3-methoxy-benzoyl)-hydrazide; 3,5-Dimethyl-benzoic acid N-(1-tert-butyl-butyl)-N′-(2-ethyl-3-methoxy-benzoyl)-hydrazide; 3,5-Dimethoxy-4-methyl-benzoic acid N-(1-tert-butyl-butyl)-N′-(2-ethyl-3-methoxy-benzoyl)-hydrazide; 2-Methoxy-nicotinic acid N-(1-tert-butyl-pentyl)-N′-(4-ethyl-benzoyl)-hydrazide; 3,5-Dimethyl-benzoic acid N-(2,2-dimethyl-1-phenyl-propyl)-N′-(4-ethyl-benzoyl)-hydrazide; 3,5-Dimethyl-benzoic acid N-(1-tert-butyl-pentyl)-N′-(3-methoxy-2-methyl-benzoyl)-hydrazide; and 3,5-Dimethoxy-4-methyl-benzoic acid N-(1-tert-butyl-pentyl)-N′-(3-methoxy-2-methyl-benzoyl)-hydrazide.

In some cases, a ligand used for dose-regulated control of ecdysone receptor-based inducible gene switch can be selected from any of, but without limitation to, an ecdysteroid, such as ecdysone, 20-hydroxyecdysone, ponasterone A, muristerone A, and the like, 9-cis-retinoic acid, synthetic analogs of retinoic acid, N,N′-diacylhydrazines such as those disclosed in U.S. Pat. Nos. 6,013,836; 5,117,057; 5,530,028; and 5,378,726 and U.S. Published Application Nos. 2005/0209283 and 2006/0020146; oxadiazolines as described in U.S. Published Application No. 2004/0171651; dibenzoylalkyl cyanohydrazines such as those disclosed in European Application No. 461,809; N-alkyl-N,N′-diaroylhydrazines such as those disclosed in U.S. Pat. No. 5,225,443; N-acyl-N-alkylcarbonylhydrazines such as those disclosed in European Application No. 234,994; N-aroyl-N-alkyl-N′-aroylhydrazines such as those described in U.S. Pat. No. 4,985,461; arnidoketones such as those described in U.S. Published Application No. 2004/0049037; each of which is incorporated herein by reference and other similar materials including 3,5-di-tert-butyl-4-hydroxy-N-isobutyl-benzamide, 8-O-acetylharpagide, oxysterols, 22(R) hydroxycholesterol, 24 (S) hydroxycholesterol, 25-epoxycholesterol, T0901317, 5-alpha-6-alpha-epoxycholesterol-3-sulfate (ECHS), 7-ketocholesterol-3-sulfate, framesol, bile acids, 1,1-biphosphonate esters, juvenile hormone III, and the like. Examples of diacylhydrazine ligands useful in the present disclosure include RG-115819 (3,5-Dimethyl-benzoic acid N-(1-ethyl-2,2-dimethyl-propyl)-N′-(2-methyl-3-methoxy-benzoyl)-hydrazide-), RG-115932 ((R)-3,5-Dimethyl-benzoic acid N-(1-tert-butyl-butyl)-N′-(2-ethyl-3-methoxy-benzoyl)-hydrazide), and RG-115830 (3,5-Dimethyl-benzoic acid N-(1-tert-butyl-butyl)-N′-(2-ethyl-3-methoxy-benzoyl)-hydrazide). See, e.g., U.S. patent application Ser. No. 12/155,111, and PCT Appl. No. PCT/US2008/006757, both of which are incorporated herein by reference in their entireties.

### Cytokines

In certain embodiments, HPV vaccine antigens provided herein may be co-delivered and/or co-expressed (e.g., as part of the same HPV antigen delivery vector or via a separate vector) along with other cytokines. Provided herein are polynucleotides encoding gene-switch polypeptides and a cytokine, or variant or derivative thereof, and methods and systems incorporating the same. Cytokine is a category of small proteins between about 5-20 kDa that are involved in cell signaling. In some instances, cytokines include chemokines, interferons, interleukins, colony-stimulating factors or tumor necrosis factors. In some embodiments, chemokines play a role as a chemoattractant to guide the migration of cells, and is classified into four subfamilies: CXC, CC, CX3C, and XC. Exemplary chemokines include chemokines from the CC subfamily: CCL1, CCL2 (MCP-1), CCL3, CCL4, CCL5 (RANTES), CCL6, CCL7, CCL8, CCL9 (or CCL10), CCL11, CCL12, CCL13, CCL14, CCL15, CCL16, CCL17, CCL18, CCL19, CCL20, CCL21, CCL22, CCL23, CCL24, CCL25, CCL26, CCL27, and CCL28; the CXC subfamily: CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, and CXCL17; the XC subfamily: XCL1 and XCL2; and the CX3C subfamily CX3CL1.

In certain embodiments, HPV vaccine antigens provided herein may be co-delivered and/or co-expressed (e.g., as part of the same HPV antigen delivery vector or via a separate vector) along with interferons. Interferons (IFNs) comprise interferon type I (e.g. IFN-α, IFN-β, IFN-ε, IFN-κ, and IFN-ω), interferon type II (e.g. IFN-γ), and interferon type III. In some embodiments, IFN-α is further classified into about 13 subtypes including IFNA1, IFNA2, IFNA4, IFNA5, IFNA6, IFNA7, IFNA8, IFNA10, IFNA13, IFNA14, IFNA16, IFNA17, and IFNA21.

In certain embodiments, HPV vaccine antigens provided herein may be co-delivered and/or co-expressed (e.g., as part of the same HPV antigen delivery vector or via a separate vector) along with other interleukins. Interleukins are expressed by leukocytes or white blood cells and they promote the development and differentiation of T and B lymphocytes and hematopoietic cells. Exemplary interleukins include IL-1, IL-2, IL-3, IL-4, IL-5, IL-6, IL-7, IL-8 (CXCL8), IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-35, and IL-36. In some embodiments, interleukins are IL-2, IL-12, IL-15, IL-21 or a fusion of IL-15 and IL-15a.

In some aspects, the interleukin can comprise IL-12. In some embodiments, the IL-12 is a single chain IL-12 (scIL-12), protease sensitive IL-12, destabilized IL-12, membrane bound IL-12, intercalated IL-12. In some instances, the IL-12 variants are as described in WO2015/095249, WO2016/048903, WO2017/062953, all of which is incorporated by reference in their entireties.

In some embodiments, an interleukin comprises mbIL-15. In some embodiments, a mbIL-15 is a membrane-bound chimeric IL-15 which can be co-expressed with a modified effector cell described herein. In some embodiments, the mbIL-15 comprises a full-length IL-15 (e.g., a native IL-15 polypeptide) or fragment or variant thereof, fused in frame with a full length IL-15Rα, functional fragment or variant thereof. In some cases, the IL-15 is indirectly linked to the IL-15Rα through a linker. In some instances, the mbIL-15 is as described in Hurton et al., “Tethered IL-15 augments antitumor activity and promotes a stem-cell memory subset in tumor-specific T cells,” PNAS 2016.

In certain embodiments, HPV vaccine antigens provided herein may be co-delivered and/or co-expressed (e.g., as part of the same HPV antigen delivery vector or via a separate vector) along with tumor necrosis factors. Tumor necrosis factors (TNFs) are a group of cytokines that modulate apoptosis. In some instances, there are about 19 members within the TNF family, including, not limited to, TNFα, lymphotoxin-alpha (LT-alpha), lymphotoxin-beta (LT-beta), T cell antigen gp39 (CD40L), CD27L, CD30L, FASL, 4-1BBL, OX40L, and TNF-related apoptosis inducing ligand (TRAIL).

In certain embodiments, HPV vaccine antigens provided herein may be co-delivered and/or co-expressed (e.g., as part of the same HPV antigen delivery vector or via a separate vector) along with colony stimulating factors. Colony-stimulating factors (CSFs) are secreted glycoproteins that interact with receptor proteins on the surface of hemopoietic stem cells, which subsequently modulates cell proliferation and differentiation into specific kind of blood cells. In some instances, a CSF comprises macrophage colony-stimulating factor, granulocyte macrophage colony-stimulating factor (GM-CSF), granulocyte colony-stimulating factor (G-CSF) or promegapoietin.

In some embodiments, the cytokine is a membrane-bound cytokine, which is co-expressed with a chimeric antigen receptor described herein. In some embodiments, one or more methods described herein further comprise administration of a cytokine. In some instances, the cytokine comprises a chemokine, an interferon, an interleukin, a colony-stimulating factor or a tumor necrosis factor. In some instances, one or more methods described herein further comprise administration of a cytokine selected from a chemokine, an interferon, an interleukin, a colony-stimulating factor or a tumor necrosis factor. In some instances, one or more methods described herein further comprise administration of a cytokine selected from IL2, IL7, IL12, IL15, a fusion of IL-15 and IL-15Rα, IL21, IFNγ or TNF-α.

### Interleukin-12

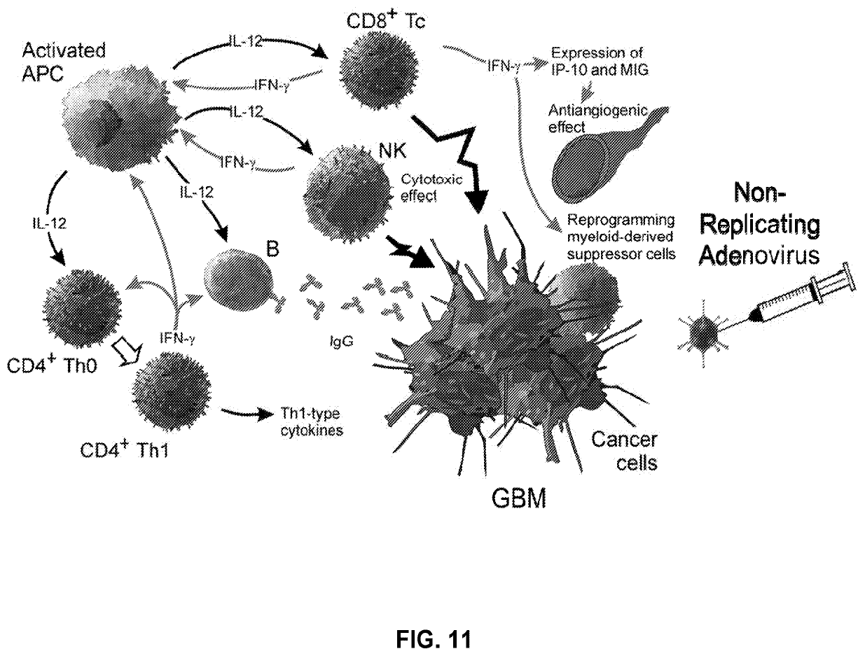

In particular embodiments, HPV vaccine antigens provided herein may be co-delivered and/or co-expressed (e.g., as part of the same HPV antigen delivery vector or via a separate vector) along with Interleukin-12. Interleukin 12 (IL-12) is an interleukin that is naturally produced by dendritic cells, macrophages, neutrophils, and human B-lymphoblastoid cells (NC-37) in response to antigenic stimulation. IL-12 is composed of a bundle of four alpha helices. It is a heterodimeric cytokine encoded by two separate genes, IL-12A (p35) and IL-12B (p40). The active heterodimer (referred to as p70), and a homodimer of p40 are formed following protein synthesis. IL-12 is the master regulator of the immune system. IL-12 promotes immune response by activating NK cells and T cells (FIG. 11).

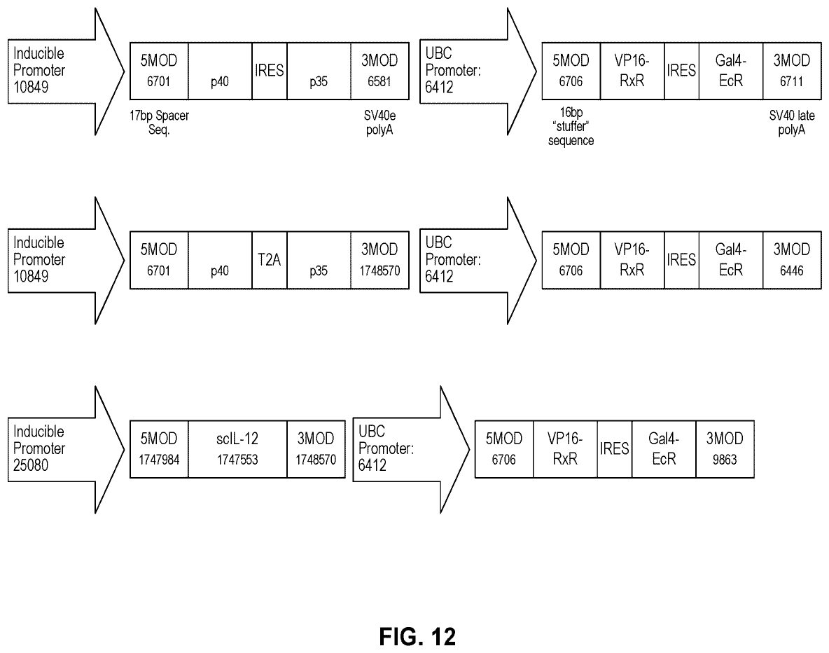

Provided herein are compositions, kits, and system comprising and methods of making HPV recombinant vaccines. The present disclosure provides HPV antigen designs (HPV designs 1-5) constructed in a multi-deleted gorilla adenovector (GC46) (SEQ ID NOS: 61-63). Also provided herein are polynucleotides encoding gene-switch polypeptides and IL-12 or variant or derivative thereof, and methods and systems incorporating the same (FIG. 12).

### Linkers

Also disclosed are constructs comprising a linker to facilitate the expression and functionality of the polynucleotides and polypeptides described herein. In some embodiments, a polynucleotide linker can be utilized in a polynucleotide described herein. A polynucleotide linker can be a double-stranded segment of DNA containing desired restriction sites that can be added to create end structures that are compatible with a vector comprising a polynucleotide described herein. In some cases, a polynucleotide linker can be useful for modifying vectors comprising polynucleotides described herein. For example, a vector modification comprising a polynucleotide linker can be a change in a multiple cloning site, or the addition of a poly-histidine tail. Polynucleotide linkers can also be used to adapt the ends of blunt insert DNA for cloning into a vector cleaved with a restriction enzyme with cohesive end termini. The use of polynucleotide linkers can be more efficient than a blunt ligation into a vector and can provide a method of releasing an insert from a vector in downstream applications. In some cases an insert can be a polynucleotide sequence encoding polypeptides useful for therapeutic applications. In some cases, a linker can be a cleavable linker.

A polynucleotide linker can be an oligomer. A polynucleotide linker can be a DNA double strand, single strand, or a combination thereof. In some cases, a linker can be RNA. A polynucleotide linker can be ligated into a vector comprising a polynucleotide described herein by a T4 ligase in some cases. To facilitate a ligation an excess of polynucleotide linkers can be added to a composition comprising an insert and a vector. In some cases, an insert and vector are pre-treated before a linker is introduced. For example, pre-treatment with a methylase can prevent unwanted cleavage of insert DNA.

In certain embodiments, two or more polypeptides encoded by a polynucleotide described herein can be separated by an intervening sequence encoding an intervening linker polypeptide. Herein the term “intervening linker polypeptide” referring to an amino acid sequence separating two or more polypeptides encoded by a polynucleotide is distinguished from the term “peptide linker” which refers to the sequence of amino acids which is optionally included in a polypeptide construct disclosed herein to connect the transmembrane domain to the cell surface polypeptide (e.g., comprising a truncated variant of a natural polypeptide). In certain cases, the intervening linker is a cleavage-susceptible intervening linker polypeptide. In some embodiments, the linker is a cleavable or ribosome skipping linker. In some embodiments, the cleavable linker or ribosome skipping linker sequence is selected from the group consisting of 2A, GSG-2A, GSG linker, SGSG linker, furinlink variants and derivatives thereof. In some embodiments, the 2A linker is a p2A linker, a T2A linker, F2A linker or E2A linker. In some embodiments, polypeptides of interest are expressed as fusion proteins linked by a cleavage-susceptible intervening linker polypeptide. In certain embodiments, cleavage-susceptible intervening linker polypeptide(s) can be any one or more of: F/T2A, T2A, p2A, 2A, GSG-p2A, GSG linker, and furinlink variants. Linkers (polynucleotide and polypeptide sequences) as disclosed in PCT/US2016/061668 (WO2017083750) published 18 May 2017 are incorporated by reference herein. In certain embodiments, the linker polypeptide comprises disclosed in the table below:

TABLE 1
Linker amino acid sequences and polynucleotide sequences

| | SEQ | Polynucleotide | SEQ | |
|---|---|---|---|---|
| | ID | Sequence (5′ to 3′ | ID | Amino Acids Sequence |
| Linker Name | NO: | where applicable) | NO: | (5′ to 3′ where applicable) |
| Whitlow | 64 | GGCAGCACCTCCGGCAGCG | 81 | GSTSGSGKPGSGEGSTKG |
| Linker | | GCAAGCCTGGCAGCGGCGA | | |
| | | GGGCAGCACCAAGGGC | | |
| Linker | 65 | TCTGGCGGAGGATCTGGAG | 82 | SGGGSGGGGSGGGGSGGG |
| | | GAGGCGGATCTGGAGGAGG | | GSGGGSLQ |
| | | AGGCAGTGGAGGCGGAGGA | | |
| | | TCTGGCGGAGGATCTCTGCA | | |
| | | G | | |
| GSG linker | 66 | GGAAGCGGA | 83 | GSG |
| SGSG linker | 67 | AGTGGCAGCGGC | 84 | SGSG |
| (G4S)3 linker | 68 | GGTGGCGGTGGCTCGGGCG | 85 | GGGGSGGGGSGGGGS |
| | | GTGGTGGGTCGGGTGGCGG | | |
| | | CGGATCT | | |
| Furin cleavage | 69 | CGTGCAAAGCGT | 86 | RAKR |
| site/ | | | | |
| Furinlink1 | | | | |
| Fmdv | 70 | AGAGCCAAGAGGGCACCGG | 87 | RAKRAPVKQTLNFDLLKL |
| | | TGAAACAGACTTTGAATTTT | | AGDVESNPGP |
| | | GACCTTCTGAAGTTGGCAGG | | |
| | | AGACGTTGAGTCCAACCCTG | | |
| | | GGCCC | | |
| Thosea asigna | 71 | GAGGGCAGAGGAAGTCTGC | 88 | EGRGSLLTCGDVEENPGP |
| virus 2A | | TAACATGCGGTGACGTCGA | | |
| region (T2A) | | GGAGAATCCTGGACCT | | |
| Furin-GSG- | 72 | AGAGCTAAGAGGGGAAGCG | 89 | RAKRGSGEGRGSLLTCGD |
| T2A | | GAGAGGGCAGAGGAAGTCT | | VEENPGP |
| | | GCTAACATGCGGTGACGTCG | | |
| | | AGGAGAATCCTGGACCT | | |
| Furin-SGSG- | 73 | AGGGCCAAGAGGAGTGGCA | 90 | RAKRSGSGEGRGSLLTCGD |
| T2A | | GCGGCGAGGGCAGAGGAAG | | VEENPGP |
| | | TCTTCTAACATGCGGTGACG | | |
| | | TGGAGGAGAATCCCGGCCC | | |
| | | T | | |
| Porcine | 74 | GCAACGAACTTCTCTCTCCT | 91 | ATNFSLLKQAGDVEENPGP |
| teschovirus-1 | | AAAACAGGCTGGTGATGTG | | |
| 2A region | | GAGGAGAATCCTGGTCCA | | |
| (P2A) | | | | |
| GSG-P2A | 75 | GGAAGCGGAGCTACTAACT | 92 | GSGATNFSLLKQAGDVEE |
| | | TCAGCCTGCTGAAGCAGGCT | | NPGP |
| | | GGAGACGTGGAGGAGAACC | | |
| | | CTGGACCT | | |
| Equine rhinitis | 76 | CAGTGTACTAATTATGCTCT | 93 | QCTNYALLKLAGDVESNP |
| A virus 2A | | CTTGAAATTGGCTGGAGATG | | GP |
| region (E2A) | | TTGAGAGCAACCCTGGACCT | | |
| Foot-and- | 77 | GTCAAACAGACCCTAAACTT | 94 | VKQTLNFDLLKLAGDVES |
| mouth disease | | TGATCTGCTAAAACTGGCCG | | NPGP |
| virus 2A | | GGGATGTGGAAAGTAATCC | | |
| region (F2A) | | CGGCCCC | | |
| FP2A | 78 | CGTGCAAAGCGTGCACCGG | 95 | RAKRAPVKQGSGATNFSLL |
| | | TGAAACAGGGAAGCGGAGC | | KQAGDVEENPGP |
| | | TACTAACTTCAGCCTGCTGA | | |
| | | AGCAGGCTGGAGACGTGGA | | |
| | | GGAGAACCCTGGACCT | | |
| Linker-GSG | 79 | GCACCGGTGAAACAGGGAA | 96 | APVKQGSG |
| | | GCGGA | | |
| Linker | 80 | GCACCGGTGAAACAG | 97 | APVKQ |

In some embodiments, an intervening linker polypeptide comprises an amino acid sequence having at least 70%, 75%, 80%, 85%, 90%, 95%, 99%, 99.5% or 100% identity with the amino acid sequence of Whitlow linker (SEQ ID NO: 64), GSG linker (SEQ ID NO: 66), SGSG linker (SEQ ID NO: 67), (G4S)3 linker (SEQ ID NO: 68), Furin cleavage site/Furlink1 (SEQ ID NO: 69), Fmdv linker (SEQ ID NO: 70), Thosea asigna virus 2A region (T2A) (SEQ ID NO: 71), Furin-GSG-T2A (SEQ ID NO: 72), Furin-SGSG-T2A (SEQ ID NO: 73), porcine teschovirus-1 2A region (P2A) (SEQ ID NO: 74), GSG-P2A (SEQ ID NO: 75), equine rhinitis A virus 2A region (E2A) (SEQ ID NO: 76), or foot-and-mouth disease virus 2A region (F2A) (SEQ ID NO: 78) (Table 1). In some cases, an intervening linker polypeptide comprises an amino acid sequence having at least 70%, 75%, 80%, 85%, 90%, 95%, 99%, 99.5% or 100% identity with the amino acid sequence of linkers (SEQ ID NOS: 65, 79 80) In some cases, a viral 2A sequence can be used. 2A elements can be shorter than IRES, having from 5 to 100 base pairs. In some cases, a 2A sequence can have 5, 10, 15, 20, 25, 30, 35, 40, 45, 50, 55, 60, 65, 70, 75, 80, 85, 90, or 100 nucleotides in length. 2A linked genes can be expressed in one single open reading frame and “self-cleavage” can occur co-translationally between the last two amino acids, GP, at the C-terminus of the 2A polypeptide, giving rise to equal amounts of co-expressed proteins.

A viral 2A sequence can be about 20 amino acids. In some cases, a viral 2A sequence can contain a consensus motif Asp-Val/Ile-Glu-X-Asn-Pro-Gly-Pro. A consensus motif sequence can act co-translationally. For example, formation of a normal peptide bond between a glycine and proline residue can be prevented, which can result in ribosomal skipping and cleavage of a nascent polypeptide. This effect can produce multiple genes at equimolar levels.

A 2A peptide can allow translation of multiple proteins in a single open reading frame into a polypeptide that can be subsequently cleaved into individual polypeptide through a ribosome-skipping mechanism (Funston et al., J. Gen. Virol. 89(Pt 2): 389-96 (2008)). In some embodiments, a 2A sequence can include: F/T2A, T2A, p2A, 2A, T2A, E2A, F2A, and BmCPV2A, BmIFV2A, and any combination thereof.

In some cases, a vector can comprise an IRES sequence and a 2A linker sequence. In other cases, expression of multiple genes linked with 2A peptides can be facilitated by a spacer sequence (GSG) ahead of the 2A peptides. In some cases, constructs can combine a spacers, linkers, adaptors, promoters, or combinations thereof. For example, a linker can have a spacer (SGSG or GSG or Whitlow linker) and furin linker (R-A-K-R) cleavage site with different 2A peptides. A spacer can be an I-Ceui. In some cases, a linker can be engineered. For example, a linker can be designed to comprise chemical characteristics such as hydrophobicity. In some cases, at least two linker sequences can produce the same protein. In other cases, multiple linkers can be used in a vector. For example, genes of interest can be separated by at least two linkers.

In certain embodiments, two or more polypeptides encoded by a polynucleotide described herein can be separated by an intervening sequence encoding a linker polypeptide. In certain cases, the linker is a cleavage-susceptible linker. In some embodiments, polypeptides of interest are expressed as fusion proteins linked by a cleavage-susceptible linker polypeptide. In certain embodiments, cleavage-susceptible linker polypeptide(s) can be any one or two of: Furinlink, fmdv, p2a, GSG-p2a, and/or fp2a described below. In some cases, a linker is APVKQGSG (SEQ ID NO: 96).

In certain cases, a linker polypeptide can comprise an amino acid sequence “RAKR” (SEQ ID NO: 86). In certain cases, a Furin linker polypeptide can be encoded by a polynucleotide sequence polynucleotide sequence comprising “CGTGCAAAGCGT” (SEQ ID NO: 69) or “AGAGCTAAGAGG” (SEQ ID NO: 112).

In some embodiments, a linker can be utilized in a polynucleotide described herein. A linker can be a flexible linker, a rigid linker, an in vivo cleavable linker, or any combination thereof. In some cases, a linker can link functional domains together (as in flexible and rigid linkers) or releasing free functional domain in vivo as in in vivo cleavable linkers.

Linkers can improve biological activity, increase expression yield, and achieving desirable pharmacokinetic profiles. A linker can also comprise hydrazone, peptide, disulfide, or thioesther.

In some cases, a linker sequence described herein can include a flexible linker. Flexible linkers can be applied when a joined domain requires a certain degree of movement or interaction. Flexible linkers can be composed of small, non-polar (e.g., Gly) or polar (e.g., Ser or Thr) amino acids. A flexible linker can have sequences consisting primarily of stretches of Gly and Ser residues (“GS” linker). An example of a flexible linker can have the sequence of (Gly-Gly-Gly-Gly-Ser)n (SEQ ID NO: 85). By adjusting the copy number “n”, the length of this exemplary GS linker can be optimized to achieve appropriate separation of functional domains, or to maintain necessary inter-domain interactions. Besides GS linkers, other flexible linkers can be utilized for recombinant fusion proteins. In some cases, flexible linkers can also be rich in small or polar amino acids such as Gly and Ser, but can contain additional amino acids such as Thr and Ala to maintain flexibility. In other cases, polar amino acids such as Lys and Glu can be used to improve solubility.

Flexible linkers included in linker sequences described herein, can be rich in small or polar amino acids such as Gly and Ser to provide good flexibility and solubility. Flexible linkers can be suitable choices when certain movements or interactions are desired for fusion protein domains. In addition, although flexible linkers cannot have rigid structures, they can serve as a passive linker to keep a distance between functional domains. The length of flexible linkers can be adjusted to allow for proper folding or to achieve optimal biological activity of the fusion proteins.

A linker described herein can further include a rigid linker in some cases. A rigid linker can be utilized to maintain a fixed distance between domains of a polypeptide. Examples of rigid linkers can be: Alpha helix-forming linkers, Pro-rich sequence, (XP)n, X-Pro backbone, A (EAAAK)nA (n=2-5), to name a few. Rigid linkers can exhibit relatively stiff structures by adopting α-helical structures or by containing multiple Pro residues in some cases.

A linker described herein can be cleavable in some cases. In other cases a linker is not cleavable. Linkers that are not cleavable can covalently join functional domains together to act as one molecule throughout an in vivo processes or an ex vivo process. A linker can also be cleavable in vivo. A cleavable linker can be introduced to release free functional domains in vivo. A cleavable linker can be cleaved by the presence of reducing reagents, proteases, to name a few. For example, a reduction of a disulfide bond can be utilized to produce a cleavable linker. In the case of a disulfide linker, a cleavage event through disulfide exchange with a thiol, such as glutathione, could produce a cleavage. In other cases, an in vivo cleavage of a linker in a recombinant fusion protein can also be carried out by proteases that can be expressed in vivo under pathological conditions (e.g. cancer or inflammation), in specific cells or tissues, or constrained within certain cellular compartments. In some cases, a cleavable linker can allow for targeted cleavage. For example, the specificity of many proteases can offer slower cleavage of a linker in constrained compartments. A cleavable linker can also comprise hydrazone, peptides, disulfide, or thioesther. For example, a hydrazone can confer serum stability. In other cases, a hydrazone can allow for cleavage in an acidic compartment. An acidic compartment can have a pH up to 7. A linker can also include a thioether. A thioether can be nonreducible A thioether can be designed for intracellular proteolytic degradation.

In certain embodiments, an fmdv linker polypeptide comprises a sequence that can be at least about 45%, 50%, 55%, 60%, 65%, 70%, 75%, 80%, 85%, 90%, 95%, 97%, 98%, 99% or 100% identical to SEQ ID NO: 87. In certain embodiments, an fmdv linker polypeptide is one or more of the linkers encoded in a single vector linking two or more fusion proteins. In certain cases, an fmdv linker polypeptide can be encoded by a polynucleotide open reading frame (ORF) nucleic acid sequence. In some cases, an ORF encoding fmdv comprises or consists of a sequence of SEQ ID NO: 70). In certain embodiments, a polynucleotide encoding fmdv is at least 45%, 50%, 55%, 60%, 65%, 70%, 75%, 80%, 85%, 90%, 95%, 97%, 98%, 99% or 100% identical to SEQ ID NO: 70).

In certain cases, a linker polypeptide can be a “p2a” linker. In certain embodiments, a p2a polypeptide can comprise a sequence that can be about at least 45%, 50%, 55%, 60%, 65%, 70%, 75%, 80%, 85%, 90%, 95%, 97%, 98%, 99% or 100% identical to SEQ ID NO: 91). In certain embodiments, the p2a linker polypeptide can be one or more of the linkers encoded in a single vector linking two or more fusion proteins. In some cases, a p2a linker polypeptide can be encoded by a polynucleotide open reading frame (ORF) nucleic acid sequence. In certain embodiments, an ORF encoding p2a comprises or consists of the sequence of SEQ ID NO: 74). In certain cases, a polynucleotide encoding p2a can be or can be about at least 45%, 50%, 55%, 60%, 65%, 70%, 75%, 80%, 85%, 90%, 95%, 97%, 98%, 99% or 100% identical to SEQ ID NO: 74).

In some cases, a linker polypeptide can be a “GSG-p2a” linker. In certain embodiments, a GSG-p2a linker polypeptide can comprise a sequence that can be about at least 45%, 50%, 55%, 60%, 65%, 70%, 75%, 80%, 85%, 90%, 95%, 97%, 98%, 99% or 100% identical to SEQ ID NO: 92). In certain embodiments, a GSG-p2a linker polypeptide can be one or more of the linkers encoded in a single vector linking two or more fusion proteins. In some cases, a GSG-p2a linker polypeptide can be encoded by a polynucleotide open-reading frame (ORF) nucleic acid sequence. An ORF encoding GSG p2a can comprise the sequence of SEQ ID NO: 75). In some cases, a polynucleotide encoding GSG-p2a can be or can be about at least 45%, 50%, 55%, 60%, 65%, 70%, 75%, 80%, 85%, 90%, 95%, 97%, 98%, 99% or 100% identical to SEQ ID NO: 75).

A linker polypeptide can be an “fp2a” linker as provided herein. In certain embodiments, a fp2a linker polypeptide can comprise a sequence that can be about at least 45%, 50%, 55%, 60%, 65%, 70%, 75%, 80%, 85%, 90%, 95%, 97%, 98%, 99% or 100% identical to SEQ ID NO: 95). In certain cases, an fp2a linker polypeptide can be one or more of the linkers encoded in a single vector linking two or more fusion proteins. In some cases, a fp2a linker polypeptide can be encoded by a polynucleotide open reading frame (ORF) nucleic acid sequence. In certain embodiments, a polynucleotide encoding an fp2a linker can be or can be about at least 45%, 50%, 55%, 60%, 65%, 70%, 75%, 80%, 85%, 90%, 95%, 97%, 98%, 99% or 100% identical to SEQ ID NO: 78).

In some cases, a linker can be engineered. For example, a linker can be designed to comprise chemical characteristics such as hydrophobicity. In some cases, at least two linker sequences can produce the same protein. A sequence can be or can be about 45%, 50%, 55%, 60%, 65%, 70%, 75%, 80%, 85%, 90%, 95%, 96%, 97%, 98%, 99%, or 100% identical to a polypeptide sequence of SEQ ID NO 82, 96 or 97. In other cases, multiple linkers can be used in a vector. For example, genes of interest, and one or more gene switch polypeptide sequences described herein can be separated by at least two linkers. In some cases, genes can be separated by 2, 3, 4, 5, 6, 7, 8, 9, or up to 10 linkers.

A linker can be an engineered linker. Methods of designing linkers can be computational. In some cases, computational methods can include graphic techniques. Computation methods can be used to search for suitable peptides from libraries of three-dimensional peptide structures derived from databases. For example, a Brookhaven Protein Data Bank (PDB) can be used to span the distance in space between selected amino acids of a linker.

In some embodiments are polynucleotides encoding a polypeptide construct comprising a furin polypeptide and a 2A polypeptide, wherein the furin polypeptide and the 2A polypeptide are connected by a polypeptide linker comprising at least three hydrophobic amino acids. In some cases, at least three hydrophobic amino acids are selected from the list consisting of glycine (Gly)(G), alanine (Ala)(A), valine (Val)(V), leucine (Leu)(L), isoleucine (Ile)(I), proline (Pro)(P), phenylalanine (Phe)(F), methionine (Met)(M), tryptophan (Trp)(W). In some cases, a polypeptide linker can also include one or more GS linker sequences, for instance (GS)n, (SG)n, (GSG)n, and (SGSG)n, wherein n can be any number from zero to fifteen.

Provided are methods of obtaining an improved expression of a polypeptide construct comprising: providing a polynucleotide encoding said polypeptide construct comprising a first functional polypeptide and a second functional polypeptide, wherein said first functional polypeptide and second functional polypeptide are connected by a linker polypeptide comprising a sequence with at least 60% identity to the sequence APVKQ; and expressing said polynucleotide in a host cell, wherein said expressing results in an improved expression of the polypeptide construct as compared to a corresponding polypeptide construct that does not have a linker polypeptide comprising a sequence with at least 60% identity to the sequence APVKQ.

### IRES Elements

Also disclosed herein are constructs comprising an IRES element to facilitate the expression and functionality of the polynucleotides and polypeptides described herein. The term “internal ribosome entry site (IRES)” as used herein can be intended to mean internal ribosomal entry site. In a vector comprising an IRES sequence, a first gene can be translated by a cap-dependent, ribosome scanning, mechanism with its own 5′-UTR, whereas translation of a subsequent gene can be accomplished by direct recruitment of a ribosome to an IRES in a cap-independent manner. An IRES sequence can allow eukaryotic ribosomes to bind and begin translation without binding to a 5′ capped end. An IRES sequence can allow expression of multiple genes from one transcript (Mountford and Smith, Trends Genet. 11(5):179-84 (1995)).

The term “CAP” or “cap” as used herein refers to a modified nucleotide, generally a 7-methyl guanosine, linked 3′ to 5′ (7meG-ppp-G), to the 5′ end of a eukaryotic mRNA, that serves as a required element in the normal translation initiation pathway during expression of protein from that mRNA.

In certain cases, an IRES region can be derived from a virus, such as picornavirus, encephalomyocarditis virus, hepatitis C virus IRES sequence. In other cases, an IRES sequence can be derived from an encephalomyocarditis virus. The term “EMCV” or “encephalomyocarditis virus” as used herein refers to any member isolate or strain of the encephalomyocarditis virus species of the genus of the family Picornaviridae. Examples are: EMCV-R (Rueckert) strain virus, Columbia-SK virus. In some cases, a cellular IRES element, such as eukaryotic initiation factor 4G, immunoglobulin heavy chain binding protein, c-myc proto-oncogene, vascular endothelial growth factor, fibroblast growth factor-1 IRES, or any combination or modification thereof can be used. In some cases, a cellular IRES can have increased gene expression when compared to a viral IRES.

An IRES sequence of viral, cellular or a combination thereof can be utilized in a vector. An IRES can be from encephalomyocarditis (EMCV) or poliovirus (PV). In some cases, an IRES element is selected from a group consisting of Poliovirus (PV), Encephalomyelitis virus (EMCV), Foot-and-mouth disease virus (FMDV), Porcine teschovirus-1 (PTV-1), Aichivirus (AiV), Seneca Valley virus (SVV), Hepatitis C virus (HCV), Classical swine fever virus (CSFV), Human immunodeficiency virus-2 (HIV-2), Human immunodeficiency virus-1 (HIV-1), Moloney murine leukemia virus (MoMLV), Feline immunodeficiency virus (FIV), Mouse mammary tumor virus (MMTV), Human cytomegalovirus latency (pUL138), Epstein-Barr virus (EBNA-1), Herpes virus Marek's disease (MDV RLORF9), SV40 polycistronic 19S (SV40 19S), Rhopalosiphum padi virus (RhPV), Cricket paralysis virus (CrPV), Ectropis obliqua picorna-like virus (EoPV), Plautia stali intestine virus (PSIV), Triatoma virus (TrV), Bee paralysis dicistrovirus (IAPV, KBV), Black currant reversion virus (BRV), Pelargonium flower break virus (PFBV), Hibiscus chlorotic ringspot virus (HCRSV), Crucifer-infecting tobamovirus (CrTMV), Potato leaf roll polerovirus (PLRV), Tobacco etch virus (TEV), Giardiavirus (GLV), Leishmania RNA virus-1 (LRV-1), and combinations or modifications thereof. In some cases, an IRES is selected from a group consisting of Apaf-1, XIAP, HIAP2/c-IAP1, DAP5, Bcl-2, c-myc, CAT-1, INR, Differentiation LEF-1, PDGF2, HIF-1a, VEGF, FGF2, BIP, BAG-1, CIRP, p53, SHMT1, PITSLREp58, CDK1, Rpr, hid, hsp70, grim, skl, Antennapedia, dFoxO, dInR, Adh-Adhr, HSP101, ADH, URE-2, GPR1, NCE102, YMR181a, MSN1, BOI1, FLO8, GIC1, and any combination or modification thereof. When an IRES element is included between two open reading frames (ORFs), initiation of translation can occur by a canonical 5′-m7GpppN cap-dependent mechanism in a first ORF and a cap-independent mechanism in a second ORF downstream of the IRES element.

In some cases, genes can be linked by an internal ribosomal entry site (IRES). An IRES can allow simultaneous expression of multiple genes. For example, an IRES sequence can permit production of multiple proteins from a single mRNA transcript. A ribosome can bind to an IRES in a 5′-cap independent manner and initiate translation.

In some cases, an IRES sequence can be or can be about 500 base pairs. An IRES sequence can be from 300 base pairs to 1000 base pairs. For example, an IRES can be 300, 350, 400, 450, 500, 550, 600, 650, 700, 750, 800, 850, 900, 950, or 1000 base pairs long.

In some cases, expression of a downstream gene within a vector comprising an IRES sequence can be reduced. For example, a gene following an IRES sequence can have reduced expression over a gene preceding an IRES sequence. Reduced expression can be from 1% to 99% reduction over a preceding gene.

### Methods of Regulating Expression

In one embodiment, a method of regulating the expression of a heterologous gene in an engineered cell is provided. Polynucleotides encoding for gene switch polypeptides for ligand inducible control of a heterologous gene expression, an antigen binding polypeptide and a heterologous gene is provided. In some instances, the polynucleotides are in one or more gene expression cassettes as depicted in any one of FIGS. 1 through 16. In another instance, the polynucleotides are incorporated into an engineered cell via viral or non-viral vectors. Viral vectors can include lentiviral vectors, retroviral vectors or adenoviral vectors. Non-viral vectors can include Sleeping Beauty transposons. In other instances, the polynucleotides are incorporated into an engineered cell via recombinases or gene editing techniques. Examples of recombinases are serine recombinases as described herein. Examples of gene editing techniques can include CRISPR or Argonaute systems. Herein a “CRISPR gene editing system” of “CRISPR system” refers to any RNA-guided Cas protein-mediated process for targeting a change in DNA sequence to a specific region of a genome. Herein “Argonaute gene editing system” refers to any single-stranded DNA guided Argonaute endonuclease-mediated process for targeting a change in DNA sequence to a specific region of a genome.

### Pharmaceutical Compostions and Dosage

The present disclosure provides a composition comprising the adenovirus or adenoviral vector described herein and a carrier therefor (e.g., a pharmaceutically acceptable carrier). The composition desirably is a physiologically acceptable (e.g., pharmaceutically acceptable) composition, which comprises a carrier, preferably a physiologically (e.g., pharmaceutically) acceptable carrier, and the adenovirus or adenoviral vector. Any suitable carrier can be used within the context of the present disclosure, and such carriers are well known in the art. The choice of carrier will be determined, in part, by the particular use of the composition (e.g., administration to an animal) and the particular method used to administer the composition. Ideally, in the context of replication-deficient adenoviral vectors, the pharmaceutical composition preferably is free of replication-competent adenovirus. The pharmaceutical composition optionally can be sterile.

Suitable compositions include aqueous and non-aqueous isotonic sterile solutions, which can contain anti-oxidants, buffers, and bacteriostats, and aqueous and non-aqueous sterile suspensions that can include suspending agents, solubilizers, thickening agents, stabilizers, and preservatives. The composition can be presented in unit-dose or multi-dose sealed containers, such as ampules and vials, and can be stored in a freeze-dried (lyophilized) condition requiring only the addition of the sterile liquid carrier, for example, water, immediately prior to use. Extemporaneous solutions and suspensions can be prepared from sterile powders, granules, and tablets. Preferably, the carrier is a buffered saline solution. More preferably, the adenovirus or adenoviral vector is part of a composition formulated to protect the adenovirus or adenoviral vector from damage prior to administration. For example, the composition can be formulated to reduce loss of the adenovirus or adenoviral vector on devices used to prepare, store, or administer the adenovirus or adenoviral vector, such as glassware, syringes, or needles. The composition can be formulated to decrease the light sensitivity and/or temperature sensitivity of the adenovirus or adenoviral vector. To this end, the composition preferably comprises a pharmaceutically acceptable liquid carrier, such as, for example, those described above, and a stabilizing agent selected from the group consisting of polysorbate 80, L-arginine, polyvinylpyrrolidone, trehalose, and combinations thereof. Use of such a composition will extend the shelf life of the adenovirus or adenoviral vector, and facilitate its administration. Formulations for adenovirus or adenoviral vector-containing compositions are further described in, for example, U.S. Pat. Nos. 6,225,289, 6,514,943, and International Patent Application Publication WO 2000/034444.

The composition also can be formulated to enhance transduction efficiency. In addition, one of ordinary skill in the art will appreciate that the adenovirus or adenoviral vector can be present in a composition with other therapeutic or biologically-active agents. For example, factors that control inflammation, such as ibuprofen or steroids, can be part of the composition to reduce swelling and inflammation associated with in vivo administration of the adenovirus or adenoviral vector. If the adenovirus or adenoviral vector is used to deliver an antigen-encoding nucleic acid sequence to a host, immune system stimulators or adjuvants, e.g., interleukins, lipopolysaccharide, or double-stranded RNA, can be administered to enhance or modify any immune response to the antigen. Antibiotics, i.e., microbicides and fungicides, can be present to treat existing infection and/or reduce the risk of future infection, such as infection associated with gene transfer procedures.

In some embodiments, disclosed herein are compositions comprising a polynucleotide or polypeptide disclosed herein for administration in a subject. In some instances, are modified effector cell compositions encoding a polynucleotide or polypeptide disclosed herein, and optionally containing a cytokine and/or an additional therapeutic agent. In some instances, also included herein are vectors encoding gene-switch polypeptides for regulating expression of a chimeric antigen receptor for modification of an effector cell.

In some instances, pharmaceutical compositions of a modified effector cell or a vector encoding gene-switch polypeptides and a chimeric antigen receptor are formulated in a conventional manner using one or more physiologically acceptable carriers including excipients and auxiliaries which facilitate processing of the active compounds into preparations which can be used pharmaceutically. Proper formulation is dependent upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

Pharmaceutical compositions are optionally manufactured in a conventional manner, such as, by way of example only, by means of conventional mixing, dissolving, granulating, dragee-making, levigating, emulsifying, encapsulating, entrapping or compression processes.

In certain embodiments, compositions can also include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

In other embodiments, compositions can also include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

The pharmaceutical compositions described herein are administered by any suitable administration route, including but not limited to, oral, parenteral (e.g., intravenous, subcutaneous, intramuscular, intracerebral, intracerebroventricular, intra-articular, intraperitoneal, or intracranial), intranasal, buccal, sublingual, or rectal administration routes. In some instances, the pharmaceutical composition is formulated for parenteral (e.g., intravenous, subcutaneous, intramuscular, intracerebral, intracerebroventricular, intra-articular, intraperitoneal, or intracranial) administration.

The pharmaceutical compositions described herein are formulated into any suitable dosage form, including but not limited to, aqueous oral dispersions, liquids, gels, syrups, elixirs, slurries, suspensions and the like, for oral ingestion by an individual to be treated, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations. In some embodiments, the pharmaceutical compositions are formulated into capsules. In some embodiments, the pharmaceutical compositions are formulated into solutions (for example, for IV administration). In some cases, the pharmaceutical composition is formulated as an infusion. In some cases, the pharmaceutical composition is formulated as an injection.

The pharmaceutical solid dosage forms described herein optionally include a compound described herein and one or more pharmaceutically acceptable additives such as a compatible carrier, binder, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof.

In still other aspects, using standard coating procedures, such as those described in Remington's Pharmaceutical Sciences, 20th Edition (2000), a film coating is provided around the compositions. In some embodiments, the compositions are formulated into particles (for example for administration by capsule) and some or all of the particles are coated. In some embodiments, the compositions are formulated into particles (for example for administration by capsule) and some or all of the particles are microencapsulated. In some embodiments, the compositions are formulated into particles (for example for administration by capsule) and some or all of the particles are not microencapsulated and are uncoated.

In certain embodiments, compositions provided herein can also include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

“Antifoaming agents” reduce foaming during processing which can result in coagulation of aqueous dispersions, bubbles in the finished film, or generally impair processing. Exemplary anti-foaming agents include silicon emulsions or sorbitan sesquoleate.

“Antioxidants” include, for example, butylated hydroxytoluene (BHT), sodium ascorbate, ascorbic acid, sodium metabisulfite and tocopherol. In certain embodiments, antioxidants enhance chemical stability where required.

Formulations described herein can benefit from antioxidants, metal chelating agents, thiol containing compounds and other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (c) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (l) pentosan polysulfate and other heparinoids, (m) divalent cations such as magnesium and zinc; or (n) combinations thereof.

“Binders” impart cohesive qualities and include, e.g., alginic acid and salts thereof; cellulose derivatives such as carboxymethylcellulose, methylcellulose (e.g., Methocel®), hydroxypropylmethylcellulose, hydroxyethylcellulose, hydroxypropylcellulose (e.g., Klucel®), ethylcellulose (e.g., Ethocel®), and microcrystalline cellulose (e.g., Avicel®); microcrystalline dextrose; amylose; magnesium aluminum silicate; polysaccharide acids; bentonites; gelatin; polyvinylpyrrolidone/vinyl acetate copolymer; crospovidone; povidone; starch; pregelatinized starch; tragacanth, dextrin, a sugar, such as sucrose (e.g., Dipac®), glucose, dextrose, molasses, mannitol, sorbitol, xylitol (e.g., Xylitab®), and lactose; a natural or synthetic gum such as acacia, tragacanth, ghatti gum, mucilage of isapol husks, polyvinylpyrrolidone (e.g., Polyvidone® CL, Kollidon® CL, Polyplasdone® XL-10), larch arabogalactan, Veegum®, polyethylene glycol, waxes, sodium alginate, and the like.

A “carrier” or “carrier materials” include any commonly used excipients in pharmaceutics and should be selected on the basis of compatibility with compounds disclosed herein, such as, compounds of ibrutinib and An anticancer agent, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. “Pharmaceutically compatible carrier materials” can include, but are not limited to, acacia, gelatin, colloidal silicon dioxide, calcium glycerophosphate, calcium lactate, maltodextrin, glycerine, magnesium silicate, polyvinylpyrrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

“Dispersing agents,” and/or “viscosity modulating agents” include materials that control the diffusion and homogeneity of a drug through liquid media or a granulation method or blend method. In some embodiments, these agents also facilitate the effectiveness of a coating or eroding matrix. Exemplary diffusion facilitators/dispersing agents include, e.g., hydrophilic polymers, electrolytes, Tween® 60 or 80, PEG, polyvinylpyrrolidone (PVP; commercially known as Plasdone®), and the carbohydrate-based dispersing agents such as, for example, hydroxypropyl celluloses (e.g., HPC, HPC-SL, and HPC-L), hydroxypropyl methylcelluloses (e.g., HPMC K100, HPMC K4M, HPMC K15M, and HPMC K100M), carboxymethylcellulose sodium, methylcellulose, hydroxyethylcellulose, hydroxypropylcellulose, hydroxypropylmethylcellulose phthalate, hydroxypropylmethylcellulose acetate stearate (HPMCAS), noncrystalline cellulose, magnesium aluminum silicate, triethanolamine, polyvinyl alcohol (PVA), vinyl pyrrolidone/vinyl acetate copolymer (S630), 4-(1,1,3,3-tetramethylbutyl)-phenol polymer with ethylene oxide and formaldehyde (also known as tyloxapol), poloxamers (e.g., Pluronics F68®, F88®, and F108®, which are block copolymers of ethylene oxide and propylene oxide); and poloxamines (e.g., Tetronic 908®, also known as Poloxamine 908®, which is a tetrafunctional block copolymer derived from sequential addition of propylene oxide and ethylene oxide to ethylenediamine (BASF Corporation, Parsippany, N.J.)), polyvinylpyrrolidone K12, polyvinylpyrrolidone K17, polyvinylpyrrolidone K25, or polyvinylpyrrolidone K30, polyvinylpyrrolidone/vinyl acetate copolymer (S-630), polyethylene glycol, e.g., the polyethylene glycol can have a molecular weight of about 300 to about 6000, or about 3350 to about 4000, or about 7000 to about 5400, sodium carboxymethylcellulose, methylcellulose, polysorbate-80, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, polysorbate-80, sodium alginate, polyethoxylated sorbitan monolaurate, polyethoxylated sorbitan monolaurate, povidone, carbomers, polyvinyl alcohol (PVA), alginates, chitosans and combinations thereof. Plasticizers such as cellulose or triethyl cellulose can also be used as dispersing agents. Dispersing agents particularly useful in liposomal dispersions and self-emulsifying dispersions are dimyristoyl phosphatidyl choline, natural phosphatidyl choline from eggs, natural phosphatidyl glycerol from eggs, cholesterol and isopropyl myristate.

Combinations of one or more erosion facilitator with one or more diffusion facilitator can also be used in the present compositions.

The term “diluent” refers to chemical compounds that are used to dilute the compound of interest prior to delivery. Diluents can also be used to stabilize compounds because they can provide a more stable environment. Salts dissolved in buffered solutions (which also can provide pH control or maintenance) are utilized as diluents in the art, including, but not limited to a phosphate buffered saline solution. In certain embodiments, diluents increase bulk of the composition to facilitate compression or create sufficient bulk for homogenous blend for capsule filling. Such compounds include e.g., lactose, starch, mannitol, sorbitol, dextrose, microcrystalline cellulose such as Avicel®; dibasic calcium phosphate, dicalcium phosphate dihydrate; tricalcium phosphate, calcium phosphate; anhydrous lactose, spray-dried lactose; pregelatinized starch, compressible sugar, such as Di-Pac® (Amstar); mannitol, hydroxypropylmethylcellulose, hydroxypropylmethylcellulose acetate stearate, sucrose-based diluents, confectioner's sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin; mannitol, sodium chloride; inositol, bentonite, and the like.

“Filling agents” include compounds such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

“Lubricants” and “glidants” are compounds that prevent, reduce or inhibit adhesion or friction of materials. Exemplary lubricants include, e.g., stearic acid, calcium hydroxide, talc, sodium stearyl fumerate, a hydrocarbon such as mineral oil, or hydrogenated vegetable oil such as hydrogenated soybean oil (Sterotex®), higher fatty acids and their alkali-metal and alkaline earth metal salts, such as aluminum, calcium, magnesium, zinc, stearic acid, sodium stearates, glycerol, talc, waxes, Stearowet®, boric acid, sodium benzoate, sodium acetate, sodium chloride, leucine, a polyethylene glycol (e.g., PEG-4000) or a methoxypolyethylene glycol such as Carbowax™, sodium oleate, sodium benzoate, glyceryl behenate, polyethylene glycol, magnesium or sodium lauryl sulfate, colloidal silica such as Syloid™, Cab-O-Sil®, a starch such as corn starch, silicone oil, a surfactant, and the like.

“Plasticizers” are compounds used to soften the microencapsulation material or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl cellulose and triacetin. In some embodiments, plasticizers can also function as dispersing agents or wetting agents.

“Solubilizers” include compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like.

“Stabilizers” include compounds such as any antioxidation agents, buffers, acids, preservatives and the like.

“Suspending agents” include compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol can have a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

“Surfactants” include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like. Some other surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40. In some embodiments, surfactants can be included to enhance physical stability or for other purposes.

“Viscosity enhancing agents” include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof.

“Wetting agents” include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like.

### Kits/Article of Manufacture

Disclosed herein, in certain embodiments, are kits and articles of manufacture for use with one or more methods described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

The articles of manufacture provided herein contain packaging materials. Examples of pharmaceutical packaging materials include, but are not limited to, blister packs, bottles, tubes, bags, containers, bottles, and any packaging material suitable for a selected formulation and intended mode of administration and treatment.

A kit typically includes labels listing contents and/or instructions for use, and package inserts with instructions for use. A set of instructions will also typically be included.

In some embodiments, a label is on or associated with the container. In one embodiment, a label is on a container when letters, numbers or other characters forming the label are attached, molded or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

### EXAMPLES

These examples are provided for illustrative purposes only and not to limit the scope of the claims provided herein.

### Example 1

### Antigenicity Bioinformatics Workflow for HPV Vaccine Designs

### Generating Consensus Sequences

Sample sequences for E5, E6, and E7 (for HPV16, HPV 17, HPV31, HPV33, and HPV45, serotypes determined to have a higher predisposition for cancer) were downloaded, converted to FASTQ files, and imported into R statistical program. Individual AA sequences were read into R using the biostrings package (readAAStringSet function). Multiple sequence alignments were performed using the ClustalW algorithm in the msa R package. For each subgroup/subtype, consensus sequences were generated and output to PDF/FASTQ files using the msaPrettyPrint function.

### Predicting Binding Affinity

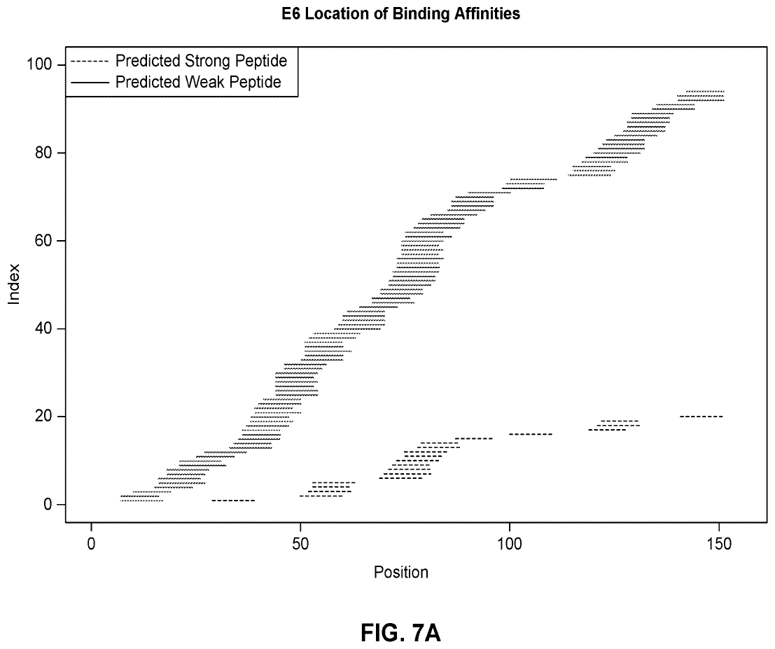

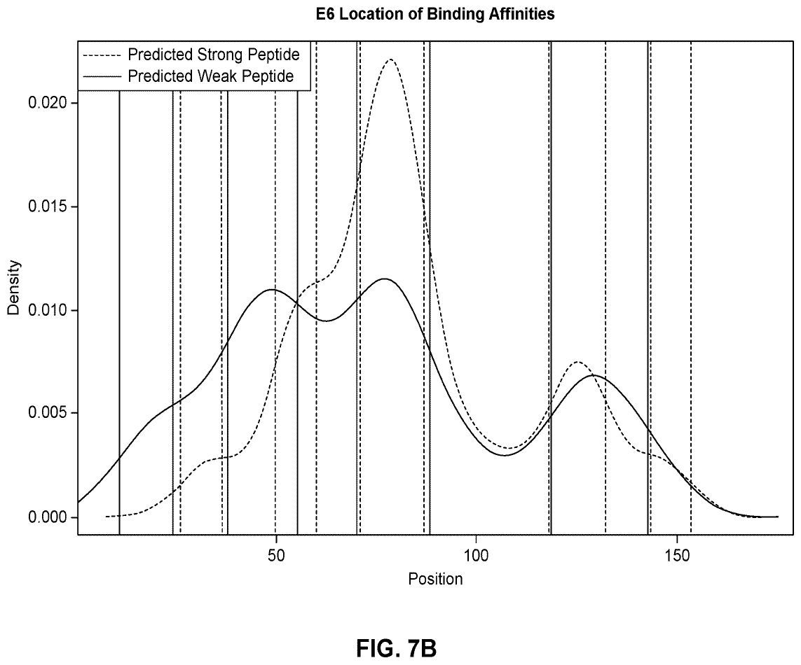

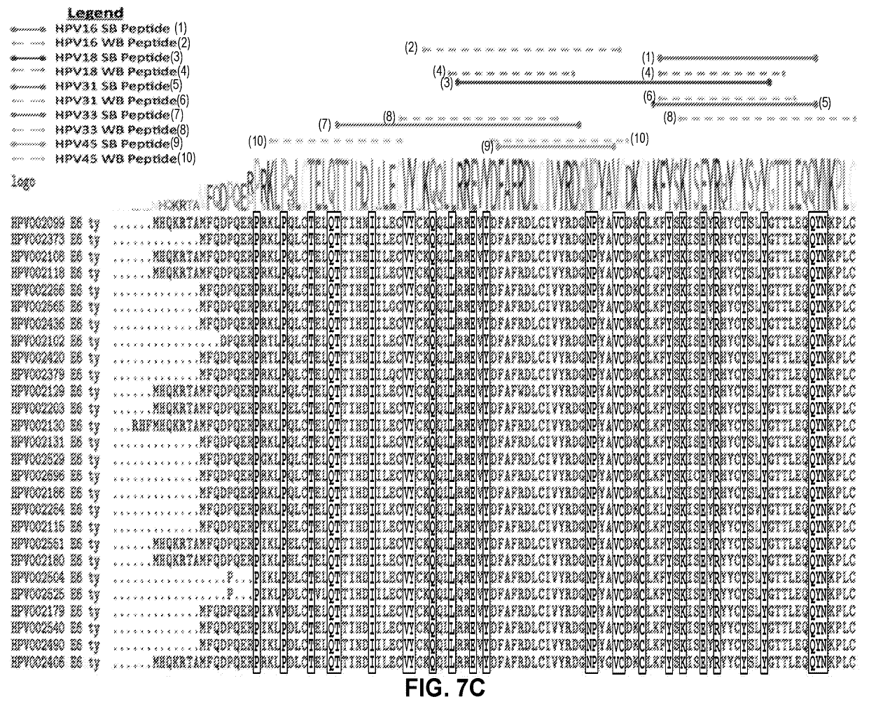

NetMHC 4.0 was applied to each consensus sequence to predict binding affinity against all major MHC-I alleles (HLA-A0101, HLA-A0201, HLA-A0301, HLA-A2402, HLA-A2601, HLA-B0702, HLA-B0801, HLA-B2705, HLA-B3901, HLA-B4001, HLA-B5801, and HLA-B1501). NetMHC 4.0 uses artificial neural networks to predict the binding affinity of peptide sequences. This analysis was performed for HPV16, HPV18, HPV31, HPV33, and HPV45. Thresholds were arbitrarily established at 0.5% (strong binders) and 2% (weak binders) ranks. Peptides with predicted binding affinity greater than 99.5% were classified as strong binders, and peptides with predicted binding affinity greater than 98% were classified as weak binders. The position of each AA within the peptide sequences were extracted and used to generate density curves (FIG. 7A and FIG. 7B). Using these density curves, first and second order differentials were calculated to determine peaks for strong and weak binders (FIG. 7C). Finally, the union of these positions was used to extract AA sequences likely to elicit a response.

### Predicting Binding Affinity in HPV16, HPV18, HPV31, HPV33, and HPV45

Previous studies have shown that HPV strains 16, 18, 31, 33, and 45 were precursors for cervical carcinoma. To identify candidate peptides with broad coverage across these strains, binding affinity was predicted within each strain. The sequences with strong/weak binding affinity predictions were extracted. Using protein blast, these sequences were aligned against the consensus sequences across all five strains. Alignments were plotted for all five serotypes and coverage was assessed.

### Example 2

### HPV Antigen Designs

Naturally existing sequence variations on HPV strains could potentially hinder the development of effective HPV vaccines. To address this concern, the present vaccine design approach utilized bioinformatics and protein engineering methods to select and design antigens with broader coverage of T cell epitopes, novel mutations, and enhancer agonist peptides. Drawing on available information of extended coverage of antigen regions with CTL-specific epitopes and in silico prediction results, the designed HPV vaccine antigens can induce robust HPV-16 and HPV-18 specific responses and potentially benefit patients at high risk of HPV-derived cancers.

The new HPV vaccines include the following engineered proteins, peptides and/or modifications:
1) truncated E5 regions that fused with E6 and E7 domains of HPV16 and HPV18;
2) newly designed point mutations that can potentially abrogate p53 binding with the known PTPN13 binding mutants (Weiking et al., Cancer Gene Ther. 19(10):667-74 (2012). doi: 10.1038/cgt.2012.55. Epub 2012 Aug. 24);
3) a novel truncation in HPV18 E6 non-immunogenic region at the C-terminus and application of p53 binding knockout mutants;
4) a novel truncation of HPV18 E7 non-immunogenic region at the N-terminus and incorporating pRb, Mi2B mutants (Weiking et al., 2012); and/or
5) agonist peptides from Tsang, et al., Vaccine 35(19):2605-2611 (2017) doi: 10.1016/j.vaccine.2017.03.025. Epub 2017 Apr. 4 in the newly designed HPV vaccine constructs.

### HPV Design 1

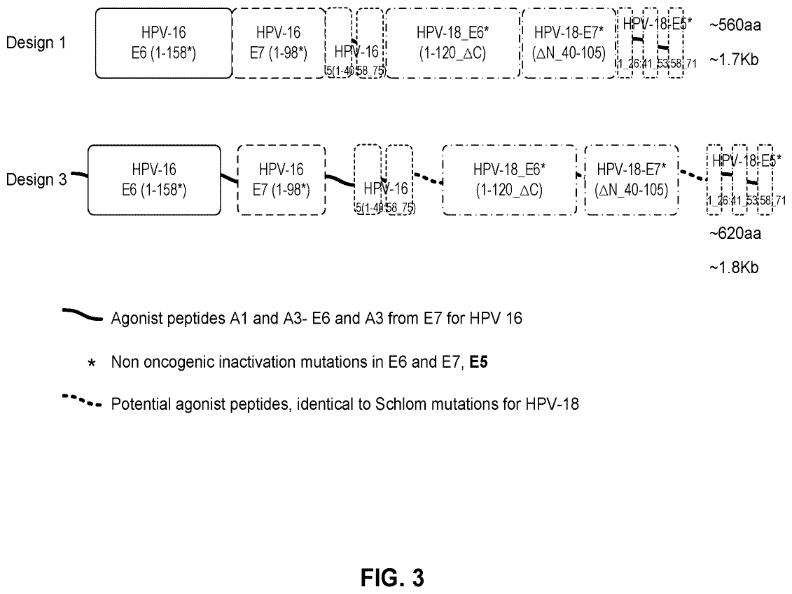

Consensus sequence information was utilized to select HPV 16/18 reference sequences for the design which included all major variants. The vaccine composition comprising different E6, E7 and E5 protein components with domain boundaries and mutation information is shown in FIG. 3. The reference sequence for E6 was obtained from the Human Papillomavirus T cell Antigen Database (Bioinformatics Core at Cancer Vaccine Center, Dana-Farber Cancer Institute). Reference sequences for E5 and E7 were obtained from the Papilloma Virus Episteme (PaVE) database.

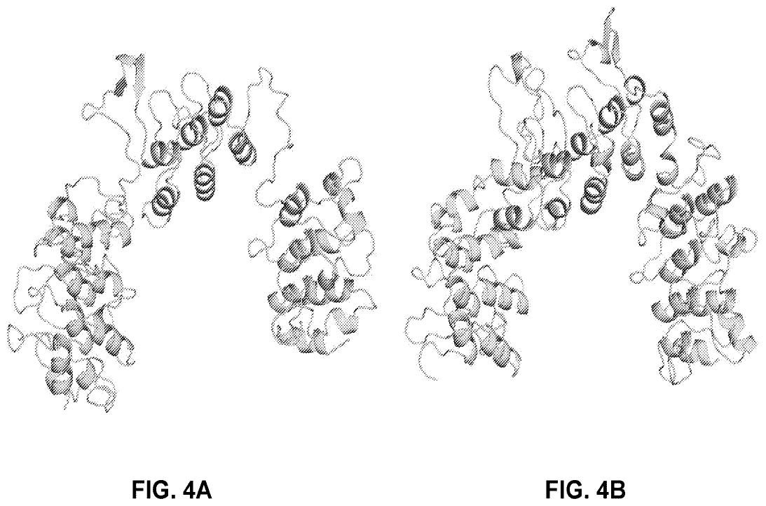

The design for HPV16 and HPV18 E5 were inspired via a combination of in silico predictions (IEDB and netMHC) and, for HPV16 also included findings of Chen et al., J Virol. 78(3):1333-43 (2004); whereas, the design for HPV18 included N terminal residues (1-26) and C terminal residues (41-53, 58-71) (see, FIGS. 4A and 4B).

Inactivating mutations for HPV16 and HPV18 were introduced into E6 and E7 peptide sequences in order prevent oncogenicity (Weiking et al., 2012 October; 19(10):667-74. doi: 10.1038/cgt.2012.55. Epub 2012 Aug. 24). For HPV16 E6, these mutations were E18A, L50G and alanine replacement from 148-151 (ETQL to AAAA). For HPV16 E7, four mutations (H2P, C24G, E46A, and L67R) were included (Weiking et al., 2012). For HPV18 E6, two mutations were included (E18A and L52G). For HPV18 E6, only the N terminus portion was predicted to contain MHC-I binding epitopes (IEBD and netMHC analysis), thus, residues starting at amino acid 121 were removed from these designs. An additional mutation was included in E6 in order to further abrogate its interaction with the p53 protein (Martinez-Zapien et al., Nature 529 (7587): 541-45 (2016)). For HPV18 E7, two mutations (Weiking et al., 2012) were included (E55A and L74R) and residues from (1-40) were removed because HPV-18 E7 contained majority of the predicted MHC-I binding epitopes (IEDB and netMHC predictions) in the C terminal region.

### HPV Design 2

For designing recombinant multi-epitope proteins as HPV vaccine antigens, a total of 32 key immunogenic peptides, as listed in Table 2, were selected. These peptides included CTL specific peptides from E6 (HPV-16/-18), E7 (HPV-16/-18) and E5 (HPV16) genes. Most of these CTL epitopes were reported with multiple immunological assays, immuno-proteomics, and were included in IEDB resource and netMHC predictions. The novelty of this antigen design is to graft the CTL peptides on to a human ankyrin repeat protein scaffold for possible generation of new protein bearing T cell epitopes and or proteasome cleavage sites.

Human Ankyrin-like repeat (“ALR”) protein (PDB code 1QYM) was selected as a scaffold on to which CTL peptides were assembled randomly, enabling different types of protein linker sequences embedded between the peptides. ALR proteins have generally high expression and high stability; the ALR protein provides a scaffold for the HPV peptides and could create novel CTLs. Caution was taken by random shuffling of the peptides to prevent any reformation of E6 and E7 oncogenic proteins.

A homology model (FIG. 4A) was used to assess the overall structural feature and to compare the design against native ankyrin repeats. This model suggested that the grafted CTL peptides on an ankyrin scaffold should retain their tertiary structure. It is expected that the scaffold amino acids connecting those peptides could serve as “linkers” and potentially create new CTL epitopes and or agonist peptide epitopes.

TABLE 2
Peptides from E5 (HPV16), E6 (HPV16 and HPV18), and E7 (HPV16 and HPV18)
along with agonist peptides used to assemble multi-epitope vaccine constructs

| SEQ | HPV16-E6 (9) | SEQ | HPV16-E7 (8) | SEQ | HPV16-E5 (1) |
|---|---|---|---|---|---|
| 113 | HLDKKQRFHNI | 122 | LCVQSTHVDI | 130 | YIIFVYIPL |
| 114 | RWTGRCMSCC | 123 | RTLEDLLMGT | | |
| 115 | TTLEQQYNKPLCDLL | 124 | TLGIVCPI | | |
| 116 | ISEYRHYCY | 125 | LLMGTLGIV | | |
| 117 | VYDFAFRDL | 126 | TLHEYMLDL | | |
| 118 | TIHDIILECV | 127 | AHYNIVTFCC | | |
| 119 | KLPQLCTEL | 128 | YMLDLQPETT | | |
| 120 | FAFRDLCIVY | 129 | CDSTLRLCV | | |
| 121 | LCIVYRDGNPYAVCD | | | | |
| | | | | | Agonist |
| SEQ | HPV18-E6 (8) | SEQ | HPV18-E7 (6) | SEQ | peptides (3) |
| 131 | KLTNTGLYNL | 139 | DDLRAFQQLFLNTLS | 145 | KLPQLCTEV |
| 132 | KCIDFYSRI | 140 | FQQLFLNTL | 146 | QLYNKPLCDV |
| 133 | FAFKDLFVV | 141 | QLFLNTLSFV | 147 | RTLEDLLMGV |
| 134 | NLLIRCLRC | 142 | LFLNTLSFVCPWCAS | | |
| 135 | KLPDLCTEL | 143 | TLQDIVLHL | | |
| 136 | ELTEVFEFA | 144 | SEEENDEIDGVNHQHLPARR | | |
| 137 | SLQDIEITCV | | | | |
| 138 | KTVLELTEV | | | | |

### HPV Design 3

Design 3 is similar to Design 1 with the addition of enhancer agonist peptides (FIG. 3). Overall, designs 1 and 3 contain all necessary sequences from each E5, E6, and E7 (FIGS. 4A and 4B). These were inspired via inventor-selected combinations using guidance from in silico epitope prediction analysis and available information on MHC-I binding and cytokine production following T cell activation.

For HPV16, this design includes: (1) peptides from Tsang et al., Vaccine 35(19):2605-2611 (2017) doi: 10.1016/j.vaccine.2017.03.025. Epub 2017 Apr. 4-three peptides that exhibit better MHC-I binding and elicit more robust cytotoxic T cell lymphocyte (CTL) response comprising mutations in E6 (L19V and Q91L/L99V) and mutation in E7 (T86V); (3) two enhancer peptides fused to the inactivated N and C terminus of E6; and (4) one enhancer peptide fused to the inactivated C terminus of E7.

For HPV18, this design includes: (1) three peptides with identical mutations to mimic enhancer agonist peptides similar to peptides of HPV16 (Tsang et al., 2017) comprising the mutations in E6 (L21V and L101V); and (2) two potential mimic enhancer agonists fused to N and C terminus regions of E6 and another agonist mimic fused to the C terminal region of E7. Since HPV18 naturally had the aforementioned mutations in E6 (Q91L) and E7 (T95V)naturally, no additional modifications were needed.

### HPV Design 4

This design was based on Design 2, utilizing the same ankyrin repeat approach. However, Design 4 includes an additional three unique agonist peptides from the peptides of Tsang et al., 2017 for a total of 35 key immunogenic peptides (Table 2). The order of peptides grafted on the scaffold was again randomized in order to be different from Design 2 and for the potential to generate different CTL epitopes. A homology model of Design 4 was generated (FIG. 4B). The homology of this design was found similar to the Design 2, but indicated local structural variations.

### HPV Design 5

This design is a multi-epitope vaccine, designed by selecting all 35 key immunogenic peptides shown in Table 2. It was assembled with a charged dipeptide KK residue. Advantages of this design include potential for cleavage at “KK” residues and random modification with the “K” residue added at CTL epitopes.

### Example 3

### Assessing the Predicted Binding Affinities of HPV Vaccine Constructs

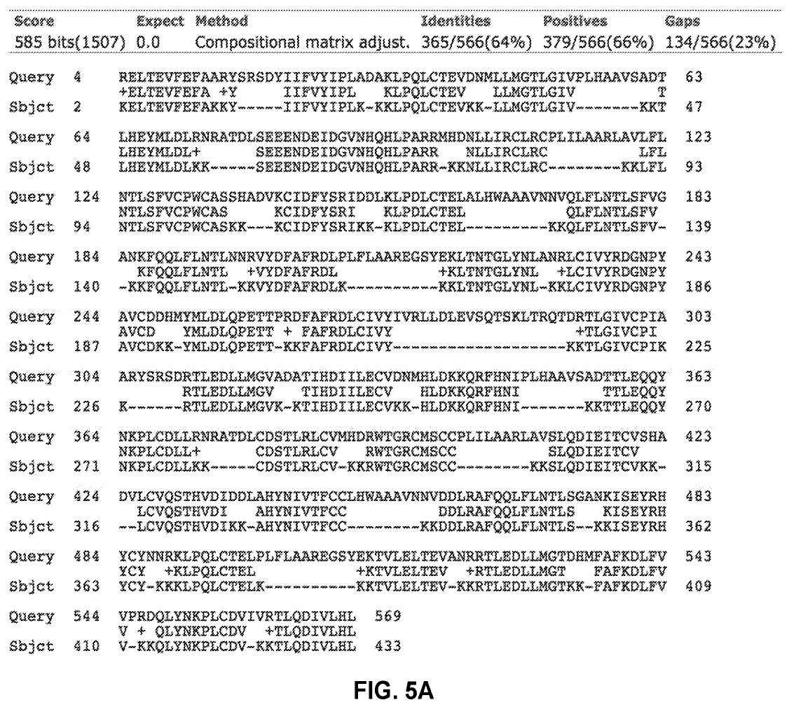

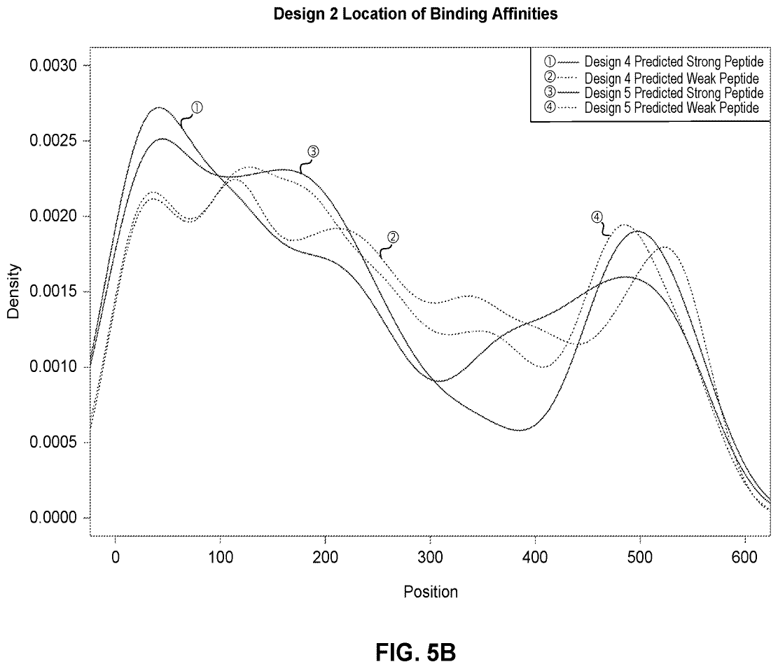

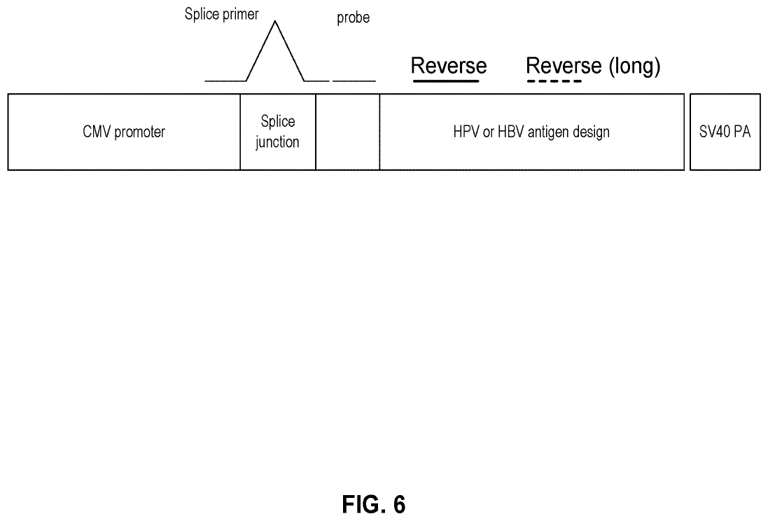

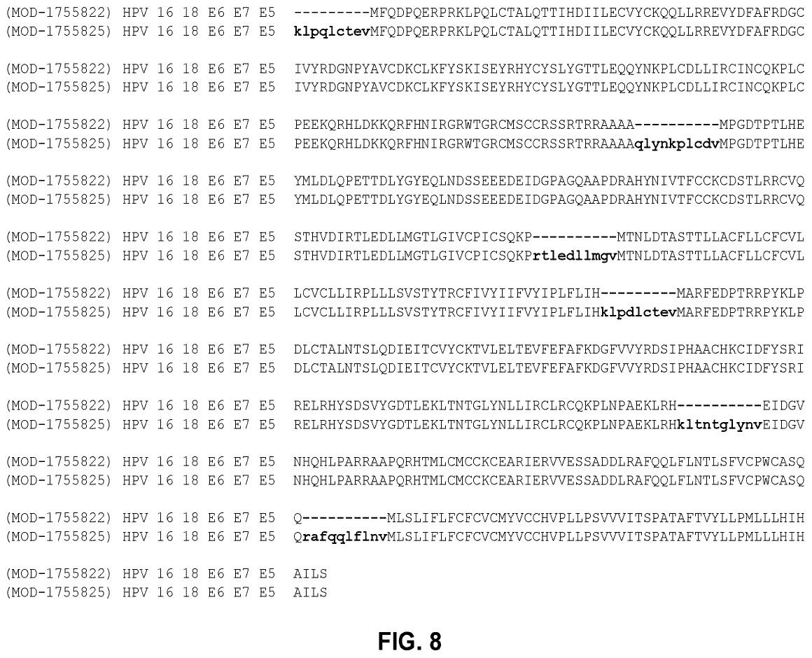

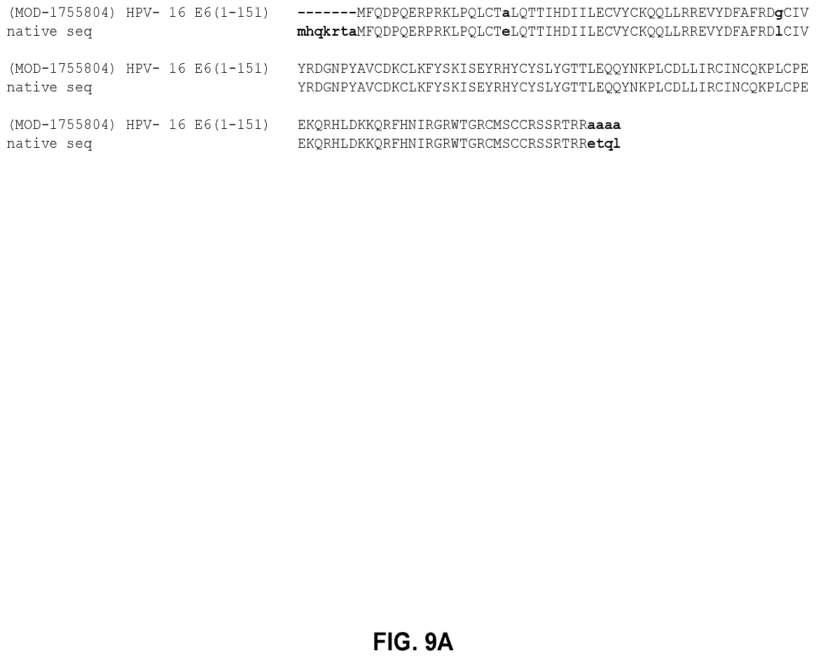

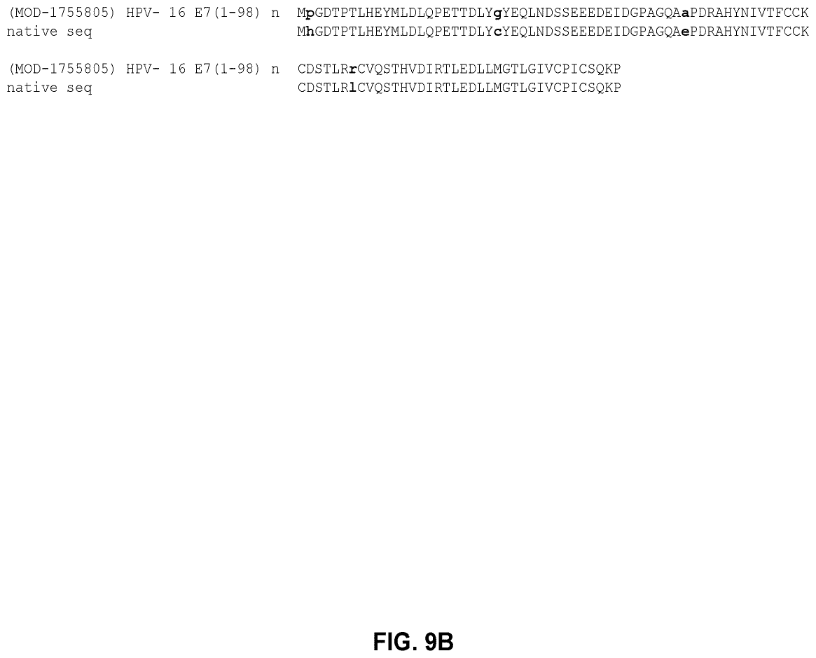

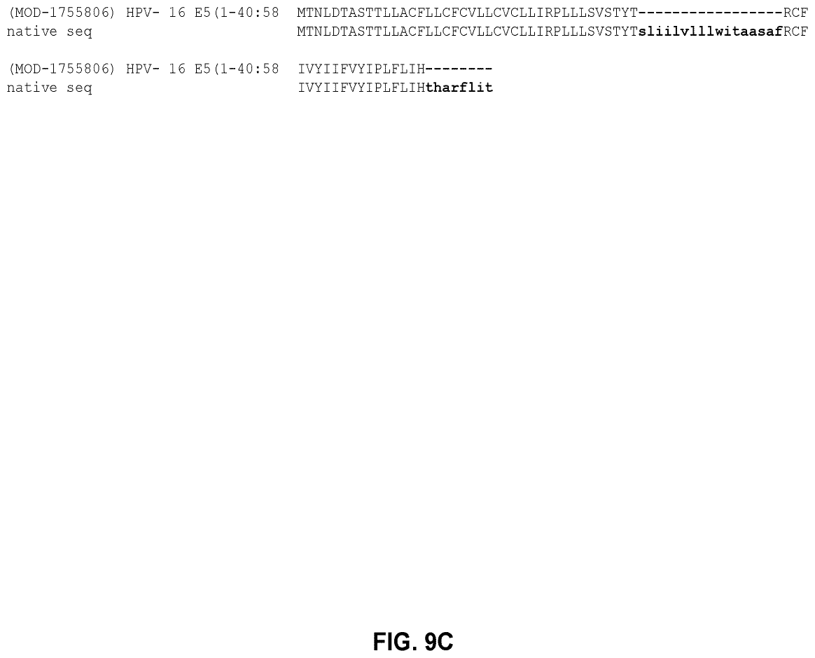

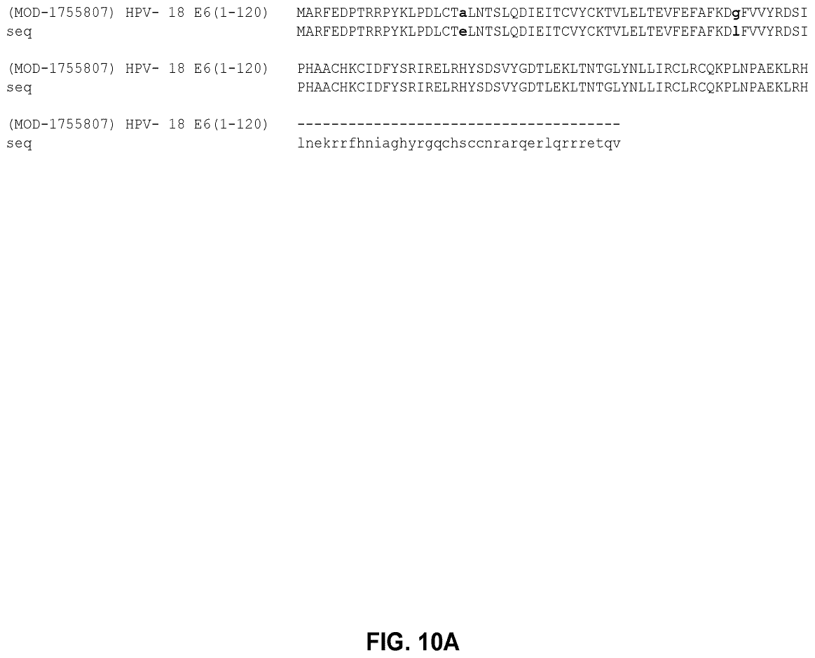

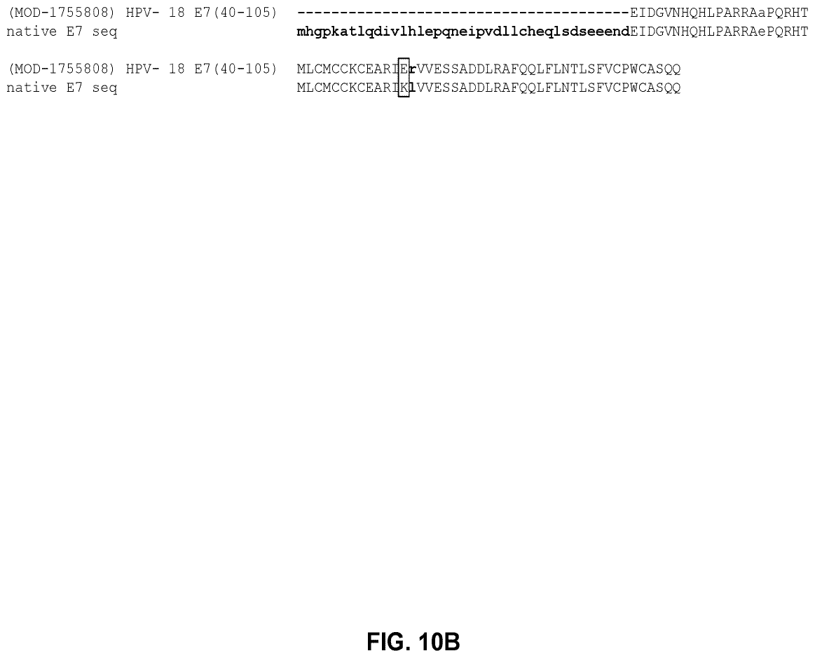

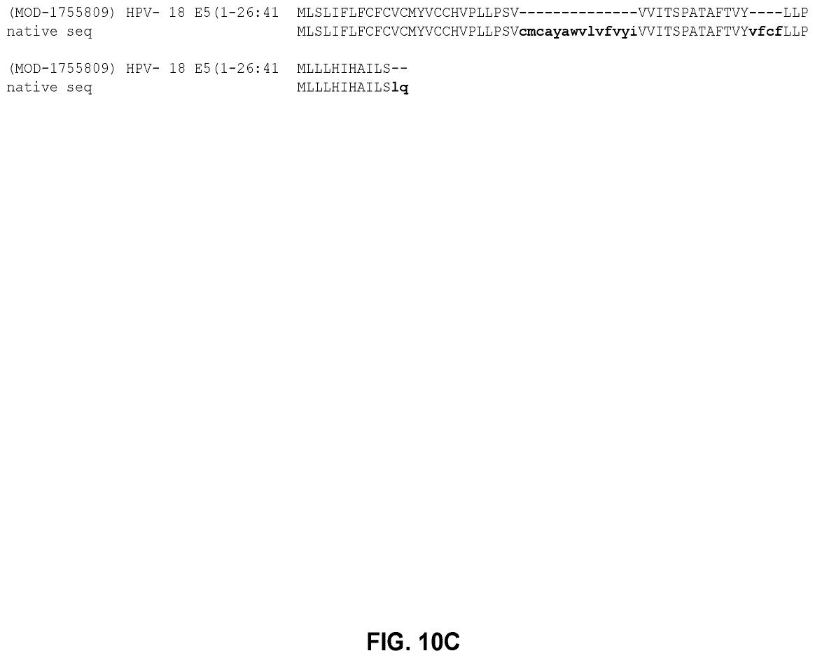

Bioinformatics predictions for the binding affinity for each design were carried out. Entire sequences were loaded into NetMHC, which was used to assess antigenicity and extent of coverage against various HPV genotypes. It should be noted that Designs 4 and 5 use the same 35 peptides. The main difference is that Design 4 peptides were grafted on Ankyrin protein and Design 5 peptides were connected by “KK” linkers (FIG. 5A). Ultimately, the binding affinity predictions on the matched regions of both designs were similar (FIG. 5B). The HPV designs were compared among themselves (Tables 3 and 4) and with reference sequences which are shown in FIG. 8, FIGS. 9A-9C, and FIGS. 10A-10C.

TABLE 3
HPV16 Design Elements (HPV Designs 1 and 3)

| MOD | HPV type | Rationale |
|---|---|---|
| 1755804 | HPV16 E6 non | Inclusion of p53 and PTPN13 binding |
| | oncogenic | site mutations [E18A, L50G, ETQL- |
| | | AAAA]. These mutations do not |
| | | allow E6/E7 to degrade p53, pRb, |
| | | PTPN13, or activate telomerase: |
| | | Martinez-Zapien et al., 2016; Weiking |
| | | et al., 2012 |
| 1755805 | HPV16 E7 non | Removal of Rb and Mi2B binding sites; |
| | oncogenic | prior art in Etubics E7 |
| 1755806 | HPV16 E5 (1-40, | Novel truncation of E5 with removal |
| | 58-75) | of 2 immuno-dominant regions |
| 1755814 | HPV16 E6 A1 | Agonistic peptides |
| | peptide (L19V) | |
| 1755815 | HPV16 E6 A3 | Agonistic peptides |
| | peptide (Q91L/ | |
| | L99V) | |
| 1755816 | HPV16 E7 A3 | Agonistic peptides |
| | peptide (T86V) | |

TABLE 4
HPV18 Design Elements (HPV Designs 1 and 3)

| MOD | HPV type | Notes |
|---|---|---|
| 1755807 | HPV18 E6 non | Novel C terminal truncated E6 domain |
| | oncogenic | (1-120) and inclusion of p53 binding |
| | | sites mutations [E18A, L50G]; |
| | | Martinez-Zapien et al., 2016; Weiking |
| | | et al., 2012 |
| 1755808 | HPV18 E7 non | Novel N terminal truncated E7 domain |
| | oncogenic | (40-105), and inclusion of Rb and Mi2B |
| | | binding mutants [E46A, L67R]; |
| | | Weiking et al., 2012 |
| 1755809 | HPV18 E5 (1-26: | Novel truncation of E5 with removal |
| | 41-53: 58-71) | of 2 immuno-dominant regions |
| 1755817 | HPV18 E6 A1 | Agonistic peptides |
| | peptide (L21V) | |
| 1755818 | HPV18 E6 A3 | Agonistic peptides |
| | peptide (L101V) | |
| 1755819 | HPV18 E7 A3 | Agonistic peptides |
| | peptide (T95V) | |

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

The present application claims benefit to U.S. Provisional Application No. 62/639,354 filed Mar. 6, 2018, which is incorporated herein by reference in its entirety.

### EMBODIMENTS

E1. A non-naturally occurring polynucleotide encoding a polypeptide comprising one or more immune response-inducing human papilloma virus (HPV) polypeptides.

E2. The polynucleotide of E1, wherein said non-naturally occurring polynucleotide encodes a polypeptide comprising two or more HPV polypeptides.

E3. The polynucleotide of E2, wherein said two or more HPV polypeptides comprise one or more HPV-16 immune response-inducing polypeptide sequences.

E4. The polynucleotide of E3, wherein said HPV-16 peptide comprises at least one of an E5 peptide, an E6 peptide or an E7 peptide.

E5. The polynucleotide of E3 or E4, wherein said HPV-16 peptide comprises an E5 peptide, and said E5 peptide has a sequence as shown in SEQ ID NO: 47.

E6. The polynucleotide of any one of E3 to E5, wherein said HPV-16 peptide comprises an E6 peptide, and said E6 peptide has a sequence as shown in SEQ ID NO: 45.

E7. The polynucleotide of any one of E3 to E6, wherein said HPV-16 peptide comprises an E7 peptide, and said E7 peptide has a sequence as shown in SEQ ID NO: 46.

E8. The polynucleotide of any one of E1 to E7, wherein said one or more HPV peptides comprises an HPV-18 peptide.

E9. The polynucleotide of E8, wherein said HPV-18 peptide comprises at least one of an E5 peptide, an E6 peptide or an E7 peptide.

E10. The polynucleotide of E8 or 9, wherein said HPV-18 peptide comprises an E5 peptide, and said E5 peptide has a sequence as shown in SEQ ID NO: 50.

E11. The polynucleotide of any one of E8 to 10, wherein said HPV-18 peptide comprises an E6 peptide, and said E6 peptide has a sequence as shown in SEQ ID NO: 48.

E12. The polynucleotide of any one of E8 to 10, wherein said HPV-18 peptide comprises an E7 peptide, and said E7 peptide has a sequence as shown in SEQ ID NO: 49.

E13. The polynucleotide of any one of E1 to E12, wherein said polypeptide has a sequence as shown in SEQ ID NO: 51.

E14. The polynucleotide of any one of E1 to E13, wherein at least one of said one or more HPV polypeptides is connected to an agonist peptide.

E15. The polynucleotide of E14, wherein said agonist peptide has a sequence comprising an agonist peptide sequence as shown in Table 2.

E16. The polynucleotide of E14 or E15, wherein said polypeptide has a sequence as shown in SEQ ID NO: 53.

E17. A polynucleotide comprising the polynucleotide of any one of E1 to E16, further comprising one or more polynucleotides encoding a gene switch system for inducible control of heterologous gene expression, wherein said heterologous gene expression is regulated by said gene switch system; and, wherein said heterologous gene comprises the polynucleotide of any one of E1 to E16.

E18. The polynucleotide of E17, wherein said gene switch system is an ecdysone receptor-based (EcR-based) gene switch system.

E19. The polynucleotide of any one of E1 to E18, wherein said one or more HPV polypeptides is for use in a vaccine.

E20. A vector comprising the polynucleotide of any one of E1 to E19.

E21. The vector of E20, wherein said vector is an adenoviral vector.

E22. The vector of E21, wherein said adenoviral vector is a gorilla adenoviral vector.

E23. A method of regulating the expression of a heterologous gene in a cell, the method comprising: introducing into said cell one or more polynucleotides that comprise (i) an repressible or inducible gene switch, and (ii) a heterologous immune response-inducing gene, wherein expression of said heterologous immune response-inducing gene is regulated by said gene switch, wherein said heterologous immune response-inducing gene encodes one or more HPV polypeptides; and exposing said cell to a compound in an amount sufficient to repress or induce expression of said heterologous immune response-inducing gene.

E24. The method of E23, wherein said target cell is a mammalian cell.

E25. The method of E23 or E24, wherein said gene switch comprises a ligand binding domain derived from at least one of an ecdysone receptor (EcR), a ubiquitous receptor, an orphan receptor 1, an NER-1, a steroid hormone nuclear receptor 1, a retinoid X receptor interacting protein-15, a liver X receptor β, a steroid hormone receptor like protein, a liver X receptor, a liver X receptor α, a farnesoid X receptor, a receptor interacting protein 14, and a famesol receptor.

E26. An E6 peptide, wherein said E6 peptide comprises an E18A amino acid substitution and at least one of an L50G, E148A, T149A, Q150A and L151A amino acid substitution as compared to a wildtype E6 peptide.

E27. The E6 peptide of E26, wherein said E6 peptide comprises said E18A amino acid substitution and said L50G, E148A, T149A, Q150A and L151A amino acid substitutions.

E28. The E6 peptide of E26, wherein said E6 peptide has a sequence as shown in SEQ ID NO: 45.

E29. The E6 peptide of any one of E26 to E28, wherein said E6 peptide is fused to an agonist peptide.

E30. The E6 peptide of E29, wherein said agonist peptide is fused to at least one of a C-terminus and an N-terminus of said E6 peptide.

E31. The E6 peptide of any one of E26 to E30, wherein said wildtype E6 peptide is from HPV-16.

E32. An E6 peptide, wherein said E6 peptide comprises a deletion as compared to a wildtype E6 peptide, wherein said deletion comprises a C-terminus of said wildtype E6 peptide.

E33. The E6 peptide of E32, wherein said deletion comprises amino acids from amino acid 121 to a C-terminus of said wildtype E6 peptide.

E34. The E6 peptide of E32 or E33, wherein said E6 peptide comprises at least one of an E18A and L50G substitution as compared to said wildtype E6 peptide.

E35. The E6 peptide of any one of E32 to E34, wherein said wildtype E6 peptide is from HPV-18.

E36. The E6 peptide of any one of E32 to E35, wherein said E6 peptide has a sequence as shown in SEQ ID NO: 48.

E37. An E7 peptide, wherein said E7 peptide comprises a deletion as compared to a wildtype E7 peptide, wherein said deletion comprises an N-terminus of said wildtype E7 peptide.

E38. The E7 peptide of E37, wherein said deletion comprises amino acids 1-39 of said wildtype E7 peptide.

E39. The E7 peptide of E37 or E38, wherein said E7 peptide comprises at least one of an E55A and L74R substitution as compared to said wildtype E7 peptide.

E40. The E7 peptide of any one of E37 to E39, wherein said wildtype E7 peptide is from HPV-18.

E41. The E7 peptide of any one of E37 to E40, wherein said E7 peptide has a sequence as shown in SEQ ID NO: 49.

E42. An E5 peptide, wherein said E5 peptide comprises a deletion as compared to a wildtype E5 peptide, wherein said deletion comprises amino acids 41-57 of said wildtype E5 peptide.

E43. The E5 peptide of E42, wherein said E5 peptide has a sequence as shown in SEQ ID NO: 47.

E44. The E5 peptide of E42 or E43, wherein said wildtype E5 peptide is from HPV-16.

E45. An E5 peptide, wherein said E5 peptide comprises a deletion as compared to a wildtype E5 peptide, wherein said deletion comprises at least one of amino acids 27-40 or amino acids 54-57 of said wildtype E5 peptide.

E46. The E5 peptide of E45, wherein said E5 peptide has a sequence as shown in SEQ ID NO: 50.

E47. The E5 peptide of E45 or E46, wherein said wildtype E5 peptide is from HPV-18.

E48. A polypeptide construct comprising the peptide of any one of E26 to E47.

E49. A polypeptide construct, wherein said polypeptide construct comprises an HPV-16 E6 peptide, wherein said HPV-16 E6 peptide comprises an E18A amino acid substitution and at least one of an L50G, E148A, T149A, Q150A and L151A amino acid substitution as compared to a wildtype HPV-16 E6 peptide.

E50. The polypeptide construct of E49, wherein said HPV-16 E6 peptide comprises said E18A amino acid substitution and said L50G, E148A, T149A, Q150A and L151A amino acid substitutions.

E51. The polypeptide construct of E49 or E50, wherein said HPV-16 E6 peptide has a sequence as shown in SEQ ID NO: 45.

E52. The polypeptide construct of any one of E49 to E51, wherein said polypeptide construct further comprises an HPV-16 E7 peptide, wherein said HPV-16 E7 peptide comprises at least one of an H2P, C24G, E46A and L67R amino acid substitution as compared to a wildtype HPV-16 E7 peptide.

E53. The polypeptide construct of E52, wherein said HPV-16 E7 peptide comprises said H2P, C24G, E46A and L67R amino acid substitutions.

E54. The polypeptide construct of E53, wherein said HPV-16 E7 peptide has a sequence as shown in SEQ ID NO: 46.

E55. The polypeptide construct of any one of E49 to E54, wherein said polypeptide construct further comprises an HPV-16 E5 peptide.

E56. The polypeptide construct of E55, wherein said HPV-16 E5 peptide comprises a deletion of one or more amino acids as compared to a wildtype HPV-16 E5 peptide.

E57. The polypeptide construct of E56, wherein said deletion comprises amino acids 41-57 of said wildtype HPV-16 E5 peptide.

E58. The polypeptide construct of any one of E55 to E57, wherein said HPV-16 E5 peptide has a sequence as shown in SEQ ID NO: 47.

E59. The polypeptide construct of any one of E49 to E58, wherein said polypeptide further comprises an HPV-18 E6 peptide.

E60. The polypeptide construct of E59, wherein said HPV-18 E6 peptide comprises an E18A and L50G substitution as compared to a wildtype HPV-18 E6 peptide.

E61. The polypeptide construct of E59 or E60, wherein said HPV-18 E6 peptide comprises a deletion of at least one C-terminus amino acid relative to said wildtype HPV-18 E6 peptide.

E62. The polypeptide construct of E61, wherein said deletion comprises amino acids from amino acid 121 to said C-terminus of said wildtype HPV-18 E6 peptide.

E63. The polypeptide construct of any one of E59 to E62, wherein said HPV-18 E6 peptide has a sequence as shown in SEQ ID NO: 48.

E64. The polypeptide construct of any one of E49 to E63, wherein said polypeptide construct further comprises an HPV-18 E7 peptide.

E65. The polypeptide construct of E64, wherein said HPV-18 E7 peptide comprises an E55A and L74R substitution as compared to a wildtype HPV-18 E7 peptide.

E66. The polypeptide construct of E64 or E65, wherein said HPV-18 E7 peptide comprises a deletion of at least one amino acid from an N-terminus of said HPV-18 E7 peptide.

E67. The polypeptide construct of E66, wherein said deletion comprises amino acids 1-40 of said wildtype HPV-18 E7 peptide.

E68. The polypeptide construct of any one of E64 to E67, wherein said HPV-18 E7 peptide has a sequence as shown in SEQ ID NO: 49.

E69. The polypeptide construct of any one of E59 to E68, wherein said polypeptide construct further comprises an HPV-18 E5 peptide.

E70. The polypeptide construct of E69, wherein said HPV-18 E5 peptide comprises a deletion of at least one amino acid as compared to a wildtype HPV-18 E5 peptide.

E71. The polypeptide construct of E70, wherein said deletion comprises amino acids 27-40 or 54-57 of said wildtype HPV-18 E5 peptide.

E72. The polypeptide construct of any one of E69 to E71, wherein said HPV-18 E5 peptide has a sequence as shown in SEQ ID NO: 50.

E73. The polypeptide construct of any one of E59 to E72, wherein said polypeptide construct has a sequence as shown in SEQ ID NO: 51.

E74. The polypeptide construct of any one of E5973, wherein said polypeptide construct further comprises at least one agonist peptide.

E75. The polypeptide construct of E74, wherein said at least one agonist peptide has a sequence comprising an agonist peptide sequence as shown in Table 2.

E76. The polypeptide construct of E74 or E75, wherein said polypeptide construct has a sequence as shown in SEQ ID NO: 53.

E77. A polypeptide construct comprising an ankyrin-like repeat domain and an HPV peptide.

E78. The polypeptide construct of E77, wherein said ankyrin-like repeat protein is a human ankyrin-like repeat protein.

E79. The polypeptide construct of E77 or E78, wherein said HPV peptide is linked to said ankyrin-like repeat protein by a linker.

E80. The polypeptide construct of any one of E77 to E79, wherein said HPV peptide comprises at least one of an HPV-16 peptide or an HPV-18 peptide.

E81. The polypeptide construct of any one of E77 to E80, wherein said HPV peptide comprises an HPV-16 peptide, and said HPV-16 peptide comprises at least one of an E5 peptide, an E6 peptide or an E7 peptide.

E82. The polypeptide construct of any one of E77 to E81, wherein said HPV peptide comprises an HPV-18 peptide, and said HPV-18 peptide comprises at least one of an E6 peptide or an E7 peptide.

E83. The polypeptide construct of any one of E77 to E82, wherein said HPV peptide comprises an HPV-16 E5 sequence, an HPV-16 E6 sequence, an HPV-16 E7 sequence, an HPV-18 E6 sequence or an HPV-18 E7 sequence as shown in Table 2.

E84. The polypeptide construct of any one of E77 to E83, wherein said polypeptide construct has a sequence as shown in SEQ ID NO: 52.

E85. The polypeptide construct of any one of E77 to E84, wherein said polypeptide construct further comprises at least one agonist peptide.

E86. The polypeptide construct of E85, wherein said polypeptide construct comprises three agonist peptides.

E87. The polypeptide construct of E86, wherein said polypeptide construct has a sequence as shown in SEQ ID NO: 54.

E88. A polypeptide construct, wherein said polypeptide construct comprises at least two HPV amino acid sequences as shown in Table 2, wherein said at least two HPV amino acid sequences are connected by a peptide linker, wherein said peptide linker is a KK linker.

E89. The polypeptide construct of E88, wherein said at least two HPV amino acid sequences comprise at least one of an HPV-16 peptide or an HPV-18 peptide as shown in Table 2.

E90. The polypeptide construct of E88 or E89, wherein said at least two HPV amino acid sequences comprise an HPV-16 peptide, and said HPV-16 peptide comprises at least one of an HPV-16 E5 peptide, an HPV-16 E6 peptide or an HPV-16 E7 peptide as shown in Table 2.

E91. The polypeptide construct of any one of E88 to E90, wherein said at least two HPV amino acid sequences comprise an HPV-18 peptide, and said HPV-18 peptide comprises at least one of an HPV-18 E6 peptide or an HPV-18 E7 peptide as shown in Table 2.

E92. The polypeptide construct of any one of E88 to E91, wherein said at least two HPV amino acid sequences comprise each of the amino acid sequences as shown in Table 2.

E93. The polypeptide construct of E92, wherein said each of the amino acid sequences is connected to another of said each of the amino acid sequences by said KK linker.

E94. The polypeptide construct of any one of E88 to E93, wherein said polypeptide construct has a sequence as shown in SEQ ID NO: 55.

E95. The polypeptide construct of any one of E48 to E94 for use in a vaccine.

E96. A polynucleotide encoding the polypeptide construct of any one of E58 to

E95.

E97. A vector comprising the polynucleotide of E96.

E98. The vector of E97, wherein said vector is an adenoviral vector.

E99. The vector of E98, wherein said adenoviral vector is a gorilla adenoviral vector.

E100. A vector, wherein said vector comprises a polynucleotide that encodes at least one HPV peptide, wherein said vector is an adenoviral vector.

E101. A vector, wherein said vector comprises a polynucleotide that encodes at least one HPV peptide, wherein said vector is an adenoviral vector, wherein said adenoviral vector is a gorilla adenoviral vector.

Provided herein is a representative reference list of certain sequences included in embodiments provided herein (Table 5).

TABLE 5
Polynucleotide/Amino Acid Sequences

| SEQ | |
|---|---|
| ID NO: | Description |
| 1 | Adenovirus pIX fragment nucleotides |
| 2 | Adenovirus DNA polymerase fragment nucleotides |
| 3 | Adenovirus penton base protein fragment nucleotides |
| 4 | Adenovirus hexon protein fragment nucleotides |
| 5 | Adenovirus fiber protein fragment nucleotides |
| 6 | Adenovirus pIX nucleotides |
| 7 | Adenovirus DNA polymerase nucleotides |
| 8 | Adenovirus penton base protein nucleotides |
| 9 | Adenovirus hexon protein nucleotides |
| 10 | Adenovirus fiber protein nucleotides |
| 11 | Adenovirus pIX protein fragment |
| 12 | Adenovirus DNA polymerase fragment |
| 13 | Adenovirus penton base protein fragment |
| 14 | Adenovirus hexon protein fragment |
| 15 | Adenovirus fiber protein fragment |
| 16 | Adenovirus pIX amino acids |
| 17 | Adenovirus DNA polymerase amino acids |
| 18 | Adenovirus penton base protein |
| 19 | Adenovirus hexon protein |
| 20 | Adenovirus fiber protein |
| 21 | Adenovirus vector nucleotide sequences |
| 22 | Adenovirus vector nucleotide sequences |
| 23 | Adenovirus vector nucleotide sequences |
| 24 | Adenovirus vector nucleotide sequences |
| 25 | Adenovirus vector nucleotide sequences |
| 26 | IL-2 core promoter |
| 27 | IL-2 minimal promoter |
| 28 | IL-2 enhancer and promoter variant |
| 29 | L-2 enhancer and promoter variant |
| 30 | (NF-κB)₁-IL2 promoter variant |
| 31 | (NF-κB)₃-IL2 promoter variant |
| 32 | (NF-κB)₆-IL2 promoter variant |
| 33 | 1X NFAT response elements-IL2 promoter variant |
| 34 | 3X NFAT response elements-IL2 promoter variant |
| 35 | 3X NFAT response elements-IL2 promoter variant |
| 36 | 6X NFAT response elements-IL2 promoter variant |
| 37 | 6X NFAT response elements-IL2 promoter variant |
| 38 | 6X NFAT response elements-IL2 promoter variant |
| 39 | 6X NFAT response elements-IL2 promoter variant |
| 40 | human EEF1A1 promoter variant |
| 41 | human EEF1A1 promoter variant |
| 42 | human EEF1A1 promoter and enhancer |
| 43 | human UBC promoter |
| 44 | synthetic minimal promoter 1 |
| 45 | HPV antigen design 1 HPV16 E6 amino acids |
| 46 | HPV antigen design 1 HPV16 E7 amino acids |
| 47 | HPV antigen design 1 HPV16 E5 amino acids |
| 48 | HPV antigen design 1 HPV18 E6 amino acids |
| 49 | HPV antigen design 1 HPV18 E7 amino acids |
| 50 | HPV antigen design 1 HPV18 E5 amino acids |
| 51 | HPV antigen design 1 amino acids |
| 52 | HPV antigen design 2 amino acids |
| 53 | HPV antigen design 3 amino acids |
| 54 | HPV antigen design 4 amino acids |
| 55 | HPV antigen design 5 amino acids |
| 56 | HPV antigen design 1 full nucleotide sequences |
| | of gorilla adenovirus shuttle plasmid |
| 57 | HPV antigen design 2 full nucleotide sequences |
| | of gorilla adenovirus shuttle plasmid |
| 58 | HPV antigen design 3 full nucleotide sequences |
| | of gorilla adenovirus shuttle plasmid |
| 59 | HPV antigen design 4 full nucleotide sequences |
| | of gorilla adenovirus shuttle plasmid |
| 60 | HPV antigen design 5 full nucleotide sequences |
| | of gorilla adenovirus shuttle plasmid |
| 61 | GCAd-RTS-IL12 design 1 |
| 62 | GCAd-RTS-IL12 design 2 |
| 63 | GCAd-RTS-IL12 design 3 |

1-31. (canceled)

32. A polypeptide construct comprising an ankyrin-like repeat domain and an HPV peptide.

33. The polypeptide construct of claim 32, wherein the ankyrin-like repeat protein is a human ankyrin-like repeat protein.

34. (canceled)

35. The polypeptide construct of claim 32, wherein the HPV peptide is an HPV-16 peptide or an HPV-18 peptide.

36. The polypeptide construct of claim 32, wherein the HPV peptide is:
an HPV-16 E5 peptide;
an HPV-16 E6 peptide;
an HPV-16 E7 peptide;
an HPV-18 E6 peptide; or
an HPV-18 E7 peptide.

37. The polypeptide construct of claim 32, wherein the HPV peptide is:
an HPV-16 E5 peptide of SEQ ID NO: 130;
an HPV-16 E6 peptide of any one of SEQ ID NOs: 113-121;
an HPV-16 E7 peptide of any one of SEQ ID NOs: 122-129;
an HPV-18 E6 peptide of any one of SEQ ID NOs: 131-138; or
an HPV-18 E7 peptide of any one of SEQ ID NOs: 139-144.

38. The polypeptide construct of claim 32, comprising the amino acid sequence of SEQ ID NO: 52.

39. The polypeptide construct of claim 32, further comprising at least one agonist peptide.

40. The polypeptide construct of claim 39, comprising the amino acid sequence of SEQ ID NO: 54.

41-45. (canceled)

46. A polynucleotide encoding the polypeptide construct of claim 32.

47. A vector comprising the polynucleotide of claim 46.

48. The vector of claim 47, wherein the vector is an adenoviral vector.

49. The vector of claim 48, wherein the adenoviral vector is a gorilla adenoviral vector.

50. (canceled)

51. The polypeptide construct of claim 32, wherein the ankyrin-like repeat protein is a human ankyrin-like repeat protein.

52. The polypeptide construct of claim 32, comprising the amino acid sequences of each of SEQ ID NOs: 113-144.

53. The vector of claim 49, wherein the gorilla adenoviral vector does not comprise an E1 region.

54. The vector of claim 49, wherein the gorilla adenoviral vector does not comprise an E4 region.

55. The vector of claim 49, wherein the gorilla adenoviral vector does not comprise an E1 region or an E4 region.

56. The vector of claim 49, wherein the gorilla adenoviral vector is a GC46 vector.

57. The vector of claim 49, wherein the polynucleotide is under the control of an immediate early cytomegalovirus (CMV) promoter.

58. A method of vaccinating a subject against human papilloma virus, the method comprising administering to the subject the vector of claim 49.
